(19) United States
(12) Patent Application Publication
(10) Pub. No.: US 2019/0119734 A1
(43) Pub. Date: Apr. 25, 2019

(54) SENSORS FOR NUCLEIC ACID BIOMARKERS

(71) Applicant: Memorial Sloan Kettering Cancer Center, New york, NY (US)

(72) Inventors: Jackson HARVEY, New York, NY (US); Daniel HELLER, New York, NY (US); Prakrit JENA, New York, NY (US)

(73) Assignee: Memorial Sloan Kettering Cancer Center, New York, NY (US)

(21) Appl. No.: 16/091,894

(22) PCT Filed: Apr. 7, 2017
(86) PCT No.: PCT/US17/26592

§ 371 Date: Oct. 5, 2018

Related U.S. Application Data
(60) Provisional application No. 62/320,126, filed on Apr. 8, 2016.

(51) Int. Cl. C12Q 1/6834

(52) U.S. Cl. CPC C12Q 1/6834; B82Y 15/00

(57) ABSTRACT

Described herein are novel devices and methods for the optical detection of oligonucleotide binding events for diagnostic, point-of-care, drug screening, and theranostic applications, for example, a robust and customizable system to detect specific DNA and RNA oligonucleotides using a carbon nanotube optical signal.

### CROSS REFERENCE TO RELATED APPLICATIONS

This application claims the benefit of U.S. Application Ser. No. 62/320,126 filed on Apr. 8, 2016, the disclosure of which is hereby incorporated by reference in its entirety. Applicant also notes it is concurrently filing a potentially related patent application entitled, “SWCNT-DNA-ANTIBODY CONJUGATES, RELATED COMPOSITIONS, AND SYSTEMS, METHODS AND DEVICES FOR THEIR USE”, which claims the benefit of U.S. Application Ser. No. 62/334,412 filed on May 10, 2016.

### GOVERNMENT FUNDING

This invention was made with government support under grant numbers HD075698 and CA008748 awarded by National Institutes of Health. The government has certain rights in this invention.

### FIELD OF INVENTION

This invention relates generally to the detection of nucleotide sequences or other biological materials. In particular embodiments, the invention relates to the combination of single-walled carbon nanotubes and DNA for the optical detection of microRNA.

### SEQUENCE LISTING

The present specification makes reference to a Sequence Listing (submitted electronically as a .txt file named “2003080-1324_SL.txt” on Apr. 7, 2017). The .txt file was generated on Mar. 29, 2017 and is 29,854 bytes in size. The entire contents of the Sequence Listing are hereby incorporated by reference.

### BACKGROUND

Detection of free oligonucleotides in body fluids holds great promise as diagnostic and prognostic markers for a variety of pathologies, including cancer, metabolic disease, organ rejection, fetal health, and infectious disease. The relative accessibility of body fluids containing these oligonucleotides has fueled progress in creating “liquid biopsies” to circumvent problems inherent to traditional, invasive biopsies. Potential oligonucleotides used for liquid biopsies include cell-free tumor DNA, mRNA, and circulating microRNA (miRNA). Somewhat surprisingly, miRNA was found to differ from other RNA types in that it is stable in body fluids, despite the presence of endogenous RNases. Encouragingly, many studies to date have identified specific expression patterns of miRNA in body fluids, including in serum and urine that are indicative of disease states. The promise of using miRNA in serum or urine for minimally invasive, early detection of a variety of diseases, either alone or in conjunction with other established biomarkers, is exciting because the early detection of cancer is associated with the best prognosis.

Because miRNA detection has tremendous potential in diagnostics and prognostics, great effort has been put forth in creating novel and reliable detection schemes. The detection of miRNA is complicated by their short length, approximately 22 nucleotides, as well as by a dynamic range that can span several orders of magnitude. Additionally, relative amounts of miRNA purified from biofluids can change depending on the protocol used. The current gold standard for miRNA detection and quantification is RT-qPCR using stem loop primers, which is based on time-consuming amplification of miRNA from purified samples. Innovative assays that avoid amplification, labeling, and purification from biofluids are needed for point-of-care diagnostics. Ideally, an implantable miRNA sensor could report changes in miRNA concentration in real-time to continuously monitor the health status of a patient.

The current standard for miRNA measurement, with limits of detection ranging from attomolar (aM) to (fM), is quantitative PCR (qPCR). However, this method requires purification and amplification of miRNA that can introduce biases and variability. Commercially available techniques that do not involve amplification, such as microarrays, suffer from poorer sensitivity (picomolar (pM) to nanomolar (nM)) and high false positive rates. Detection strategies that avoid amplification, labeling, and purification from biofluids are under investigation, but in vivo detection strategies are sparse. The detection of nucleic acid biomarkers in real-time and in situ within living tissues and organisms remains an important challenge.

Nanotechnology-based solutions for miRNA detection represent a promising strategy for amplification-free and label-free detection of miRNA. In particular, individually-dispersed semiconducting single-walled carbon nanotubes (SWCNTs) exhibit ideal qualities as optical biomedical sensors. SWCNTs are fluorescent in the near-infrared, a wavelength range penetrant to tissue, raising the possibility of implantable sensors. Additionally, SWCNTs do not photobleach due to their excitonic nature of fluorescence. The emission wavelength and intensity is exquisitely sensitive to the immediate SWCNT environment, allowing changes at the surface to be transduced in an optical signal. Sensitivity to some analytes has been measured at the single-molecule level. It has been shown that single-strand DNA has an affinity for the nanotube surface and can be used as a dispersant to prepare optically active, single nanotube dispersions. Additionally, DNA-DNA hybridization between nanotube-associated DNA and free single-strand DNA in solution can mediate a solvatochromic shift in the nanotube emission.

The use of SWCNTs as optical sensors is complicated by the inability to use covalent chemistry for functionalization, as too many sp3 defects along the nanotube sidewall will quench their optical properties. Thus, non-covalent functionalization schemes are required for their application as biosensors. Using such strategies, sensors have been developed for Beta-D-glucose, DNA hybridization, divalent metal cations, assorted genotoxins, nitroaromatics, nitric oxide, pH, and the protein avidin. More recently, specific recognition of target analytes using changes in the corona phase of an adsorbed polymer has been developed. A major challenge in developing non-covalent, colloidally stable sensors for use in biological systems is imparting appropriate specificity for the target analyte while resisting non-specific interactions with other biological material.

Therefore, there remains a need for accurate and sensitive biosensing platforms.

### SUMMARY OF INVENTION

Described herein are devices and methods for the optical detection of oligonucleotide binding events for diagnostic, point-of-care, drug screening, and theranostic applications, for example, a robust and customizable system to detect specific DNA and RNA oligonucleotides, using a carbon nanotube optical signal. This optically based detection scheme is useful, e.g., for detecting circulating oligonucleotides that have diagnostic and prognostic value for cancer, metabolic disease, organ rejection, fetal health, and infectious disease. Potential targets include cell-free tumor DNA, circulating mRNA, and circulating microRNA (miRNA). Because this platform is compatible with biofluids, the platform provides, in various embodiments, purification-free, point-of-care diagnostics. Further described are implants comprising the sensing platform in live organisms (e.g., humans, rodents etc.), and methods to detect oligonucleotides in vivo with a noninvasive method. Thus, this platform can be used as an implantable sensor for biomarkers, allowing for real-time, non-invasive monitoring in vivo. Primarily, the devices are, or comprise, a sensor comprising a single-walled carbon nanotube (SWCNT) and a polymer associated with the SWCNT, wherein the polymer comprises a first domain and a second domain, e.g., wherein the first domain has a sequence complementary to a target nucleotide sequence and wherein the second domain is a stabilizing domain.

Moreover, described herein are engineered carbon-nanotube-based sensors capable of real-time optical quantification of hybridization events of microRNA and other oligonucleotides. The mechanism of the sensors arise from competitive effects between displacement of both oligonucleotide charge groups and water from the nanotube surface, which result in a solvatochromism-like response. The sensors, which allow for detection via single-molecule sensor elements and for multiplexing by using multiple nanotube chiralities, can monitor toehold-based strand-displacement events, which reverse the sensor response and regenerate the sensor complex. It is also shown that the sensors function in whole urine and serum, and can non-invasively measure DNA and microRNA after implantation in live mice.

In certain embodiments, a distinguishing features is that the polymer on the nanotube includes both a nanotube-binding domain and a target domain that hybridizes with a target/analyte. The target domain can be complementary to a target that is DNA, miRNA, lncRNA, mRNA, and the like. In various embodiments, the sensor can be used to detect DNA, miRNA, mRNA, lnRNA, and the like, of any length, e.g., fewer than 30 nucleotides, or 30 nucleotides or longer.

In one aspect, the invention is directed to a single-walled carbon nanotube (SWCNT) sensor, comprising: a SWCNT; a polymer associated with the SWCNT (e.g., conjugated non-covalently or covalently to the SWCNT (e.g., directly or via a linker) (e.g., wrapped around the SWCNT), or otherwise associated with the SWCNT), (e.g., wherein the polymer comprises DNA, LNA, PNA, an amino-acid sequence, or a synthetic monomer), wherein the polymer comprises two or more domains ((e.g., wherein the sensor is capable of detecting species in a sample, e.g., the species having a target nucleotide sequence (e.g., microRNA) (e.g., wherein the target nucleotide sequence has fewer than 30 nucleotides, e.g., wherein the target nucleotide sequence has 30 or more nucleotides)).

In certain embodiments, the two or more domains comprise: a first domain comprising a stabilizing domain; and a second domain (e.g., or additional domains) comprising a sequence complementary to a target nucleotide sequence. In certain embodiments, the two or more domains comprise: a third domain that has a sequence complementary to a target sequence (e.g., wherein the first domain and the third domain are positioned on each end of the stability domain).

In certain embodiments, the linker comprises nucleic acid-based, hydrocarbon-based, or polymer-based (e.g., comprises polyethylene glycol (PEG)).

In certain embodiments, the polymer is single-stranded DNA. In certain embodiments, the polymer comprises a single-stranded DNA binding component containing a sequence complementary to a target nucleotide sequence.

In certain embodiments, the target nucleotide sequence has fewer than 30 nucleotides. In certain embodiments, the target nucleotide sequence has 30 or more nucleotides. In certain embodiments, the target nucleotide sequence has from about 5 nucleotides to about 30 nucleotides. In certain embodiments, the target nucleotide sequence has from about 10 nucleotides to about 25 nucleotides.

In certain embodiments, the first domain has a sequence complementary to the target nucleotide sequence. In certain embodiments, the first domain has a sequence complementary to a target miRNA sequence (or a truncated sequence of the target miRNA sequence). In certain embodiments, the target miRNA is a mammalian miRNA member selected from the group consisting of the miRNAs listed in Table 12.

In certain embodiments, the first domain has a sequence complementary to a target DNA sequence (or a truncated sequence of the target DNA sequence or to a complementary region in a longer strand with non-complementary regions). In certain embodiments, the second domain is a stabilizing domain (e.g., wherein the stabilization domain provides adequate nanotube dispersion). In certain embodiments, stabilizing means prevents/reduces agglomeration of SWCNTs and/or promotes stability of a suspension of the SWCNTs.

In certain embodiments, the second domain is an oligonucleotide sequence (e.g., a short oligonucleotide sequence) (e.g., a single-strand DNA that forms water soluble complexes with SWCNT).

In certain embodiments, the oligonucleotide sequence comprises a member selected from the group consisting of (GT)6 (SEQ ID NO: 2), (GT)15 (SEQ ID NO: 1), (AT)15 (SEQ ID NO: 3), (TAT)6 (SEQ ID NO: 4), (TCC)10 (SEQ ID NO: 5), (TGA)10 (SEQ ID NO: 6), (CCA)10 (SEQ ID NO: 7), (TTA)4TT (SEQ ID NO: 8), (TTA)3TTGTT (SEQ ID NO: 9), (TTA)5TT (SEQ ID NO: 10), (TAT)4 (SEQ ID NO: 11), (CGT)3C (SEQ ID NO: 12), (ATT)4 (SEQ ID NO: 13), (ATT)4AT (SEQ ID NO: 14), (TATT)2TAT (SEQ ID NO: 15), (ATTT)3 (SEQ ID NO: 16), (GTC)2GT (SEQ ID NO: 17), (CCG)4 (SEQ ID NO: 18), (GTT)3G (SEQ ID NO: 19), (TGT)4T (SEQ ID NO: 20), (TATT)3T (SEQ ID NO: 22), (TCG)10 (SEQ ID NO: 23), (GTC)3 (SEQ ID NO: 24), (TCG)2TC (SEQ ID NO: 25), (TCG)4TC (SEQ ID NO: 26), (GTC)2 (SEQ ID NO: 27), (TGTT)2TGT (SEQ ID NO: 28), (TTTA)3T (SEQ ID NO: 29), (CCG)2CC (SEQ ID NO: 30), (TCG)4TC (SEQ ID NO: 31), T3C6T3 (SEQ ID NO: 32), (GTC)2GT (SEQ ID NO: 33), CTTC2TTC (SEQ ID NO: 34), TTA(TAT)2ATT (SEQ ID NO: 35), TCT(CTC)2TCT (SEQ ID NO: 36), (ATT)4 (SEQ ID NO: 37), GC11 (SEQ ID NO: 38), (TC)3CTCCCT (SEQ ID NO: 39), CTTC3TTC (SEQ ID NO: 40), (GT)20 (SEQ ID NO: 41), CTC3TC (SEQ ID NO: 42), (TCT)2 (SEQ ID NO: 43), C5TC6 (SEQ ID NO: 44), T4C4T4 (SEQ ID NO: 45), and C5TTC5 (SEQ ID NO: 46).

In certain embodiments, the polymer comprises three or more domains. In certain embodiments, the domains have sequences complementary to one or more target nucleotide sequences. In certain embodiments, the domains have sequences complementary to one or more target miRNA sequences.

In certain embodiments, the sensor further comprises a surfactant. In certain embodiments, the sensor further comprises a surfactant, wherein the surfactant is selected from a group consisting of SDS, SDBS, SDC, SPAN-80, Brij 52, BSA, Triton X-100, Pluronic, Pyrene-PEG, TPGS, IGEPAL, and Phospholipid-PEG-NH₂. In certain embodiments, the sensor further comprises SDBS.

In another aspect, the invention is directed to a method for detecting a target using a single-walled carbon nanotube (SWCNT) sensor, the method comprising: contacting a sample comprising a species having a target nucleotide sequence with the SWCNT sensor; exposing the sample to excitation electromagnetic radiation (excitation EMR) to produce an emission of electromagnetic radiation (emission EMR) by the SWCNT sensor; detecting the electromagnetic radiation emitted by the SWCNT sensor; and identifying the presence of the species having the target nucleotide sequence (e.g., a polynucleotide, oligonucleotide, radionucleotide, DNA, RNA, long non-coding RNA; microRNA, circulating microRNA, messenger RNA (mRNA), cell-free tumor DNA, or a fragment, an analogue, or a compound thereof) in the sample based at least in part on the detected emission EMR.

In certain embodiments, the method comprises detecting a wavelength shift (e.g., a blueshift or a redshift) in the emission EMR and/or an intensity shift (e.g., amplitude shift) or other changes in the spectral characteristics of the emission EMR or non-emission EMR changes, thereby identifying the presence of the species having the target nucleotide sequence in the test sample.

In certain embodiments, the other changes in the spectral characteristics of the emission EMR include ratiometric intensity changes (e.g., relative changes of one nanotube chirality intensity versus another), changes in full-width half-max (e.g., a measure of the “thickness” of the spectral peak), changes in exiciton energy transfer (a unique spectral signature from energy exchange between nanotubes in close-contact), and combinations thereof.

In certain embodiments, the non-emission EMR changes include changes in light absorbance (such as bleaching), blueshift or redshift in the excitation EMR, changes in dynamic light scattering (sample bundling), visible flocculation (aggregation) of nanotubes in sample, and combinations thereof.

In certain embodiments, the method comprises detecting an intensity shift between an emission center wavelength (e.g., a peak) of the sample and an emission center wavelength (e.g., a peak) of a reference sample, wherein the reference sample is devoid of the species having the target nucleotide sequence.

In certain embodiments, the method comprises contacting the sample comprising multiple species having target nucleotide sequences with multiple SWCNT sensors, wherein the SWCNTs have different chiralities.

In certain embodiments, the excitation EMR has a wavelength between 100 nm and 3000 nm, 200 nm and 2000 nm, between 300 and 1500 nm, or between 500 and 1000 nm. In certain embodiments, the emission EMR has a wavelength between 300 nm and 3000 nm, between 400 and 2000 nm, between 500 and 1500 nm, between 600 nm and 1400 nm, or between 700 and 1350 nm. In certain embodiments, the emission wavelength shift is between 1 nm and 100 nm, between 2 nm and 100 nm, between 3 and 50 nm, or between 4 and 20 nm.

In certain embodiments, the wavelength shift is a blue shift.

In certain embodiments, the species having the target nucleotide sequence is microRNA.

In certain embodiments, the method comprises identifying a molecule or organism having, or associated with, the target nucleotide sequences. In certain embodiments, the molecule or organism comprises a member selected from the group consisting of a peptide, a polypeptide, a protein, a biologic, a biomolecule, a biosimilar, an aptamer, a virus, a bacterium, a toxin, a cell, an antibody, and a fragment thereof.

In certain embodiments, the sample is a biological sample (e.g., in vitro, ex vivo, or in vivo, e.g., wherein the biological sample is a subject). In certain embodiments, the sample is a member selected from the group consisting of a cell culture sample, a laboratory sample, a tissue sample (e.g., muscle tissue, nervous tissue, connective tissue, and epithelial tissue), and a bodily fluid sample (e.g., Amniotic fluid, Aqueous humour and vitreous humour, Bile, Blood serum, Breast milk, Cerebrospinal fluid, Cerumen (earwax), Chyle, Chyme, Endolymph and perilymph, Exudates, Feces, Female ejaculate, Gastric acid, Gastric juice, Lymph, Menstrual fluid, Mucus (including nasal drainage and phlegm), Pericardial fluid, Peritoneal fluid, Pleural fluid, Pus, Rheum, Saliva, Sebum (skin oil), Serous fluid, Semen, Smegma, Sputum, Synovial fluid, Sweat, Tears, Urine, Vaginal secretion, Vomit., etc.).

In certain embodiments, the SWCNT sensor is the sensor.

In another aspect, the invention is directed to a kit for use in a laboratory setting, the kit comprising: at least one container (e.g., an ampule, a vial, a cartridge, a reservoir, a lyo-j ect, or a pre-filled syringe); and a single-walled carbon nanotube (SWCNT) sensor as described herein.

In another aspect, the invention is directed to a system for the detection of microRNA, comprising a single-walled carbon nanotube (SWCNT) sensor, a source of electromagnetic radiation, and an electromagnetic radiation detector.

In another aspect, the invention is directed to an implantable detection device comprising the SWCNT sensor. In certain embodiments, the device is a point-of-care medical device (e.g., a urine dipstick, a test strip, a membrane, a skin patch, a skin probe, a gastric band, a stent, a catheter, a needle, a contact lens, a prosthetic, a denture, a vaginal ring, or other implant). In certain embodiments, the device is a device for monitoring environmental conditions. In certain embodiments, the device comprises a microfluidic chamber containing a surface-immobilized SWCNT sensor, or an SWCNT sensor contained in a semi-permeable enclosure.

In another aspect, the invention is directed to a dynamic DNA nanotechnology device comprising a single-walled carbon nanotube (SWCNT) sensor. In certain embodiments, the device is a circuit, a catalytic amplifier, an autonomous molecular motor, or a reconfigurable nanostructure.

Elements of the embodiments involving one aspect of the invention (e.g., methods) can be applied in embodiments involving other aspects of the invention (e.g., devices), and vice versa.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

“Affinity”: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

“Amphipathic” or “Amphiphilic”: The terms “amphipathic” and “amphiphilic” are interchangeably used herein, and each termrefers to a molecule containing both a hydrophilic (and/or charged) domain and a hydrophobic domain.

“Analog”: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

“Aptamer”: As used herein, the term “aptamer” refers to a macromolecule composed of nucleic acid (e.g., RNA, DNA) that binds tightly to a specific molecular target (e.g., an umbrella topology glycan). A particular aptamer may be described by a linear nucleotide sequence and is typically about 15-60 nucleotides in length. Without wishing to be bound by any theory, it is contemplated that the chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers typically have very high affinities for their targets (e.g., affinities in the picomolar to low nanomolar range for proteins). In many embodiments, aptamers are chemically stable and can be boiled or frozen without loss of activity. Because they are synthetic molecules, aptamers are amenable to a variety of modifications, which can optimize their function for particular applications. For example, aptamers can be modified to dramatically reduce their sensitivity to degradation by enzymes in the blood for use in in vivo applications. In addition, aptamers can be modified to alter their biodistribution or plasma residence time.

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated moieties are covalently linked to one another. In some embodiments, associated entities are non-covalently linked. In some embodiments, associated entities are linked to one another by specific non-covalent interactions (e.g., by interactions between interacting ligands that discriminate between their interaction partner and other entities present in the context of use, such as, for example, streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, electrostatic interactions, hydrogen bonding, affinity, metal coordination, physical adsorption, host-guest interactions, hydrophobic interactions, pi stacking interactions, van der Waals interactions, magnetic interactions, electrostatic interactions, dipole-dipole interactions, etc.

“Nucleic acid”: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

“Polypeptide”: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

“Prevent or prevention”: As used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

“Protein”: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

“Sample”: As used herein, the term “sample” typically refers to a biological sample obtained or derived from a source of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

“Substantially”: As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Subject”: As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Drawings are presented herein for illustration purposes, not for limitation.

### BRIEF DESCRIPTION OF DRAWINGS

FIG. 1 shows results of a screen of a certain number of nucleotide sequences for stability and resistance to non-specific oligonucleotide interactions.

FIG. 2A is a schematic for the construction of the SWCNT sensing platform, according to an illustrative embodiment of the invention. “(GT)15” disclosed as SEQ ID NO: 1.

FIG. 2B shows wavelength shifts from miR-19 and R23 sequences.

FIG. 2C shows atomic force microscopy (AFM) images of a sensor complex upon incubation with non-complementary (R23HP) or complementary (miR-19HP) hairpin DNA.

FIG. 2D shows fluorescence restoration in the -presence of 1 μM miR-19 DNA vs. 1 μM R23.

FIG. 2E is a graphical representation of snapshot images of molecular dynamics simulations of the GT15mir19 sensor (Unhyb) and GT15mir19 sensor hybridized with miR-19 (Hyb) after equilibrating for 250 ns, according to an illustrative embodiment of the invention. Teal color denotes the (GT)₁₅ (SEQ ID NO: 1), nanotube-binding sequence, and orange denotes the miR-19 capture sequence. The purple strand denotes miR-19.

FIG. 2F shows a spectra from 730 nm excitation with and without target miR-19 in buffer only conditions in the top panel, and in the presence of 0.2% SDBS in the bottom panel.

FIG. 2G shows wavelength shifts from miR-19 and R23 (+presence of surfactant).

FIG. 2H is a graphic representation of the assembly of supramolecular complexes of SDBS, triggered by the detection of target RNA or DNA, according to an illustrative embodiment of the invention.

FIG. 2I shows density of water as a function of radial distance from the nanotube, calculated for both simulations.

FIG. 2J shows density of phosphate groups as a function of radial distance from the nanotube, calculated for the final frame of both simulations.

FIG. 3 shows complete photoluminescence spectroscopy (PL) plots for DNA-based and RNA-based target miR-19/miR-23 sequences.

FIG. 4 shows intensity changes for selected chirality indexes following binding to DNA miR-19 and RNA miR-19.

FIG. 5 shows shifts in the excitation wavelength against shifts in emission wavelengths following binding to DNA miR-19 and RNA miR-19.

FIG. 6 shows AFM derived height maps (bottom) for hairpin decorated miR-19 (SEQ ID NO: 111) and R-23 (SEQ ID NO: 110) sequences (top).

FIG. 7 shows blueshifts for three different chiralities of GT6mir19.

FIG. 8 is a graphic representation of restoration of Cy5 fluorescence upon binding of miR-19, according to an illustrative embodiment of the invention.

FIG. 9 shows blueshifts for three different chiralities of GT6mir19-Cy5.

FIG. 10 shows representative PL plots—miR-19 RNA vs. Buffer.

FIG. 11 shows an impact of surfactant—Fold change of intensity for miR-19 and R-23 sequences.

FIG. 12 shows a correlation between the excitation wavelength shift and the emission wavelength shift for the ensemble of chiralities.

FIGS. 13A and 13B show an emission energy change (FIG. 13A) and intensity shift (FIG. 13B) in relation to nanotube diameter for mod 2 nanotube.

FIG. 14 show characteristics of emission of GT15mir19+SDBS, with 1:2 serial dilution from 4% to 0.004% SDBS (128 mM to 0.0625 mM).

FIG. 15 shows an impact of presence of 1 μM of random sequences on wavelength for three measured chiralities.

FIGS. 16A-16L show characterization data of the described sensors comprising SDBS.

FIG. 16A shows blueshift behavior.

FIG. 16B shows an effect of concentration.

FIG. 16C shows a wavelength shift for (7,5) nanotube.

FIG. 16D shows blueshift rates for different chiralities.

FIG. 16E shows truncated target sequences ranging from 10 to 15 nucleotides that can bind either from the 3′ end or the 5′ portion in the middle of the recognition sequence (SEQ ID NOS 47, 55, 47 and 112, respectively, in order of appearance).

FIG. 16F shows wavelength shift for truncated target sequences.

FIG. 16G shows modelled orientation upon binding and resulting spectral shift of two sequences, R23-mir19 and mir19-R23 that have R23 at either the 3′ end or 5′ end.

FIG. 16H shows wavelength shift for R23-mir19 and mir19-R23 that have R23 at either the 3′ end or 5′ end.

FIG. 16I shows spectral responses (blue-shifts), of the sensor composed of the specified capture sequences, to related miR-200 family sequences (shown in the table below the graph of FIG. 16I; differences shown in red). miR-141: TAACACTGTCTGGTAAAGATGG (SEQ ID NO: 89); miR-200b: TAATACTGCCTGGTAATGATGA (SEQ ID NO.: 90); miR-429: TAATACTGTCTGGTAAAACCGT (SEQ ID NO: 91). Sequences disclosed as SEQ ID NOS 89-91, respectively, in order of appearance.

FIG. 16J shows emission response of the sensor to a series of truncated sequences (length specified in the x axis) designed to hybridize to either the middle or 3′ end of the capture sequence.

FIG. 16K is a cartoon illustrating a modified analyte sequence and expected configuration upon binding to the GT15mir19 sensor, according to an illustrative embodiment of the invention.

FIG. 16L shows spectral response of the (8,6) nanotube species upon introduction of long analyte sequences to the sensor.

FIG. 17 shows dose-response curves to determine the limit and range of detection with various concentrations of nanotube.

FIG. 18 is a graphic representation of blueshift kinetics, according to an illustrative embodiment of the invention.

FIG. 19 shows PL plots after addition of miR-19 DNA or miR-19 RNA to measure eleven different chiralities.

FIG. 20 is a graphic representation of rate of blueshifting with miR-19 DNA across the measured chiralities, according to an embodiment of the invention.

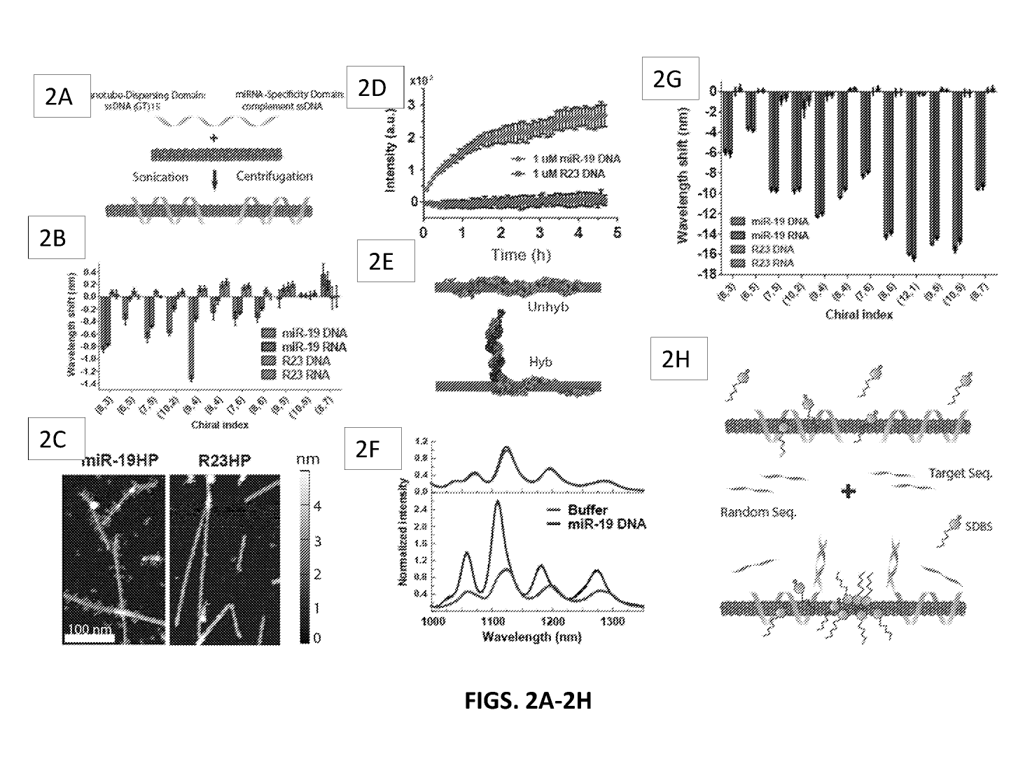

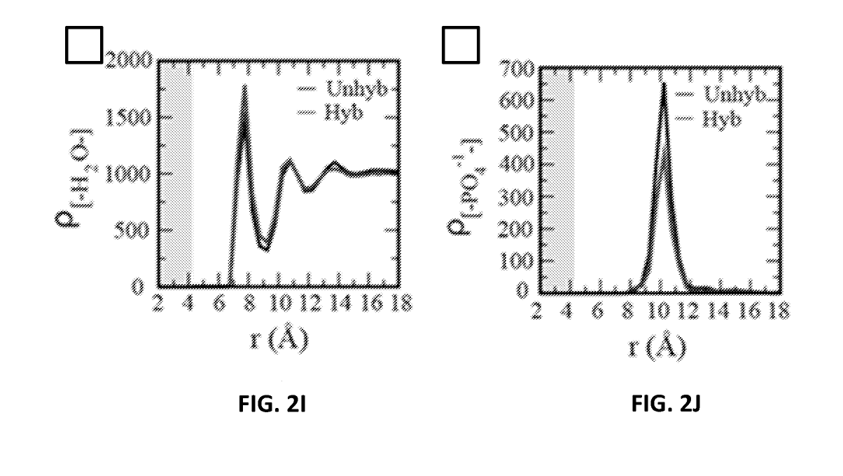

FIG. 2I is graphic representations of rate of blueshifting for 8 different miR for chirality (8,6), according to an embodiment of the invention.

FIG. 22 is graphic representations of rate of blueshifting for 8 different miR for chirality (9,4), according to an embodiment of the invention.

FIG. 23 shows a redshift of R23-mir19-R23 compared with R23R23R23 and R23.

FIG. 24A shows an image of nanotubes absorbed on lysine coated plate.

FIG. 24B show wavelength shifts of spectra 50 min after addition of miR-19 RNA or R23.

FIG. 24C show single nanotube spectra before and after addition of miR-19 RNA,

FIG. 24D show single nanotube spectra before and after addition of R23 to surface-absorbed GT15mir19 nanotubes.

FIGS. 25A and 25B show single nanotube spectra before and after addition of miR-19 RNA (FIG. 25A) or R23 (FIG. 25B) to surface-absorbed GT15mir19 nanotubes.

FIG. 26A shows a PL plot for a HiPCO preparation (NanoC) that is almost totally devoid of (6,5) suspended with GT15mir19.

FIG. 26B shows a PL plot for a CoMoCAT preparation of nanotubes, which is mostly the (6,5) and almost no (8,6) suspended with GT15mir509.

FIG. 26C shows wavelength shifts for both sensors employed separately or together.

FIG. 26D is a graphical representation of components of GT15cReporter-sensor detection system, according to an illustrative embodiment of the invention.

FIG. 26E shows wavelength shifts of components of GT15cReporter-sensor detection system.

FIG. 27 shows normalized absorbance for a preparation that is almost totally devoid of (6,5) suspended with GT15mir19, but with (8,6) present, and a preparation of nanotubes, which is mostly the (6,5) and almost no (8,6), suspended with GT15mir509.

FIG. 28 show characterization of GT15cReporter-sensor detecting a reporter strand of DNA released from a structure-switching aptamer.

FIGS. 29A and 29B show wavelength shift (FIG. 29A) for GT15mir19 with 6 bases removed from the end of the complementary binding region, compared with miR-19 DNA and RNA and R23 DNA and RNA (FIG. 29B) intensity fold shift for GT15mir19 with 6 bases removed from the end of the complementary binding region, compared with miR-19 DNA and RNA and R23 DNA and RNA.

FIGS. 29C and 29D depict a time course taken after adding miR-19 DNA, measuring wavelength shifts and intensity (arrow: addition of the removing strand to the solution).

FIG. 29E depicts a model of toehold mediated strand displacement on the nanotube, beginning with the hybridized DNA containing a 6 nucleotide overhang

FIG. 30 shows center wavelengths for GT15mir19 tested in a solution of 10% fetal bovine serum (FBS) or buffer for three chiralities.

FIGS. 31A-31D show wavelength shifts (FIG. 31A) and changes of intensity (FIG. 31B) for GT15mir19 in various concentrations in urine; wavelength shifts (FIG. 31C) and changes of intensity (FIG. 31D) for GT15mir19 in various concentrations in 10% fetal bovine serum (FBS).

FIG. 31E shows SDBS-pretreated GT15mir19 nanotubes loaded into an implantable semipermeable membrane with a molecular weight cut off (500 kDa).

FIG. 31F shows a nanotube implant inserted into the peritoneum medially over mouse intestines.

FIG. 31G shows a graphical representation of excitation of the nanotubes with 730 nm light and collection of the nanotube emission, according to an illustrative embodiment of the invention.

FIG. 31H shows center wavelengths for two control groups and target microRNA group.

FIG. 31I shows center wavelengths for two control groups and target microRNA group (repeat experiment).

FIG. 32 shows characterization of implantable sensor subjected to dialysis against buffer for 6 hours with three buffer changes.

FIG. 33 shows GT15mir19 blueshifts upon specific recognition.

FIG. 34 shows a representative model of SDBS enhancement, according to an illustrative embodiment of the invention. SDBS associates with a nanotube as described herein, and gives a partial SDBS/DNA wrapped character. As hybridization occurs, the ratio of SDBS to DNA covered nanotube surface changes and increases SDBS.

FIG. 35 shows that binding of miRNA target to the nanotube displays a blueshift.

FIG. 36 shows that some miRNA targets in combination with some nanotubes produce an intensity increase.

FIG. 37 shows wavelength shifts of 6 different nanotube chiralities upon addition of DNA sequence analogues of miR-19 with truncated lengths. R23 is the full length, random sequence control, and mir19 is the complete sequence length. The suffix after mir19 indicates the length of the truncated strand.

FIG. 38 shows kinetic traces of wavelength shifts and intensity changes of 3 different nanotube chiralities upon addition of DNA sequence analogues of miR-19 with truncated lengths. R23 is the full length, random sequence control, and mir19 is the complete sequence length. The suffix after mir19 indicates the length of the truncated strand.

FIG. 39 shows intensity fold change for various target miRNAs using various nanotube chiralities.

FIG. 40 shows blueshift for various chiralities as a function of delta G (kcal/mole).

FIG. 41 shows wavelength shifts for truncated DNA analogues for the (8,6) nanotube, and a PL plot depicting wavelength shifts for 12 chiralities of nanotubes as a function of target DNA length.

FIGS. 42A-42F each show a kinetic response of the GT15mirX sensor to three closely-related sequences. The response of the (9,4) chirality is shown.

FIGS. 42A-42B each show a wavelength shift and intensity change of the sensor specific for miR-141 (GT15mir141).

FIGS. 42C and 42D each show wavelength shift and intensity change over time for sensor specific for miR-200b (GT15mir200b).

FIGS. 42E and 42F each show wavelength shift and intensity fold change over time for sensor specific for miR-429 (GT15mir429). Error bars represent standard error of the mean for n=3 technical replicates.

FIG. 43A shows wavelength shifts of 6 different chiralities upon addition of long strands of DNA with a short portion of complementary in the middle.

FIG. 43B depicts wavelength shifting for complementary DNA strands with non-complementary DNA on either the 5′ or 3′ end, which results in a blue or red shift

FIGS. 44A-44C show atomic force microscopy of the GT15mir19 complex under aqueous conditions.

FIG. 44A shows the complementary binding partner after incubation with miR-19HP (SEQ ID NO: 113).

FIG. 44B shows a non-complementary control after incubation with R23HP (SEQ ID NO: 21).

FIG. 44C shows after incubation with buffer only.

FIGS. 45A-45F show structural parameters of the GT15mir19 sensor complex computed for the (9,4) nanotube via molecular dynamics simulations.

FIGS. 45A and 45B show distribution of radial distance and (FIG. 45B) stacking angle relative the nanotube for nucleobases from the GT15 (SEQ ID NO: 1) nanotube binding domain and miR-19 miRNA capture sequence domain when hybridized to target miR-19.

FIGS. 45C and 45D show distribution of radial distance and (FIG. 45D) stacking angle relative to the nanotube for nucleobases from the hybridized target miR-19 when hybridized with the miRNA capture sequence.

FIG. 45E shows distributions of radial distance from the nanotube of the miR-19 miRNA capture sequence when target miR-19 is not hybridized. “GT15” disclosed as SEQ ID NO: 1.

FIG. 45F shows stacking angle of miR-19 miRNA capture sequence when target miR-19 is not hybridized. “GT15” disclosed as SEQ ID NO: 1.

FIGS. 46A-46B show starting configurations of molecular dynamics simulations involving the duplex miRNA capture sequence+miR-19 without the GT₁₅ (SEQ ID NO: 1) nanotube binding domain.

FIG. 46A shows miRNA capture sequence/miR-19 duplex initially configured parallel to the axial vector of the nanotube.

FIG. 46B shows miRNA capture sequence/miR-19 duplex initially configured perpendicular to the axial vector of the nanotube.

FIG. 47 shows two calculations of hybridization free energy of DNA on the nanotube surface. Graphics are illustrative examples of the reference states and G_{binding} values are taken the work by Jung et al. Case A depicts the scenario where single stranded DNA on a nanotube hybridizes with complementary DNA in solution. Case B depicts the scenario were both strands are first adsorbed to the nanotube surface.

FIGS. 48A and 48B show (FIG. 48A) mean peak wavelength and (FIG. 48B) intensity values of the GT15mir19 complex after incubation with amphipathic molecules. Data is shown for the (7,5) nanotube species. Error bars represent standard deviation from three technical replicates.

FIGS. 49A and 49B show change of the GT15mir19 sensor response to miRNA upon interrogation with a panel of amphiphilic molecules. (FIG. 49A) Wavelength shift from buffer control and (FIG. 49B) intensity fold enhancement over buffer control are shown following incubation with the target oligonucleotide or non-complementary control after 4 hours.

FIG. 50 shows emission wavelength response of GT15mirX sensors to their complementary miR biomarker sequence or R23 non-complementary control (DNA). The responses of four nanotube chiralities are shown. Error bars represent standard error of the mean for n=3 technical replicates.

FIGS. 51A-51F each show a kinetic response of the GT15mirX sensor to three closely-related sequences. The response of the (8,6) chirality is shown.

FIGS. 51A and 51B each show wavelength shift and intensity fold change over time for sensor specific for miR-141 (GT15mir141).

FIGS. 51C and 51D each show wavelength shift and intensity fold change over time for sensor specific for miR-200b (GT15mir200b).

FIGS. 51E and 51F each show wavelength shift and intensity fold change over time for sensor specific for miR-429 (GT15mir429). Error bars represent standard error of the mean for n=3 technical replicates.

FIGS. 52A and 52B show GT15mirX sensor response rates vs. guanine content of the miRNA capture sequences.

FIG. 52A shows a response of the (9,4) nanotube chirality.

FIG. 52B shows a response of the (8,6) nanotube chirality. Pearson correlation coefficients are indicated.

FIG. 53 shows GT15mirX sensor response rates vs. thymine, adenosine, and cytosine content of the miRNA capture sequence, or free energy of hybridization of the miRNA capture sequence. Response of the (9,4) chirality was measured. No statistically significant correlations were found.

FIG. 54 shows GT15mirX sensor response rates vs. thymine, adenosine, and cytosine content of the miRNA capture sequence, or free energy of hybridization of the miRNA capture sequence. Response of the (8,6) chirality was measured. No statistically significant correlations were found.

FIGS. 55A-55B show sensor response in urine from healthy donors.

FIG. 55A shows wavelength shift as a function of miR-19 RNA or non-complementary control R23 concentration for each individual donor.

FIG. 55B shows intensity fold enhancement as a function of added miR-19 RNA or non-complementary control R23 concentration. Error bars represent standard deviation of three technical replicates.

FIGS. 56A-56B show data of the sensor response in serum.

FIG. 56A shows wavelength shift of the GT15mir19 sensor in whole serum with 0.2% SDBS and upon addition of proteinase K. The response of the (8,6) nanotube is shown.

FIG. 56B shows intensity change in the same conditions. Error bars represent standard deviation of three technical replicates.

FIG. 57 shows intensity response of the GT15mir21 sensor after introducing the miR-21 RNA oligonucleotide in serum with proteinase K. Error bars represent standard deviation of three technical replicates.

FIGS. 58A-58B show persistence of wavelength shifting of the GT15mir19 sensor upon dialysis of SDBS.

FIG. 58A shows emission wavelength response of the sensor, interrogated after the indicated dialysis time. Buffer changes are indicated by the arrows.

FIG. 58B shows average emission wavelength the sensor in response to miR-19 DNA and buffer control at all timepoints.

FIG. 59 shows response of the implanted sensor device to 1 nanomole of miR-19 RNA within live mice. The (8,6) nanotube chirality was measured; 3-4 spectra per animal were taken; 3 animals were measured per group (p<0.0001, Dunnet's multiple comparison test, ordinary one-way ANOVA).

FIG. 60 shows a dose-response curve of the GT15mir19 sensor capillary device measured in vitro.

FIG. 61 shows emission from the implantable devices removed from one animal in each group after injection of buffer, 500 pmol miR-19 RNA, or 500 pmol R23 RNA. Error bars represent standard error of the mean for 3-4 measurements.

FIGS. 62A-62H show detection of miRNA in biofluids and non-invasively within live mice.

FIG. 62A shows response of the GT15mir19 sensor emission wavelength to miR-19 spiked into urine from 5 healthy donors. The (7,6) nanotube chirality was measured. Error bars represent standard deviation of technical triplicates.

FIG. 62B shows intensity response of the sensor in urine. Error bars represent standard deviation of technical triplicates.

FIG. 62C shows wavelength response of the nanotube sensor complex to miR-21 and miR-19 miRNA in fetal bovine serum (FBS). Error bars represent standard deviation of technical triplicates.

FIG. 62D shows semi-permeable membrane encapsulating the GT15mir19 sensor for implantation.

FIG. 62E is a diagram of NIR probe apparatus for illuminating and measuring the sensor response in vivo, according to an illustrative embodiment of the invention.

FIG. 62F shows an image of a NIR probe system measuring the nanotube response within a live mouse.

FIG. 62G shows a response of the implanted sensor device to miR-19 DNA within the live mouse (3-4 measurements per mouse; 3 mice per group). The (9,4) nanotube species was measured (p<0.0001, Dunnet's multiple comparison test, ordinary one-way ANOVA).

FIG. 62H shows a response of the implanted sensor device to 500, 100, and 50 pmol of miR-19 RNA or R23 RNA injected into mice intraperitoneally (3-4 measurements per animal; 3 animals per group), shown for the (8,7) nanotube species. 50 pmol R23 was slightly red-shifted compared to buffer control. Error bars represent standard deviation. Statistical significance was calculated with Dunnet's multiple comparison test. Ordinary ANOVA was used to compare the mean of each group to the mean of the buffer control. Sidak's multiple comparison test with an alpha of 0.05 was used to compare miR-19 groups.

FIGS. 63A-63C show that use of a nanotube sensor with two or more binding domains (e.g., first and second binding domains) leads to a greater magnitude of shift, but not an increase in sensitivity, compared to a nanotube sensor with one binding domain. GT15mir19 comprises two domains: a stability and a binding domain; and mir19GT15mir19 comprises three domains: two binding domains and a stability domain. Data is shown for 5 nanotube chiralities comparing the dose-response of added DNA oligonucleotide taret to the two-domain construct versus the three-domain construct.

### DETAILED DESCRIPTION

Described herein are devices and methods for the detection of microRNA (miRNA) and other oligonucleotides in biofluids based on the triggered assembly of a surfactant supramolecular complex on DNA-dispersed SWCNTs. This triggered assembly results in a highly specific emission blueshift shift and an increase in quantum yield based on the resulting decrease in the effective solvent dielectric constant immediately surrounding the nanotube. In certain embodiments, it is possible to detect miRNA with a threshold of 10 pM, with a tunable dynamic range over 5 orders of magnitude (10 pM to 10 μM). Without wishing to be bound by theory, by imaging single nanotube shifting on a surface, it is possible to reduce the threshold theoretically to 10's of copies. In other embodiments, for example by using a structure-switching aptamer for ATP that releases a target oligonucleotide upon binding, it is possible to expand the platform for indirect detection of other biologically relevant analytes.

Described herein are label-free sensors that detect hybridization events of miRNA and other oligonucleotides transiently and in vivo. Included are sensors which transduce the hybridization of small DNA and RNA oligonucleotides into spectral changes of carbon nanotube photoluminescence. Without wishing to be bound by any particular theory, the mechanism of action of the sensors was determined via experiments and molecular dynamics simulations to be a competitive response to local dielectric and electrostatic factors. Accordingly, a scheme was designed where amphiphilic moieties undergo triggered assembly on the nanotube surface upon binding of target miRNA, resulting in a markedly enhanced spectral response. As provided herein, it is shown that the sensors enable multiplexed detection using different nanotube chiralities and real-time monitoring of toehold-mediated DNA-strand displacement, causing a reversal of the signal response. The sensors are highly resistant to non-specific interactions with biological molecules, allowing for direct detection in urine and serum. Further, described herein is the first in vivo optical detection of target DNA and miRNA by encasing the sensor within an implantable device through which hybridization is detected non-invasively via near-infrared fluorescence in live mice.

In certain embodiments, SWCNTs can be used for chirality specific sensing for multiplexed miRNA detection. Importantly, the triggered assembly of surfactant allows for specific and sensitive detection of oligonucleotides in the complex biological environments found in serum and urine, allowing for direct optical measurement of oligonucleotides in these biofluids without the need for purification or labeling. In certain embodiments, the nanotube sensor is encapsulated in a semi-permeable membrane. In certain specific embodiments, this encapsulated sensor can be used for the specific detection of a cancer biomarker miRNA in a live animal.

Individually-dispersed semiconducting single-walled carbon nanotubes (SWCNTs) exhibit exciting properties for use as optical biomedical sensors. Semiconducting carbon nanotubes are fluorescent in the near-infrared spectral region, a wavelength range penetrant to tissue, and they do not photobleach. Their emission wavelength and intensity are sensitive to the local environment, allowing perturbations at the nanotube surface to be transduced via modulation of their emission, with up to single-molecule sensitivity. Moreover, there are about 17 distinct nanotube (n,m) species (chiralities) with unique and resolvable emission wavelengths that can be measured, potentiating multiplexed detection schemes.

### Sensors

Described herein are devices and methods comprising a single-walled carbon nanotube (SWCNT) sensor. In certain embodiments, the sensor comprises a SWCNT and a nucleotide attached to the SWCNT. In certain embodiments, the sensor further comprises a surfactant.

### Single-Walled Carbon Nanotubes (SWCNTs)

Described herein are devices and methods comprising single-walled carbon nanotubes (SWCNTs). SWCNTs are rolled sheets of graphene with nanometer-sized diameters. SWCNTs are defined by their chirality. The sheets that make up the SWCNTs are rolled at specific and discrete, i.e., “chiral” angles. This rolling angle in combination with the nanotube radius determines the nanotube's properties. SWCNTs of different chiralities have different electronical properties. These electronic properties are correlated with respective differences in optical properties. Thus, individually-dispersed semiconducting SWCNTs exhibit ideal qualities as optical biomedical sensors.

Semiconducting SWCNTs are fluorescent in the near-infrared (NIR, 900-1600 nm) due to their electronic band-gap between valence and conduction band. The semiconducting forms of SWNTs, when dispersed by surfactants in aqueous solution, can display distinctive near-infrared (IR) photoluminescence arising from their electronic band gap. IR is a wavelength range penetrant to tissue, and thus potentially suitable for implantable sensors or other devices. The band-gap energy is sensitive to the local dielectric environment around the SWNT, and this property can be exploited in chemical sensing. Among the molecules that can bind to the surface of SWNTs is DNA, which adsorbs as a double-stranded (ds) complex. Certain DNA oligonucleotides will transition from the native, right-handed B form to the left-handed Z form as cations adsorb onto and screen the negatively charged backbone. Additionally, SWCNTs do not photobleach due to their excitonic nature of fluorescence. DNA-DNA hybridization between nanotube-associated DNA and free single-strand DNA in solution can mediate a solvatochromic shift in the nanotube emission.

### Nucleotides

In certain embodiments, the sensor as described herein comprises a polymer capable of being non-covalently or covalently conjugated to the SWCNT. In certain embodiments, the polymer is DNA, RNA, an artificial nucleic acid including peptide nucleic acid (PNA), Morpholino, locked nucleic acid (LNA), glycol nucleic acid (GNA), threose nucleic acid (TNA), an amino-acid sequence, or a synthetic monomer

In certain embodiments, the sensor as described herein comprises a nucleotide attached to the SWCNT. In certain embodiments, the nucleotide can have fewer than 100,000, fewer than 50,000, fewer than 25,000, fewer than 10,000, fewer than 5,000, fewer than 1,000, fewer than 500, fewer than 250, fewer than 100, fewer than 75, fewer than 50, fewer than 30, fewer than 25, fewer than 20, 15, 12, 10, 8, 6 or 4 nucleotides.

In certain embodiments, the nucleotide can have a random sequence. In certain embodiments, the nucleotide can have an ordered sequence. In certain embodiments, the ordered sequence can be a predetermined sequence. In certain embodiments, the ordered sequence can be a repeating sequence. In certain embodiments, the repeat sequence can include fewer than 500, fewer than 400, fewer than 300, fewer than 200, fewer than 100, fewer than 50, fewer than 30, fewer than 25, fewer than 20, 15, 14, 13, 12, 11, 10, 9, 8, 7, 6, 5, 4, 3 or 2 nucleotides. In certain embodiments, the polynucleotide can be poly(AT), poly(GT), poly(CT), poly(AG), poly(CG), or poly(AC). In certain embodiments, the polynucleotide can have a content. In certain embodiments, the content can be a percentage of a unique nucleotide present in the sequence.

In certain embodiments, the nucleotide sequence is a single-stranded DNA molecule. In certain embodiments, the single-stranded DNA (ssDNA) has a sequence complementary to a target nucleotide sequence. In certain embodiments, the ssDNA has a sequence complementary to sequence to miRNA. In certain embodiments, the miRNA is an endogenous piece of RNA with a 21-23 nucleotide sequence. In certain embodiments, the miRNA is mir19, mir21, mir39, mir96, mir126, mir152, mir182, mir183, mir494, or mir509. In certain embodiments, the miRNA is a nucleotide described in Appendix B.

In certain embodiments, the nucleotide has a first domain and a second domain. In certain embodiments, the first domain has a sequence complementary to a target nucleotide sequence as described below. In certain embodiments, the first domain has a sequence complementary to a target miRNA.

In certain embodiments, the second domain is a stabilizing domain, e.g., wherein stabilizing means prevents/reduces agglomeration of SWCNTs and/or promotes stability of a suspension of the SWCNTs. In certain embodiments, the second nucleotide sequence is a short oligonucleotide sequence, e.g., (GT)6 (SEQ ID NO: 2), (GT)15 (SEQ ID NO: 1), (AT)15 (SEQ ID NO: 3), (TAT)6 (SEQ ID NO: 4), (TCC)10 (SEQ ID NO: 5), (TGA)10 (SEQ ID NO: 6), (CCA)10 (SEQ ID NO: 7), (TTA)4TT (SEQ ID NO: 8), (TTA)3TTGTT (SEQ ID NO: 9), (TTA)5TT (SEQ ID NO: 10), (TAT)4 (SEQ ID NO: 11), (CGT)3C (SEQ ID NO: 12), (ATT)4 (SEQ ID NO: 13), (ATT)4AT (SEQ ID NO: 14), (TATT)2TAT (SEQ ID NO: 15), (ATTT)3 (SEQ ID NO: 16), (GTC)2GT (SEQ ID NO: 17), (CCG)4 (SEQ ID NO: 18), (GTT)3G (SEQ ID NO: 19), (TGT)4T (SEQ ID NO: 20), (TATT)3T (SEQ ID NO: 22), (TCG)10 (SEQ ID NO: 23), (GTC)3 (SEQ ID NO: 24), (TCG)2TC (SEQ ID NO: 25), (TCG)4TC (SEQ ID NO: 26), (GTC)2 (SEQ ID NO: 27), (TGTT)2TGT (SEQ ID NO: 28), (TTTA)3T (SEQ ID NO: 29), (CCG)2CC (SEQ ID NO: 30), (TCG)4TC (SEQ ID NO: 31), T3C6T3 (SEQ ID NO: 32), (GTC)2GT (SEQ ID NO: 33), CTTC2TTC (SEQ ID NO: 34), TTA(TAT)2ATT (SEQ ID NO: 35), TCT(CTC)2TCT (SEQ ID NO: 36), (ATT)4 (SEQ ID NO: 37), GC11 (SEQ ID NO: 38), (TC)3CTCCCT (SEQ ID NO: 39), CTTC3TTC (SEQ ID NO: 40), (GT)20 (SEQ ID NO: 41), CTC3TC (SEQ ID NO: 42), (TCT)2 (SEQ ID NO: 43), C5TC6 (SEQ ID NO: 44), T4C4T4 (SEQ ID NO: 45), C5TTC5 (SEQ ID NO: 46), and/or other single-strand DNA that form water soluble complexes with SWCNT.

In certain embodiments, the nucleotide has two, three, four, five, six, seven, eight, or more domains. In certain embodiments, the domains have sequences complementary to one or more target nucleotide sequences.

### Surfactants

In certain embodiments, the methods and devices described herein comprise one or more colloidal stabilization agents. A colloidal stabilization agent is any substance that hinders or prevents aggregation and sedimentation of liquid suspended particles. In certain embodiments, the colloidal stabilization agent is a surfactant. Surfactants are compounds that lower the surface tension (or interfacial tension) between two liquids or between a liquid and a solid. In certain embodiments, the surfactant is a detergent. In certain embodiments, the surfactant is an anionic surfactant, a carboxylate, a cationic surfactant, a zwitterionic surfactant, or a non-ionic surfactant. In certain embodiments, the methods and devices described herein comprise one or more of SDS, SDBS, SDC, SPAN-80, Brij 52, BSA, Triton X-100, Pluronic, Pyrene-PEG, TPGS, IGEPAL, and Phospholipid-PEG-NH2.

### Targets and Analytes

Target conditions and diseases that can be diagnosed, treated and/or prevented using the devices and methods described herein include all cancers, metabolic disease, fetal health condition, kidney disease, organ rejection, hereditary diseases, nervous disease, obesity, and infectious disease. In certain embodiments, the condition or disease is at least in part characterized by a substance, i.e., an analyte.

In certain embodiments, the analytes that can be detected or otherwise manipulated using the devices and methods described herein include nucleotide sequences, e.g., polynucleotides, oligonucleotides, radionucleotides, DNA, RNA, long non-coding RNA, microRNA (miRNA), circulating microRNA, messenger RNA (mRNA), circulating messenger RNA, cell-free tumor DNA, or fragments, analogues, or compounds thereof. Analytes that can be detected or otherwise manipulated using the devices and methods described herein include any molecule or organism having or being associated with the target nucleotide sequences, including peptides, polypeptides, proteins, biologics, biomolecules, biosimilars, aptamers, viruses, bacteria, toxins, cells, antibodies, or fragments thereof.

In certain embodiments, the analyte is a nucleotide with the sequence mir19, mir21, mir39, mir96, mir126, mir152, mir182, mir183, mir494, or mir509, or a nucleotide described in Table 12.

### Systems, Devices, and Methods

In certain embodiments, the device is a sensing platform. In certain embodiment, the device is a sensor. In certain embodiments, the device is in contact with a biofluid or bodily fluid sample. In certain embodiments, the bodily fluid sample is e.g., Amniotic fluid, Aqueous humour and vitreous humour, Bile, Blood serum, Breast milk, Cerebrospinal fluid, Cerumen (earwax), Chyle, Chyme, Endolymph and perilymph, Exudates, Feces, Female ejaculate, Gastric acid, Gastric juice, Lymph, Menstrual fluid, Mucus (including nasal drainage and phlegm), Pericardial fluid, Peritoneal fluid, Pleural fluid, Pus, Rheum, Saliva, Sebum (skin oil), Serous fluid, Semen, Smegma, Sputum, Synovial fluid, Sweat, Tears, Urine, Vaginal secretion, Vomit., etc. In certain embodiments, the bodily fluid in contact with the device is not treated or purified prior to contact with the device.

In certain embodiments, the device is a sensor, or comprises a sensor, as described herein, wherein the device is placed outside of an organism to be treated or diagnosed. In certain embodiments, the device is a point-of-care diagnostic device, a wearable device, or a piece of laboratory equipment. In certain embodiments, the device can be positioned on the surface of the organism, such as the arm, and, e.g., worn like a wristwatch. In certain embodiments, the device is implantable into the organism. In certain embodiments, the devices is a point-of-care medical device, e.g., a (urine) dipstick, a test strip, a membrane, a skin patch, a skin probe, a gastric band, a stent, a catheter, a needle, a contact lens, a prosthetic, a denture, a vaginal ring, or other implant. In certain embodiments, the device comprises a solid support, a membrane, a gel, or a microfluidic component. In certain embodiments, the device comprises a microfluidic chamber containing a sensor. In certain embodiments, the device comprises a sensor contained in a semi-permeable enclosure.

In certain embodiments, the organism to be treated or diagnosed is a mammal, a human, a dog, a rodent, or a farm animal. In certain embodiments, the device is used in to detect oligonucleotides in vivo with a noninvasive method. In certain embodiments, the method is a real-time, non-invasive monitoring in vivo.

In certain embodiments, the device is a sensor, or comprises a sensor, as described herein, and is exposed excitation electromagnetic radiation (excitation EMR) to produce an emission of electromagnetic radiation (emission EMR) by the SWCNT sensor. In certain embodiments, the excitation EMR is ultraviolet light, infrared light, or near-infrared light (NIR). In certain embodiments, the excitation EMR is visible light. In certain embodiments, the excitation EMR has a wavelength between 100 nm and 3000 nm, 200 nm and 2000 nm, between 300 and 1500 nm, or between 500 and 1000 nm.

In certain embodiments, the emission EMR is ultraviolet light, infrared light, or near-infrared light (NIR). In certain embodiments, the emission EMR is visible light. In certain embodiments, the emission EMR has a wavelength between 300 nm and 3000 nm, between 400 and 2000 nm, between 500 and 1500 nm, between 600 nm and 1400 nm, or between 700 and 1350 nm.

In certain embodiments, the methods described herein can be used for diagnostic or therapeutic purposes to diagnose, prevent, or treat any condition or disease characterized by or associated with an analyte as described herein. In certain embodiments, the method comprises contacting a test sample comprising a species having a target nucleotide sequence with the SWCNT sensor; exposing the test sample to excitation electromagnetic radiation (excitation EMR) to produce an emission of electromagnetic radiation (emission EMR) by the SWCNT sensor; detecting the electromagnetic radiation emitted by the SWCNT sensor; and identifying the presence of the species having the target nucleotide sequence (e.g., a polynucleotide, oligonucleotide, radionucleotide, DNA, RNA, microRNA, circulating microRNA, messenger RNA (mRNA), cell-free tumor DNA, or a fragment, an analogue, or a compound thereof) in the test sample based at least in part on the detected emission EMR. Sources of excitation EMR can be any such source known in the art, e.g., a laser, a light emitting diode, or a lamp. Detectors of emission EMR can be any such detector known in the art, e.g., a fluorometer. In certain embodiments, the method comprises detecting a wavelength shift (e.g., a blue or red shift) in the emission EMR and/or an intensity shift (e.g., amplitude shift), or other changes in the spectral characteristics of in the emission EMR, thereby identifying the presence of the species having the target nucleotide sequence in the test sample.

In certain embodiments, a photoluminescence plot (PL plot), as previously described in Bachilo, S. M. et al. Science 298, 2361-6 (2002) can be generated from the emission EMR data. Without wishing to be bound by theory, from the complete PL plots, the peaks can be fit using Gaussian lineshapes to identify the peak center, which then can be used to calculate the magnitude of emission and excitation wavelength shifts relative to a control. In certain embodiments, the method comprises detecting an intensity shift between an emission center wavelength (e.g., a peak) of the test sample and an emission center wavelength (e.g., a peak) of a reference sample, wherein the reference sample is devoid of the species having the target nucleotide sequence. In certain embodiments, the emission wavelength shift is between 1 nm and 100 nm, between 2 nm and 100 nm, between 3 and 50 nm, or between 4 and 20 nm. In certain embodiments, the wavelength shift is a color shift, e.g., a redshift or a blueshift. In certain embodiments, the wavelength shift is a blueshift.

In certain embodiments, the methods comprise the use of a structure-switching aptamer. Without wishing to be bound by theory, in certain embodiments, ATP causes the aptamer to release a target oligonucleotide upon binding. The released target oligonucleotide is detected using the sensors and methods described herein.

In certain embodiments, the device is a sensor, or comprises a sensor, as described herein, and is a device for a non-medical application. In certain embodiments, the device is a device for monitoring environmental conditions. In certain embodiments, the device comprises a solid support, a membrane, a gel, or a microfluidic component, or a combination thereof. In certain embodiments, the device comprises a microfluidic chamber containing a sensor. In certain embodiments, the device comprises a sensor contained in a semi-permeable enclosure.

Dynamic DNA nanotechnology using strand-displacement reactions has recently emerged as an attractive engineering system for various devices, including reconfigurable nanostructures, based on the specificity and versatility of DNA oligonucleotides. Strand displacement reactions can occur through the use of “toeholds,” single-strand overhangs on duplexed DNA that facilitate binding of an invader strand to displace the shorter bound strand. In certain embodiments, the methods and devices described herein relate to Dynamic DNA nanotechnology. In certain embodiments, the device is a component of a nucleic acid system with designed dynamic functionalities related to its overall structures, such as computation and mechanical motion. DNA base pairing allows for the construction of DNA nanostructures and nanodevices through the programmed hybridization of complementary strands. Structures include (logic) circuits, catalytic amplifiers, autonomous molecular motors and reconfigurable nanostructures. Without wishing to be bound by theory, in certain embodiments, the devices described herein can be used in DNA strand-displacement reactions, in which two strands with partial or full complementarity hybridize, displacing in the process one or more pre-hybridized strands, causing dynamic behavior in the system of interest.

### Experimental Examples

### Development of Sensor

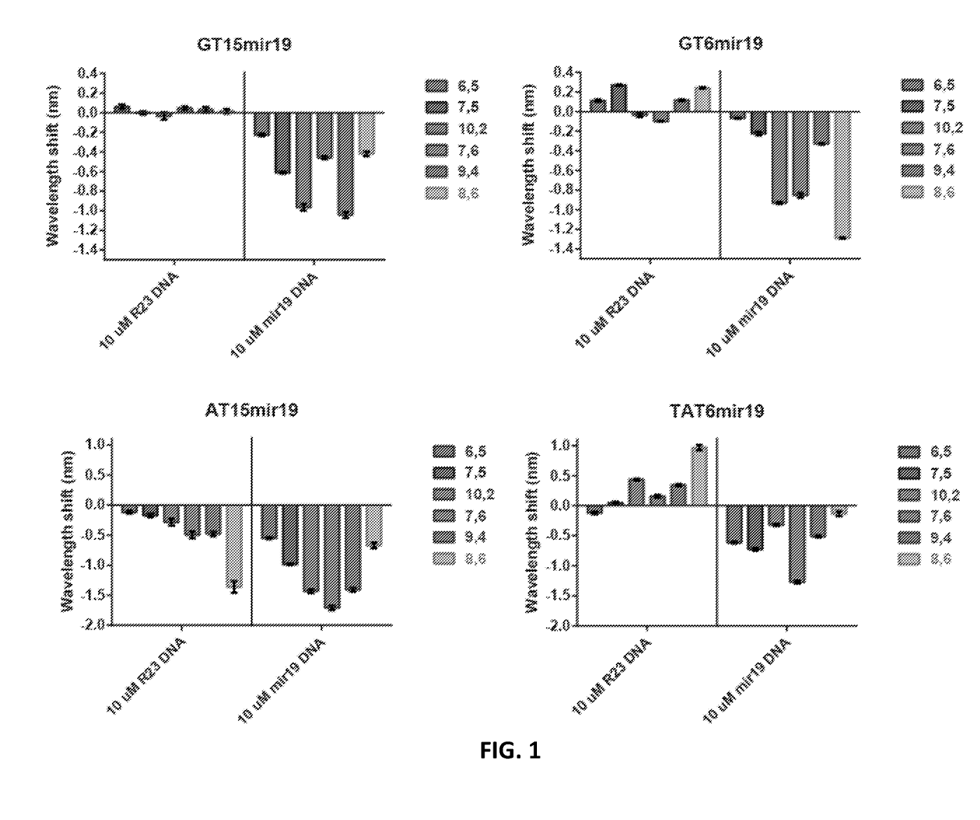

In this example, the basic design of the sensor/sensing platform uses a DNA oligonucleotide to both disperse and stabilize the SWCNTs as well as to provide specificity to target oligonucleotides. The DNA oligonucleotide has a modular design containing two domains; a domain to impart colloidal stability, and a contiguous domain complementary to a target oligonucleotide. A screen of a certain number of sequences found to stably disperse SWCNTs showed that (GT)₁₅ (SEQ ID NO: 1) provides the best stability and resistance to non-specific oligonucleotide interactions (FIG. 1). For the targeting domain, the complementary sequence for the microRNA (miRNA) miR-19 was chosen as a model target, due to the importance of miR-19 miRNA in oncogenesis.

Sequences of miRNA used herein are provided in Table 1.

TABLE 1

| | | ΔG |
|---|---|---|
| Name | Sequence | (kcal/mole) |
| GT 15mir19 | 5′-(GT)₁₅TCAGTTTTGCATAGATTTGC | N/A |
| | ACA-3′ | |
| | (SEQ ID NO: 47) | |
| DNA miR-19 | 3′-AGTCAAAACGTATCTAAACGTGT-5′ | −40.66 |
| | (SEQ ID NO: 48) | |
| RNA miR-19 | 3′-AGUCAAAACGUAUCUAAACGUGU-5′ | −40.66 |
| | (SEQ ID NO: 49) | |
| DNA R23 | 3′-TCGGTCAGTGGGTCATTGCTAGT-5′ | −5.09 |
| | (SEQ ID NO: 50) | |
| RNA R23 | 3′-UCGGUCAGUGGGUCAUUGCUAGU-5′ | −5.09 |
| | (SEQ ID NO: 51) | |

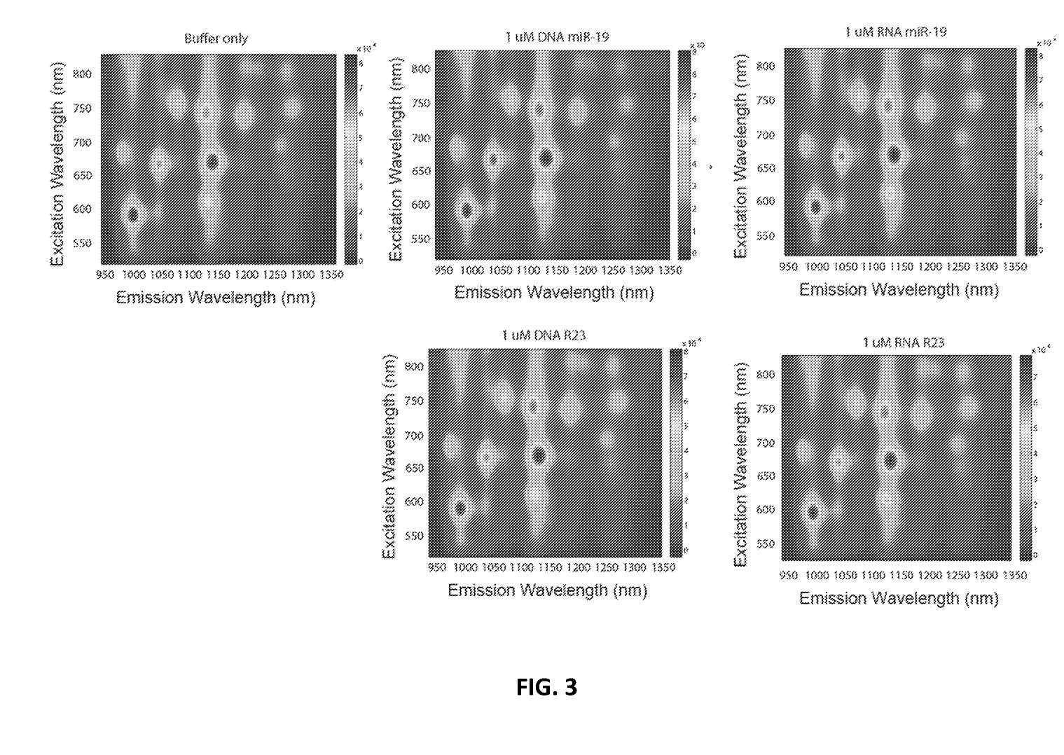

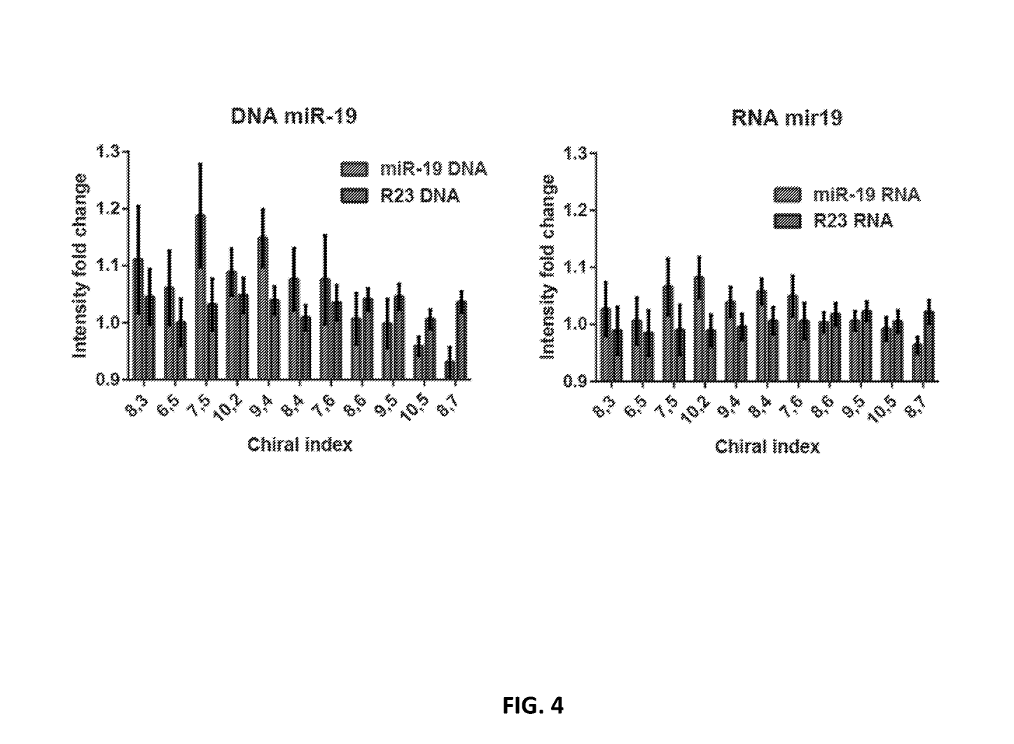

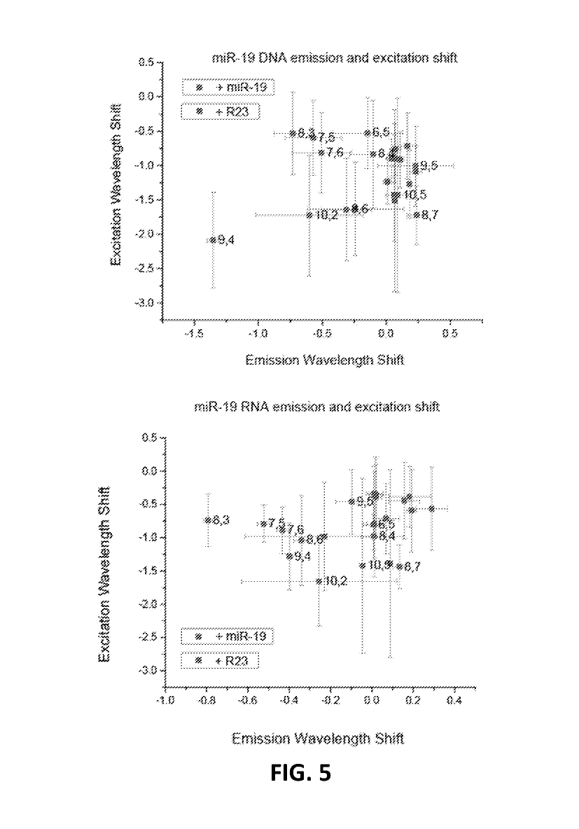

The optical response of the GT15mir19 sensor was tested using both a DNA-based and RNA-based analyte miR-19 sequence, as well as a length-matched, randomly generated, non-complementary control (R23). After incubation with miR-19 or R23, eleven different nanotube chiralities were measured via two-dimensional excitation/emission photoluminescence spectroscopy (“PL plots”, FIG. 3). Each nanotube emission peak exhibited a shift in wavelength which was specific to the miR-19 target sequence over the R23 control (FIG. 2B). In general, nanotube emission peak wavelengths blue-shifted and intensity increased upon introduction of the target oligonucleotide (FIGS. 4A and 4B); excitation peaks (E₂₂ transitions) also blue-shifted (FIG. 5).

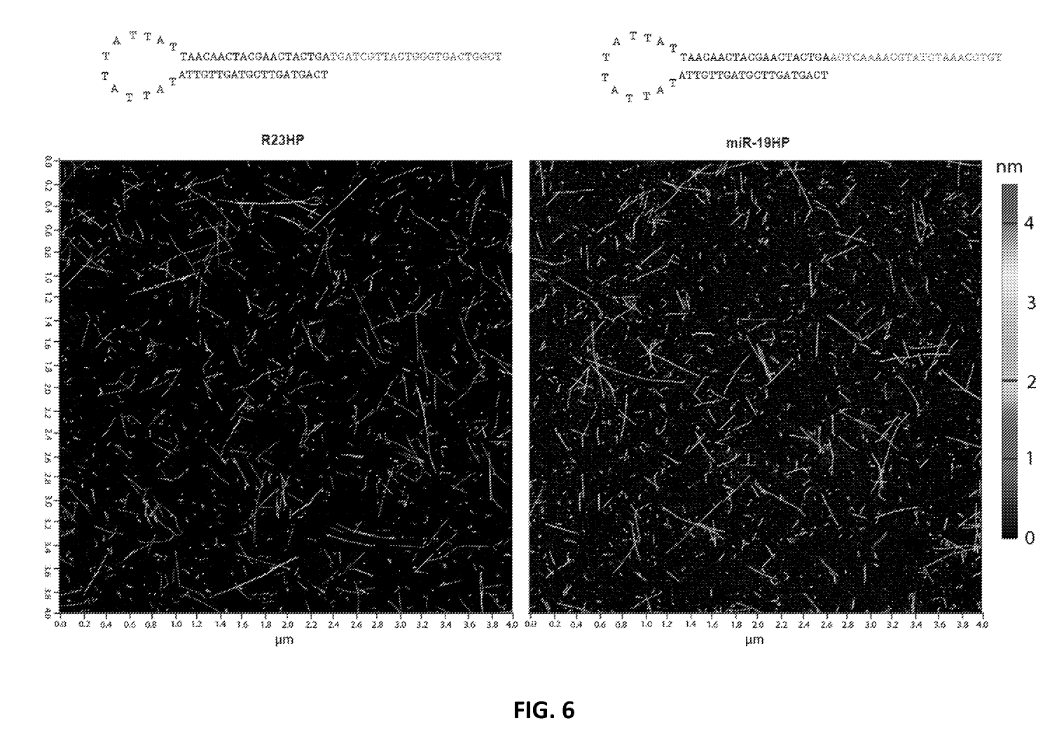

To verify that hybridization to the GT15mir19 sensor occurred upon introduction of the target, a hairpin oligonucleotide was designed which would make binding of the target more apparent by atomic force microscopy (AFM). The oligonucleotide was composed of the miR-19 or R23 sequence, a short spacer, and a 52-nucleotide hairpin region (FIG. 6). After incubation with the miR-19-hairpin, the average height of the sample increased by ˜0.6 nm, as measured by AFM in dry conditions (FIG. 6). Upon imaging in aqueous conditions, it was observed distinct protrusions from the nanotubes which were absent in the R23 hairpin-treated sample and buffer controls (FIG. 2C, FIGS. 44A-44C-46C). Based on this pattern and other AFM studies, it was estimated that the GT15mir19 sensor presents 5-10 binding sites per 100 nm of nanotube. The preparation method yielded nanotubes with a mean length of 166 nm (SD 149 nm); thus it was calculated that an average single nanotube could potentially bind approximately 8-17 copies of miRNA.

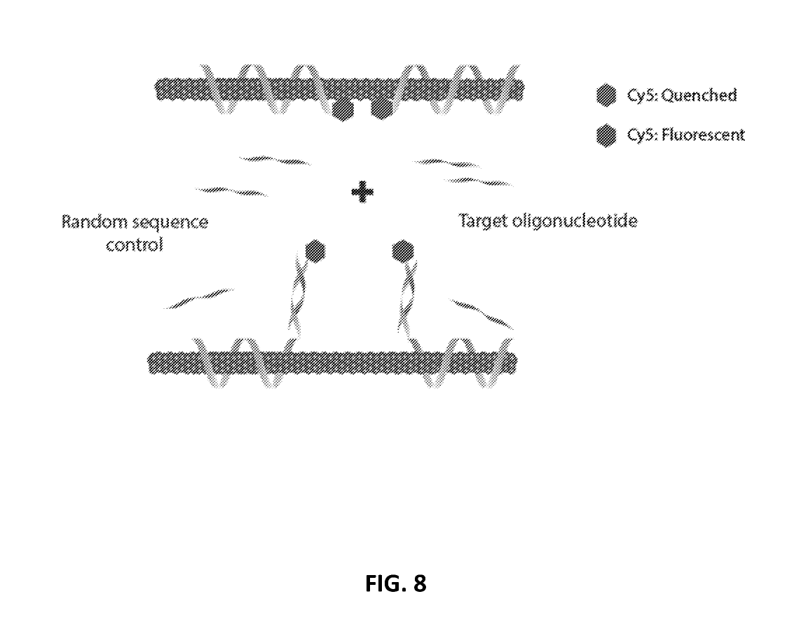

Because the mechanism of nanotube spectral changes induced by oligonucleotide hybridization is poorly understood, a set of experiments was designed to better determine the structural changes of the sensor induced by hybridization. It was first investigated whether the hybridized duplex remained near the nanotube surface after the binding of target miRNA. An assay was developed using an organic fluorophore conjugated to the miRNA capture sequence under the premise that the fluorophore intensity would increase upon hybridization if the fluorophore desorbed from the nanotube surface, as organic fluorophores are known to quench upon interaction with the nanotube surface via an energy transfer mechanism. Nanotubes with the sequence GT6mir19 were suspended, (shortened due to synthesis constraints) containing the Cy5 dye conjugated to the 3′ end of the miR-19-binding domain (scheme in FIG. 8).

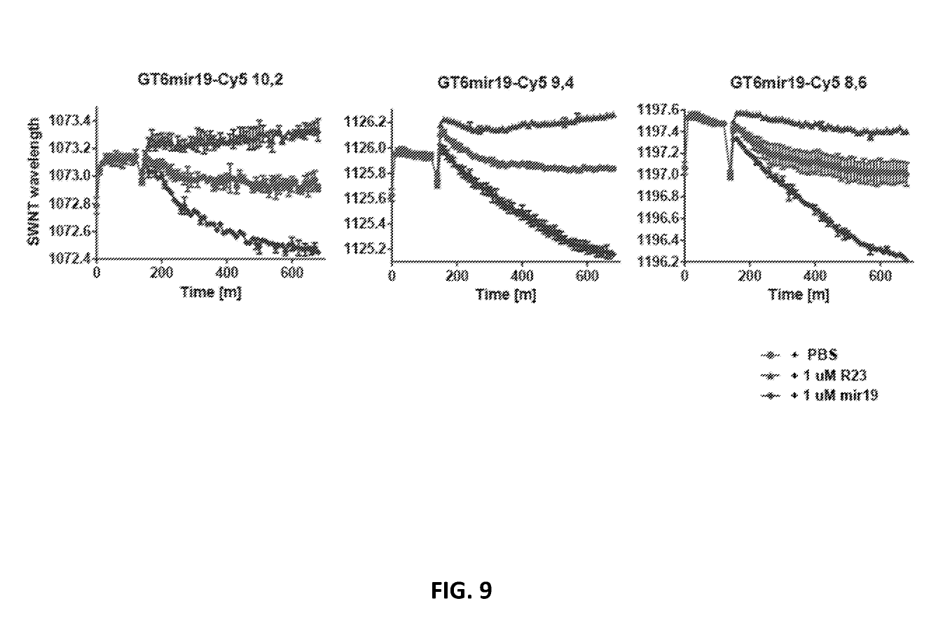

Upon addition of miR-19 to the modified complex, it was found that Cy5 fluorescence increased over time, while the R23 sequence caused no change in Cy5 fluorescence (FIG. 2D). To validate GT6mir19-Cy5 as a proxy for the GT15mir19 sequence, the nanotube emission was measured upon introduction of the miR-19 sequence to the fluorophore-labeled complex. Again, blue-shifting was seen upon hybridization with the target oligonucleotide, suggesting the same sensor function despite the shortened nanotube-binding domain (FIG. 9). In agreement with the Cy5 fluorescence change, it was found that nanotube fluorescence emission to blue-shift occurred at a slower rate compared to GT6mir19 without Cy5 (FIG. 9). Without having to be bound to any theory, this relatively slow rate may be a result of the affinity of Cy5 for the nanotube surface, based on π stacking interactions between the Cy5 dye, which is rich with π electrons, and the graphitic π electrons of the nanotube. The fluorophore de-quenching and AFM together suggest a final hybridized structure consisting of a partial duplex dissociating from the nanotube surface.

Using all-atom molecular dynamics simulations, it was assessed whether the GT15mir19 sequence could remain stable on the nanotube upon partial hybridization. The pre-hybridized sequence was placed in the vicinity of the (9,4) nanotube with explicit water and counterions, and a simulation was run for 250 ns (as provided herein). The single-stranded portion of the oligomer bound to the nanotube and the hybridized construct remained stable on the nanotube surface for the remainder of the simulation (FIG. 2E “Hyb”). A second simulation was run in absence of the hybridization strand. During the simulation, the entire oligomer bound to the surface and wrapped the nanotube, with the nucleobases orienting closely to the nanotube surface in a parallel orientation (FIG. 2E, “Unhyb”).

The simulations allowed the quantification of nucleobase adsorption to the nanotube surface. The radial distance of the nucleobases was measured from the nanotube surface and their stacking angles relative to the nanotube surface (FIGS. 45A-45F). It was observed that all bases of the (GT)₁₅ (SEQ ID NO: 1) nanotube-binding domain remained adsorbed on the nanotube surface, whereas only 1-2 terminal bases of the double-stranded miR-19/miRNA capture sequence adsorbed to the nanotube surface. In the simulation without the complementary strand, all bases of the (GT)₁₅ (SEQ ID NO: 1) nanotube binding domain adsorbed to the nanotube surface, as well as most of the bases of the miR-19 miRNA capture sequence (FIGS. 45A-45F).

The thermodynamic concerns regarding the stability of the hybridized duplex were assessed in the presence of the nanotube. Molecular dynamics simulations of hybridized miR-19, without the (GT)₁₅ (SEQ ID NO: 1) nanotube binding domain, in the presence of the nanotube were run using several different initial conditions (FIGS. 46A-46B). In all simulations, no de-hybridization of the duplex was observed, suggesting that the nanotube would not destabilize the hybridized duplex. To better determine how the partial hybridized state of the DNA is stable on the nanotube (or preferred over single strand adsorption on the nanotube), a free energy analysis was conducted (FIG. 47). The analysis suggests that hybridization of the dsDNA is favored if the analyte strand is not initially adsorbed on the nanotube surface, as is the case in the described experiments.

The molecular dynamics simulations were also analyzed to gain a quantitative determination of the carbon nanotube spectral response upon hybridization. Comparing the water density as a function of distance at the end of the two simulations, it was found that a slight increase in the water concentration near the nanotube in the hybridized structure (FIG. 2I). In addition, it was found that the density of phosphate ions as a function of distance from the nanotube decreased upon hybridization (FIG. 2J). While an increase in local water density can cause red-shifting of the nanotube emission wavelength, a decrease in local anionic charge density in the local environment of the nanotube was found to cause a blue-shifting response. As the nanotube emission exhibited a net blue-shift upon hybridization, it was determined that the effect of the removal of phosphate charges from the nanotube surface out-competed the effects of increased local water density.

Table 2 shows surfactant and polymer suspended nanotubes spectral properties. Note that numbers in parentheses indicate the molecular weight of polyethylene glycol; these surfactants share polyethlene glycol as a component. Accordingly, the numbers in parentheses are included for comparison.

TABLE 2

| | | | | |
|---|---|---|---|---|
| | | | | (8,3) |
| | | mass | (8,3) | flour. Pos. |
| Surfactant/ | molecular | percent | fluor. G- | relative to |
| polymer | weight | conversion | peak | SDS (cm⁻¹) |

anionic

| | | | | |
|---|---|---|---|---|
| SDS | 288.4 | 3.3 ± 0.5 | 3.6 ± 0.5 | 0 ± 10 |
| SDBS | 348.5 | 3.9 | 4.6 | 2 |
| SDSA | 272.4 | 6.0 | 4.5 | −30 |
| Sarkosyl | 293.4 | 2.8 | 4.3 | −117 |
| TREM | 428 | 4.0 | 3.0 | −47 |
| PSS-70 | 70,000 | 4.7 | 1.4 | −214 |

cationic

| | | | | |
|---|---|---|---|---|
| DTAB | 308.4 | 5.6 | 2.3 | −129 |
| CTAB | 364.5 | 5.1 | 2.3 | −124 |

nonionic

| | | | | |
|---|---|---|---|---|
| Brij 78 | 1,198 | 4.3 | 1.3 | −203 |
| Brij 700 | 4,670 | 6.4 | 2.5 | −106 |
| Tween 85^{c} | 1,839 | 3.9 | 1.8 | −79 |
| Triton X-405^{c} | 1,966 | 5.0 | 2.8 | −119 |
| PVP-1300^{c} | 1,300,000 | 4.1 | 0.4 | −211 |
| EBE | 4,970 | 6.4 | 3.6 | −75 |
| Pluronic P 103 | 4,950 (1,485) | 1.9 | 0.7 | −68 |
| Pluronic P 104 | 5,900 (2,360) | 3.0 | 0.8 | −69 |
| Pluronic P 105 | 6,500 (3,250) | 4.8 | 1.4 | −70 |
| Pluronic F 108 | 14,600 (11,680) | 8.7 | 1.2 | −95 |
| Pluronic F 98 | 13,000 (10,400) | 9.4 | 1.1 | −97 |
| Pluronic F 68 | 8,400 (6,720) | 5.8 | 1.2 | −103 |
| Pluronic F 127 | 12,600 (8,820) | 7.1 | 1.6 | −84 |
| Pluronic F 87 | 7,700 (5,390) | 8.8 | 1.5 | −105 |
| Pluronic F 77 | 6,600 (4,620) | 2.5 | 0.5 | −208 |
| Pluronic F 85 | 4,620 (2,310) | 0 | — | — |

As the simulations showed an increase in available nanotube surface area upon hybridization, it was hypothesized that additional small amphipathic molecules might assemble on this newly exposed nanotube surface to enhance the optical response. Low concentrations of several candidate surfactants (Table 3) were tested to determine whether they changed the optical response of the GT15mir19 sensor (FIGS. 48A-48B and 49A-49B).

TABLE 3

| | Abbre- | Class of amphipathic |
|---|---|---|
| Name | viation | molecule |
| Sodium deoxycholate | SDC | ionic surfactant |
| Sodium dodecyl sulfate | SDS | ionic surfactant |
| Sodium | SDBS | ionic surfactant |
| dodecylbenenesulfonate | | |
| Pluronic F-68 | Pluronic | non-ionic triblock copolymers |
| Triton X-100 | n/a | non-ionic surfactant |
| IGEPAL CO-530 | IGEPAL | non-ionic surfactant |
| Span 80 | n/a | non-ionic surfactant |
| Birj 52 | n/a | non-ionic surfactant |
| D-α Tocopherol polyethylene | TPGS | non-ionic surfactant |
| glycol 1000 succinate | | (vitamin E) |
| 1,2-distearoyl-sn-glycero-3- | Lipid-PEG | PEGylated lipid |
| phosphoethanolamine- | | |
| N[methoxy(polyethylene | | |
| glycol)-1000] (ammonium | | |
| salt)) | | |
| Bovine serum albumin | BSA | protein |

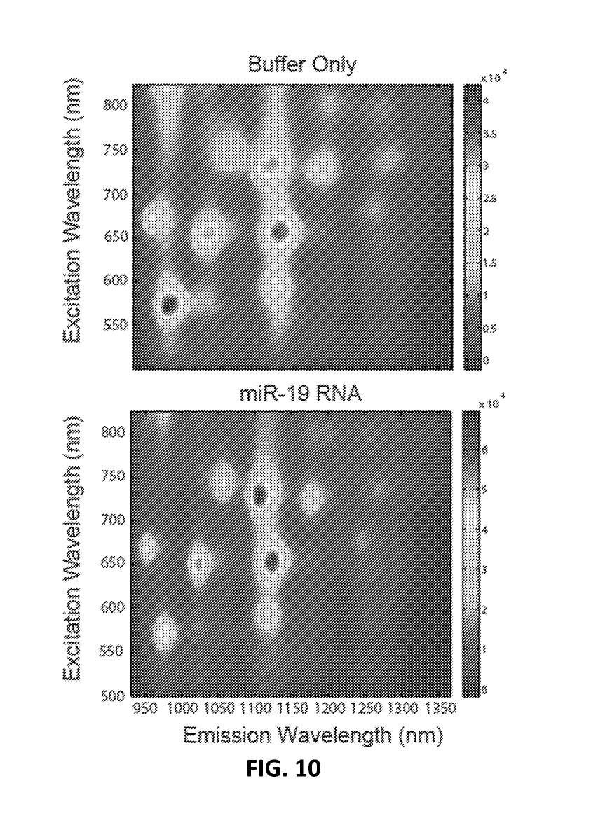

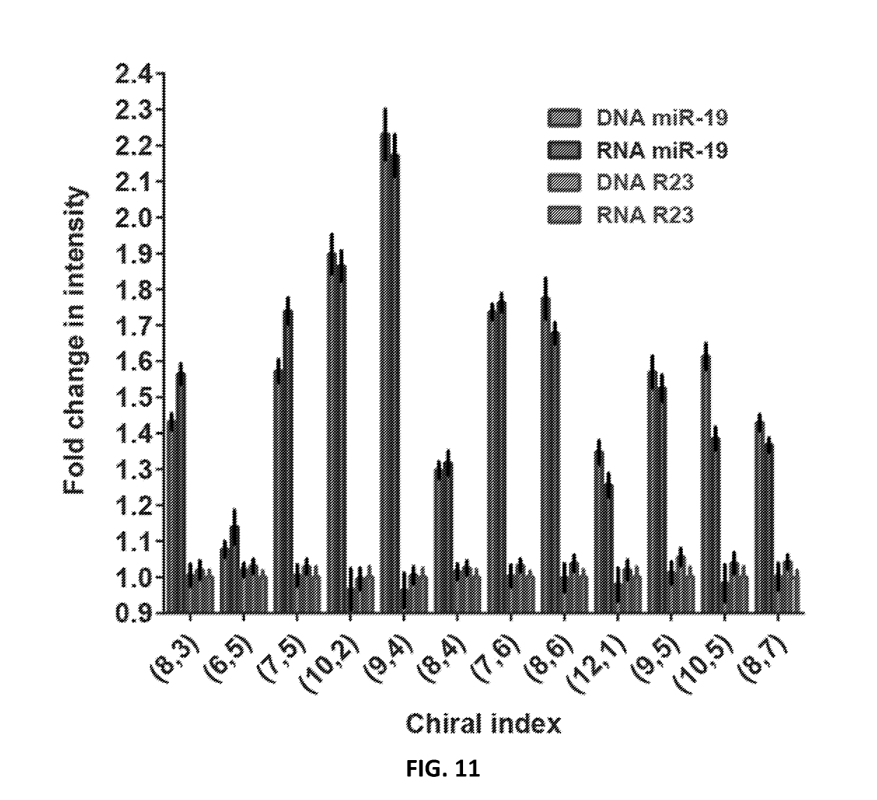

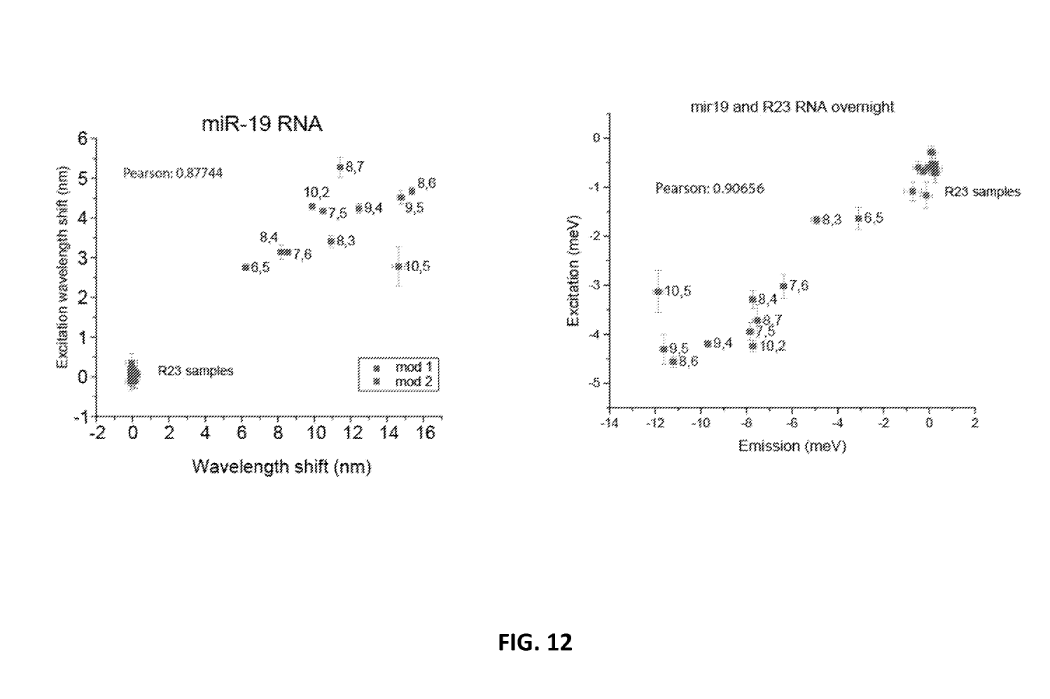

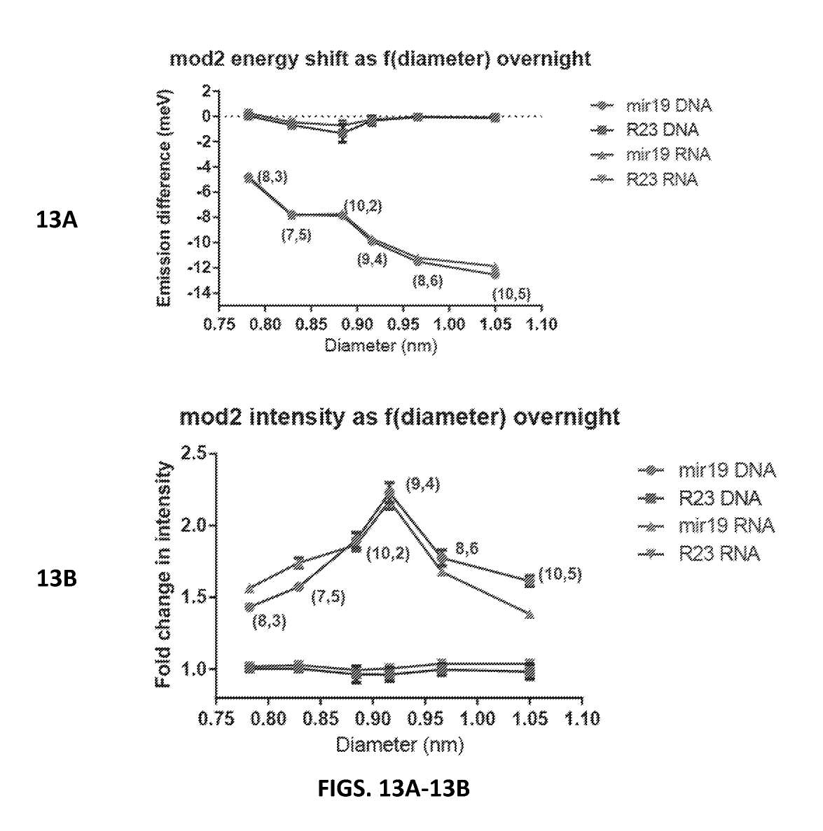

The study found that a low concentration (0.2% wt/vol, or 5.7 mM) of sodium dodecylbenzenesulfonate (SDBS), a mild surfactant known to associate with nanotubes, resulted in an increase in the degree of hybridization-dependent blue-shifting and intensity enhancement by an order of magnitude (FIG. 2F). In the SDBS-supplemented buffer-only condition and in the presence of the R23 control, the emission bands broadened slightly but did not increase or shift appreciably. Upon hybridization in the presence of SDBS, all nanotube chiralities exhibited a greatly enhanced blue-shift (FIG. 2G, FIGS. 10-14), even those that that did not blue-shift in the absence of SDBS (FIG. 2B). A significant blue-shift in the excitation wavelength was also observed (FIG. 12). The magnitude of blue-shifting and intensity enhancement (FIG. 11) upon hybridization of DNA and RNA were identical. In the absence of the target oligonucleotide, the GT15mir19 sensor emission remained stable over a wide-range of SDBS concentrations (FIGS. 13A-13B). A model of SDBS-mediated hybridization-dependent signal enhancement is presented in FIG. 2H, wherein hybridization triggers SDBS assembly on the newly-exposed nanotube surface. For a more detailed analysis of the observed spectroscopic changes induced by SDBS, see the below experiments.

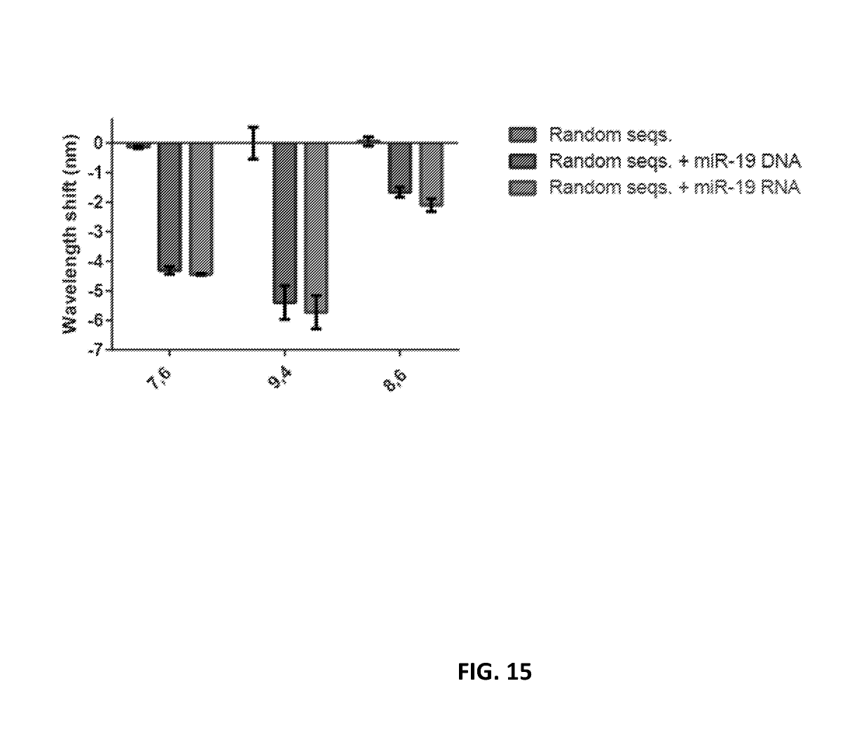

To further assess the specificity of the sensor response, an ensemble of randomly generated oligonucleotides was introduced. A random library of 23 nt oligonucleotides, with a diversity of approximately 4²³ different sequences, was introduced to the GT15mir19 sensor, resulting in no response (FIG. 15). In the presence of the random library, the GT15mir19 sensor maintained sensitivity to miR-19.

FIG. 2A shows a schematic for the construction of the sensing platform. Briefly, the DNA oligonucleotide was sonicated by probe tip with HiPCO SWCNTs, followed by centrifugation to remove poorly suspended SWCNTs. The resulting construct, henceforth referred to as GT15mir19 (GT15-encapsulation sequence (SEQ ID NO: 1) and miR-19 complementary sequence), showed a high degree of stability over at least several months (data not shown).

Functionality of the sensor was tested using both a DNA-based and an RNA-based target miR-19 sequence, as well as a length-matched random DNA and RNA sequence control (R23). After incubation with miR-19 or R23, eleven different nanotubes were sampled by measuring fluorescence intensity as a function of excitation wavelength and emission wavelength in a photoluminescence plot (PL plot). From the complete PL plots (FIG. 3), the peaks were fit using Gaussian line shapes to identify the peak center, which were then used to calculate the magnitude of emission and excitation wavelength shifts relative to a control that received buffer only. The resulting emission wavelength shifts from miR-19 and R23 are shown in FIG. 2B, arranged by nanotube chirality from smallest to largest diameter. For the DNA-based miR-19 sequence, the magnitude of blueshifting relative to the control is between 0.5 and 1 nm for most nanotubes, with the largest diameters showing no shift. Blueshifting was consistent with previous reports of DNA-hybridization on carbon nanotubes. The random sequence control elicited no change or in some cases a small redshift. For the RNA-based miR-19 target, a smaller degree of blueshifting was observed for most chiralities, with the largest diameter nanotubes again showing little response. Overall, the pattern of shifting for DNA and RNA was similar, except that RNA hybridization produced a smaller magnitude of shifting. For some chiralities, an enhancement in intensity followed binding of DNA target (FIGS. 4A-4B). Shifts in the excitation wavelength were minor, and showed a similar pattern of sensitivity (FIG. 5).

To verify that the sensor was only interacting with the complementary oligonucleotide, the height-profile changes were measured with AFM after incubation with the target sequence or with the random sequence control. To exacerbate a change in height after binding, hairpins were designed with a 20 nucleotide long stem and 12 nucleotide loop that contained the single strand miR-19 or miR-23 sequence at the end of the stem. After overnight incubation and washing, the sample was adsorbed to mica and measured with AFM. In both samples, the helical wrapping pattern of GT15mir19 was visible, as reported previously for single-stranded DNA (see Gigliotti, B., Nano Lett. 6, 159-64 (2006)). A comparison of the average heights between the sample that had received miR-19-hairpin versus the R23-hairpin showed that the miR-19-hairpin increased the average height by about 0.6 nm, consistent with the miR-19-hairpin being bound to the surface (FIG. 2C, FIG. 6).

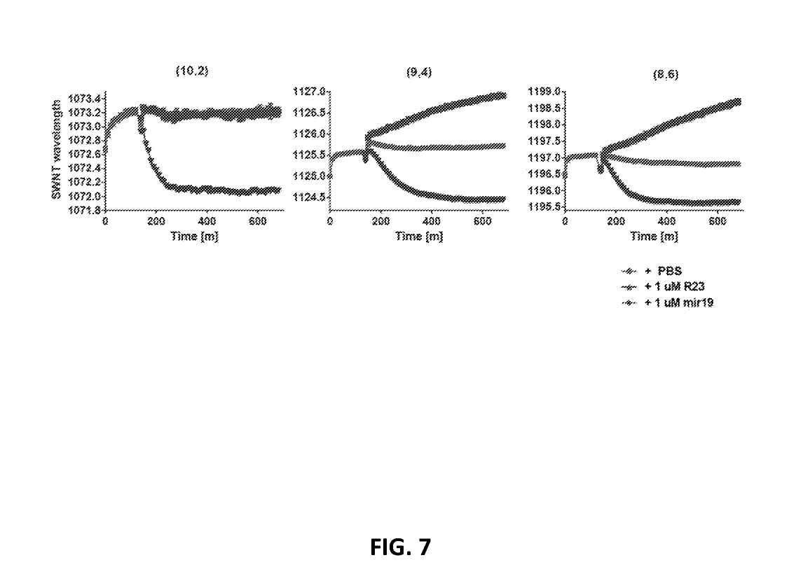

Without wishing to be bound by theory, mechanistically, the change in nanotube optical response may be due to the hybridized duplex remaining on the nanotube surface after complementary base-pairing, or due to the newly formed duplex partially dissociating from the surface. To test this, SWCNTs were suspended with GT6mir19 containing the fluorophore Cy5 conjugated to the end of the miR-19-binding domain. GT6 (SEQ ID NO: 2) was chosen as the dispersing domain due to length restraints for oligonucleotide functionalization with fluorophores. While less effective at providing resistance to R23 binding at high concentrations, GT6mir19 still specifically blueshifted upon hybridization with target oligonucleotide (FIG. 7). Without wishing to be bound by theory, SWCNTs may be effective quenchers of organic fluorophores through energy transfer when held in close proximity to the nanotube surface. Thus, without wishing to be bound by theory, restoration of Cy5 fluorescence upon binding of miR-19 would indicate removal from the surface, and support the hypothesis of the newly formed duplex partially dissociating from the surface (FIG. 8). With GT6mir19-Cy5, it was found that Cy5 fluorescence was restored in the presence of 1 μM miR-19 DNA, while 1 μM R23 had no effect (FIG. 2D). Concurrent with the measurement of Cy5 fluorescence, the SWCNT fluorescence emission changes from three different chiralities were measured, as well as from control GT6mir19 that did not have the Cy5 fluorophore. With Cy5, all nanotubes blueshifted (FIG. 9), indicating that the Cy5 dequenching was concurrent with nanotube blueshifting. When compared to GT6mir19-suspended nanotubes without Cy5, the rate of blueshifting was severely hampered (FIG. 7, FIG. 9). Without wishing to be bound by theory, because Cy5 is rich with π electrons, the relatively slow rate may be due to the affinity of Cy5 for the nanotube surface based on π stacking interactions, hindering the complementary base-pairing induced removal of the duplex. Without wishing to be bound by theory, these data suggest that the mechanism of detection of target sequences is from duplex formation and partial dissociation from the nanotube at the recognition domain. Using all atom molecular dynamics, it was tested if the hypothesized partial duplex would remain stable with the SWCNT present in the simulated conditions (FIG. 2E).

It was hypothesized that the observed blueshifting could be enhanced by adding a small amount of amphipathic molecules to interact with and assemble upon the newly exposed carbon nanotube surface. After screening several amphipathic molecules, sodium dodecylbenzenesulfonate (SDBS), a surfactant known to associate with SWCNTs, was found to greatly enhance the magnitude of blueshifting and intensity enhancement. FIG. 2F shows spectra from 730 nm excitation with and without target miR-19 in buffer only conditions in the top panel, and in the presence of 0.2% SDBS in the bottom panel, revealing a dramatic impact on wavelength and intensity. By taking PL plots (FIG. 10), twelve different chiralities were compared with target miR-19 DNA and RNA or R23 DNA and RNA controls. Each chirality displayed a greatly enhanced blueshift, even for chiralities that did not blueshift in buffer only conditions (FIG. 2G, compare FIG. 2B), as well as intensity enhancement for each chirality (FIG. 11). In all cases, R23 had no effect. From the PL plots, it was also found that there was a blueshift in excitation wavelength. Without wishing to be bound by theory, a change in the excitation wavelength reflects a change in the E22 transition, indicating a change in solvation energy associated with stabilizing the ground state. Thus, without wishing to be bound by theory, binding of miR-19 RNA and DNA can affect the ground state absorption energies in addition to the excited state, adding yet another detection modality for target miR binding. FIG. 12 shows the correlation between the excitation wavelength shift and the emission wavelength shift for the ensemble of chiralities. Excluding the outliers (8,7) and (10,5), a Pearson correlation coefficient of 0.87744 (p=0.00188, **) was found. When plotted as change in energy (FIG. 12), the Pearson correlation coefficient was similar 0.90656 (p=0.0007). The environmental effects on nanotube optical properties can depend at least in part on the mod type of the nanotube. Stratifying the nanotubes by mod type, it was found that the mod 2 nanotubes had an emission energy change that increased nearly linearly (R²=0.9272) with nanotube diameter (FIG. 13A). Interestingly, the intensity enhancement as a function of diameter for the mod 2 nanotubes did not show the same linear relationship; although all nanotubes increased in intensity, a maximum was found for nanotubes around 0.9 nm in diameter (FIG. 13B). A slight difference also became apparent between DNA and RNA, with RNA showing a slightly enhanced intensity increase for small diameter nanotubes and a slightly dampened enhancement for larger diameter nanotubes. Without wishing to be bound by theory, this small, diameter dependent difference may be related to the difference in binding strength and hydration between DNA-DNA hybrids and DNA-RNA hybrids.

Optical transition energies for DNA-wrapped SWCNTs are red-shifted by 10-20 meV compared to nanotubes suspended entirely in surfactants like SDS or SDBS (See Haggenmueller, R. et al. Langmuir 24, 5070-8 (2008); Fantini, C. et al. Chem. Phys. Lett. 473, 96-101 (2009)). This is due to incomplete coverage of the nanotube surface by DNA, which allows for greater accessibility of water and a resulting larger dielectric in the immediate vicinity of the nanotube (See Miyauchi, Y. et al. Chem. Phys. Lett. 442, 394-399 (2007). Additionally, SDBS suspended nanotube have been shown to produce a higher quantum yield than DNA-suspended nanotubes (See Fantini et al.). Without wishing to be bound by theory, the blueshifted shoulder-feature that SDBS produced on the spectra of DNA-wrapped nanotubes in the photoluminescence plots (FIG. 2F, FIG. 10) suggested that for each chirality, SDBS is binding to the exposed surfaces on the DNA-suspended nanotube. Without wishing to be bound by theory, the differing dielectric microenvironments from the DNA covered surface (relatively redshifted) and SDBS covered surface (relatively blueshifted) both contribute to the emission character, creating the observed elongation in emission. Without wishing to be bound by theory, when target RNA or DNA is bound and the duplex dissociates from the surface, more bare nanotube surface is exposed. Without wishing to be bound by theory, the newly exposed nanotube surfaces allow SDBS greater access to the nanotube, becoming the dominant determinate of the nanotube wavelength emission, excitation, and intensity character. Without wishing to be bound by theory, the remaining DNA covered portion of the nanotube now only contributes a minor red-shifted shoulder. Without wishing to be bound by theory, the net-effect is a dramatic blueshift (4-12 meV blueshift, depending on chirality (FIG. 12) and intensity increase from the assembly of supramolecular complexes of SDBS, triggered by the detection of target RNA or DNA (FIG. 2H).

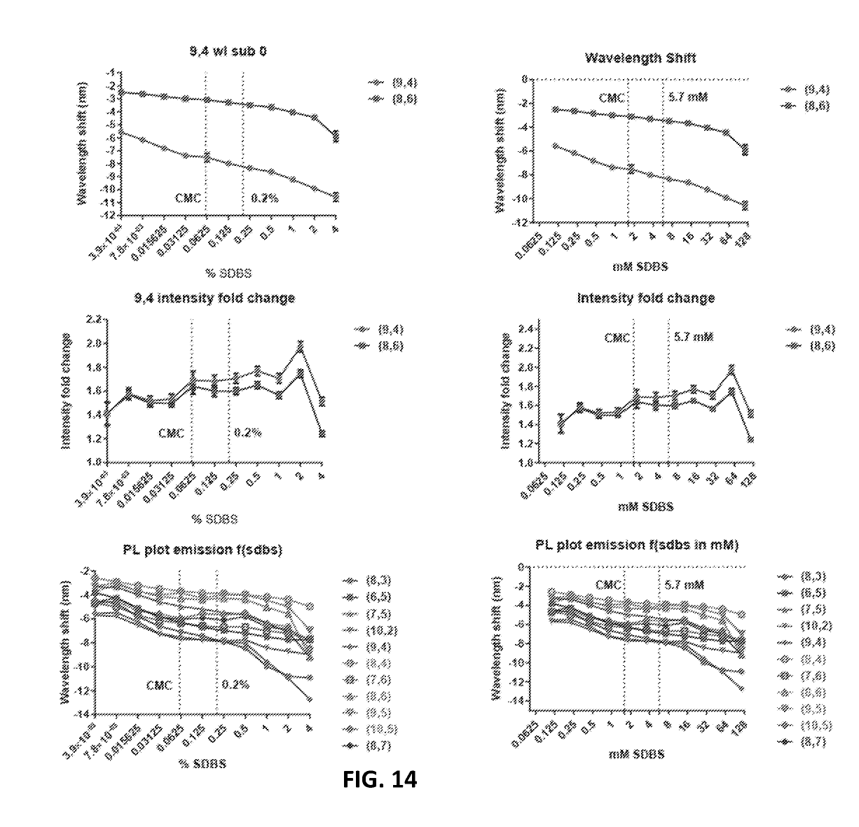

To test the stability of GT15mir19 with SDBS, PL plots after a 1:2 serial dilution from 4% to 0.004% SDBS (128 mM to 0.0625 mM) were measured. Increasing SDBS showed only minor changes in the baseline emission of GT15mir19, except at high concentrations for some large-diameter chiralities (above about 2%). GT15mir19, for most chiralities, was remarkably stable over 4 orders of magnitudes of SDBS in the absence of target miR (FIG. 14).

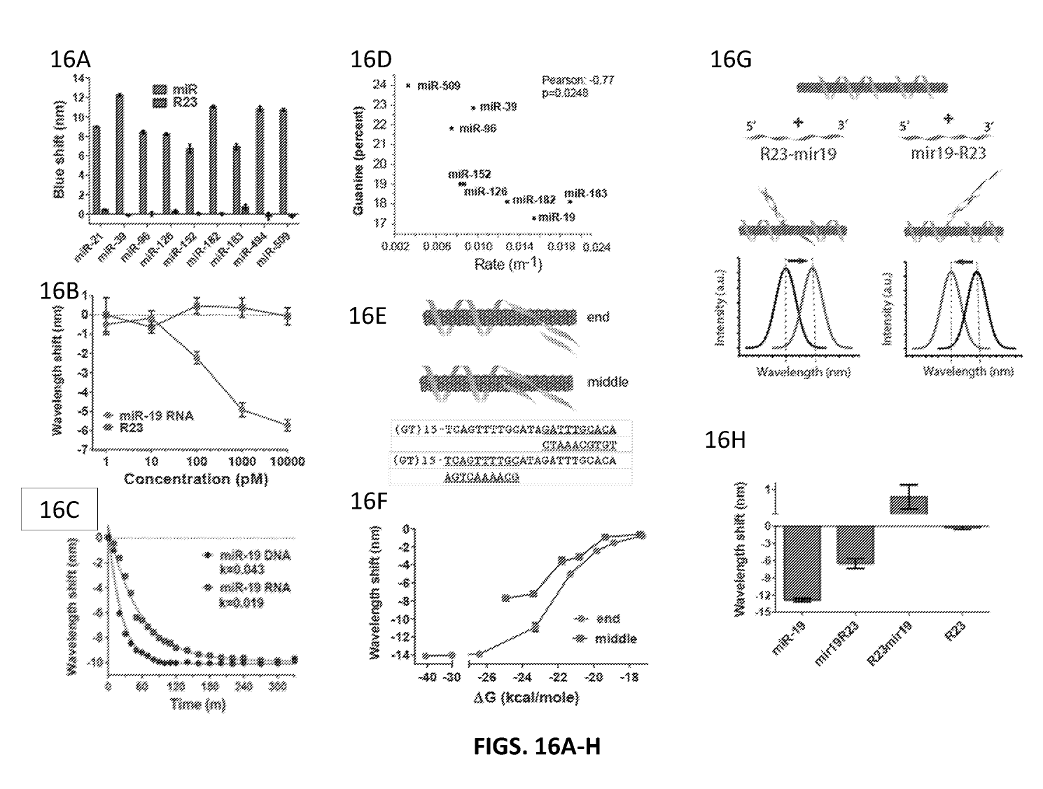

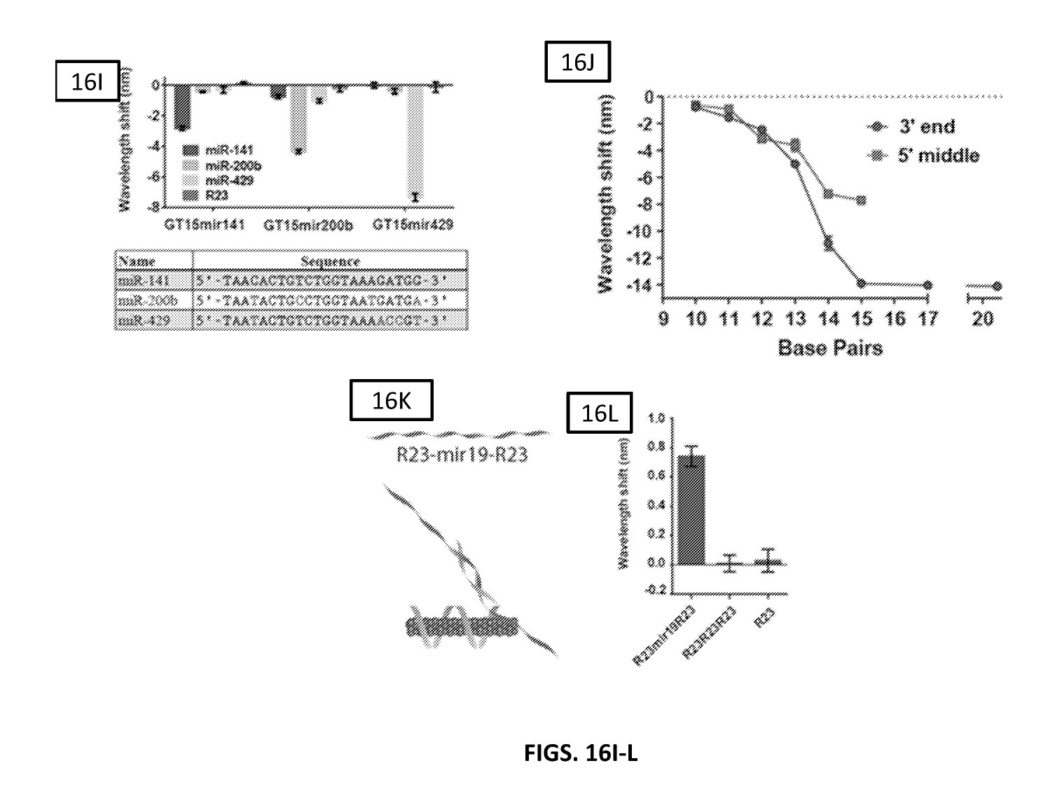

The hybridization-triggered supramolecular assembly and resulting enhancement in blueshifting and quantum yield now provided a rationally designed platform for the detection of target RNA or DNA oligonucleotide. This was further characterized in terms of specificity. Because only one random sequence control was used, i.e., R23, random permutations of oligonucleotides each 23 bases long were generated to verify that the sensing platform could recognize the miR-19 target in the context of many random sequences. In the presence of 1 μM of the many random sequences, there was no significant change in wavelength for the measured chiralities (FIG. 15). Additionally, each chirality was able to recognize miR-19 in this context. To verify that the platform was extendible to any miR of interest, a panel of miR sequences that have been found to serve as biomarkers in the serum and urine was tested, as well as a miR sequence that is not found in human samples (miR-39) that could serve as a control. For each DNA miR sequence, the extent of blueshifting upon specific recognition was greatly enhanced by the addition of SDBS to 0.2% (FIG. 16A).

### Detection Limit, Kinetics, and Breadth of Applicability

Given the variety of potential miRNA biomarkers, it was sought to assess the modularly of the sensor. The miRNA capture sequence was substituted with several sequences specific to 9 different serum or urine miRNA biomarkers, as well as a sequence not found in humans (C. elegans miR-39) used for standardization in clinical applications (Table 4). Each GT15mirX sensor was treated with SDBS and interrogated with its respective miRNA target sequence, resulting in a wavelength shift which was comparable to that of the original miR-19 sensor, with slight sequence-to-sequence variations (FIG. 16A and FIG. 50). Intensity was similarly enhanced (FIG. 65). In all of the sensors, no appreciable responses from the control sequence (R23) were observed.

Table 4 shows name, disease relevance, and biofluid of miCRNAs tested in FIG. 16A.

TABLE 4

| Name | Disease relevance | Biofluid |
|---|---|---|
| miR-21 | diffuse large B-cell lymphoma | serum |
| miR-96 | Urothelial carcinoma | urine sediment |
| miR-183 | Urothelial carcinoma | urine sediment |
| miR-126 | Urinary bladder cancer | voided urine |
| miR-182 | Urinary bladder cancer | voided urine |
| miR-152 | Healthy control | voided urine |
| miR-494 | Acute kidney injury | voided urine |
| miR-509 | Healthy control, highly | voided urine |
| | expressed | |
| miR-39 | Found only in C. elegans; | N/A |
| | common spike-in control | |

To determine if the SDBS-GT15mirX sensor could discriminate among similar sequences, three related sequences from the miR-200 family were selected. The miR-200 family plays an essential role in the epithelial-to-mesenchymal transition (EMT) in cancer. Focusing on the wavelength response of the (9,4) nanotube chirality, a high degree of discrimination between the three sequences after one hour of incubation was observed (FIG. 16I). Complete time-course data for both the (9,4) and (8,6) nanotubes (FIGS. 42A-44F and FIGS. 51A-51F, respectively) revealed that the intensity increase provided near-perfect discrimination in most cases. Although the SDBS-GT15mirX sensor responded to target miRNA via both wavelength shifting and intensity changes, detection limits, kinetics, and other sensor characteristics were assessed using the wavelength response, due to the inherent quantifiability and internal standard provided by this mode.

To determine the limit and range of detection, a dose-response curve of the sensor was constructed over several orders of magnitude of miR-19 concentrations. At a minimal sensor concentration of 0.02 mg/L, the limit of detection of miRNA was between 10 and 100 pM (500 attomoles to 5 femtomoles) (FIG. 16B). Signal saturation occurred at a high concentration between 1 and 10 nM. The dynamic range was tuned by adjusting the concentration of the GT15mir19 sensor to cover at least 5 orders of magnitude, from 10 pM to 1 μM (FIG. 16B). The number of binding sites was calculated using the mass of DNA used to suspend 1 mg of nanotubes (see below). It was estimated that 2.117 nM of miR-19 binding sites were available in a solution of 0.02 mg/L of the GT15mir19 sensor, consistent with the observed saturating range of concentrations (between 1 and 10 nM).

Table 5 shows SWCNT concentration, limit of detection, binding sites, and saturating range of values shown in FIG. 16B.

TABLE 5

| SWCNT conc. | | LOD | Binding sites | Saturating range | |
|---|---|---|---|---|---|
| 2 | mg/L | 1 nM to 10 nM | 211.7 nM | 100-1000 | nM |
| 0.2 | mg/L | 100 pM to 10 nM | 21.17 nM | 10-100 | nM |
| 0.02 | mg/L | 10 pM to 100 pM | 2.117 nM | 1-10 | nM |

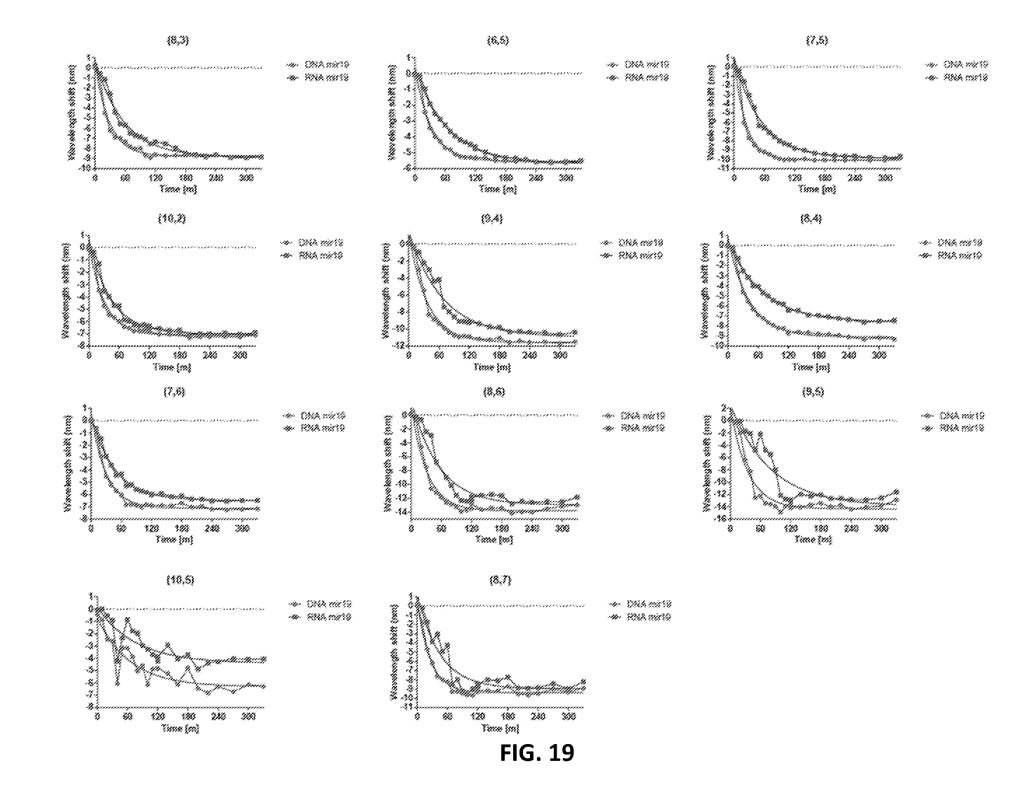

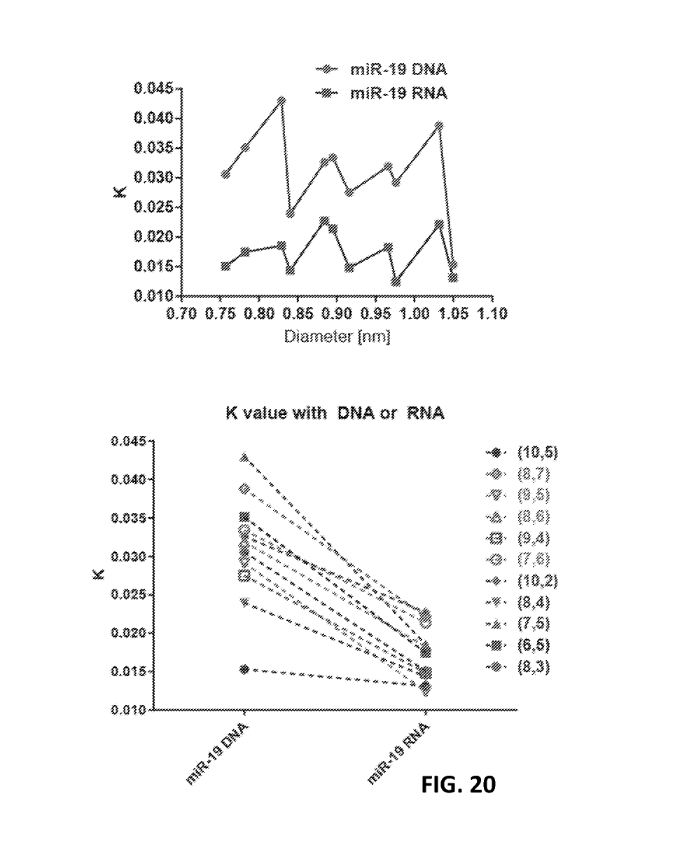

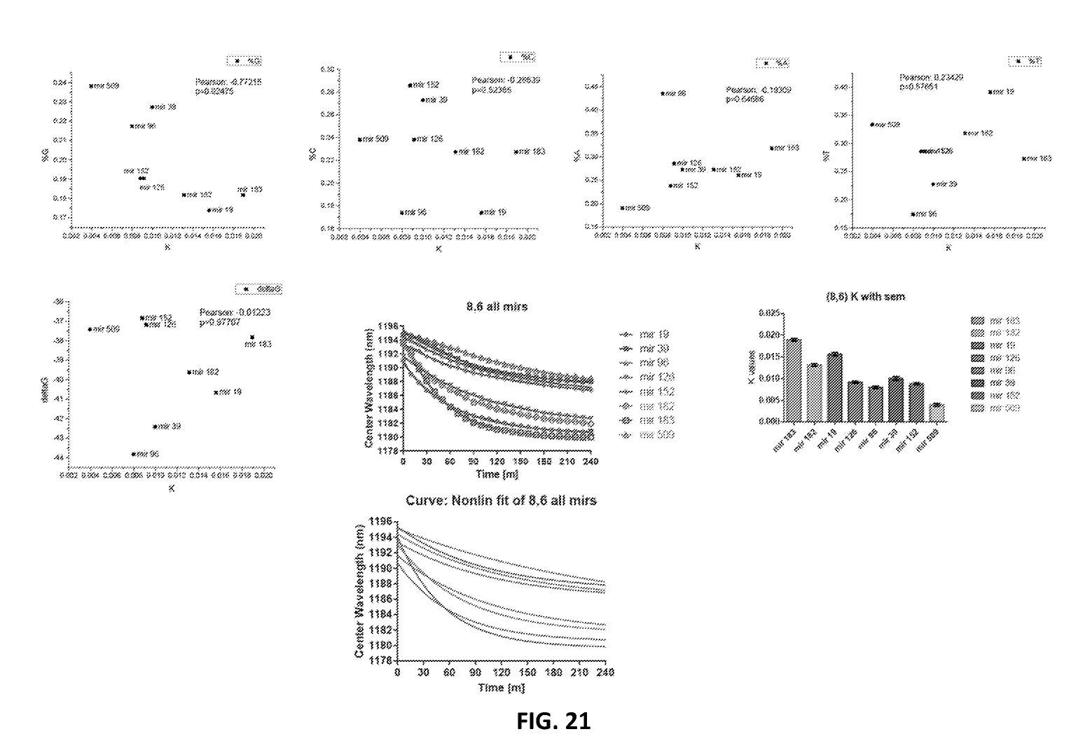

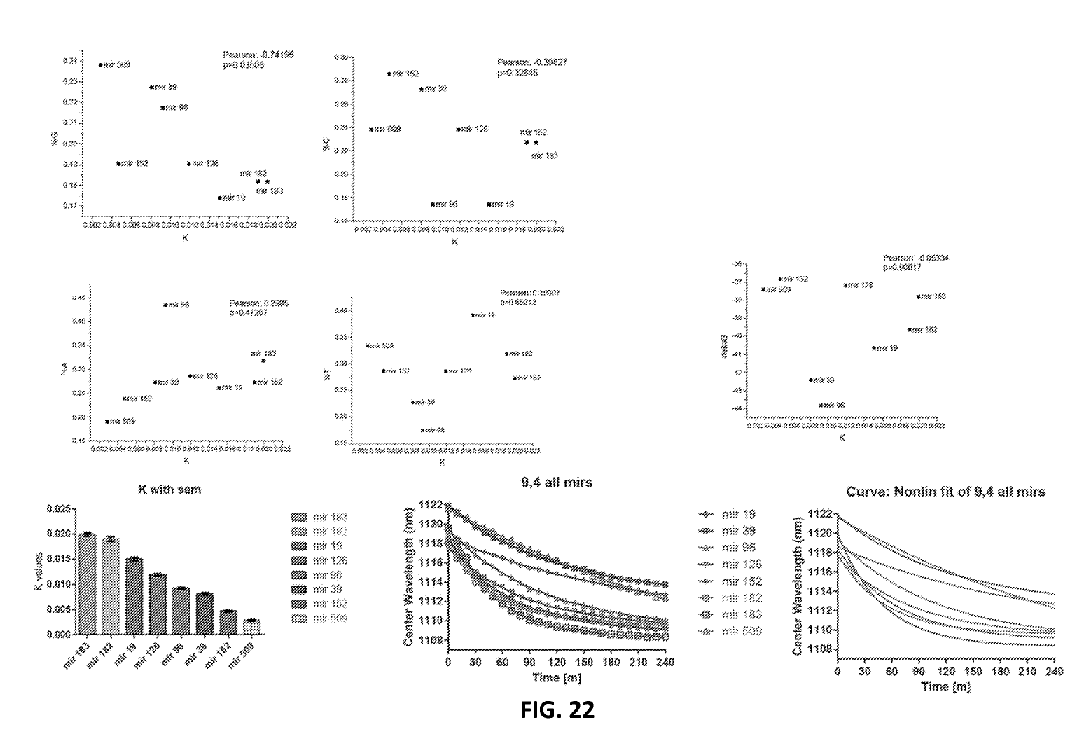

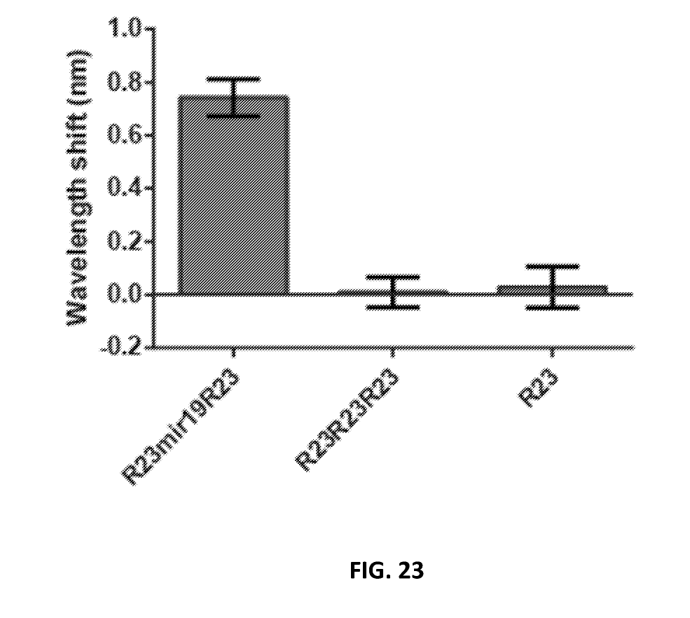

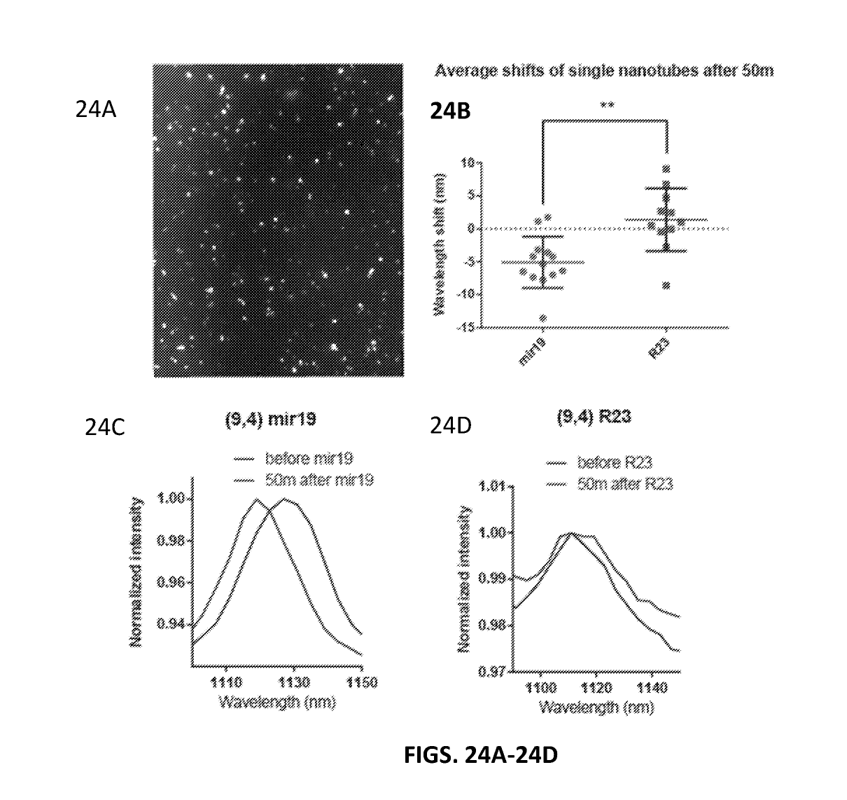

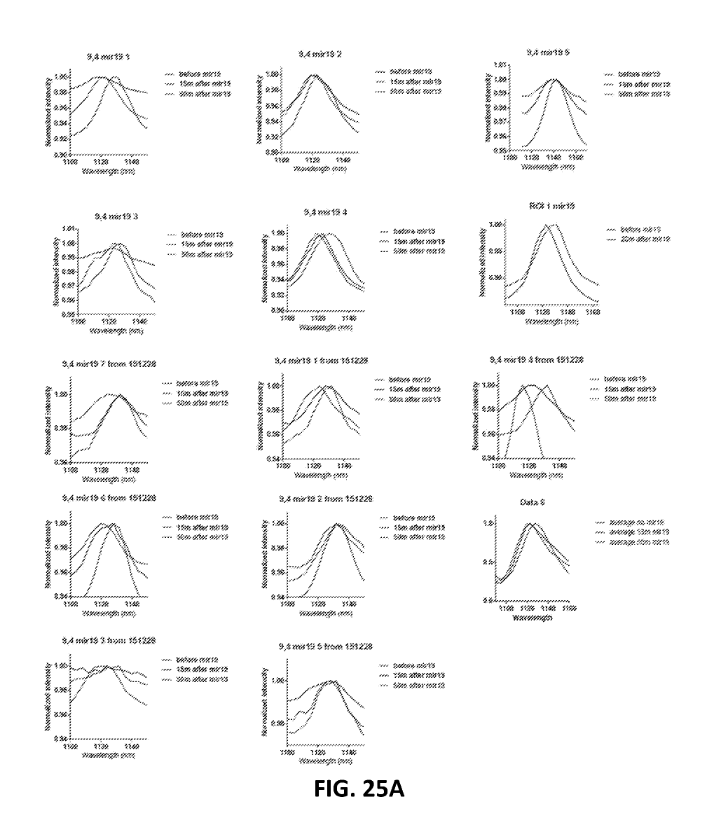

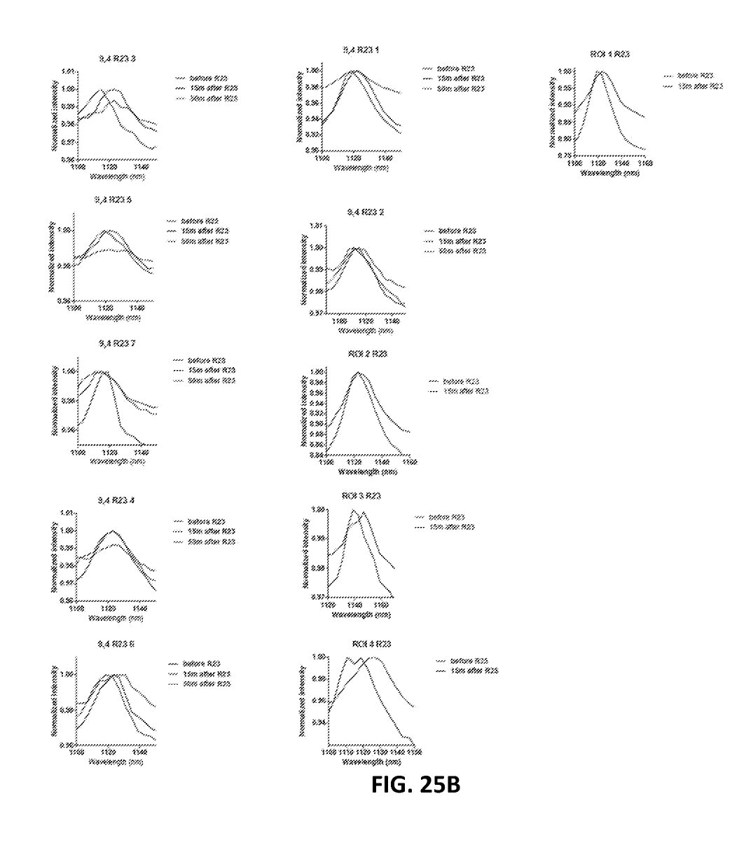

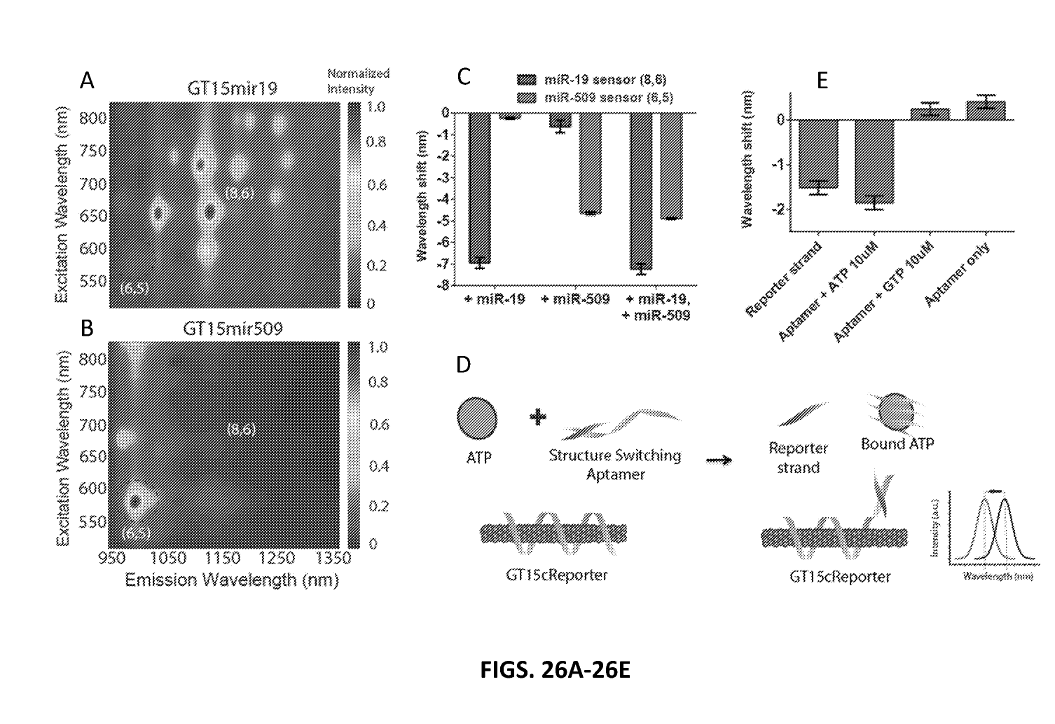

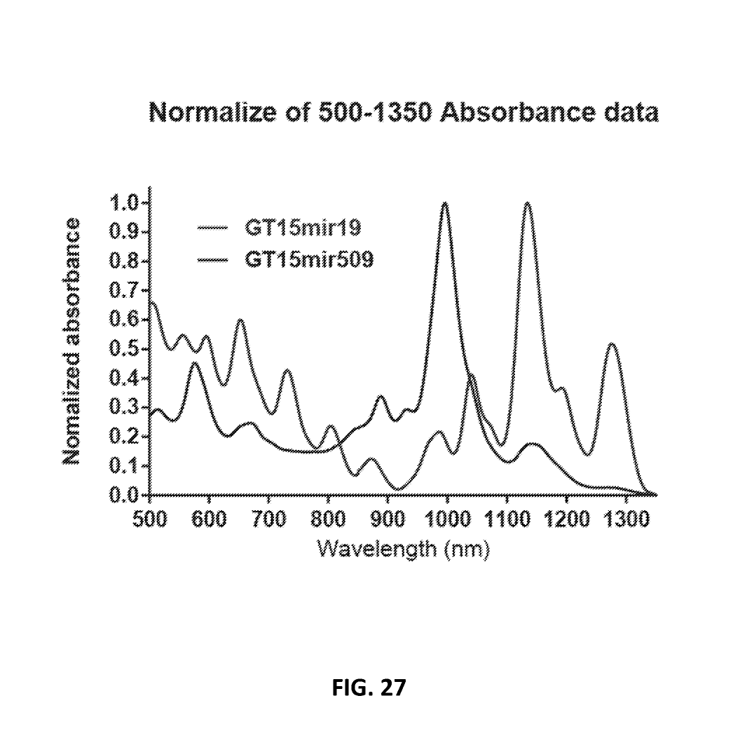

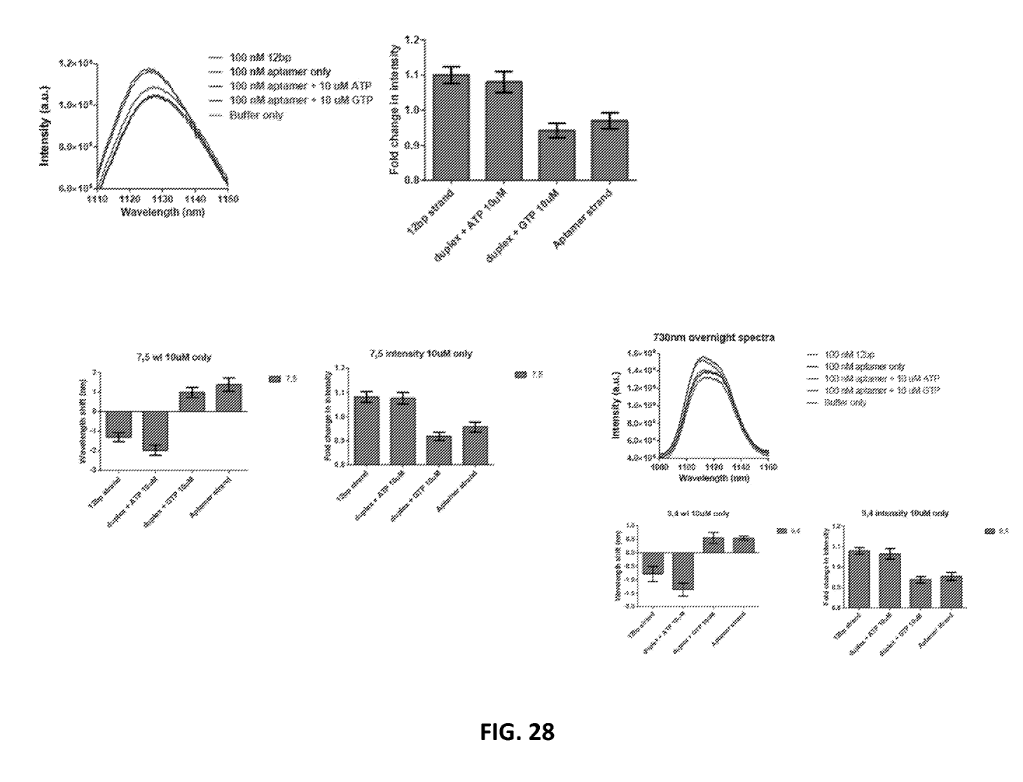

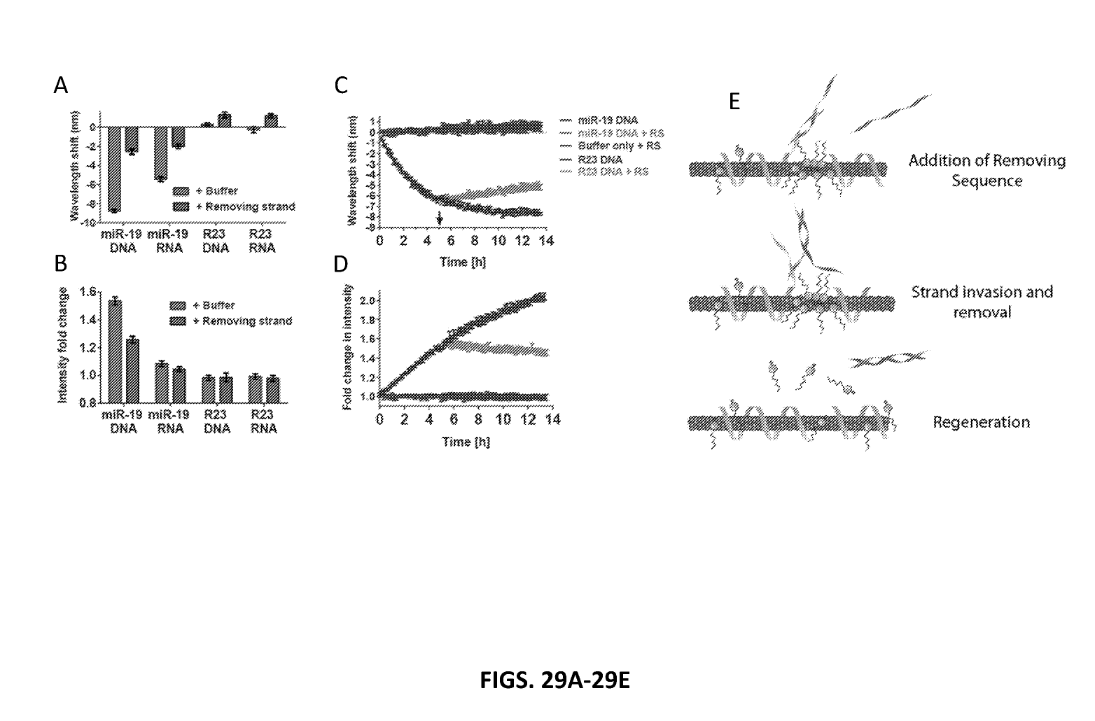

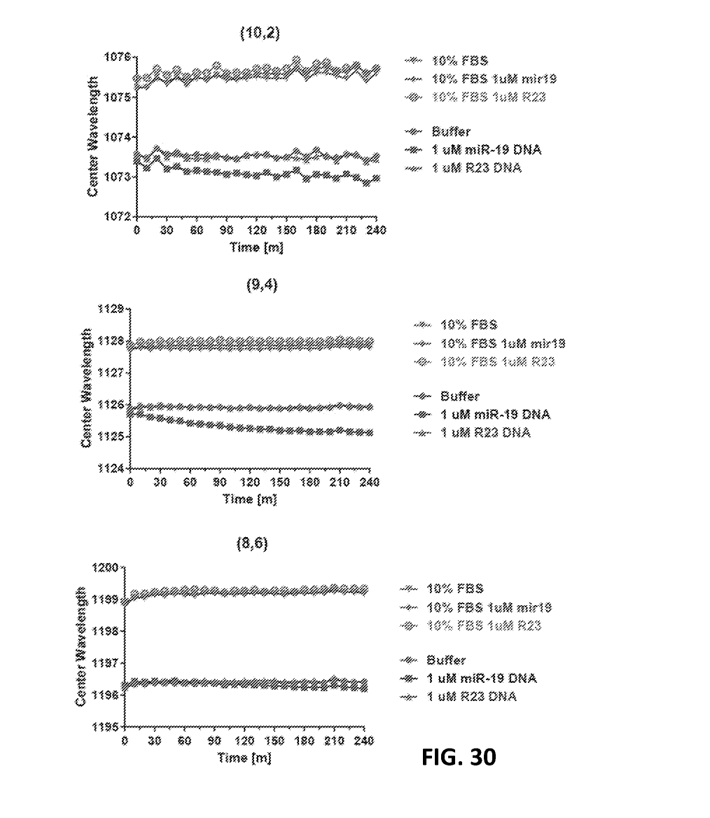

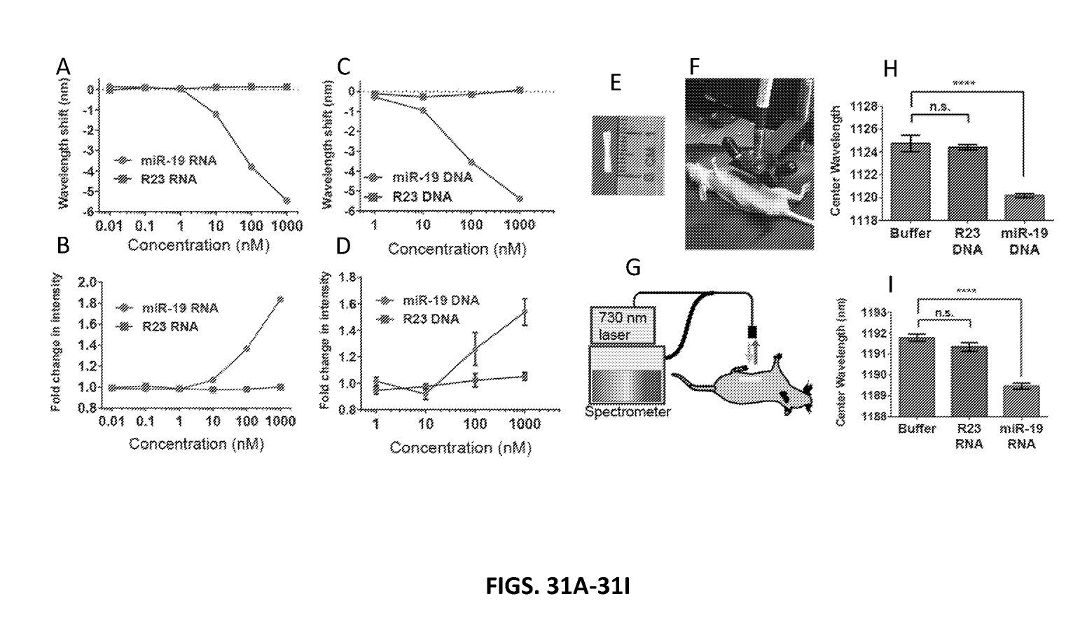

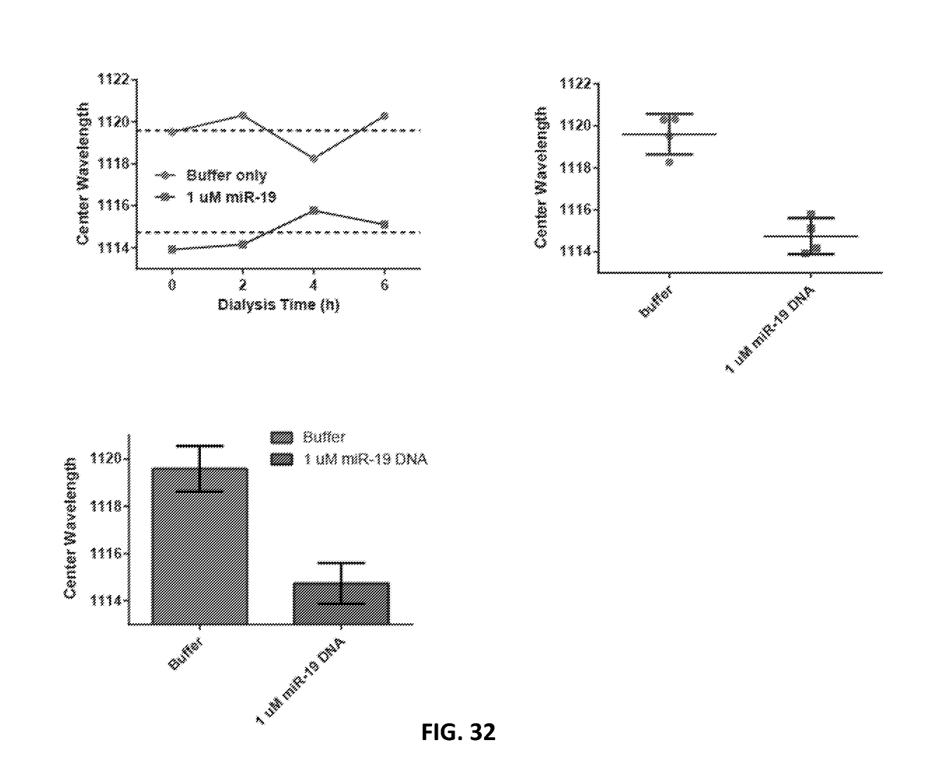

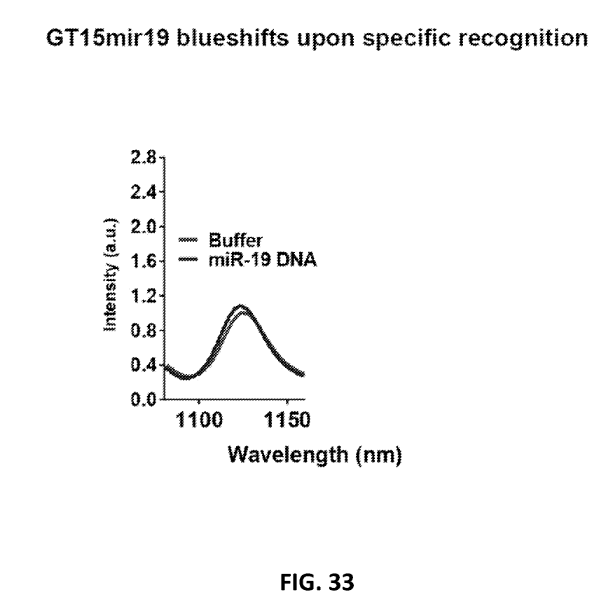

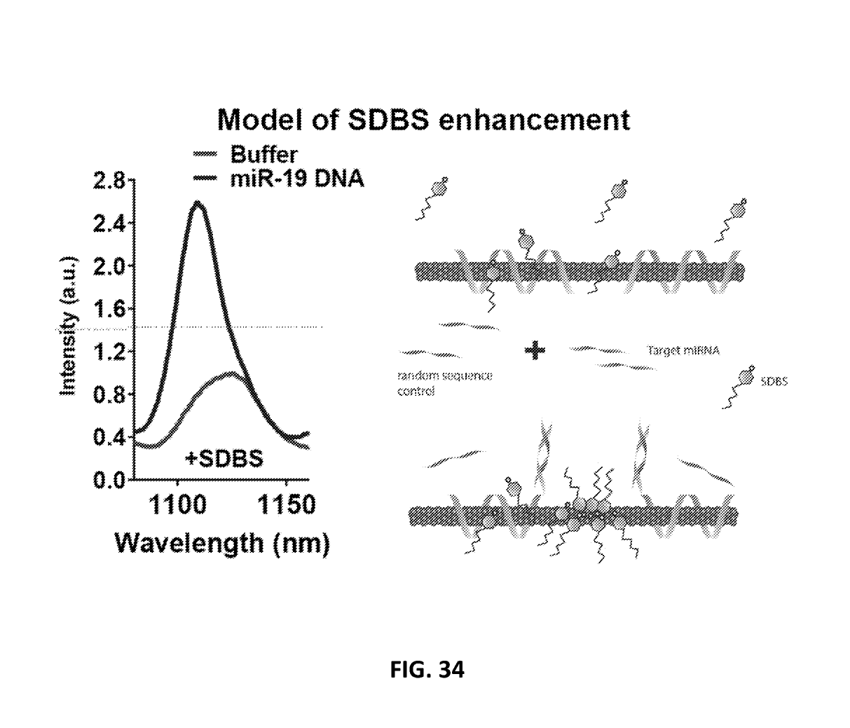

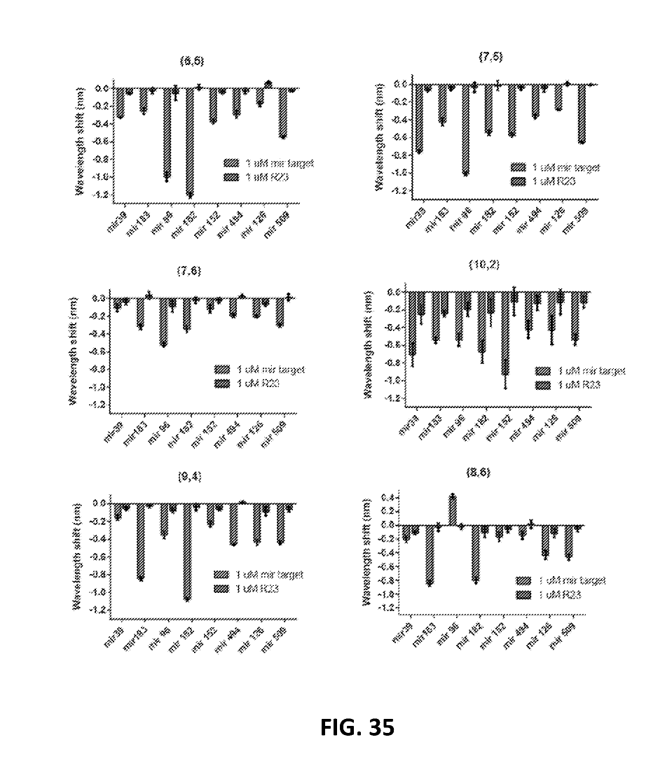

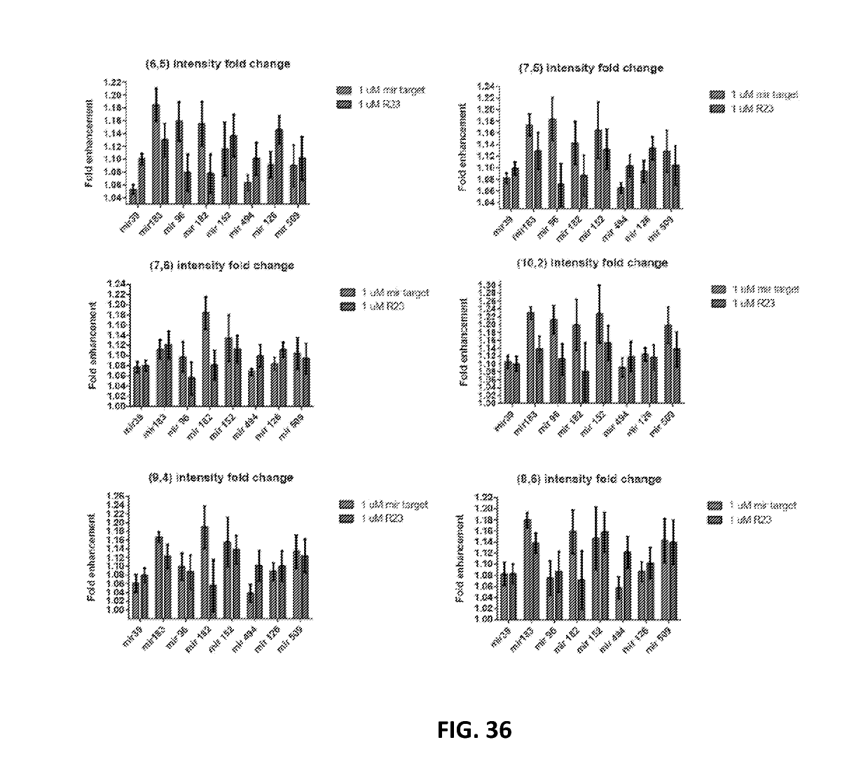

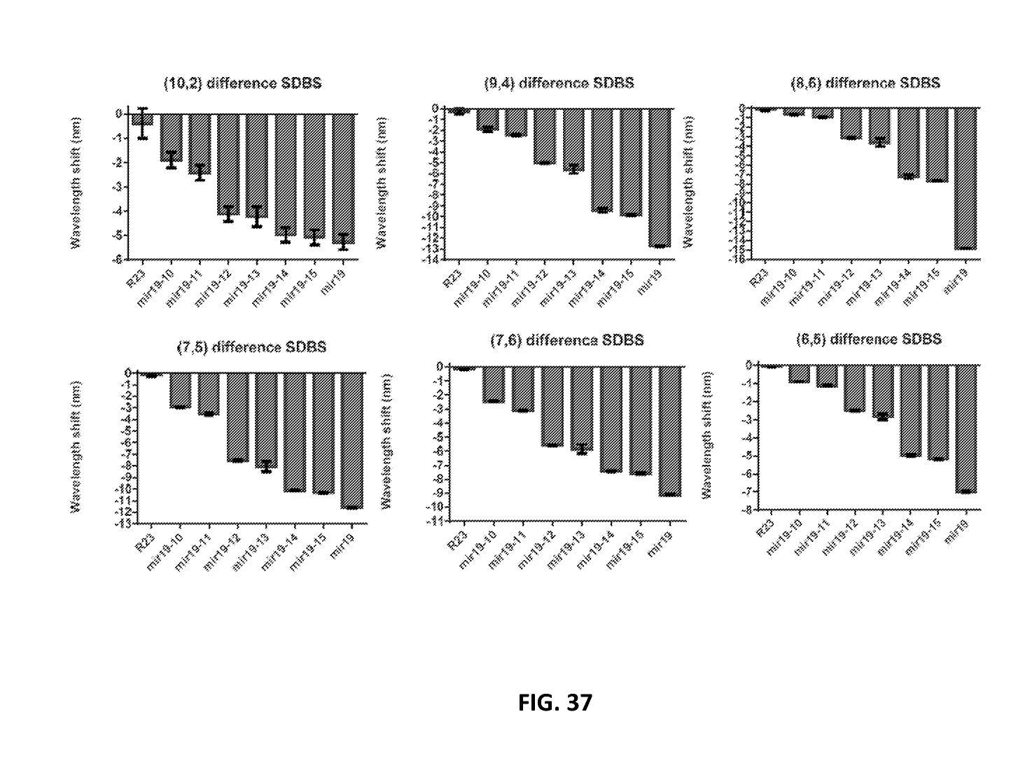

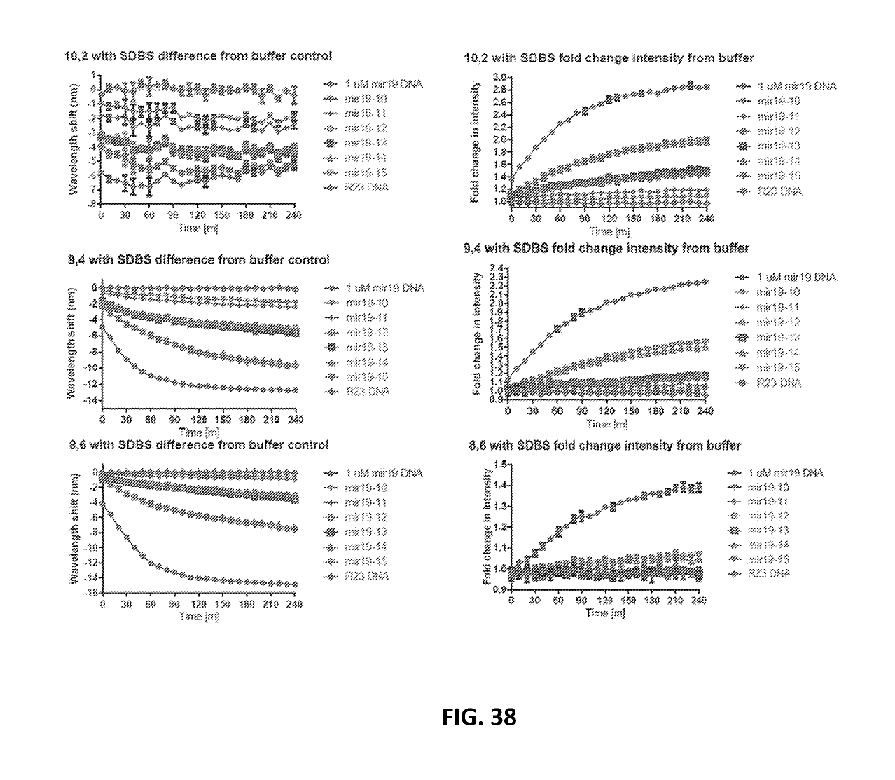

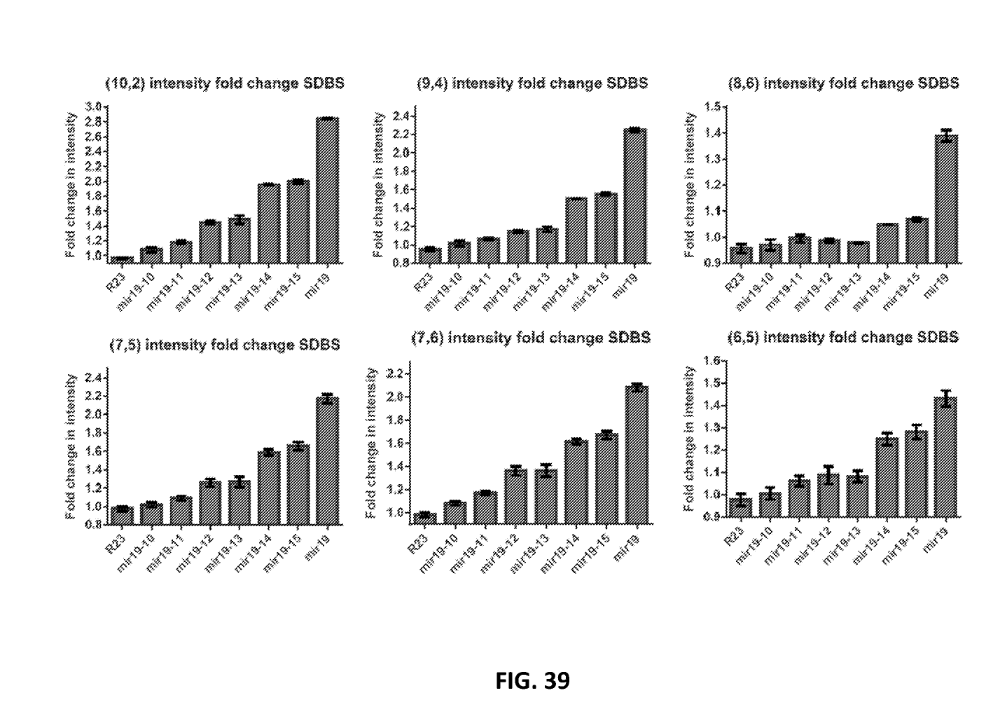

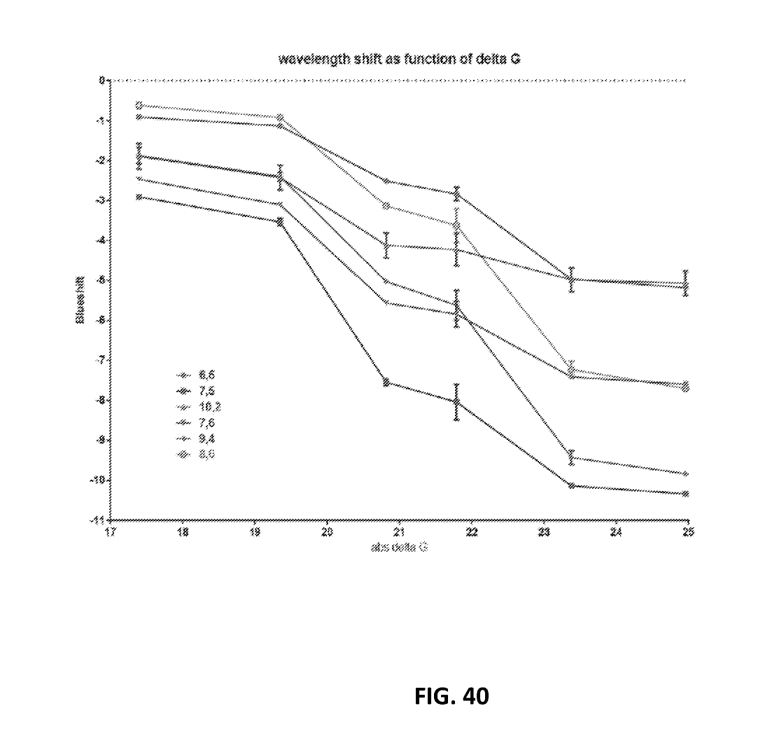

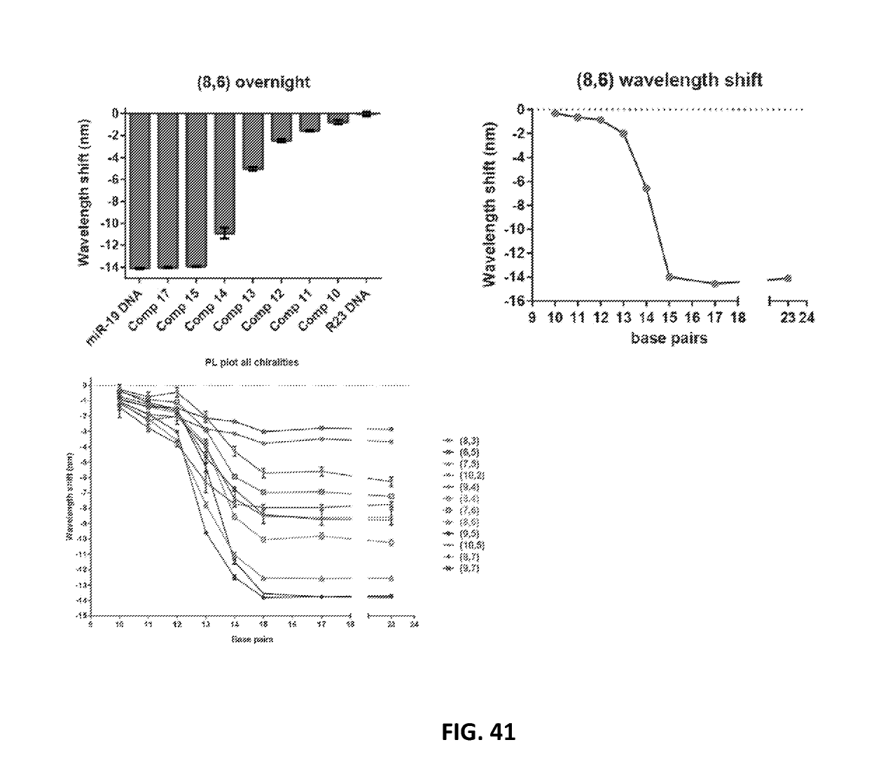

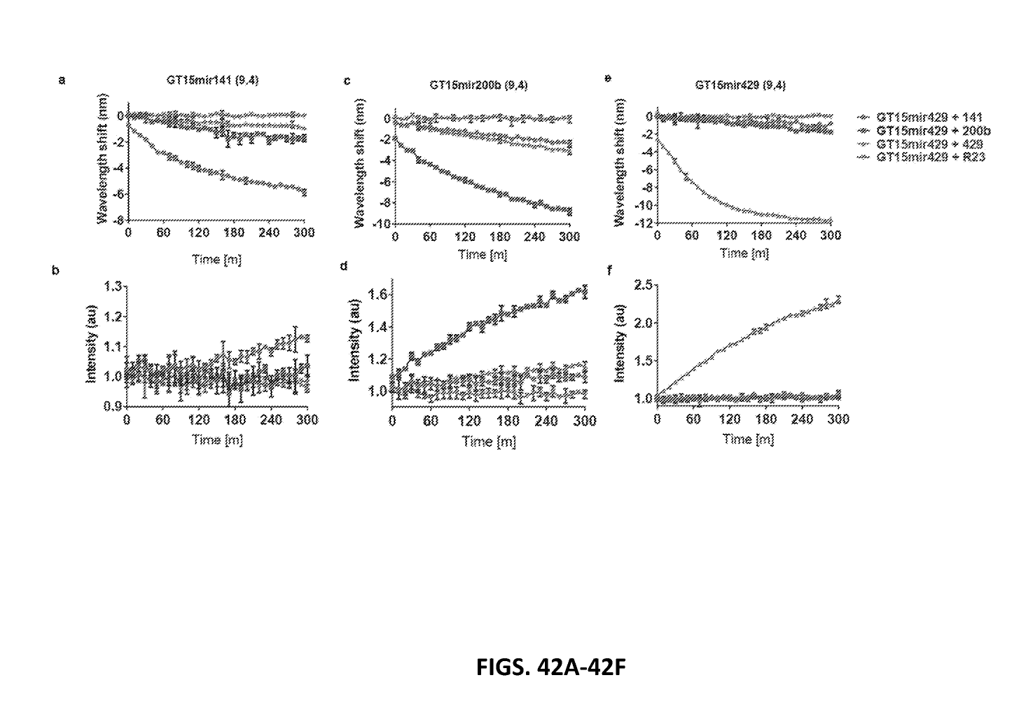

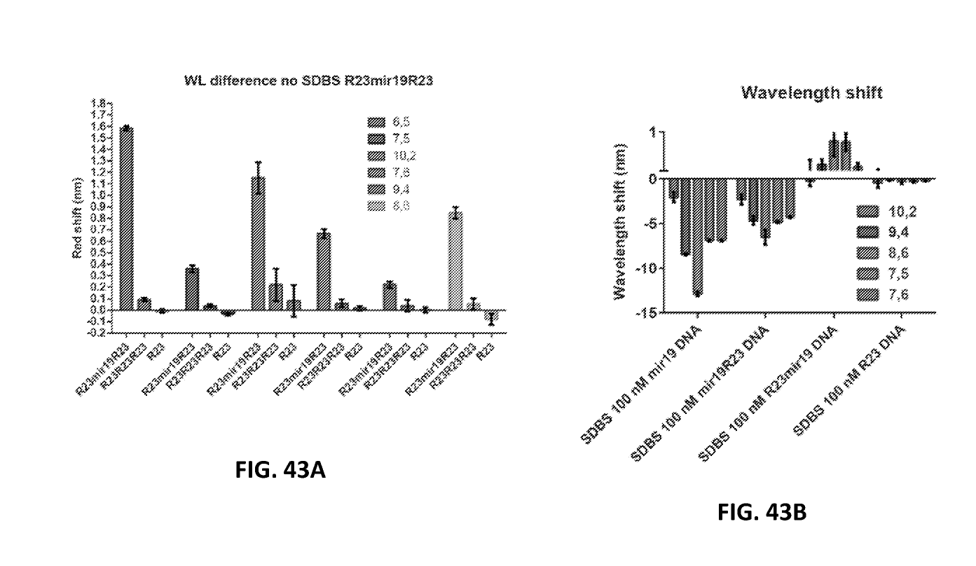

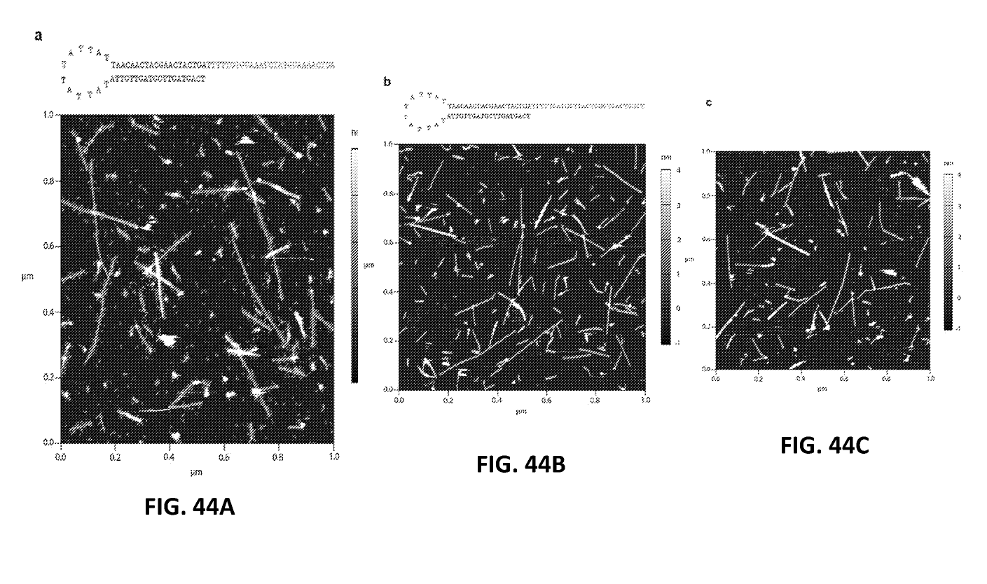

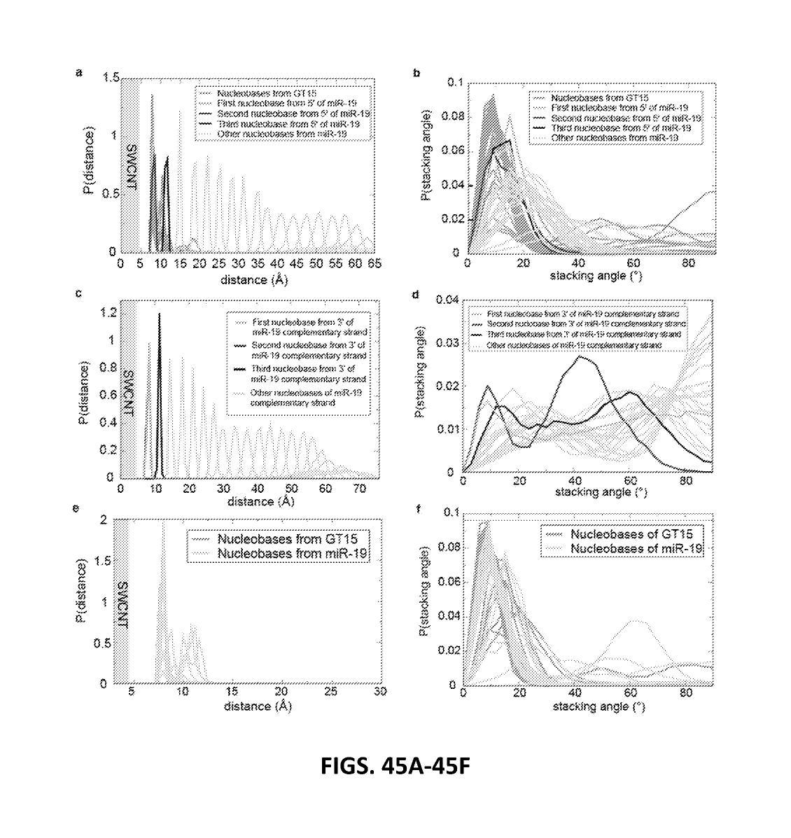

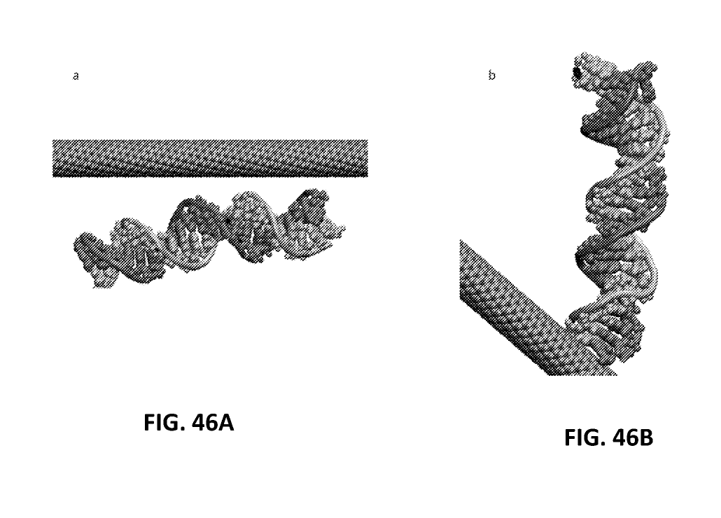

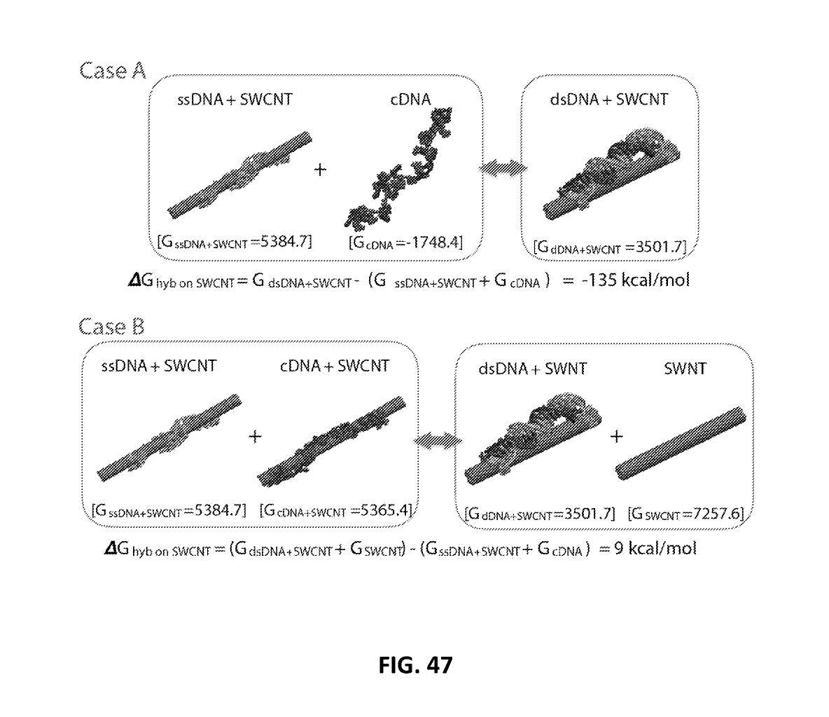

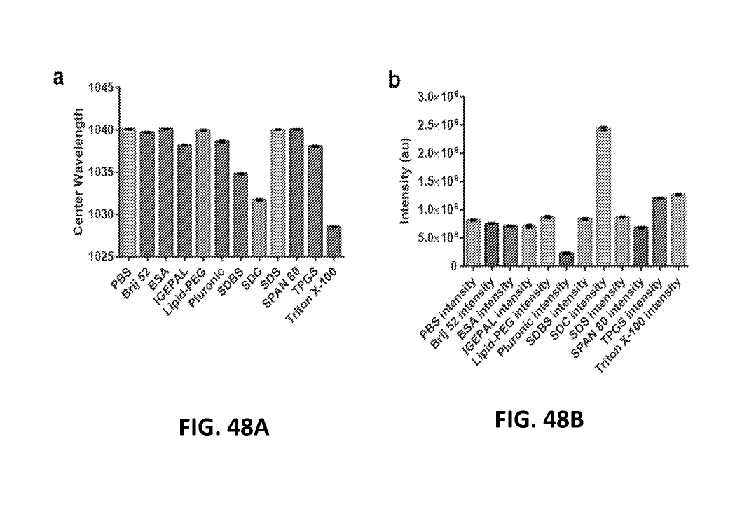

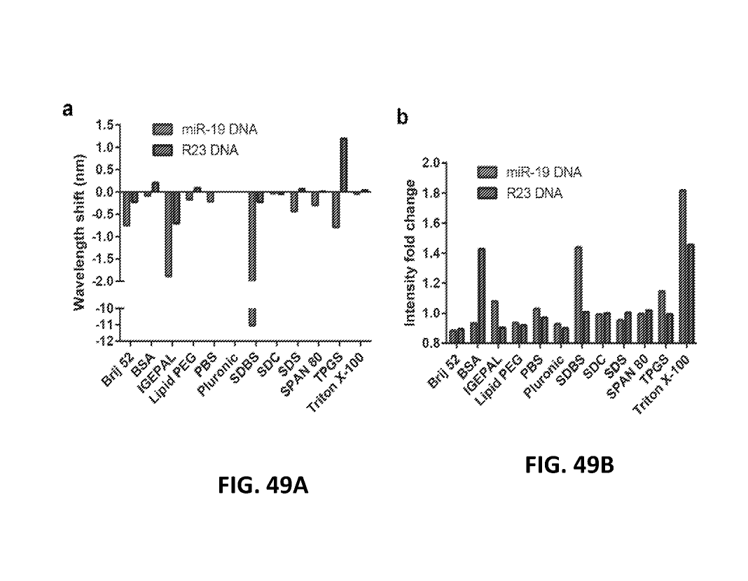

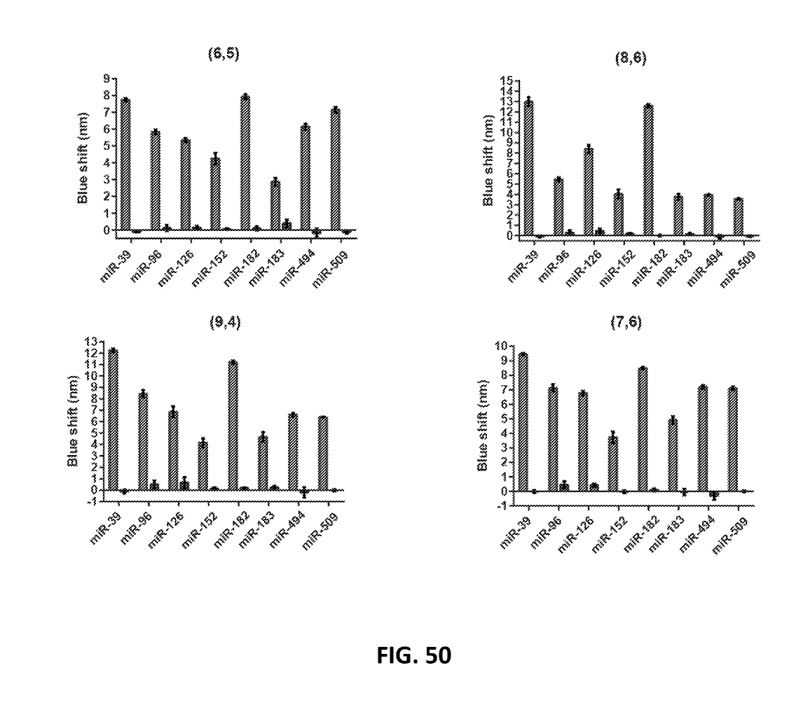

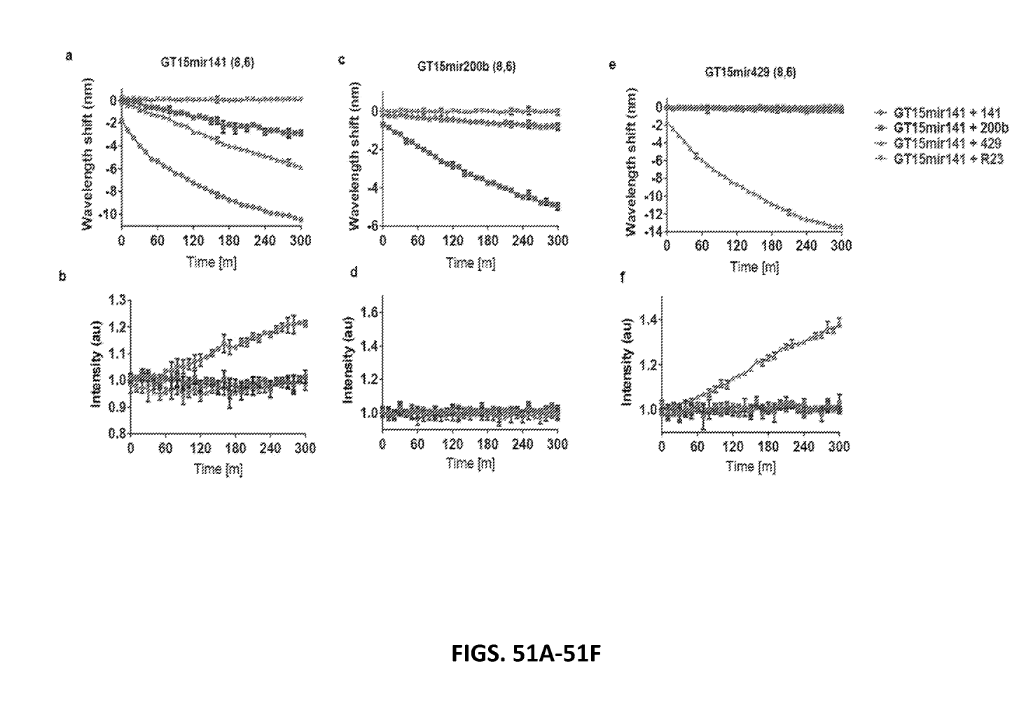

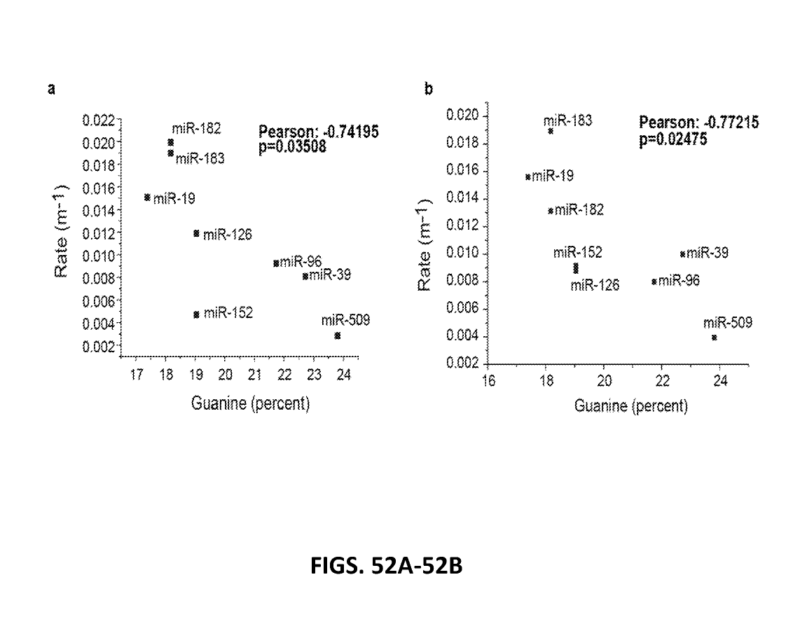

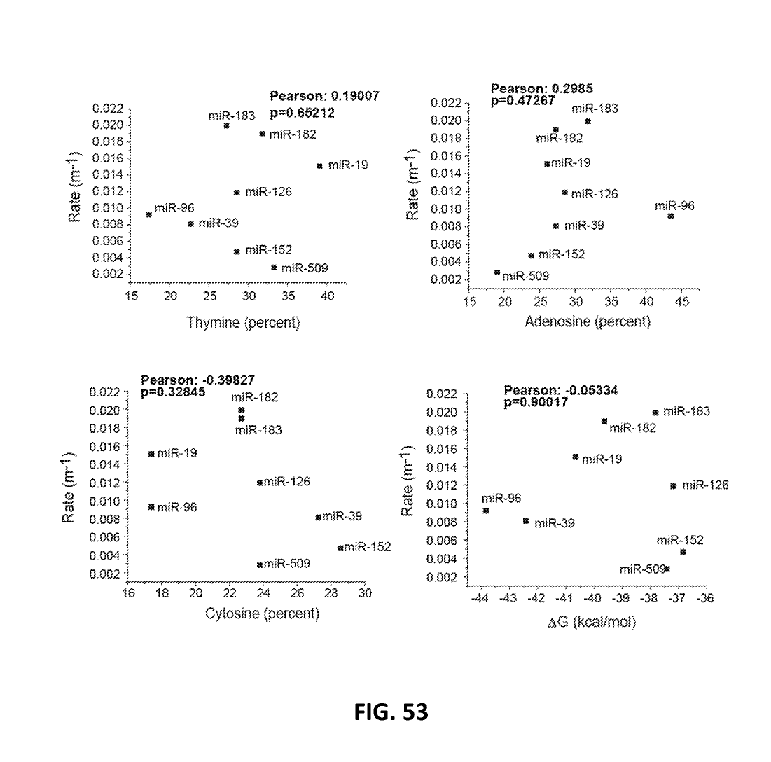

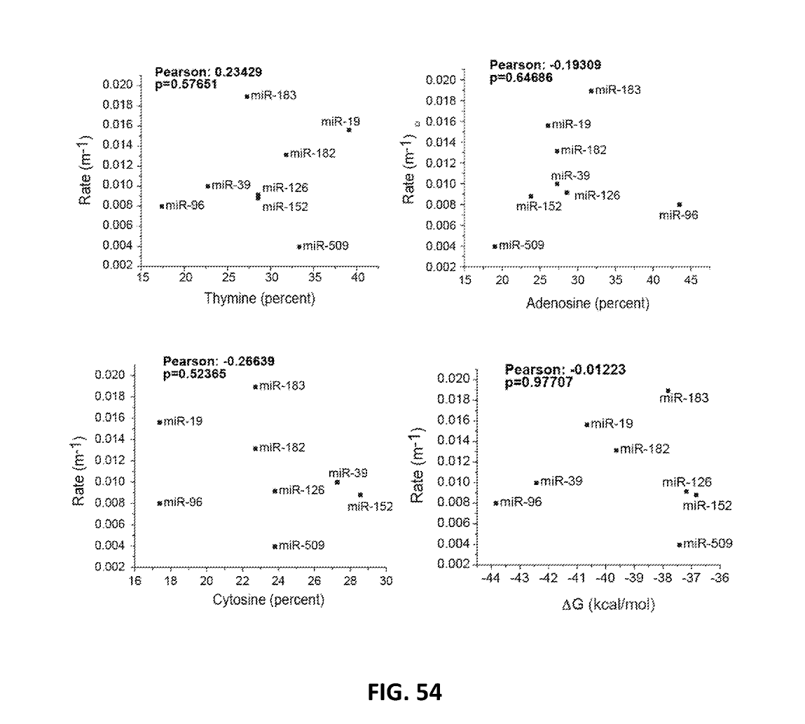

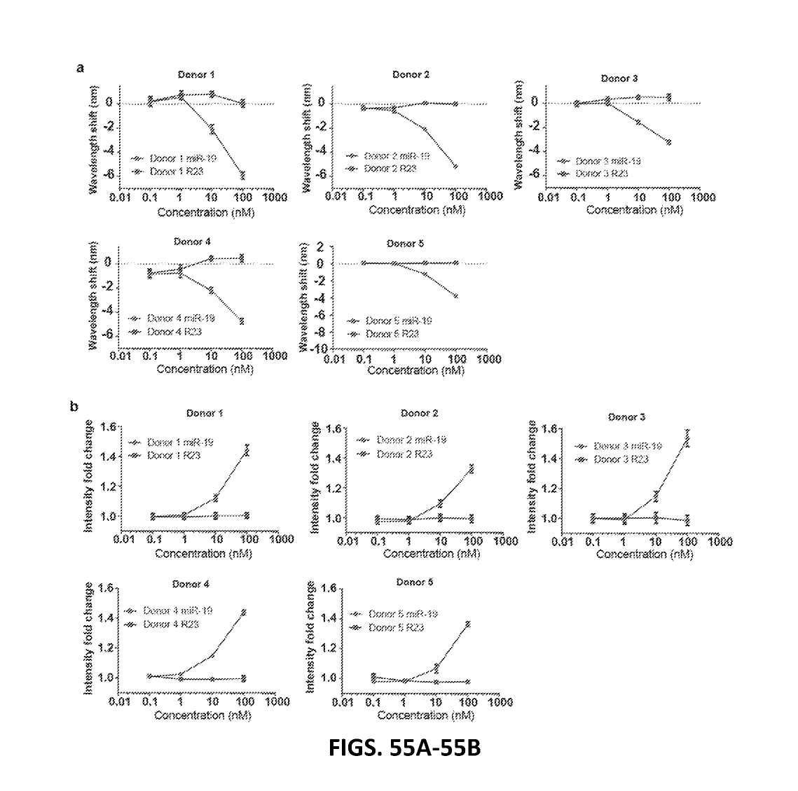

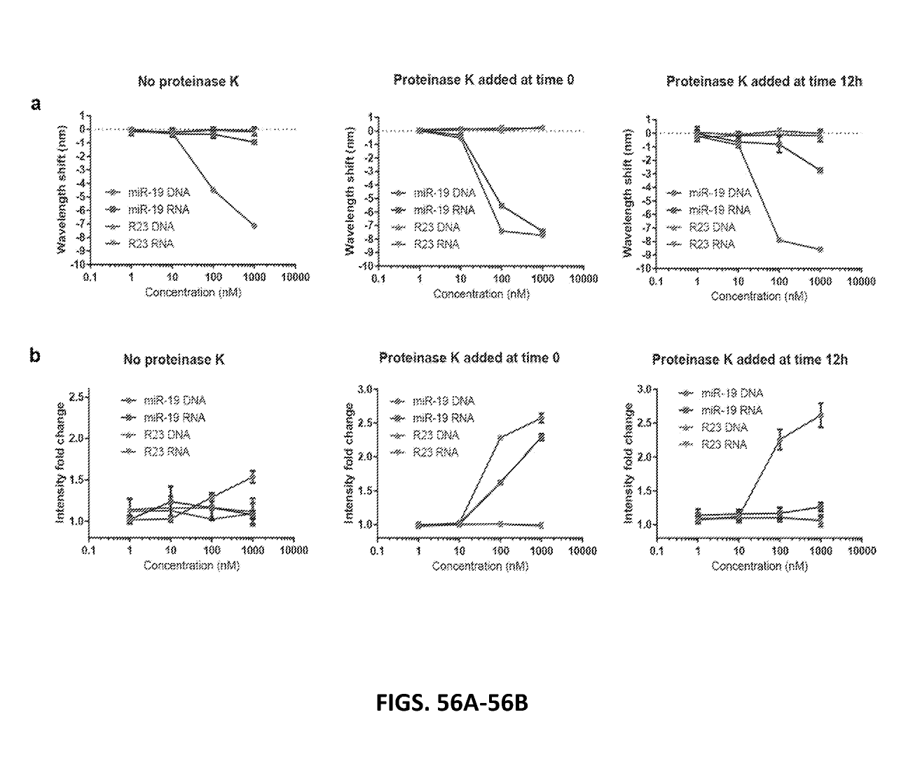

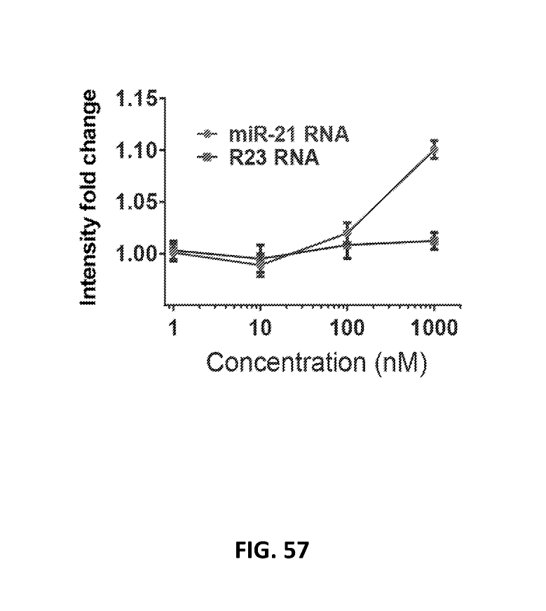

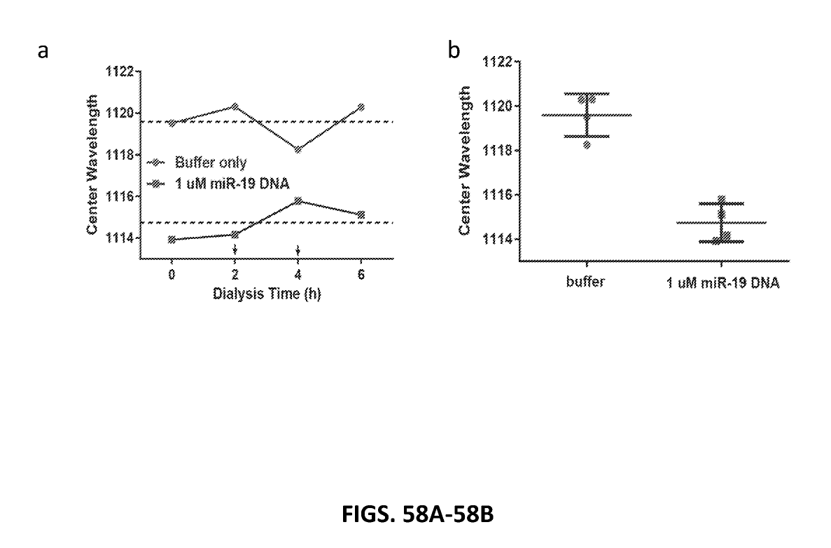

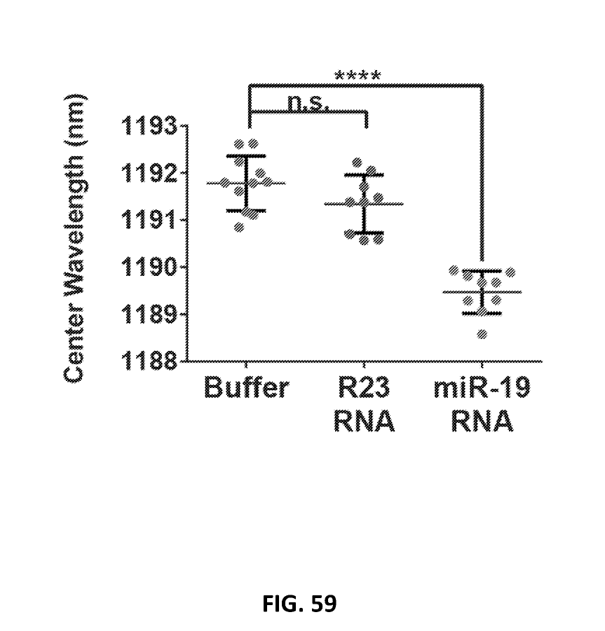

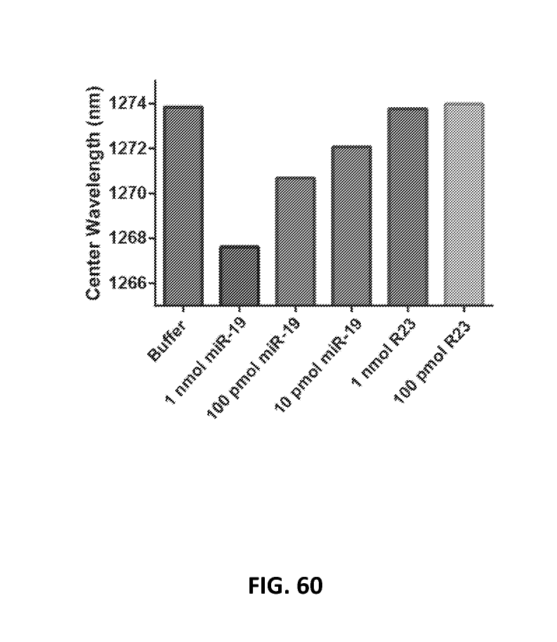

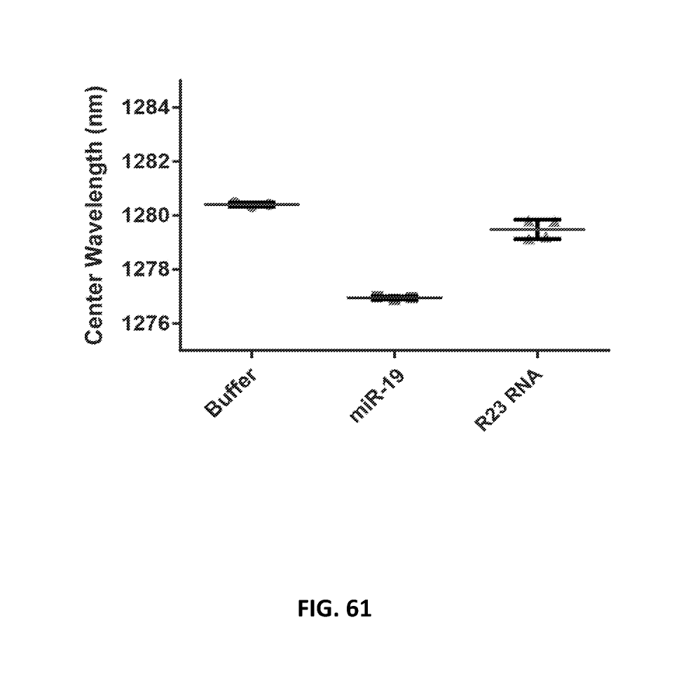

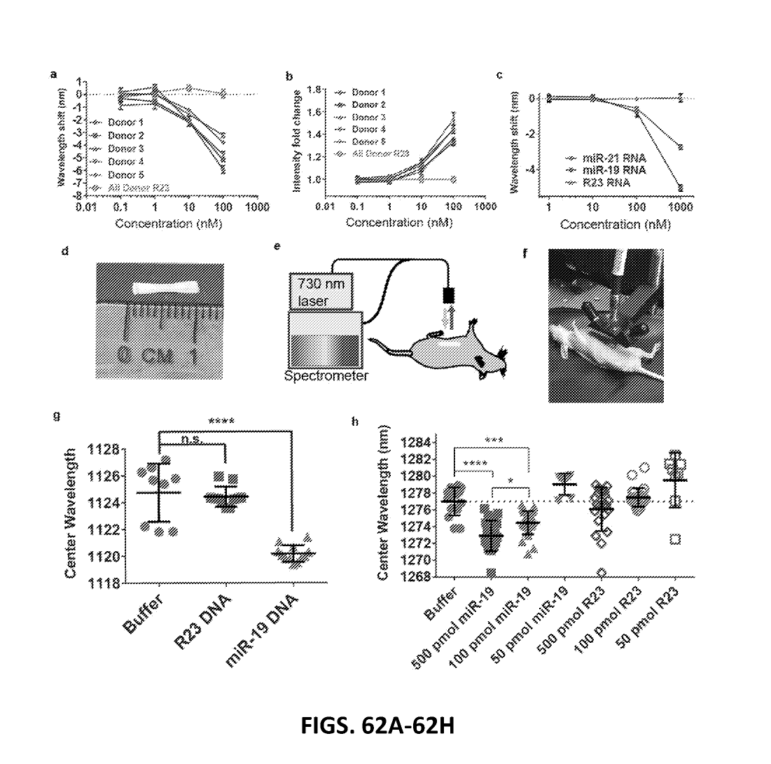

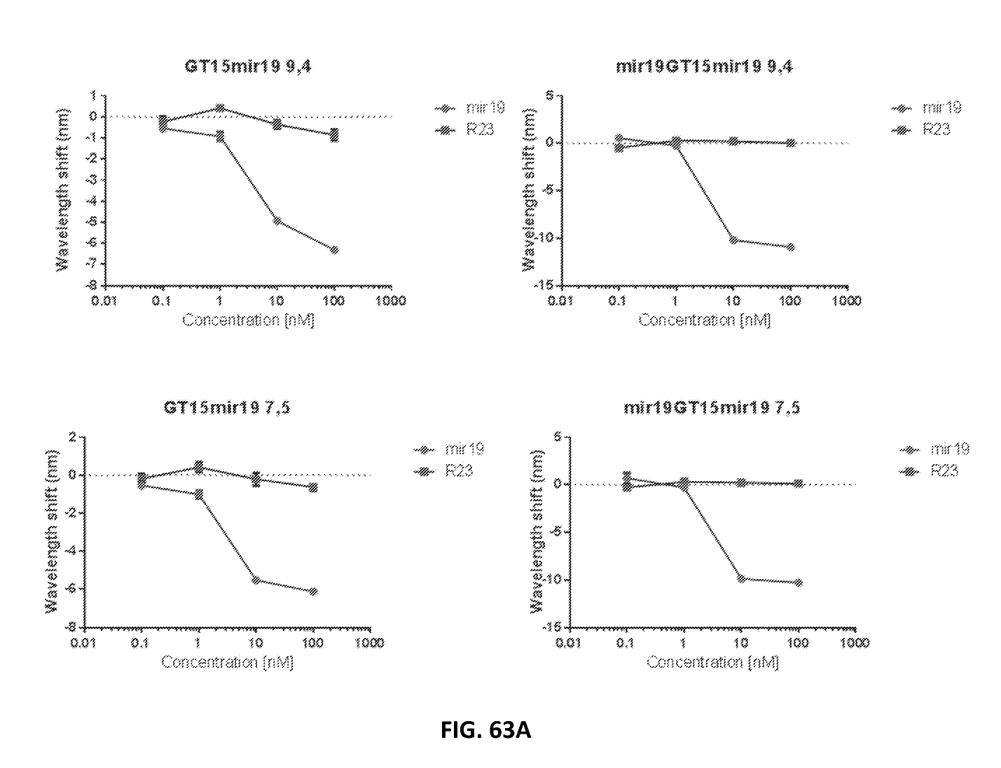

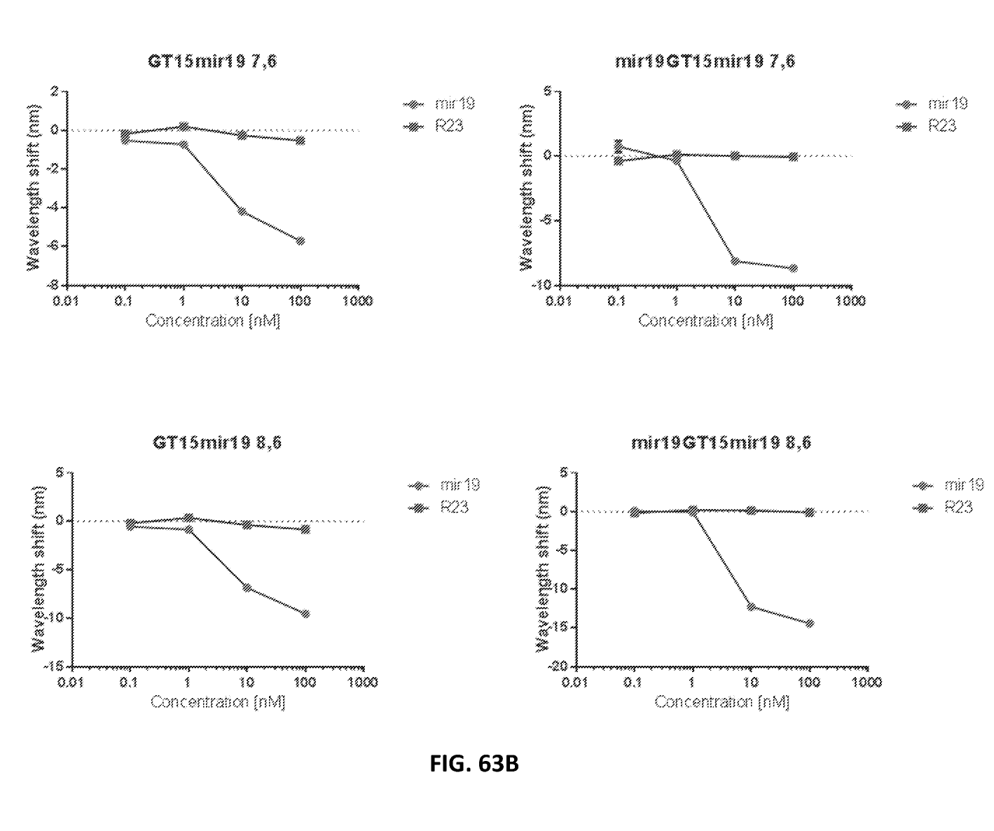

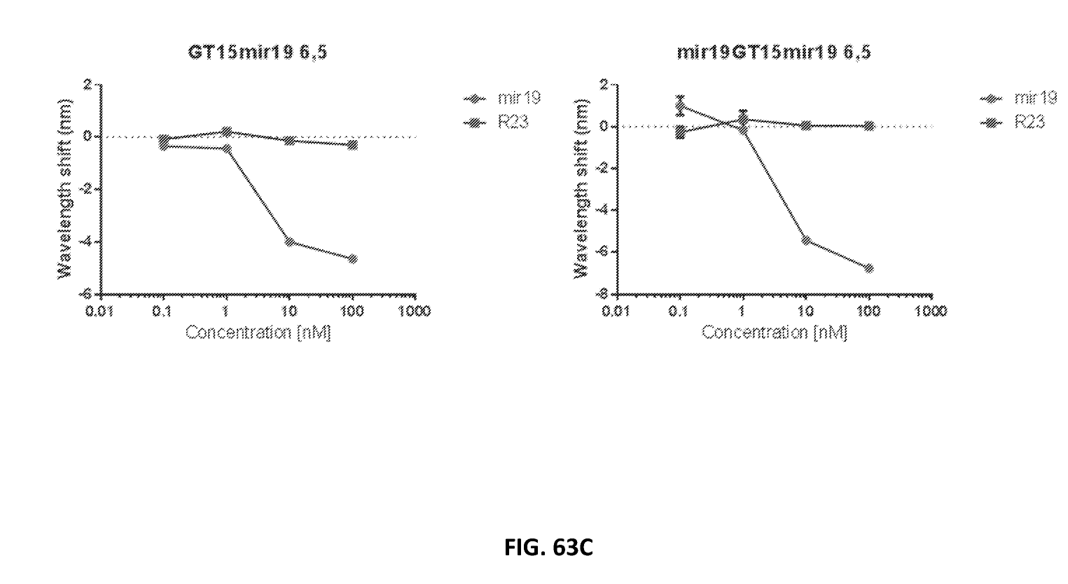

The kinetics of both DNA and miRNA detection were assessed via transient measurements. The kinetics of eleven different nanotube chiralities were measured by excitation/emission spectroscopy (FIG. 19). Within 10 minutes of introducing the analyte to the sensor, significant blue-shifting was discerned. The rate of blue-shifting behaved with pseudo-first order kinetics and showed no obvious dependence on nanotube structure (FIG. 20). The sensor kinetics were consistently faster for DNA (1.8× on average), as compared to RNA (FIG. 16C). Without wishing to be bound to any theory, this difference may be due to the longer persistence length and higher rigidity of single-stranded RNA as compared to single-strand DNA.

To test if the composition of the miRNA capture sequence influenced sensor kinetics, the response rates for the sensor was compared using 8 different miRNA capture sequences (FIGS. 21-22). On comparing the sensor kinetics as a function of guanine content, a significant correlation was found with Pearson coefficients of −0.74195 (p=0.035) for the (9,4) nanotube and −0.77215 (p=0.0248) for the (8,6) nanotube (FIGS. 52A-52B). Without wishing to be bound to any theory, this result may be explained by the high affinity of guanine for the nanotube surface, which was determined previously via both molecular dynamics and ab initio calculations to fall in the order: G>A >T >C. Thus, the affinity of guanine to the nanotube surface may slow the hybridization process. The content of other nucleotide bases, as well as the overall AG of hybridization, did not show any statistically significant correlations (FIGS. 55 and 56).

To better determine how the length and thermodynamics of hybridization relate to the optical response of the nanotube, several experiments were conducted using modified analyte oligonucleotides. The G15mir19 sensor was interrogated using analyte sequences between 10 and 23 nucleotides long which were complementary to either the 3′ terminal end of the miRNA capture sequence, or the middle of the sequence, as depicted in FIG. 16E. It was found that, in general, a shorter analyte sequence resulted in a smaller blue-shifting response of the nanotube, down to approximately 10 nucleotides, where there was virtually no response (FIG. 16J). Additionally, the magnitude of the blue-shifting response was consistently smaller when the analyte sequence was designed to hybridize to the middle of the capture sequence. Without wishing to be bound to any theory, this difference may be explained by the affinity of the capture sequence to the nanotube, as suggested by the results of the fluorophore quenching experiment, MD simulations, and free energy analyses.

To assess its broad applicability for the detection of different nucleic acid types, it was determined whether the sensor could detect oligonucleotides longer than miRNA sequences. First, it was determined how the GT15mir19 sensor would respond to a long oligonucleotide designed to contain a complementary sequence flanked by non-complementary sequences (FIG. 16K). On interrogating the sensor with R23-mir19-R23, a 69-bp oligonucleotide with 23 complementary bases in the middle of the sequence, the sensor emission red-shifted—opposite of the blue-shifting response—even in the presence of SDBS (FIG. 16L, shown for the (8,6) chirality). Without wishing to be bound to any theory, it was therefore hypothesized that, when R23-mir19-R23 hybridizes to the recognition sequence, the R23 portion at the 5′ end may disrupt the sensor function by increasing the phosphate content near the nanotube surface to cause a red-shift of the nanotube emission. To test this hypothesis, two long oligonucleotide sequences, R23-mir19 and mir19-R23, were designed by placing the R23 portion at either the 3′ end or 5′ end. The proposed orientation upon binding and predicted spectral shifts are shown in FIG. 16G—the two sensors were hypothesized to give the opposite spectral responses. Upon interrogating the sensor, the R23-mir19 oligonucleotide produced a red-shifting response, and the mir19-R23 oligonucleotide produced a blue-shift, as predicted (FIG. 16H). The magnitude of the blue-shift in response to mir19-R23 was smaller than that produced by the miR-19 control, thereby suggesting that the unhybridized single-stranded nucleotides may bind to the nanotube surface, diminishing the response.

The sequence for R23mir19R23 is as follows:
(SEQ ID NO: 52)
TGATCGTTACTGGGTGACTGGCTAGTCAAAACGTATCTAAACGTGTGATC
GTTACTGGGTGACTGGCT.
The sequence for mir19R23 is as follows:
(SEQ ID NO: 53)
AGTCAAAACGTATCTAAACGTGTGATCGTTACTGGGTGACTGGCT.
The sequence for R23mir19 is as follows:
(SEQ ID NO: 54)
TGATCGTTACTGGGTGACTGGCTAGTCAAAACGTATCTAAACGTGT.

Sensors based on the GT15 (SEQ ID NO: 1) nanotube binding domain and a general capture sequence can be extended to detect longer nucleic acid sequences, but the orientation of the oligonucleotide is critical for eliciting a desired spectral response.

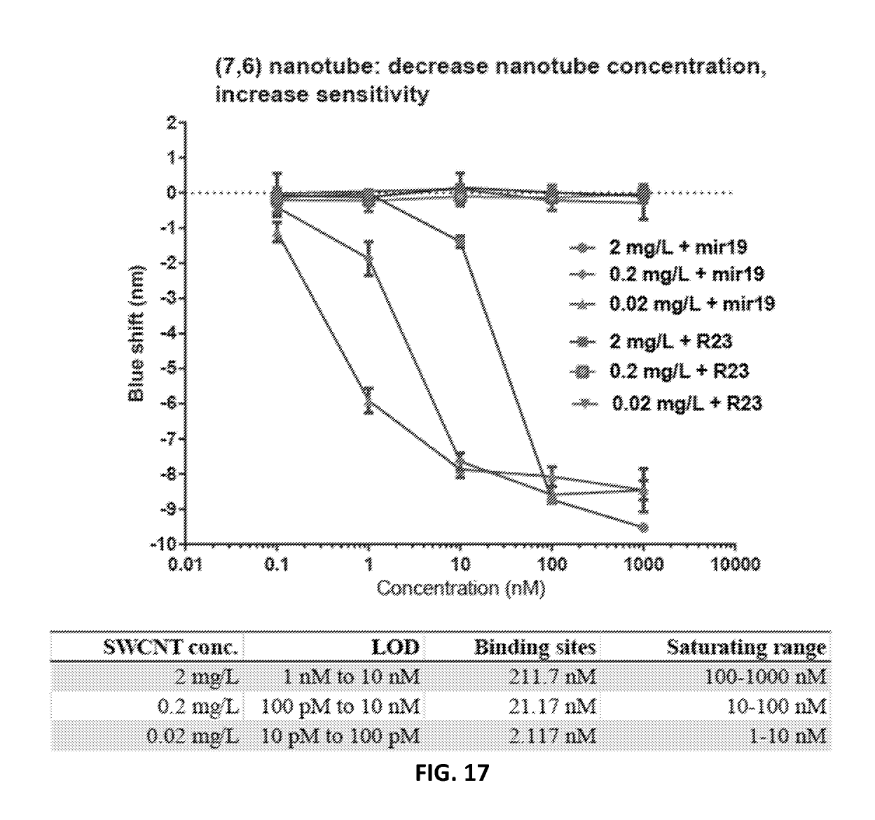

To determine the limit and range of detection, a dose-response over several orders of magnitude was constructed (FIG. 17). By varying the concentration of nanotube, it was found that the dynamic range and limit of detection depends on the ratio of nanotube to target miRNA. At the lowest nanotube concentration that could be practically detected in solution, 0.02 mg/L, the limit of detection was found to be between 10 and 100 pM (FIG. 16B), with saturation (no additional blueshift) occurring between 1 and 10 nM. By varying the concentration of the nanotubes, it was possible to cover 5 orders of magnitude, from 10 pM to 1 μM (FIG. 17). To better determine this binding behavior, the number of binding sites in a given concentration of nanotubes was estimated by quantifying the amount of DNA used to suspend the nanotubes and the mass of unbound DNA that is removed by spin-filtration. From three suspensions, it was found that 3.5 (+/−1.8) mg of DNA suspends 1 mg of SWCNT, matching previous reports of 2.5 to 5 mg of DNA per 1 mg of SWCNT (See Zheng, M. et al. Nat. Mater. 2, 338-42 (2003)). For a SWCNT concentration of 0.02 mg/L, this corresponds to 2.117 nM of miR-19 binding sites, consistent with the observed saturating range. Without wishing to be bound by theory, when there are too many unoccupied binding sites relative to occupied binding sites, a net-blueshift is no longer distinguishable. Based on the observed limit of detection, when less than 5 to 0.5% of binding sites are occupied, a blueshift is no longer observable.

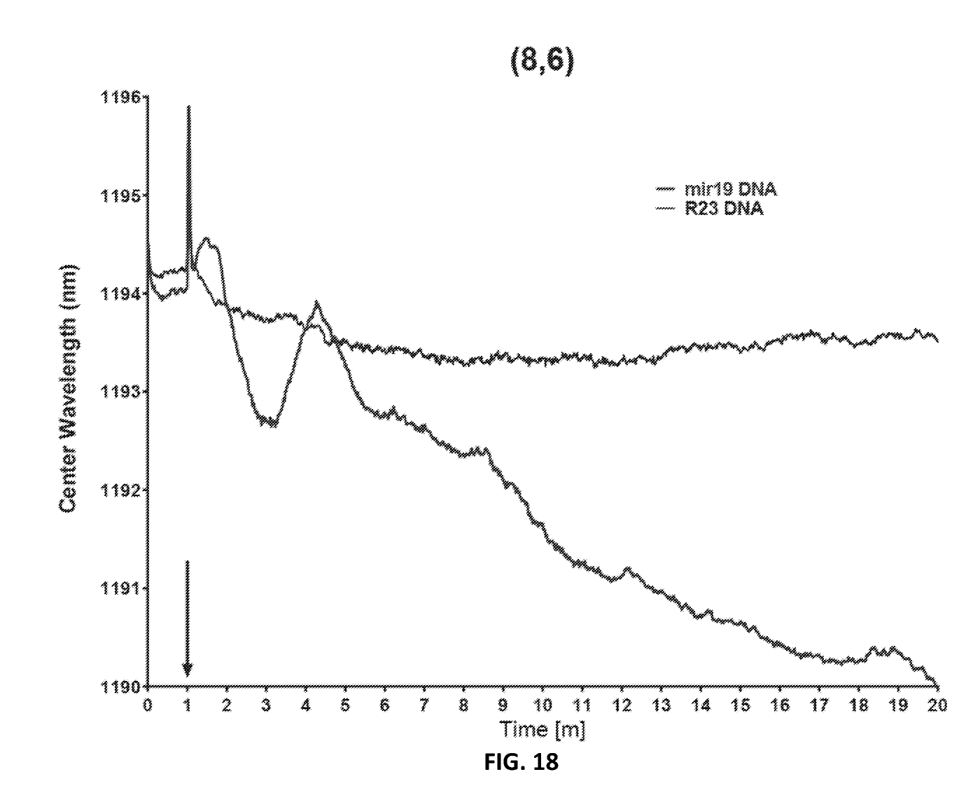

The kinetics of blueshifting were rapid, with changes evident within 5 minutes of miR-19 DNA addition (FIG. 18). To investigate potential chirality-dependent effects on kinetics, a series of PL plots after addition of miR-19 DNA and RNA to measure eleven different chiralities were fit to exponential decay functions (FIG. 19). As a representative plot, the kinetics of blueshifting and fits for the (7,5) nanotube are shown in FIG. 16C. The rate of blueshifting with miR-19 DNA was relatively constant across the measured chiralities (FIG. 20), with maximum blueshifting occurring around 90 minutes. The rate of blueshifting with miR-19 RNA was also fairly constant, but for every chirality measured the blueshifting was modestly slower than the matched DNA (FIG. 16C and FIG. 20). Without wishing to be bound by theory, the slower kinetics for RNA may be related to the shorter contour length, longer persistence length, and higher rigidity of single-strand RNA than single strand DNA.

Both molecular dynamics and ab initio calculations of nucleic acid interaction strengths with carbon nanotubes predict binding strengths in the order of G>A>T>C (See Johnson, R. R., et al. Small 6, 31-4 (2010)). To test if the base composition of the target recognition sequence initially bound to the nanotube played a role in the kinetics of blueshifting, the fitted rates for 8 different miR sequences whose recognition sequences had varying amounts of each base were compared. For the two chiralities measured, (9,4) and (8,6), a statistically significant correlation with the percent of guanine in the recognition sequence and the rate of blueshifting was found (FIG. 16D), showing that a higher percentage of guanine is negatively correlated with the rate. Other bases and the AG of hybridization did not show any statistically significant correlations (FIG. 2I and FIG. 22).

To better determine how the length and thermodynamics of hybridization relates to blueshifting of the nanotubes, truncated target sequences ranging from 10 to 15 nucleotides that can bind either from the 3′ end or the 5′ portion in the middle of the recognition sequence were used (depicted in FIG. 16E). For the same AG of binding, sequences that start in the middle of the recognition sequence had an attenuated response, whereas sequences that bind from the end of the recognition sequence steadily increased in blueshifting with more negative ΔG (FIG. 16F). Without wishing to be bound by theory, binding from the end of the recognition sequence appeared to facilitate greater blueshifting, possibly due to an unhindered cooperative “unzipping” effect from starting at the end.

Table 6 shows truncated target sequences used for experiments depicted in FIGS. 16A-16H.

TABLE 6

| | GTGTGTGTGTGTGTGTGTGTGTGTGTGTGTT | |
|---|---|---|
| | CAGTTTTGCATAGATTTGCACA | kcal/ |
| GT15mir19 | (SEQ ID NO: 47) | mole |
| mir19-10 | CTAAACGTGT | −17.28 |
| | (SEQ ID NO: 55) | |
| mir19-11 | TCTAAACGTGT | −18.88 |
| | (SEQ ID NO: 56) | |
| mir19-12 | ATCTAAACGTGT | −19.84 |
| | (SEQ ID NO: 57) | |
| mir19-13 | TATCTAAACGTGT | −21.31 |
| | (SEQ ID NO: 58) | |
| mir19-14 | GTATCTAAACGTGT | −23.27 |
| | (SEQ ID NO: 59) | |
| mir19-15 | CGTATCTAAACGTGT | −26.41 |
| | (SEQ ID NO: 60) | |
| mir19-17 | AACGTATCTAAACGTGT | −30.31 |
| | (SEQ ID NO: 61) | |
| mir19 | AGTCAAAACGTATCTAAACGTGT | −40.66 |
| | (SEQ ID NO: 62) | |

It was tested if GT15mir19 could detect a long sequence of ssDNA by addressing a smaller sequence in the middle. Using R23-mir19-R23, a 69 bp oligonucleotide with 23 complementary bases in the middle, it was found that even in the presence of SDBS, hybridization resulted in a small red-shift (FIG. 23). Because red-shifting from non-specific DNA interactions with other constructs was observed (FIG. 7), it was hypothesized that when R23mir19R23 hybridizes to the recognition sequence, the R23 at the 5′ end is held in close proximity to the nanotube surface due to the polarity of the wrapping sequence on the nanotube, and recapitulates the effect of non-specific oligonucleotide interactions. To test this, two sequences, R23-mir19 and mir19-R23 that have R23 at either the 3′ end or 5′ end, were designed. The orientation upon binding and resulting spectral shift is depicted in FIG. 16G. The results are shown for the (8,6) in FIG. 16H; both the miR-19 DNA control and mir19R23 produced a blueshift, whereas R23mir19 produced a redshift as predicted. Without wishing to be bound by theory, for detecting longer sequences, the orientation is critical in interpreting spectral shifts.

Without wishing to be bound by theory, the solution-phase dose-response data suggested that the limit of detection for miR-19 RNA is determined by ratio of nanotube binding sites to target RNA (FIG. 16B, FIG. 17)). Without wishing to be bound by theory, the best possible sensitivity then is at the single nanotube-level, which would represent the intrinsic threshold of detection. To image single nanotubes over time, adsorbing them to a glass surface provides a stable platform for imaging. However, adsorption was found to prevent recognition of target oligonucleotide, even in the presence of SDBS (data not shown). Direct adsorption to glass may result in disruption of the DNA-wrapping and the necessary tertiary structure that is adopted upon binding the target miR. To circumvent the problems caused by adsorption, a lysine coated plate was first treated with SDS to create a hydrophobic layer for the nanotubes associate with. When SDS-treated GT15mir19 nanotubes were added, a small percentage were able to loosely associate with the SDS layer for the duration of the experiment.

### Measurements of Single Sensor Complexes

The sensor function on the single-nanotube level was assessed via spectral imaging. The sensor was deposited on a lysine-coated glass surface with sodium dodecyl sulfate (SDS). Hyperspectral microscopy was used to spectroscopically image the (9,4) nanotube (FIG. 24A). By following single nanotubes on the surface before and after addition of miR-19 or R23 (FIG. 24A), it was possible to demonstrate microRNA detection on single nanotubes using wavelength shifting (FIG. 24B). FIGS. 24C and D show representative single nanotube spectra before and after miR-19 RNA or R23 (complete set of spectra in FIGS. 25A and B).

The blue-shifting of single nanotubes was apparent upon interrogating the sensor with miR-19 RNA, but not upon introducing R23 RNA (FIGS. 24B-24D, 25A-25B). Using the number of binding sites per nanotube length determined from AFM measurements (FIG. 2C), it was attempted to estimate the number of copies of miRNA detected per nanotube. Based on the banding pattern from AFM data provided herein and other AFM reports, 10-20 binding sites per 200 nm of nanotube were estimated. Because a diffraction-limited spot could contain a nanotube up to −600 nm long, a range of detection was estimated between 1-60 miRNA molecules.

### Sensor Multiplexing

Ideally, each chirality of SWCNT could act as a specific sensor for a given miR, with potentially 11-12 SWCNTs that can be easily measured in a PL plot for multiplexed detection of 11-12 miR species. Multiplexed detection of several miRs is advantageous due to increased specificity and sensitivity when using multiple miRs as a biomarker for disease conditions. For an implantable sensor, this would be an especially valuable feature. Using two nanotube preparations differentially enriched for different chiralities, multiplexed detection of two miR sequences was demonstrated.

The potential for the multiplexed detection of several miRNA sequences via the use of different nanotube chiralities was assessed. Two nanotube preparations enriched for different nanotube chiralities were suspended with binding sequences for either miR-19 or miR-509. A preparation enriched in large diameter species, (Nano-C APT-200) was suspended by the GT15mir19 sequence, and a CoMoCAT preparation enriched in small diameter species was suspended using the GT15mir509 sequence. Excitation/emission plots found that the GT15mir19 sensor, encapsulating the APT-200 nanotubes, effectively lacked the (6,5) species (FIG. 26A), while the GT15mir509 sensor, encapsulating the narrow-diameter enriched CoMoCAT preparation, lacked the (8,6) species (FIG. 26B). Absorbance spectra verified the differential enrichment of nanotube chiralites between these samples (FIG. 27). After mixing the two nanotube preparations, each miRNA sequence added individually was recognized by the appropriate nanotube chirality (FIG. 26C). When miR-19 and miR-509 were added together, the spectral shift almost identically recapitulated the shifts seen when either miRNA was added alone.

As purity of production methods improves, more chiralities can be used for greater multiplexing.

It was then assessed whether the platform could be extended to other analytes of interest by linking target recognition with DNA release from a structure-switching aptamer. As a model, a structure-switching aptamer that recognizes ATP was chosen, due to its role in extracellular communication and as a marker of bacterial growth. Because the aptamer was designed so that ATP binding releases a 12 bp reporter strand of DNA, the miR recognition sequence was substituted for a reporter recognition sequence (GT15cReporter, FIG. 26D). Addition of the reporter strand alone or addition of the aptamer in the presence of ATP produced a blueshift, while the structure-switching aptamer with GTP or alone elicited no blueshift (FIG. 26E and FIG. 28). The ability to link structure-switching aptamers with the unique optical properties of SWCNTs expands the repertoire of targets for this sensor platform.

### Toehold-Mediated Strand Displacement

Dynamic DNA nanotechnology using strand-displacement reactions has recently emerged as an attractive engineering system for various devices, including reconfigurable nanostructures, based on the specificity and versatility of DNA oligonucleotides.

It was determined whether the spectral response of the sensor could be reversed via toehold-mediated strand displacement. Strand displacement reactions occur through the use of “toeholds,” single-strand overhangs on duplexed DNA that facilitate binding of a complementary strand, which is thermodynamically favored due to complete complementarity, and is thus able to displace the shorter bound strand.

Accordingly, the miRNA capture sequence of the GT15mir19 sensor was truncated to leave a 6 nucleotide overhang after hybridization with the target strand to test whether the addition of a removing strand (RS) to bind the toehold and displace the target would reverse the spectral shift, according to the scheme depicted in FIG. 29C. Upon addition of miR-19 to the modified GT15mir19 sensor, the nanotube emission blue-shifted and the intensity increased (FIGS. 29D-29E). After 5 hours, the removing strand was added, at which point the blue-shifting ceased and the emission began to undergo a steady red-shift (FIG. 29D). The emission intensity exhibited a similar reversal (FIG. 29E). It is noted that the signal reversal was slower than detection in the forward direction, which is likely due to the energetic barrier for the truncated capture sequence to displace SDBS from the nanotube surface.

Because SWCNTs are extremely sensitive to their immediate environment, they are prone to non-specific interactions in complex biological environments. When GT15mir19 was tested in a solution of 10% fetal bovine serum (FBS), there was a 2 nm redshift across all conditions, and target DNA could not be detected (FIG. 30). To test if SDBS-treated GT15mir19 nanotubes can function in complex biological environments, both urine and FBS were tested due to their potential clinical value as sources of microRNA biomarkers. Using whole urine from a healthy donor, target microRNA was spiked in at various concentrations to recapitulate how a sample might be received clinically. A concentrated stock solution of SDBS was then added to the whole urine to bring the final concentration to 0.2%, and GT15mir19 nanotubes were added to a final concentration of 0.02 mg/L. The resulting dose-response is shown in FIG. 31A; based on the nanotube blueshift, miR-19 RNA was clearly detectable in whole urine to a threshold of 1 nM. The intensity enhancement also persisted in this biofluid, showing a similar threshold between 1 and 10 nM (FIG. 31B). In the more biologically complex situation found in FBS, SDBS enabled the detection of miR-19 DNA via blueshifting (FIG. 31C) and intensity enhancement (FIG. 31D). When miR-19 RNA was tested under identical conditions, no blueshift was observed for any concentration. Without wishing to be bound by theory, the detection of DNA, but not RNA, targets in serum could be due to RNA degradation or sequestration by components in the serum. The effect of bovine serum albumin, the major protein constituent in FBS, was tested at the low end and high end of the physiologically normal range (35 mg/L BSA and 50 mg/L BSA). At 35 mg/L BSA, it was found that RNA targets could be detected, but not with the same sensitivity as DNA targets (data not shown). At 50 mg/L BSA, RNA could not be detected at the same concentration, but the sensitivity to DNA target was unaffected (data not shown). Without wishing to be bound by theory, these data suggest that RNA has sensitivity to the total albumin content, while DNA is unaffected.

An application for the sensor/sensing platform is an implantable sensor for real-time monitoring of microRNA biomarkers. To demonstrate the utility of this platform for in vivo sensing, SDBS-pretreated GT15mir19 nanotubes were loaded into an implantable semipermeable membrane with a molecular weight cut off (500 kDa) small enough to keep the nanotubes inside, but to also allow sampling of small oligonucleotides in the environment (FIG. 31E). To verify that SDBS stays associated with the nanotubes in the membrane, the implantable sensor was subjected to dialysis against buffer for 6 hours with three buffer changes, and it was found that the characteristic blue-shifted shoulder of SDBS interacting with the nanotube persisted and provided enhanced blueshifting (FIG. 32). The nanotube implant was inserted into the peritoneum medially over the intestines (FIG. 31F). Two control groups received an IP injection of 1 μM R23 DNA or buffer only, and one experimental group received 1 μM of target miR-19 DNA. After incubation in the animals for 90 minutes, the mice were anesthetized and spectra were measured from the implanted sensor using a reflectance probe to both excite the nanotubes with 730 nm light and collect the nanotube emission (FIG. 31G). The two control groups did not show any significant difference, whereas the target microRNA group showed a significant blueshift (FIG. 31H). This experiment was repeated using miR-19 RNA, and again a statistically significant blueshift was found (FIG. 31I). It is believed that this is the first demonstration of a solvatochromism-based carbon nanotube sensor for non-invasive in vivo detection of both microRNA and short DNA oligonucleotides.

Detection of miRNA in Biofluids

The ability of the GT15mir19 sensor to detect miRNA binding events in common biofluids—urine and serum—due to their clinical value was assessed as sources of microRNA biomarkers. The GT15mir19 sensor and SDBS were introduced concomitantly to whole urine from 5 healthy donors before interrogating with miR-19 RNA. The wavelength shifting response was clearly detectable against controls down to 1 nM of miRNA, and intensity enhancement gave a similar sensitivity, between 1 and 10 nM (FIGS. 62A-62B, FIGS. 55A-55B). Variation from sample-to-sample was minimal. In whole serum, it was found that target miR-19 DNA was similarly detectable in the presence of SDBS (FIGS. 56A-56B).

When target miR-19 RNA was introduced to the sensor, it was found that only a small response at the highest tested concentration (FIGS. 56A-56B). It was hypothesized that the RNA detection was complicated by RNases in the serum which might degrade the analyte sequence, as reported elsewhere for synthetic RNA sequences. Therefore, proteinase K, a detergent-stable protease used to deactivate RNase, was introduced into the serum. Introduction of proteinase K allowed the detection of miR-19 RNA with the same sensitivity as for the DNA analogue (FIG. 62C, FIGS. 56A-56B). When proteinase K was introduced 12 h after mixing miR-19 with serum, the sensitivity of the response to miR-19 RNA was not improved, suggesting that the RNA had been destroyed. To verify broad applicability with this method, miR-21 was also used as a target, due to its significance as a serum colorectal cancer biomarker. Similarly, GT15mir21 sensor was also tested in whole serum treated with proteinase K. It was found that miR-21 RNA could be detected directly in minimally-treated serum via both blue-shifting and intensity enhancement (FIG. 62C, FIG. 57).

Detection of miRNA Detection In Vivo

The present Example provides the ability of the system to detect miRNA in vivo via a minimally-invasive implantable device. The SDBS-treated GT15mir19 sensor was loaded into a semipermeable membrane capillary with a MWCO of 500 kDa (FIG. 62D). To determine whether this cutoff would to prevent the diffusion of the GT15mir19 sensor complexes outside of the membrane, the molecular weight of the GT15mir19 sensor was calculated. It was estimated that the sensor complexes composed of a small diameter (e.g., 0.8 nm) and average length of about 166 nm fall within the range of 701 kDa to 839 kDa. It was surmised that the miR-19 miRNA, with a molecular weight of 7.055 kDa, would pass through the membrane.

The likelihood that the enhanced signal response provided by SDBS would continue after device implantation was also assessed. Thus, the semi-permeable capillary was filled with SDBS-pretreated GT15mir19 sensor and was placed in buffer dialysate for 6 hours. The buffer was changed and the sensor response was assessed with miR-19 every 2 hours (FIGS. 58A-58B). It was found that the GT15mir19 sensor exhibited a nearly identical blue-shifting response after 6 hours of dialysis, suggesting that the SDBS remained associated with the sensor even under these conditions.

The sensor response was tested in vivo after surgically implanting the membrane into the peritoneal cavity of NU/J (nude) mice. The membrane was placed medially over the intestines and sutured to the parietal peritoneum to immobilize the device. It was first tested whether DNA could be detected intraperitoneally by injecting 1 nanomole of miR-19 DNA, R23, or the vehicle control. The mice exhibited no obvious adverse effects or changes in behavior following the implantation or injection. After 90 min, the mice were anesthetized using isofluorane. A fiber optic-based probe system was developed to excite an 0.8 cm² area with a 730 nm CW laser (FIG. 62E), collect the emitted near-infrared light through the same fiber bundle, disperse the light with a Czerny-Turner spectrograph, and detect the light via a 1D InGaAs array (FIG. 6F). Using the nanotube emission signal collected from the mouse, it was found that the target miR-19 DNA exhibited a significant blue-shifting response as compared to controls (FIG. 6G). The experiment was repeated using the RNA version of the analyte, resulting in a similar response (FIG. 59).

The implantable device was tested in vitro by immersing the filled capillary into buffer containing RNA, finding that the threshold of detection was below 10 pmol (FIG. 60). To determine the limit of detection in vivo, 500 pmol, 100 pmol, or 50 pmol of miR-19 RNA was injected intraperitoneally into mice implanted with the devices. After 120 min, significant wavelength shifting responses were measured down to 100 pmol (FIG. 64H). The devices were removed from the animals and measured ex vivo, resulting in similar results (FIG. 61). Without wishing to be bound to any theory, it is suspected that the higher limit of detection of the device in vivo as compared to in vitro was due to the degradation of microRNA in the peritoneal fluid as well as fluid exchange out of the peritoneal cavity. The measurement of endogenous microRNA targets, which are highly stable due in part to their association with proteins such as Ago2, may help improve sensor performance.

### Thermodynamic Analysis of Nucleic Acid Hybridization on the Sensor

Two schemes shown in FIG. 47 were used to estimate the difference in free energy of ssDNA adsorption and dsDNA hybridization at the nanotube surface. As parameters needed for such a calculation are available for a 17-mer duplex strand, analysis was focued on this particular DNA length and sequence.

For case A, one ssDNA is already adsorbed on the nanotube surface and its complementary partner ssDNA is introduced in the solution like the experimental setup as provided herein. The change in free energy upon hybridization is approximately −135 kcal/mol (at (300 K, 1 bar), which clearly indicates that hybridization is preferred over adsorption).

Similarly for case B, where both strands are initially adsorbed on the nanotube surface, the change in free energy upon hybridization is approximately +9 kcal/mol. This indicates that when both strands are initially adsorbed (FIG. 47, Case B), ssDNA adsorption is slightly more favorable than dsDNA hybridization. In this experimental setup of miR-19 hybridization on the nanotube, case A was the relevant analysis as complementary strand is introduced after ssDNA and surfactant are allowed to adsorb on the nanotube surface. Thus, these analysis findings are similar to the observed hybridization leading to the function of biosensor/reporter.

### Effects of Amphipathic Molecules on Sensor Response

Several classes of amphipathic molecules were introduced to the GT15mir19 sensor to assess their potential to modulate the optical response to hybridization. Selected molecules included ionic surfactants, non-ionic triblock copolymers, non-ionic surfactants, PEG-functionalized lipid, and BSA due to their variety of steric and electrostatic properties (Table 3). After treatment for 4 hours with each amphipathic molecule, but before addition of target oligonucleotide, emission spectra were measured to assess the effect of each molecule in the absence of target miRNA. The impact on center wavelength and intensity are shown for the (7,5) nanotube, which was similar to the responses of other chiralities (FIGS. 48A-48B). All molecules either elicited a blue-shift to varying degrees or had no apparent effect. SDC was an outlier in that the intensity was enhanced 2-3 fold. While other molecules were found to also enhance intensity to different degrees, none matched the effect of SDC.

For each set of surfactant-treated nanotubes, complementary and non-complementary target oligonucleotides were introduced and incubated for 4 hours. Each amphipathic molecule was tested at a final concentration of 0.2% wt/vol with 2 mg/L of GT15mir19. Endpoint data showed that SDBS and IGEPAL provided the greatest enhancement of target miRNA-induced blue-shifting, followed by SDS, Brij52, and lipid-PEG to a smaller extent (FIGS. 49A-49B). The presence of Pluronic, SDC, and Triton X-100 resulted in no apparent blue-shift of the sensor upon introduction of target miRNA, although it is noted that SDC and Triton X-100 substantially blue-shifted the nanotube before target oligonucleotides were added. The initial blue-shift suggests that these amphiphiles likely coated the nanotube so efficiently as to displace water from the nanotube surface and prevent the capture sequence of the GT15mir19 oligonucleotide from interacting with the nanotube surface prior to hybridization. There are no obvious patterns relating the structure of the amphiphiles to the modulation of the response to miRNA, although there are certain factors that can be noted. SDC caused an initial blue-shift and prevented the response to miRNA, for example, because it is a very strong surfactant that is known to efficiently suspend nanotubes and enhance nanotube emission. Pluronic and Triton X-100 are fairly large/bulky surfactant molecules which may have similarly prevented interactions of the capture sequence with the nanotube surface. It is also noted that the structural similarity between SDBS and IGEPAL, the two surfactants that resulted in the largest hybridization-induced enhancements. It is also noted that supramolecular interactions of the surfactant molecules with each other and the nanotube surface are complex.

### SDBS-Induced Spectroscopic Changes

Optical transition energies for DNA-wrapped nanotubes are red-shifted by 10-20 meV (14-22 nm, depending on chirality) and quenched as compared to nanotubes suspended entirely with small molecule anionic surfactants like SDS or SDBS. A proposed mechanism has attributed this finding to incomplete coverage of the nanotube surface by DNA, which allows for greater accessibility of water, resulting in an increased polarity of the local solvent environment (higher local dielectric constant) in the immediate vicinity of the nanotube. However, a blue-shifted shoulder in the spectrum of the GTmir19 sensor was observed in the absence of the complementary miR-19 strand upon introduction of SDBS (FIG. 2F and FIG. 10). Without wishing to be bound to any theory, this spectral change suggests that SDBS binds to the exposed surfaces on the DNA-suspended nanotube, causing the displacement of water from the nanotube surface, which produces a slight blue-shift in the emission. When target RNA or DNA hybridizes and the duplex dissociates from the surface, bare nanotube surface is exposed, allowing SDBS to bind and become the dominant factor determining of the nanotube emission peak wavelength, and intensity. The net effect was a dramatic blue-shift (4-17 nm, depending on the nanotube chirality) and intensity increase (1.3-2.2 fold) from the assembly of supramolecular complexes of SDBS, triggered by the introduction of target RNA or DNA.

From spectroscopic studies of the GT15mir19 sensor response, a blue shift in nanotube excitation wavelengths was observed, suggesting that the binding of miR-19 RNA and DNA affects the ground state absorption energies in addition to the excited state. FIG. 12 shows the correlation between the excitation wavelength shift and the emission wavelength shift for the ensemble of chiralities, yielding a Pearson correlation coefficient of 0.87744 (p=0.00188). When plotted as change in energy (FIG. 12), the Pearson correlation coefficient is similar 0.90656 (p=0.0007). The environmental effects on nanotube optical properties have been shown to depend at least in part on the mod type of the nanotube. On stratifying the nanotubes by mod type, defined for any nanotube as mod(n−m,3), it was found that mod 2 nanotubes exhibited an emission energy modulation that increased nearly linearly (R²=0.9272) with nanotube diameter (FIG. 11). Interestingly, for the mod 2 nanotubes, the intensity enhancement did not show the same linear relationship with nanotube diameter, although all nanotubes increased in intensity. A maximum was found for nanotubes ˜0.9 nm in diameter (FIG. 11). A slight difference also became apparent between the responses to target DNA and RNA, with RNA eliciting a slightly enhanced intensity increase for small diameter nanotubes and a slightly dampened enhancement for larger diameter nanotubes. This small, diameter-dependent difference may be related to the difference in binding strength and hydration between DNA-DNA hybrids and DNA-RNA hybrids.

### Molecular Weight of the Sensor Complex

The molecular weight of the sensor was estimated using the lower limit of the nanotube diameters to be 0.8 nm, wherein there are 20 carbons around the nanotube circumference. Thus, 80 carbon atoms are present for every 0.283 nm in nanotube length. Taking the average length of the nanotube, as measured via AFM, to be 166 nm, the resulting molecular weight of the nanotube is 564 kDa. The molecular weight of the GT15mir19 DNA sequence is 16.5 kDa. From AFM measurements, it was estimated that 5-10 copies of DNA per 100 nm, and thus 8.3 to 16.6 copies per 166 nm, adding 137 kDa to 275 kDa to the total complex. Thus, for an average length GT15mir19 sensor with diameter near the lower limit, the molecular weight would be between 701 kDa and 839 kDa.

### Applications

Herein, label-free, amplification-free optical sensors were engineered for the quantitative detection of oligonucleotide hybridization events in vitro and non-invasively in vivo. The sensor mechanism, resulting from competitive effects of the displacement of both electrostatic charge and water from the carbon nanotube surface, has implications for the improvement of carbon nanotube-based optical and electronic sensors. A better determination of the effects of length, mismatches in sequence, and orientation of longer oligonucleotides on the optical response of the carbon nanotube was gained. The GT15mirX sensor enabled detection via single-molecule sensor elements and multiplexing using multiple nanotube chiralities. The monitoring of toehold-based strand displacement events portends use in nucleic acid-based logic circuits and also allowed the reversal of the sensor response and regeneration of the sensor complex, which may potentially be exploited for continuous use.

In vitro applications such as point-of-care diagnostics may provide the most immediate route to clinical use. It was found that the sensor can directly detect oligonucleotides in heterogeneous biofluids such as urine and serum with minimal pre-treatment, potentially circumventing biases and variability related to typical pre-analytical steps required for RT-qPCR. Regarding sensor parameters pertinent to clinical measurements, microRNA content in 12 body fluids were surveyed, providing useful quantitative information to estimate the physiological range of microRNA. The limit of detection of the provided sensor in bulk solution is in the picomolar range (e.g., greater than the picomolar range), although the threshold of detection and dynamic range depends on several factors, including binding site coverage. The ability to measure single-nanotube responses representing 1-60 copies of microRNA binding was also demonstrated, suggesting that sensitivities down to 10's of copies of microRNA can be obtained.

An implantable optical sensor device for the non-invasive detection of biomarkers such as miRNA may potentially be used in conjunction with wearable devices to facilitate the optical readout and data recording. The described sensor implants quantified miRNA down to 100 pmol in vivo. Further, miRNA is often found associated with the small protein Ago2, which makes it physiologically stable. Functionally, Ago2 binds to microRNA in a conformation to favor hybridization with target sequences, especially over an 8 nucleotide section called the seed sequence, but steric hindrance or charge interactions of the protein with miRNA could slow access to the protein-bound sections of the strand.

### Sequences

Table 7 shows GT15mirX sequences used herein.

TABLE 7

| Name | Sequence (5′ to 3′) |
|---|---|
| GT15mir19 | GTGTGTGTGTGTGTGTGTGTGTGTGTGTGTTCAGTTTTG |
| | CATAGATTTGCACA (SEQ ID NO: 47) |
| GT15mir126 | GTGTGTGTGTGTGTGTGTGTGTGTGTGTGTGCATTATTA |
| | CTCACGGTACGA (SEQ ID NO: 63) |
| GT15mir182 | GTGTGTGTGTGTGTGTGTGTGTGTGTGTGTTGTGAGTTC |
| | TACCATTGCCAAA (SEQ ID NO: 64) |
| GT15mir152 | GTGTGTGTGTGTGTGTGTGTGTGTGTGTGTCCAAGTTCT |
| | GTCATGCACTGA (SEQ ID NO: 65) |
| GT15mir509 | GTGTGTGTGTGTGTGTGTGTGTGTGTGTGTTGATTGCCA |
| | CTGTCTGCAGTA (SEQ ID NO: 66) |
| GT15mir96 | GTGTGTGTGTGTGTGTGTGTGTGTGTGTGTAGCAAAAAT |
| | GTGCTAGTGCCAAA (SEQ ID NO: 67) |
| GT15mir183 | GTGTGTGTGTGTGTGTGTGTGTGTGTGTGTAGTGAATTC |
| | TACCAGTGCCATA (SEQ ID NO: 68) |
| GT15mir494 | GTGTGTGTGTGTGTGTGTGTGTGTGTGTGTGAGGTTTCC |
| | CGTGTATGTTTCA (SEQ ID NO: 69) |
| GT15 mir39 | GTGTGTGTGTGTGTGTGTGTGTGTGTGTGTCAAGCTGAT |
| | TTACACCCGGTGA (SEQ ID NO: 70) |
| GT15mir21 | GTGTGTGTGTGTGTGTGTGTGTGTGTGTGTTCAACATCA |
| | GTCTGATAAGCTA (SEQ ID NO: 71) |
| GT15mir141 | GTGTGTGTGTGTGTGTGTGTGTGTGTGTGTCCATCTTTA |
| | CCAGACAGTGTTA (SEQ ID NO: 72) |
| GT15mir429 | GTGTGTGTGTGTGTGTGTGTGTGTGTGTGTACGGTTTTA |
| | CCAGACAGTATTA (SEQ ID NO: 73) |
| GT15mir200 | GTGTGTGTGTGTGTGTGTGTGTGTGTGTGTTCATCATTA |
| b | CCAGGCAGTATTA (SEQ ID NO: 74) |
| GT15mir19- | GTGTGTGTGTGTGTGTGTGTGTGTGTGTGTTCAGTTTTG |
| minus6 | CATAGATT (SEQ ID NO: 75) |
| GT6mir19- | GTGTGTGTGTGTTCAGTTTTGCATAGATTTGCACA- |
| Cy5 | Cy5 (SEQ ID NO: 76) |

Table 8 shows analyte/target sequences used herein.

TABLE 8

| Name | Sequence (5′ to 3′) |
|---|---|
| miR-19 DNA | TGTGCAAATCTATGCAAAACTGA |
| | (SEQ ID NO: 48) |
| miR-19 RNA | UGUGCAAAUCUAUGCAAAACUGA |
| | (SEQ ID NO: 49) |
| miR-21 DNA | TAGCTTATCAGACTGATGTTG |
| | (SEQ ID NO: 77) |
| miR-21 RNA | UAGCUUAUCAGACUGAUGUUG |
| | (SEQ ID NO: 78) |
| R23 DNA | TCGGTCAGTGGGTCATTGCTAGT |

| | |
|---|---|
| | (SEQ ID NO: 79) |
| R23 RNA | UCGGUCAGUGGGUCAUUGCUAGU |
| | (SEQ ID NO: 80) |
| miR-126 | TCGTACCGTGAGTAATAATGC |
| | (SEQ ID NO: 81) |
| miR-182 | TTTGGCAATGGTAGAACTCACA |
| | (SEQ ID NO: 82) |
| miR-152 | TCAGTGCATGACAGAACTTGG |
| | (SEQ ID NO: 83) |
| miR-509 | TACTGCAGACAGTGGCAATCA |
| | (SEQ ID NO: 84) |
| miR-96 | TTTGGCACTAGCACATTTTTGCT |
| | (SEQ ID NO: 85) |
| miR-183 | TATGGCACTGGTAGAATTCACT |
| | (SEQ ID NO: 86) |
| miR-494 | TGAAACATACACGGGAAACCTC |
| | (SEQ ID NO: 87) |
| miR-39 | TCACCGGGTGTAAATCAGCTTG |
| | (SEQ ID NO: 88) |
| miR-141 | TAACACTGTCTGGTAAAGATGG |
| | (SEQ ID NO: 89) |
| miR-200b | TAATACTGCCTGGTAATGATGA |
| | (SEQ ID NO: 90) |
| miR-429 | TAATACTGTCTGGTAAAACCGT |
| | (SEQ ID NO: 91) |
| Removing Seq. | TCAGTTTTGCATAGATTTGCACA |
| | (SEQ ID NO: 92) |

Table 9 shows truncated miR analyte sequences designed to hybridize to the middle of miRNA capture sequence.

| Name | Sequence |
|---|---|
| GT15mir19 | 5′-GTGTGTGTGTGTGTGTGTGTGTGTGTGTGTTCAGTTT |
| | TGCATAGATTTGCACA-3′ (SEQ ID NO: 47) |
| mir19-10 | 3′-AGTCAAAACG-5′ (SEQ ID NO: 93) |
| mir19-11 | 3′-AGTCAAAACGT-5′ (SEQ ID NO: 94) |
| mir19-12 | 3′-AGTCAAAACGTA-5′ (SEQ ID NO: 95) |
| mir19-13 | 3′-AGTCAAAACGTAT-5′ (SEQ ID NO: 96) |
| mir19-14 | 3′-AGTCAAAACGTATC-5′ (SEQ ID NO: 97) |
| mir19-15 | 3′-AGTCAAAACGTATCT-5′ (SEQ ID NO: 98) |

Table 10 shows truncated miR analyte sequences designed to hybridize to the 5′ end of miRNA capture sequence.

TABLE 10

| Name | Sequence |
|---|---|
| GT15mir19 | 5′-GTGTGTGTGTGTGTGTGTGTGTGTGTGTGTTCAGTTT |
| | TGCATAGATTTGCACA-3′ (SEQ ID NO: 47) |
| mir19-10 | 3′-CTAAACGTGT-5′ (SEQ ID NO: 99) |
| mir19-11 | 3′-TCTAAACGTGT-5′ (SEQ ID NO: 100) |
| mir19-12 | 3′-ATCTAAACGTGT-5′ (SEQ ID NO: 101) |
| mir19-13 | 3′-TATCTAAACGTGT-5′ (SEQ ID NO: 102) |
| mir19-14 | 3′-GTATCTAAACGTGT-5′ (SEQ ID NO: 103) |
| mir19-15 | 3′-CGTATCTAAACGTGT-5′ (SEQ ID NO: 104) |
| Mir19-17 | 3′-AACGTATCTAAACGTGT-5′ (SEQ ID NO: 105) |

Table 11 shows elongated analyte sequences used herein.

TABLE 11

| Name | Sequence (5′ to 3′) |
|---|---|
| R23mir19R23 | TCGGTCAGTGGGTCATTGCTAGTGTGCAAATCTATGCA |
| | AAACTGATCGGTCAGTGGGTCATTGCTAGT |
| | (SEQ ID NO: 106) |
| mirl9R23 | TCGGTCAGTGGGTCATTGCTAGTGTGCAAATCTATGCA |
| | AAACTGA |
| | (SEQ ID NO: 107) |
| R23mir19 | TGTGCAAATCTATGCAAAACTGATCGGTCAGTGGGTCA |
| | TTGCTAGT |
| | (SEQ ID NO: 108) |
| R23R23R23 | TCGGTCAGTGGGTCATTGCTAGTCGGTCAGTGGGTCAT |
| | TGCTAGTTCGGTCAGTGGGTCATTGCTAGT |
| | (SEQ ID NO: 109) |

### Methods

### DNA-Suspension of Carbon Nanotubes

Carbon nanotubes produced by the HiPco process (Unidym, Sunnyvale, Calif.), CoMoCAT process (SG65i grade, Sigma-Aldrich, St. Louis, Mo., US), or a combustion process (APT-200, Nano-C, Westwood, Mass.) were mixed with DNA oligonucleotides (IDT DNA, Coralville, Iowa) at a 2:1 mass ratio in 1 mL of saline-sodium citrate (SSC) buffer and ultrasonicated for 30 minutes at 40% amplitude (Sonics & Materials, Inc.). The complete list of DNA sequences used for suspension can be found in Supplementary Methods. Following ultrasonication, the dispersions were ultracentrifuged (Sorvall Discovery 90SE) for 30 minutes at 280,000×g. The top 80% of the supernatant was collected. Absorbance spectra were acquired using a UV/Vis/nIR spectrophotometer (Jasco V-670, Tokyo, Japan). The concentration was calculated using the extinction coefficient Abs₉₁₀=0.02554 L mg⁻¹ cm⁻¹. To remove free DNA, 100 kDa Amicon centrifuge filters (Millipore) were used. The DNA-nanotube complexes were re-suspended in saline-sodium citrate buffer (G Biosciences, St. Louis, Mo.).

### Fluorescence Spectroscopy of Carbon Nanotubes in Solution

Fluorescence emission spectra from aqueous nanotube solutions were acquired using a home-built apparatus consisting of a tunable white light laser source, inverted microscope, and InGaAs nIR detector. The SuperK EXTREME supercontinuum white light laser source (NKT Photonics) was used with a VARIA variable bandpass filter accessory capable of tuning the output 500-825 nm with a bandwidth of 20 nm. The light path was shaped and fed into the back of an inverted IX-71 microscope (Olympus) where it passed through a 20× nIR objective (Olympus) and illuminated a 50-100 μL nanotube sample in a 96-well plate (Corning). The emission from the nanotube sample was collected through the 20× objective and passed through a dichroic mirror (875 nm cutoff, Semrock). The light was f/# matched to the spectrometer using several lenses and injected into an Isoplane spectrograph (Princeton Instruments) with a slit width of 410 μm which dispersed the emission using a 86 g/mm grating with 950 nm blaze wavelength. The spectral range was 930-1369 nm with a resolution of ˜0.7 nm. The light was collected by a PIoNIR InGaAs 640×512 pixel array (Princeton Instruments). A HL-3-CAL-EXT halogen calibration light source (Ocean Optics) was used to correct for wavelength-dependent features in the emission intensity arising from the spectrometer, detector, and other optics. A Hg/Ne pencil style calibration lamp (Newport) was used to calibrate the spectrometer wavelength. Background subtraction was conducted using a well in a 96-well plate filled with DI H2O. Following acquisition, the data was processed with custom code written in Matlab which applied the aforementioned spectral corrections, background subtraction, and was used to fit the data with Lorentzian functions.

### Atomic Force Microscopy

The GT15mir19 sensor was incubated overnight at 20 mg/L with 10 μM of the miR-19-hairpin or 10 μM of the R23-hairpin in saline sodium citrate diluted 20× in 20 mM HEPES+5 mM MgCl₂. The sample was plated on a freshly cleaved mica substrate (SPI) for 4 minutes before washing with 10 mL of dH₂O and blowing dry with argon gas. An Asylum Research MFP-3D-Bio instrument was used with an Olympus AC240TS AFM probe in AC mode. Data was captured at 2.93 nm/pixel XY resolution and 15.63 pm Z resolution. For AFM under aqueous conditions, 20 mg/L of the GT15mir19 sensor was incubated with 10 μM of the miR-19-hairpin, R23-hairpin, or buffer overnight. All three conditions were spin-filtered 3× with 100 kDa Amicon centrifuge filters, and resuspended with 5 mM NiCl₂, 20 mM HEPES pH 6.7 buffer. The samples were plated onto freshly cleaved mica for 2 minutes before gently washing with the same buffer. Samples were imaged in a droplet of the buffer using an Asylum Research Cypher ES+BlueDrive AFM with an Olympus AC55 probe and imaged using BlueDrive excitation at the ambient temperature of 31° C. within the AFM enclosure. All three samples were imaged with the same probe, consecutively, with the same scan settings, starting with the miR-19-hairpin sample, followed by the R23-hairpin control and the buffer control.

### Hybridization Experiments in Buffer Conditions and Biofluids

Hybridization experiments were conducted with 2 mg/L of the GT15mir19 sensor in saline-sodium citrate buffer at room temperature. Target DNA or RNA was introduced to reach a final concentration of 1 μM. Samples were incubated for 4 hours, unless otherwise noted. Free energy of hybridization was predicted using OligoAnalyzer 3.1 (IDT). Kinetics experiments were measured every 10 minutes using custom LabView code. Hybridization experiments with sodium dodecylbenzenesulfonate (SDBS) were conducted using a final concentration 0.2% wt/v. SDBS was added to the GT15mir19 sensor and allowed to equilibrate overnight at room temperature before target oligonucleotides were added. Toehold-mediated strand displacement experiments were performed with 1 μM of target miR-19 DNA, and 10 of the removing strand, composed of an ssDNA oligonucleotide with the complementary sequence to miR-19. Hybridization experiments in urine were conducted in samples from 5 healthy volunteers and stored on ice until the experiment. Concentrated GT15mir19 was added to each sample to final concentration of 0.2 mg/L and SDBS to final concentration of 0.2%. Concentrated DNA and RNA target were added to the indicated concentrations and incubated at room temperature overnight. Serum experiments used fetal bovine serum (Life Sciences) with GT15mir19 added to final concentration 0.2 mg/L and SDBS at 0.2%. Where indicated, proteinase K (New England Biolabs) was added to a final concentration of 0.5 mg/mL. Spectra were acquired after overnight incubation at room temperature.

### Single-Nanotube Measurements

Single-nanotube measurements were performed by incubating SDS-treated GT15mir19 sensor (0.2% SDS in SSC buffer) on a poly-D-lysine coated glass bottom plate (Mattek, Ashland, Mass.) for 10 minutes before gently washing with 0.2% SDS in SSC buffer. A final volume of 1 mL SDS-buffer was left in the plate during hyperspectral imaging measurements of the surface-bound nanotubes. A small volume (1 uL) of 1 mM solutions of miR-19 RNA or R23 RNA were then mixed with the buffer. Hyperspectral imaging measurements were repeated after 15 minutes and 50 minutes. Single nanotube emission spectra were collected via a custom near-infrared hyperspectral microscope. Data was processed with ImageJ software. Peaks were fit to Voigt functions using custom Matlab code to obtain center wavelength values.

### Molecular Dynamics Simulations

Molecular dynamics (MD) simulations were conducted using the (9,4) nanotube chirality in explicit water. The DNA molecule for GT15mir19 (without complementary strand) was generated as an unstructured single stranded DNA and placed near the (9,4) nanotube, followed by a sufficiently long equilibration MD simulation enhanced with a replica-exchange based method to let the entire strand adsorb on (9,4) nanotube surface. Analysis of an additional 250 ns long MD simulation is presented herein. The DNA molecule for GT15mir19 hybridized with the complementary strand was created in a partially double stranded form. miR-19 was generated in the double stranded form using NAB program of AmberTools and was appropriately bonded via phosphodiester bond to the ss(GT)₁₅ segment (SEQ ID NO: 1) of the GT15mir19 DNA. The ss(GT)15 (SEQ ID NO: 1) nanotube binding portion of the first strand was adsorbed to the nanotube. The entire DNA and nanotube construct was solvated in a 10.65×10.65×14.7179 nm water-box containing approximately 55,000 water molecules and 74 sodium counter-ions, placed randomly, to balance the negative charges from phosphates on the DNA. The total system was approximately 170,000 atoms. The nanotube extended to the edges of the water box and was kept frozen in place during the entire equilibration and simulation time. The nanotube atoms were modeled as sp2 hybridized carbon. All structures were visualized in VMD⁶⁰.

The Gromacs 4.6.7 simulation package was used with the Charmm36/TIP3P nucleic acid/water model. Long-range electrostatics were calculated using the particle mesh Ewald method with a 0.9 nm real space cutoff. For van der Waals interactions, a cutoff value of 1.2 nm was used. The energy minimized simulation box was then subjected to 100 ps equilibration in an NVT (T=300 K) ensemble where the number of water molecules were fine-tuned to make average pressure approximately equivalent to atmospheric pressure. Further equilibration runs were performed for 100-200 ns in NVT (T=300 K) ensemble. Systems were propagated with stochastic Langevin dynamics with a time step of 2 fs. The trajectories were saved every 10 ps, yielding a total of 25,000 snapshots for production analysis. Homemade python scripts calling MDAnalysis module were used for all other analysis presented.

### Quantification of DNA on the Nanotube Complex

The GT15mir19 sequence was used to suspend nanotubes as described earlier. After each of 4 centrifugation filter steps using the Amicon centrifuge filter (100 kDa MWCO), the concentration of the filtered DNA was measured using Abs₂₆₀ on a NanoDrop spectrophotometer (ThermoScientific, Waltham, Mass.). The pellet from centrifugation was also filtered to measure free DNA. The final mass of DNA from the combined values was calculated from the concentration and subtracted from the initial value. From three suspensions, it was found that 3.5 (+/−1.8) mg of DNA suspended 1 mg of nanotube.

### Device Implantation and In Vivo Spectroscopy

All animal experiments were approved by the Institutional Animal Care and Use Committee at Memorial Sloan Kettering Cancer Center. KrosFlo Implant Membranes (500 kD MWCO) were obtained from Spectrum Labs (Rancho Dominguez, Calif.). The membrane was cut to about 1 cm in length and filled with approximately 15 μL of 2 mg/L GT15mir19-nanotubes. Each end was heat sealed. A total of 36 NU/J (nude) mice (Jackson Labs) were anesthetized with 2% isoflurane and implanted with the membrane. Nine mice were divided into three cohorts of three mice to receive miR-19 DNA, R23 DNA, or buffer vehicle via an intraperitoneal injection of 1 nanomole in 1 mL sodium saline citrate buffer. An identical experiment was performed with miR-19 RNA, R23 RNA, or buffer vehicle at 1 nanomole, 500 picomole, 100 picomole, or 50 picomole in 1 mL sodium saline citrate buffer. The mice were removed from anesthesia and allowed to regain consciousness. After 90 or 120 minutes, mice were anesthetized and measured using a custom-built reflectance probe-based spectroscopy system. The system consisted of a continuous wave 1 watt 730 nm diode laser (Frankfurt). The laser light was injected into a bifurcated fiber optic reflection probe bundle. The sample leg of the bundle included one 200 μm, 0.22 NA fiber optic cable for sample excitation located in the center of six 200 μm, 0.22 NA fiber optic cables for collection of the emitted light. Emission below 1050 nm was filtered using longpass filters, and the light was focused into the slit of a Czerny-Turner spectrograph with 303 mm focal length (Shamrock 303i, Andor). The slit width of the spectrograph was set at 410 μm. The light was dispersed using a 85 g/mm grating with 1350 nm blaze wavelength and collected with an iDus InGaAs camera (Andor). Spectra were fit to Voigt functions using custom Matlab code.

Table 12 shows a list of mammalian miRNAs that can be used with the sensor described herein.

TABLE 12

| Name | Disease | Expression level |
|---|---|---|
| hsa-let-7f-2 | kidney cancer | up-regulated |
| hsa-let-7g | hepatocellular carcinoma (HCC) | down-regulated |
| hsa-let-7g | lung cancer | down-regulated |
| hsa-let-7g | non-small cell lung cancer (NSCLC) | down-regulated |
| hsa-let-7g | ovarian cancer (OC) | down-regulated |
| hsa-let-7g | colorectal cancer | up-regulated |
| hsa-let-7g | head and neck squamous cell carcinoma (HNSCC) | up-regulated |
| hsa-let-7g | hepatocellular carcinoma (HCC) | down-regulated |
| hsa-let-7g | malignant melanoma | down-regulated |
| hsa-let-7g | Oral Squamous Cell Carcinoma (OSCC) | down-regulated |
| hsa-let-7g | prostate cancer | down-regulated |
| hsa-let-7g | prostate cancer | up-regulated |
| hsa-let-7i | Alzheimer's disease | down-regulated |
| hsa-let-7i | breast cancer | up-regulated |
| hsa-let-7i | head and neck squamous cell carcinoma (HNSCC) | up-regulated |
| hsa-miR-1 | cardiac hypertrophy | down-regulated |
| hsa-miR-1 | cardiac hypertrophy | down-regulated |
| hsa-miR-203 | pancreatic ductal adenocarcinoma (PDAC) | up-regulated |
| hsa-miR-203 | uterine leiomyoma (ULM) | down-regulated |
| hsa-miR-204 | acute myeloid leukemia (AML) | down-regulated |
| hsa-miR-204 | breast cancer | down-regulated |
| hsa-miR-382 | Duchenne muscular dystrophy (DMD) | up-regulated |
| hsa-miR-382 | limb-girdle muscular dystrophies types 2A (LGMD2A) | up-regulated |
| hsa-miR-382 | miyoshi myopathy (MM) | up-regulated |
| hsa-miR-204 | Insulinoma | up-regulated |
| hsa-let-7d | acute promyelocytic leukemia (APL) | up-regulated |
| hsa-miR-133a | cardiomyopathy | down-regulated |
| hsa-miR-21 | lung cancer | up-regulated |
| hsa-miR-16-1 | acute promyelocytic leukemia (APL) | up-regulated |
| hsa-miR-635 | ovarian cancer (OC) | down-regulated |
| hsa-miR-10a | diffuse large B-cell lymphoma (DLBCL) | down-regulated |
| hsa-miR-184 | acute myeloid leukemia (AML) | down-regulated |
| hsa-miR-19a | hepatocellular carcinoma (HCC) | down-regulated |
| hsa-miR-24 | prostate cancer | up-regulated |
| hsa-miR-302c | epithelial ovarian cancer (EOC) | down-regulated |
| hsa-miR-30d | Oral Squamous Cell Carcinoma (OSCC) | down-regulated |
| hsa-miR-368 | Duchenne muscular dystrophy (DMD) | up-regulated |
| hsa-miR-150 | lung cancer | up-regulated |
| hsa-miR-125a | breast cancer | down-regulated |
| hsa-miR-146a | lung cancer | up-regulated |
| hsa-miR-146a | Oral Squamous Cell Carcinoma (OSCC) | up-regulated |
| hsa-miR-146a | pancreatic cancer | up-regulated |
| hsa-miR-146a | pancreatic cancer | up-regulated |
| hsa-miR-146a | prostate cancer | up-regulated |
| hsa-miR-146a | psoriasis | up-regulated |
| hsa-miR-146b | breast cancer | down-regulated |
| hsa-miR-146b | autism spectrum disorder (ASD) | down-regulated |
| hsa-let-7d | lung cancer | down-regulated |
| hsa-let-7d | ovarian cancer (OC) | down-regulated |
| hsa-let-7d | breast cancer | down-regulated |
| hsa-let-7d | epithelial ovarian cancer (EOC) | down-regulated |
| hsa-let-7d | epithelial ovarian cancer (EOC) | down-regulated |
| hsa-let-7d | hepatocellular carcinoma (HCC) | down-regulated |
| hsa-let-7d | malignant melanoma | down-regulated |
| hsa-let-7d | Oral Squamous Cell Carcinoma (OSCC) | down-regulated |
| hsa-let-7d | pancreatic cancer | up-regulated |
| hsa-let-7d | prostate cancer | down-regulated |
| hsa-let-7d* | cardiac hypertrophy | down-regulated |
| hsa-let-7e | lung cancer | down-regulated |
| hsa-let-7e | acute lymphoblastic leukemia (ALL) | down-regulated |
| hsa-let-7e | acute myeloid leukemia (AML) | up-regulated |
| hsa-let-7e | acute myeloid leukemia (AML) | up-regulated |
| hsa-let-7e | diffuse large B-cell lymphoma (DLBCL) | down-regulated |
| hsa-let-7e | head and neck squamous cell carcinoma (HNSCC) | up-regulated |
| hsa-let-7e | hepatocellular carcinoma (HCC) | down-regulated |
| hsa-let-7e | malignant melanoma | down-regulated |
| hsa-let-7e | Oral Squamous Cell Carcinoma (OSCC) | down-regulated |
| hsa-let-7e | ovarian cancer (OC) | down-regulated |
| hsa-let-7e | pituitary adenoma | down-regulated |
| hsa-let-7e | psoriasis | down-regulated |
| hsa-let-7f | lung cancer | down-regulated |
| hsa-miR-210 | glioblastoma multiforme (GBM) | up-regulated |
| hsa-miR-125a | acute lymphoblastic leukemia (ALL) | down-regulated |
| hsa-miR-125a | acute myeloid leukemia (AML) | up-regulated |
| hsa-miR-125a | breast cancer | down-regulated |
| hsa-miR-125a | chronic pancreatitis | up-regulated |
| hsa-miR-125a | colorectal cancer | down-regulated |
| hsa-miR-125a | epithelial ovarian cancer (EOC) | down-regulated |
| hsa-miR-125a | hepatocellular carcinoma (HCC) | down-regulated |
| hsa-miR-125a | lung cancer | down-regulated |
| hsa-miR-125a | neuroblastoma (NB) | down-regulated |
| hsa-miR-125a | pancreatic cancer | up-regulated |
| hsa-miR-125a | prostate cancer | down-regulated |
| hsa-miR-125a | serous ovarian cancer | down-regulated |
| hsa-miR-125a | vascular disease | down-regulated |
| hsa-miR-125b | anaplastic thyroid carcinoma (ATC) | down-regulated |
| hsa-miR-125b | breast cancer | down-regulated |
| hsa-miR-125b | breast cancer | down-regulated |
| hsa-miR-125b | prostate cancer | down-regulated |
| hsa-miR-125b | prostate cancer | up-regulated |
| hsa-miR-125b | Alzheimer's disease | up-regulated |
| hsa-miR-125b | cardiac hypertrophy | up-regulated |
| hsa-miR-125b | Cerebellar neurodegeneration | down-regulated |
| hsa-miR-125b | heart failure | up-regulated |
| hsa-miR-135a | malignant melanoma | down-regulated |
| hsa-miR-135b | colorectal cancer | up-regulated |
| hsa-miR-135b | malignant melanoma | down-regulated |
| hsa-miR-150 | pancreatic ductal adenocarcinoma (PDAC) | up-regulated |
| hsa-miR-150 | polycythemia vera (PV) | down-regulated |
| hsa-miR-150 | primary biliary cirrhosis (PBC) | down-regulated |
| hsa-miR-150 | uterine leiomyoma (ULM) | down-regulated |
| hsa-miR-151 | acute myeloid leukemia (AML) | down-regulated |
| hsa-miR-151 | cardiac hypertrophy | down-regulated |
| hsa-miR-151 | diffuse large B-cell lymphoma (DLBCL) | down-regulated |
| hsa-miR-151 | hepatocellular carcinoma (HCC) | up-regulated |
| hsa-miR-151 | nasopharyngeal carcinoma (NPC) | up-regulated |
| hsa-miR-151 | prostate cancer | up-regulated |
| hsa-miR-151* | acute lymphoblastic leukemia (ALL) | up-regulated |
| hsa-miR-151* | acute myeloid leukemia (AML) | down-regulated |
| hsa-miR-152 | asthma | normal |
| hsa-miR-152 | breast cancer | down-regulated |
| hsa-miR-152 | breast cancer | down-regulated |
| hsa-miR-152 | epithelial ovarian cancer (EOC) | down-regulated |
| hsa-miR-153 | B-cell chronic lymphocytic leukemia | up-regulated |
| hsa-miR-154 | B-cell chronic lymphocytic leukemia | up-regulated |
| hsa-miR-154 | dermatomyositis (DM) | up-regulated |
| hsa-miR-133a | tongue squamous cell carcinoma | down-regulated |
| hsa-miR-133a | epithelial ovarian cancer (EOC) | down-regulated |
| hsa-miR-133a | glioblastoma multiforme (GBM) | down-regulated |
| hsa-miR-133a | primary biliary cirrhosis (PBC) | down-regulated |
| hsa-miR-133a | retinitis pigmentosa (RP) | down-regulated |
| hsa-miR-133a | vascular disease | down-regulated |
| hsa-miR-133b | Parkinson's disease | down-regulated |
| hsa-miR-133b | tongue squamous cell carcinoma | down-regulated |
| hsa-miR-133b | colorectal cancer | down-regulated |
| hsa-miR-133b | glioblastoma multiforme (GBM) | down-regulated |
| hsa-miR-133b | Oral Squamous Cell Carcinoma (OSCC) | down-regulated |
| hsa-miR-133b | primary biliary cirrhosis (PBC) | down-regulated |
| hsa-miR-133b | psoriasis | down-regulated |
| hsa-miR-133b | testicular germ cell tumor | up-regulated |
| hsa-miR-134 | acute promyelocytic leukemia (APL) | down-regulated |
| hsa-miR-134 | B-cell chronic lymphocytic leukemia | up-regulated |
| hsa-miR-134 | Duchenne muscular dystrophy (DMD) | up-regulated |
| hsa-miR-134 | epithelial ovarian cancer (EOC) | down-regulated |
| hsa-miR-134 | limb-girdle muscular dystrophies types 2A (LGMD2A) | up-regulated |
| hsa-miR-134 | nemaline myopathy (NM) | up-regulated |
| hsa-miR-134 | ovarian cancer (OC) | down-regulated |
| hsa-miR-136 | breast cancer | up-regulated |
| hsa-miR-136 | hepatocellular carcinoma (HCC) | down-regulated |
| hsa-miR-21 | miyoshi myopathy (MM) | up-regulated |
| hsa-miR-21 | ovarian cancer (OC) | down-regulated |
| hsa-miR-21 | pancreatic cancer | up-regulated |
| hsa-miR-21 | pancreatic cancer | up-regulated |
| hsa-miR-21 | pancreatic cancer | up-regulated |
| hsa-miR-21 | papillary thyroid carcinoma (PTC) | up-regulated |
| hsa-miR-21 | polymyositis (PM) | up-regulated |
| hsa-miR-21 | prostate cancer | up-regulated |
| hsa-miR-21 | prostate cancer | up-regulated |
| hsa-miR-21 | psoriasis | up-regulated |
| hsa-miR-21 | serous ovarian cancer | up-regulated |
| hsa-miR-21 | uterine leiomyoma (ULM) | up-regulated |
| hsa-miR-21 | uterine leiomyoma (ULM) | up-regulated |
| hsa-miR-210 | breast cancer | up-regulated |
| hsa-miR-210 | diffuse large B-cell lymphoma (DLBCL) | up-regulated |
| hsa-miR-210 | acute lymphoblastic leukemia (ALL) | up-regulated |
| hsa-miR-210 | acute myeloid leukemia (AML) | down-regulated |
| hsa-miR-210 | Alzheimer's disease | down-regulated |
| hsa-miR-210 | breast cancer | up-regulated |
| hsa-miR-210 | dermatomyositis (DM) | up-regulated |
| hsa-miR-210 | diffuse large B-cell lymphoma (DLBCL) | up-regulated |
| hsa-miR-210 | Duchenne muscular dystrophy (DMD) | up-regulated |
| hsa-miR-210 | follicular lymphoma (FL) | up-regulated |
| hsa-miR-210 | head and neck squamous cell carcinoma (HNSCC) | up-regulated |
| hsa-miR-9 | Hodgkin's lymphoma | up-regulated |
| hsa-miR-31 | malignant melanoma | down-regulated |
| hsa-miR-31 | pancreatic ductal adenocarcinoma (PDAC) | up-regulated |
| hsa-miR-368 | facioscapulohumeral muscular dystrophy (FSHD) | up-regulated |
| hsa-miR-368 | limb-girdle muscular dystrophies types 2A (LGMD2A) | up-regulated |
| hsa-miR-368 | miyoshi myopathy (MM) | up-regulated |
| hsa-miR-368 | nemaline myopathy (NM) | up-regulated |
| hsa-miR-368 | Oral Squamous Cell Carcinoma (OSCC) | down-regulated |
| hsa-miR-369-5p | dermatomyositis (DM) | up-regulated |
| hsa-miR-369-5p | Duchenne muscular dystrophy (DMD) | up-regulated |
| hsa-miR-369-5p | facioscapulohumeral muscular dystrophy (FSHD) | up-regulated |
| hsa-miR-369-5p | nemaline myopathy (NM) | up-regulated |
| hsa-miR-370 | cholangiocarcinoma | down-regulated |
| hsa-miR-370 | Oral Squamous Cell Carcinoma (OSCC) | down-regulated |
| hsa-miR-370 | prostate cancer | up-regulated |
| hsa-miR-371 | Oral Squamous Cell Carcinoma (OSCC) | down-regulated |
| hsa-miR-371 | testicular germ cell tumor | up-regulated |
| hsa-miR-372 | non-small cell lung cancer (NSCLC) | up-regulated |
| hsa-miR-372 | testicular germ cell tumor | up-regulated |
| hsa-miR-372 | acute myeloid leukemia (AML) | down-regulated |
| hsa-miR-372 | Oral Squamous Cell Carcinoma (OSCC) | down-regulated |
| hsa-miR-372 | testicular germ cell tumor | up-regulated |
| hsa-miR-373 | breast cancer | up-regulated |
| hsa-miR-373 | testicular germ cell tumor | up-regulated |
| hsa-miR-182 | prostate cancer | up-regulated |
| hsa-miR-182* | prostate cancer | up-regulated |
| hsa-miR-183 | lung cancer | up-regulated |
| hsa-miR-183 | prostate cancer | up-regulated |
| hsa-miR-184 | prostate cancer | down-regulated |
| hsa-miR-206 | rhabdomyosarcoma | down-regulated |
| hsa-miR-206 | rhabdomyosarcoma | down-regulated |
| hsa-miR-208 | myocardial injury | up-regulated |
| hsa-miR-20a | kidney cancer | up-regulated |
| hsa-miR-20a | lung cancer | up-regulated |
| hsa-miR-20b | kidney cancer | up-regulated |
| hsa-miR-21 | kidney cancer | up-regulated |
| hsa-miR-21 | myocardial infarction | down-regulated |
| hsa-miR-21 | hepatocellular carcinoma (HCC) | up-regulated |
| hsa-miR-21 | lung cancer | up-regulated |
| hsa-miR-21 | cervical cancer | up-regulated |
| hsa-miR-21 | cholesteatoma | up-regulated |
| hsa-miR-21 | lung cancer | up-regulated |
| hsa-miR-210 | pancreatic cancer | up-regulated |
| hsa-miR-210 | lung cancer | up-regulated |
| hsa-miR-210 | kidney cancer | up-regulated |
| hsa-miR-214 | kidney cancer | down-regulated |
| hsa-miR-218 | lung cancer | down-regulated |
| hsa-miR-22 | lung cancer | down-regulated |
| hsa-miR-221 | prostate cancer | down-regulated |
| hsa-miR-221 | hepatocellular carcinoma (HCC) | up-regulated |
| hsa-miR-221 | prostate cancer | down-regulated |
| hsa-miR-221 | hepatocellular carcinoma (HCC) | up-regulated |
| hsa-miR-222 | pancreatic cancer | up-regulated |
| hsa-miR-222 | hepatocellular carcinoma (HCC) | up-regulated |
| hsa-miR-222 | prostate cancer | down-regulated |
| hsa-miR-223 | chronic lymphocytic leukemia (CLL) | down-regulated |
| hsa-miR-23a | cardiac hypertrophy | up-regulated |
| hsa-miR-26a | kidney cancer | down-regulated |
| hsa-miR-27a | breast cancer | up-regulated |
| hsa-miR-27a | kidney cancer | up-regulated |
| hsa-miR-29a | kidney cancer | up-regulated |
| hsa-miR-29a | neuroblastoma (NB) | down-regulated |
| hsa-miR-29b | neuroblastoma (NB) | down-regulated |
| hsa-miR-29b | kidney cancer | up-regulated |
| hsa-miR-34a | pancreatic cancer | down-regulated |
| hsa-miR-34b | pancreatic cancer | down-regulated |
| hsa-miR-34c | pancreatic cancer | down-regulated |
| hsa-miR-375 | prostate cancer | up-regulated |
| hsa-miR-378 | kidney cancer | down-regulated |
| hsa-miR-423 | lung cancer | up-regulated |
| hsa-miR-424 | kidney cancer | up-regulated |
| hsa-miR-424 | chronic lymphocytic leukemia (CLL) | down-regulated |
| hsa-miR-425-5p | lung cancer | up-regulated |
| hsa-miR-451 | lung cancer | down-regulated |
| hsa-miR-489 | kidney cancer | up-regulated |
| hsa-miR-497 | lung cancer | down-regulated |
| hsa-miR-511 | lung cancer | down-regulated |
| hsa-miR-532-5p | kidney cancer | down-regulated |
| hsa-miR-599 | multiple sclerosis | up-regulated |
| hsa-miR-661 | breast cancer | down-regulated |
| hsa-miR-7 | Parkinson's disease | down-regulated |
| hsa-miR-7 | lung cancer | up-regulated |
| hsa-miR-720 | kidney cancer | down-regulated |
| hsa-miR-107 | non-small cell lung cancer (NSCLC) | down-regulated |
| hsa-miR-107 | chronic lymphocytic leukemia (CLL) | down-regulated |
| hsa-miR-107 | schizophrenia | up-regulated |
| hsa-miR-122 | hepatocellular carcinoma (HCC) | down-regulated |
| hsa-miR-122 | hepatocellular carcinoma (HCC) | down-regulated |
| hsa-miR-122 | kidney cancer | up-regulated |
| hsa-miR-125a | type 2 diabetes | up-regulated |
| hsa-miR-125a- | lung cancer | down-regulated |
| hsa-miR-125b | prostate cancer | down-regulated |
| hsa-miR-122a | gastric cancer (stomach cancer) | down-regulated |
| hsa-miR-122a | hepatocellular carcinoma (HCC) | down-regulated |
| hsa-miR-126 | kidney cancer | up-regulated |
| hsa-miR-126 | lung cancer | down-regulated |
| hsa-miR-126* | lung cancer | down-regulated |
| hsa-miR-128 | neuroblastoma | down-regulated |
| hsa-miR-130a | lung cancer | down-regulated |
| hsa-miR-133b | lung cancer | down-regulated |
| hsa-miR-135a | Hodgkin's lymphoma | down-regulated |
| hsa-miR-139 | lung cancer | down-regulated |
| hsa-miR-141 | lung cancer | up-regulated |
| hsa-miR-199a | Intrahepatic cholangiocarcinoma (ICC) | down-regulated |
| hsa-miR-199a* | Intrahepatic cholangiocarcinoma (ICC) | down-regulated |
| hsa-miR-214 | Intrahepatic cholangiocarcinoma (ICC) | down-regulated |
| hsa-miR-22 | Intrahepatic cholangiocarcinoma (ICC) | down-regulated |
| hsa-miR-155 | pancreatic ductal adenocarcinoma (PDAC) | up-regulated |
| hsa-miR-196a | pancreatic ductal adenocarcinoma (PDAC) | up-regulated |
| hsa-miR-122 | hepatocellular carcinoma (HCC) | down-regulated |
| hsa-miR-208a | cardiac hypertrophy | down-regulated |
| hsa-miR-200c | lung cancer | down-regulated |
| hsa-miR-429 | lung cancer | down-regulated |
| hsa-miR-221 | bladder cancer | up-regulated |
| hsa-miR-21 | pancreatic cancer | up-regulated |
| hsa-miR-221 | pancreatic cancer | up-regulated |
| hsa-miR-140 | colorectal cancer | down-regulated |
| hsa-miR-140 | osteosarcoma | down-regulated |
| hsa-miR-21 | colorectal cancer | up-regulated |
| hsa-miR-21 | prostate cancer | up-regulated |
| hsa-miR-125b | breast cancer | normal |
| hsa-miR-16 | prostate cancer | down-regulated |
| hsa-miR-100 | nasopharyngeal carcinoma (NPC) | down-regulated |
| hsa-miR-10a | pancreatic cancer | up-regulated |
| hsa-miR-1 | lung cancer | down-regulated |
| hsa-miR-143 | lung cancer | down-regulated |
| hsa-miR-130a | lung cancer | up-regulated |
| hsa-miR-146b | lung cancer | up-regulated |
| hsa-miR-21 | lung cancer | up-regulated |
| hsa-miR-31 | lung cancer | up-regulated |
| hsa-miR-377 | lung cancer | up-regulated |
| hsa-miR-141 | lung cancer | down-regulated |
| hsa-miR-200a | lung cancer | down-regulated |
| hsa-miR-200b | lung cancer | down-regulated |
| hsa-miR-221 | acute promyelocytic leukemia (APL) | down-regulated |
| hsa-miR-128b | acute promyelocytic leukemia (APL) | down-regulated |
| hsa-miR-127 | Intrahepatic cholangiocarcinoma (ICC) | down-regulated |
| hsa-miR-376a | Intrahepatic cholangiocarcinoma (ICC) | down-regulated |
| hsa-miR-424 | Intrahepatic cholangiocarcinoma (ICC) | down-regulated |
| hsa-miR-17-3p | prostate cancer | down-regulated |
| hsa-miR-34a | glioblastoma | down-regulated |
| hsa-miR-34a | glioma | down-regulated |
| hsa-miR-34a | medulloblastoma | down-regulated |
| hsa-miR-1 | myocardial infarction | up-regulated |
| hsa-miR-130b | glioma | down-regulated |
| hsa-miR-140 | glioma | down-regulated |
| hsa-miR-15a | glioma | down-regulated |
| hsa-miR-16 | glioma | down-regulated |
| hsa-miR-184 | glioma | down-regulated |
| hsa-miR-19a | glioma | down-regulated |
| hsa-miR-20a | glioma | down-regulated |
| hsa-miR-21 | glioma | down-regulated |
| hsa-miR-210 | glioma | down-regulated |
| hsa-miR-25 | glioma | down-regulated |
| hsa-miR-28 | glioma | down-regulated |
| hsa-miR-328 | glioma | down-regulated |
| hsa-miR-9 | glioma | down-regulated |
| hsa-miR-17 | glioma | up-regulated |
| hsa-miR-200a | Hodgkin's lymphoma | down-regulated |
| hsa-miR-520a | Hodgkin's lymphoma | down-regulated |
| hsa-miR-614 | Hodgkin's lymphoma | down-regulated |
| hsa-miR-140 | Hodgkin's lymphoma | up-regulated |
| hsa-miR-155 | Hodgkin's lymphoma | up-regulated |
| hsa-miR-16 | Hodgkin's lymphoma | up-regulated |
| hsa-miR-186 | Hodgkin's lymphoma | up-regulated |
| hsa-miR-18a | Hodgkin's lymphoma | up-regulated |
| hsa-miR-196a | Hodgkin's lymphoma | up-regulated |
| hsa-miR-20a | Hodgkin's lymphoma | up-regulated |
| hsa-miR-21 | Hodgkin's lymphoma | up-regulated |
| hsa-miR-30a-5p | Hodgkin's lymphoma | up-regulated |
| hsa-miR-181b | Oral Carcinoma | up-regulated |
| hsa-miR-21 | Oral Carcinoma | up-regulated |
| hsa-miR-345 | Oral Carcinoma | up-regulated |
| hsa-miR-30b | Hodgkin's lymphoma | up-regulated |
| hsa-miR-374 | Hodgkin's lymphoma | up-regulated |
| hsa-miR-9 | Hodgkin's lymphoma | up-regulated |
| hsa-miR-10b | non-alcoholic fatty liver disease (NAFLD) | up-regulated |
| hsa-let-7c | non-alcoholic fatty liver disease (NAFLD) | down-regulated |
| hsa-let-7d | non-alcoholic fatty liver disease (NAFLD) | down-regulated |
| hsa-let-7e | non-alcoholic fatty liver disease (NAFLD) | down-regulated |
| hsa-miR-103 | non-alcoholic fatty liver disease (NAFLD) | down-regulated |
| hsa-miR-107 | non-alcoholic fatty liver disease (NAFLD) | down-regulated |
| hsa-miR-130a | non-alcoholic fatty liver disease (NAFLD) | down-regulated |
| hsa-miR-140 | non-alcoholic fatty liver disease (NAFLD) | down-regulated |
| hsa-miR-183 | non-alcoholic fatty liver disease (NAFLD) | down-regulated |
| hsa-miR-200c | non-alcoholic fatty liver disease (NAFLD) | down-regulated |
| hsa-miR-203 | non-alcoholic fatty liver disease (NAFLD) | down-regulated |
| hsa-miR-375 | esophageal cancer | down-regulated |
| hsa-miR-192 | esophageal cancer | up-regulated |
| hsa-miR-194 | esophageal cancer | up-regulated |
| hsa-miR-21 | esophageal cancer | up-regulated |
| hsa-miR-21 | non-alcoholic fatty liver disease (NAFLD) | down-regulated |
| hsa-miR-26a-1 | non-alcoholic fatty liver disease (NAFLD) | down-regulated |
| hsa-miR-27b | non-alcoholic fatty liver disease (NAFLD) | down-regulated |
| hsa-miR-29b | non-alcoholic fatty liver disease (NAFLD) | down-regulated |
| hsa-miR-30a | non-alcoholic fatty liver disease (NAFLD) | down-regulated |
| hsa-miR-122a | non-alcoholic fatty liver disease (NAFLD) | up-regulated |
| hsa-miR-126 | non-alcoholic fatty liver disease (NAFLD) | up-regulated |
| hsa-miR-132 | non-alcoholic fatty liver disease (NAFLD) | up-regulated |
| hsa-miR-151 | non-alcoholic fatty liver disease (NAFLD) | up-regulated |
| hsa-miR-154 | non-alcoholic fatty liver disease (NAFLD) | up-regulated |
| hsa-miR-16-1 | non-alcoholic fatty liver disease (NAFLD) | up-regulated |
| hsa-miR-17 | non-alcoholic fatty liver disease (NAFLD) | up-regulated |
| hsa-miR-187 | non-alcoholic fatty liver disease (NAFLD) | up-regulated |
| hsa-miR-22 | non-alcoholic fatty liver disease (NAFLD) | up-regulated |
| hsa-miR-29c | non-alcoholic fatty liver disease (NAFLD) | up-regulated |
| hsa-miR-210 | head and neck cancer | down-regulated |
| hsa-let-7a | laryngeal carcinoma | down-regulated |
| hsa-miR-203 | esophageal cancer | down-regulated |
| hsa-miR-30c | non-alcoholic fatty liver disease (NAFLD) | up-regulated |
| hsa-miR-30d | non-alcoholic fatty liver disease (NAFLD) | up-regulated |
| hsa-miR-31 | non-alcoholic fatty liver disease (NAFLD) | up-regulated |
| hsa-miR-33 | non-alcoholic fatty liver disease (NAFLD) | up-regulated |
| hsa-miR-34a | non-alcoholic fatty liver disease (NAFLD) | up-regulated |
| hsa-miR-95 | non-alcoholic fatty liver disease (NAFLD) | up-regulated |
| hsa-miR-223 | esophageal cancer | up-regulated |
| hsa-miR-22 | acute lymphoblastic leukemia (ALL) | down-regulated |
| hsa-miR-223 | essential thrombocythemia (ET) | up-regulated |
| hsa-miR-146b | primary myelofibrosis | up-regulated |
| hsa-miR-223 | primary myelofibrosis | up-regulated |
| hsa-miR-26a | hepatocellular carcinoma (HCC) | down-regulated |
| hsa-miR-26b | hepatocellular carcinoma (HCC) | down-regulated |
| hsa-miR-29a | lung cancer | down-regulated |
| hsa-let-7a | lung cancer | down-regulated |
| hsa-miR-17 | Polycystic Kidney Disease | up-regulated |
| hsa-miR-150 | sepsis | down-regulated |
| hsa-miR-106b | colorectal cancer | up-regulated |
| hsa-miR-130b | colorectal cancer | up-regulated |
| hsa-miR-181b | colorectal cancer | up-regulated |
| hsa-miR-20b | multiple sclerosis | down-regulated |
| hsa-miR-1275 | multiple sclerosis | up-regulated |
| hsa-miR-142-3p | multiple sclerosis | up-regulated |
| hsa-miR-145 | multiple sclerosis | up-regulated |
| hsa-miR-186 | multiple sclerosis | up-regulated |
| hsa-miR-223 | multiple sclerosis | up-regulated |
| hsa-miR-422a | multiple sclerosis | up-regulated |
| hsa-miR-491-5p | multiple sclerosis | up-regulated |
| hsa-miR-584 | multiple sclerosis | up-regulated |
| hsa-miR-664 | multiple sclerosis | up-regulated |
| hsa-miR-181a | breast cancer | down-regulated |
| hsa-miR-26a | breast cancer | down-regulated |
| hsa-miR-21 | colorectal cancer | up-regulated |
| hsa-miR-141 | colorectal cancer | down-regulated |
| hsa-miR-34a | malignant melanoma | down-regulated |
| hsa-miR-15 | malignant melanoma | up-regulated |
| hsa-miR-210 | malignant melanoma | up-regulated |
| hsa-miR--196a-2 | gastric cancer (stomach cancer) | down-regulated |
| hsa-miR-326 | multiple sclerosis | up-regulated |
| hsa-miR-143 | colorectal cancer | down-regulated |
| hsa-miR-200b | breast cancer | down-regulated |
| hsa-miR-200c | breast cancer | down-regulated |
| hsa-miR-1 | colorectal cancer | down-regulated |
| hsa-miR-10b | colorectal cancer | down-regulated |
| hsa-miR-125a | colorectal cancer | down-regulated |
| hsa-miR-133a | colorectal cancer | down-regulated |
| hsa-miR-139 | colorectal cancer | down-regulated |
| hsa-miR-143 | colorectal cancer | down-regulated |
| hsa-miR-145 | colorectal cancer | down-regulated |
| hsa-miR-195 | colorectal cancer | down-regulated |
| hsa-miR-30a-3p | colorectal cancer | down-regulated |
| hsa-miR-30a-5p | colorectal cancer | down-regulated |
| hsa-miR-30c | colorectal cancer | down-regulated |
| hsa-miR-378* | colorectal cancer | down-regulated |
| hsa-miR-422a | colorectal cancer | down-regulated |
| hsa-miR-422b | colorectal cancer | down-regulated |
| hsa-miR-497 | colorectal cancer | down-regulated |
| hsa-miR-203 | colorectal cancer | down-regulated |
| hsa-miR-34a | colorectal cancer | down-regulated |
| hsa-miR-95 | colorectal cancer | down-regulated |
| hsa-miR-106a | colorectal cancer | up-regulated |
| hsa-miR-19a | colorectal cancer | up-regulated |
| hsa-miR-19b | colorectal cancer | up-regulated |
| hsa-miR-20a | colorectal cancer | up-regulated |
| hsa-miR-21 | colorectal cancer | up-regulated |
| hsa-miR-224 | colorectal cancer | up-regulated |
| hsa-miR-25 | colorectal cancer | up-regulated |
| hsa-miR-29a | colorectal cancer | up-regulated |
| hsa-miR-96 | colorectal cancer | up-regulated |
| hsa-miR-17-5p | colorectal cancer | up-regulated |
| hsa-miR-182 | colorectal cancer | up-regulated |
| hsa-miR-183 | colorectal cancer | up-regulated |
| hsa-miR-18a | colorectal cancer | up-regulated |
| hsa-miR-29b | colorectal cancer | up-regulated |
| hsa-miR-93 | colorectal cancer | up-regulated |
| hsa-miR-31 | colorectal cancer | up-regulated |
| hsa-miR-124 | hepatocellular carcinoma (HCC) | down-regulated |
| hsa-miR-203 | hepatocellular carcinoma (HCC) | down-regulated |
| hsa-miR-126 | asthma | up-regulated |
| hsa-miR-21 | bladder cancer | up-regulated |
| hsa-miR-100 | bladder cancer | down-regulated |
| hsa-miR-99a | bladder cancer | down-regulated |
| hsa-miR-29b | acute myelogeneous leukemia (AML) | down-regulated |
| hsa-let-7b | multiple myeloma (MM) | down-regulated |
| hsa-miR-140-3p | multiple myeloma (MM) | down-regulated |
| hsa-let-7e | multiple myeloma (MM) | up-regulated |
| hsa-miR-125a-5p | multiple myeloma (MM) | up-regulated |
| hsa-miR-99b | multiple myeloma (MM) | up-regulated |
| hsa-miR-15a | papillary thyroid carcinoma (PTC) | down-regulated |
| hsa-miR-16 | papillary thyroid carcinoma (PTC) | down-regulated |
| hsa-miR-199b | papillary thyroid carcinoma (PTC) | down-regulated |
| hsa-miR-26a | papillary thyroid carcinoma (PTC) | down-regulated |
| hsa-miR-29 | papillary thyroid carcinoma (PTC) | down-regulated |
| hsa-miR-34 | papillary thyroid carcinoma (PTC) | down-regulated |
| hsa-let-7b | papillary thyroid carcinoma (PTC) | up-regulated |
| hsa-miR-106 | papillary thyroid carcinoma (PTC) | up-regulated |
| hsa-miR-193 | papillary thyroid carcinoma (PTC) | up-regulated |
| hsa-miR-200a | papillary thyroid carcinoma (PTC) | up-regulated |
| hsa-miR-222 | papillary thyroid carcinoma (PTC) | up-regulated |
| hsa-miR-141 | ovarian cancer (OC) | up-regulated |
| hsa-miR-200a | ovarian cancer (OC) | up-regulated |
| hsa-miR-200b | ovarian cancer (OC) | up-regulated |
| hsa-miR-200c | ovarian cancer (OC) | up-regulated |
| hsa-miR-429 | ovarian cancer (OC) | up-regulated |
| hsa-miR-214 | cervical cancer | down-regulated |
| hsa-miR-500 | hepatocellular carcinoma (HCC) | up-regulated |
| hsa-miR-31a | breast cancer | down-regulated |
| hsa-miR-125a | breast cancer | down-regulated |
| miR-BART21 | nasopharyngeal carcinoma (NPC) | up-regulated |
| miR-BART22 | nasopharyngeal carcinoma (NPC) | up-regulated |
| hsa-miR-125a | ovarian cancer (OC) | down-regulated |
| hsa-miR-16-1 | chronic lymphocytic leukemia (CLL) | down-regulated |
| hsa-miR-16-1 | chronic lymphocytic leukemia (CLL) | down-regulated |
| hsa-miR-16-1 | mantle cell lymphoma (MCL) | normal |
| hsa-miR-16-1 | pituitary adenoma | down-regulated |
| hsa-miR-16-1 | pancreatic cancer | up-regulated |
| hsa-miR-16-2 | chronic lymphocytic leukemia (CLL) | up-regulated |
| hsa-miR-164 | pituitary adenoma | down-regulated |
| hsa-miR-170 | breast cancer | up-regulated |
| hsa-miR-172a-2 | hepatocellular carcinoma (HCC) | up-regulated |
| hsa-miR-17-3p | lung cancer | up-regulated |
| hsa-miR-17-3p | anaplastic thyroid carcinoma (ATC) | up-regulated |
| hsa-miR-17-3p | lung cancer | up-regulated |
| hsa-miR-17-3p | MYC-rearranged lymphoma | up-regulated |
| hsa-miR-17-3p | hepatocellular carcinoma (HCC) | up-regulated |
| hsa-miR-17-3p | malignant lymphoma | up-regulated |
| hsa-miR-409-3p | dermatomyositis (DM) | up-regulated |
| hsa-miR-409-3p | Duchenne muscular dystrophy (DMD) | up-regulated |
| hsa-miR-409-3p | miyoshi myopathy (MM) | up-regulated |
| hsa-miR-409-3p | nemaline myopathy (NM) | up-regulated |
| hsa-miR-419 | epithelial ovarian cancer (EOC) | down-regulated |
| hsa-miR-422a | ovarian cancer (OC) | down-regulated |
| hsa-miR-422b | head and neck squamous cell carcinoma (HNSCC) | down-regulated |
| hsa-miR-423 | Duchenne muscular dystrophy (DMD) | down-regulated |
| hsa-miR-423 | nemaline myopathy (NM) | up-regulated |
| hsa-miR-423 | primary biliary cirrhosis (PBC) | down-regulated |
| hsa-miR-637 | head and neck squamous cell carcinoma (HNSCC) | up-regulated |
| hsa-miR-637 | ovarian cancer (OC) | up-regulated |
| hsa-miR-642 | uterine leiomyoma (ULM) | down-regulated |
| hsa-miR-648 | ovarian cancer (OC) | down-regulated |
| hsa-miR-652 | autism spectrum disorder (ASD) | down-regulated |
| hsa-miR-652 | uveal melanoma | up-regulated |
| hsa-miR-657 | ovarian cancer (OC) | down-regulated |
| hsa-miR-662 | ovarian cancer (OC) | down-regulated |
| hsa-miR-663 | breast cancer | down-regulated |
| hsa-miR-663 | breast cancer | down-regulated |
| hsa-miR-663 | ovarian cancer (OC) | up-regulated |
| hsa-miR-7 | glioblastoma | down-regulated |
| hsa-miR-7 | autism spectrum disorder (ASD) | up-regulated |
| hsa-miR-7 | head and neck squamous cell carcinoma (HNSCC) | up-regulated |
| hsa-miR-7 | schizophrenia | down-regulated |
| hsa-miR-7-1 | pituitary adenoma | down-regulated |
| hsa-miR-7-2 | kidney cancer | up-regulated |
| hsa-miR-7-3 | chronic pancreatitis | up-regulated |
| hsa-miR-7-3 | colorectal cancer | up-regulated |
| hsa-miR-7-3 | pituitary adenoma | down-regulated |
| hsa-miR-802 | Down syndrome (DS) | up-regulated |
| hsa-miR-9 | metabolic disease | up-regulated |
| hsa-miR-9 | recurrent ovarian cancer | down-regulated |
| hsa-miR-9 | Alzheimer's disease | up-regulated |
| hsa-miR-10a | Oral Squamous Cell Carcinoma (OSCC) | up-regulated |
| hsa-miR-10a | pancreatic cancer | up-regulated |
| hsa-miR-10a | prostate cancer | up-regulated |
| hsa-miR-10a | psoriasis | down-regulated |
| hsa-miR-10a | uterine leiomyoma (ULM) | down-regulated |
| hsa-miR-10b | breast cancer | up-regulated |
| hsa-miR-10b | B-cell chronic lymphocytic leukemia | up-regulated |
| hsa-miR-10b | breast cancer | down-regulated |
| hsa-miR-10b | cardiac hypertrophy | down-regulated |
| hsa-miR-10b | chronic pancreatitis | up-regulated |
| hsa-miR-10b | glioblastoma | up-regulated |
| hsa-miR-10b | ovarian cancer (OC) | up-regulated |
| hsa-miR-10b | pancreatic cancer | up-regulated |
| hsa-miR-10b | prostate cancer | up-regulated |
| hsa-miR-10b | serous ovarian cancer | down-regulated |
| hsa-miR-1-2 | hepatocellular carcinoma (HCC) | down-regulated |
| hsa-miR-1-2 | cardiomyopathy | down-regulated |
| hsa-miR-122a | hepatocellular carcinoma (HCC) | down-regulated |
| hsa-miR-122a | HCV infection | up-regulated |
| hsa-miR-122a | hepatocellular carcinoma (HCC) | down-regulated |
| hsa-miR-122a | metabolic disease | down-regulated |
| hsa-miR-122a | breast cancer | up-regulated |
| hsa-miR-122a | head and neck squamous cell carcinoma (HNSCC) | down-regulated |
| hsa-miR-122a | hepatocellular carcinoma (HCC) | up-regulated |
| hsa-miR-122a | hepatocellular carcinoma (HCC) | down-regulated |
| hsa-miR-184 | epithelial ovarian cancer (EOC) | down-regulated |
| hsa-miR-184 | malignant melanoma | down-regulated |
| hsa-miR-184 | Oral Squamous Cell Carcinoma (OSCC) | down-regulated |
| hsa-miR-184 | prostate cancer | up-regulated |
| hsa-miR-185 | hepatocellular carcinoma (HCC) | up-regulated |
| hsa-miR-185 | bladder cancer | up-regulated |
| hsa-miR-185 | cardiac hypertrophy | down-regulated |
| hsa-miR-185 | cardiac hypertrophy | down-regulated |
| hsa-miR-185 | glioblastoma multiforme (GBM) | down-regulated |
| hsa-miR-185 | kidney cancer | up-regulated |
| hsa-miR-185 | malignant melanoma | down-regulated |
| hsa-miR-130a | limb-girdle muscular dystrophies types 2A (LGMD2A) | up-regulated |
| hsa-miR-130a | miyoshi myopathy (MM) | up-regulated |
| hsa-miR-130a | nemaline myopathy (NM) | up-regulated |
| hsa-miR-130a | polymyositis (PM) | up-regulated |
| hsa-miR-130a-1 | hepatocellular carcinoma (HCC) | up-regulated |
| hsa-miR-130b | acute lymphoblastic leukemia (ALL) | up-regulated |
| hsa-miR-130b | acute myeloid leukemia (AML) | down-regulated |
| hsa-miR-130b | pancreatic ductal adenocarcinoma (PDAC) | down-regulated |
| hsa-miR-130b-1 | hepatocellular carcinoma (HCC) | up-regulated |
| hsa-miR-132 | Huntington's disease (HD) | down-regulated |
| hsa-miR-132 | autism spectrum disorder (ASD) | up-regulated |
| hsa-miR-132 | B-cell chronic lymphocytic leukemia | up-regulated |
| hsa-miR-139 | uterine leiomyoma (ULM) | down-regulated |
| hsa-miR-132 | colorectal cancer | up-regulated |
| hsa-miR-302c | malignant melanoma | down-regulated |
| hsa-miR-302c* | Oral Squamous Cell Carcinoma (OSCC) | down-regulated |
| hsa-miR-302c* | prostate cancer | up-regulated |
| hsa-miR-302d | acute myeloid leukemia (AML) | down-regulated |
| hsa-miR-302d | malignant melanoma | down-regulated |
| hsa-miR-302d | testicular germ cell tumor | up-regulated |
| hsa-miR-30a | hepatocellular carcinoma (HCC) | down-regulated |
| hsa-miR-30a | uterine leiomyoma (ULM) | up-regulated |
| hsa-miR-30a-3p | acute myeloid leukemia (AML) | down-regulated |
| hsa-miR-30a-3p | cardiac hypertrophy | down-regulated |
| hsa-miR-30a-3p | colorectal cancer | up-regulated |
| hsa-miR-30a-3p | colorectal cancer | down-regulated |
| hsa-miR-30a-3p | dermatomyositis (DM) | down-regulated |
| hsa-miR-30a-3p | Duchenne muscular dystrophy (DMD) | down-regulated |
| hsa-miR-30a-3p | limb-girdle muscular dystrophies types 2A (LGMD2A) | down-regulated |
| hsa-miR-30a-3p | miyoshi myopathy (MM) | down-regulated |
| hsa-miR-30a-3p | nemaline myopathy (NM) | down-regulated |
| hsa-miR-30a-3p | polymyositis (PM) | down-regulated |
| hsa-miR-30a-5p | anaplastic thyroid carcinoma (ATC) | down-regulated |
| hsa-miR-30a-5p | cardiac hypertrophy | down-regulated |
| hsa-miR-30a-5p | Duchenne muscular dystrophy (DMD) | down-regulated |
| hsa-miR-30a-5p | lung cancer | down-regulated |
| hsa-miR-200b | cancer | down-regulated |
| hsa-miR-30a-5p | colorectal cancer | up-regulated |
| hsa-miR-30d | primary biliary cirrhosis (PBC) | down-regulated |
| hsa-miR-30d | schizophrenia | down-regulated |
| hsa-miR-30e | hepatocellular carcinoma (HCC) | down-regulated |
| hsa-miR-30e | cardiac hypertrophy | down-regulated |
| hsa-miR-30e | cardiac hypertrophy | down-regulated |
| hsa-miR-30e | schizophrenia | down-regulated |
| hsa-miR-30e* | cardiac hypertrophy | down-regulated |
| hsa-miR-30e-5p | Duchenne muscular dystrophy (DMD) | down-regulated |
| hsa-miR-30e-5p | head and neck squamous cell carcinoma (HNSCC) | down-regulated |
| hsa-miR-30e-5p | Oral Squamous Cell Carcinoma (OSCC) | down-regulated |
| hsa-miR-30e-5p | ovarian cancer (OC) | up-regulated |
| hsa-miR-30e-5p | psoriasis | up-regulated |
| hsa-miR-31 | cardiac hypertrophy | up-regulated |
| hsa-miR-31 | colorectal cancer | up-regulated |
| hsa-miR-31 | colorectal cancer | up-regulated |
| hsa-miR-31 | head and neck squamous cell carcinoma (HNSCC) | up-regulated |
| hsa-miR-31 | hepatocellular carcinoma (HCC) | up-regulated |
| hsa-miR-31 | Oral Squamous Cell Carcinoma (OSCC) | up-regulated |
| hsa-miR-31 | psoriasis | up-regulated |
| hsa-miR-32 | PFV-1 infection | down-regulated |
| hsa-miR-32 | colorectal cancer | up-regulated |
| hsa-miR-19a | anaplastic thyroid carcinoma (ATC) | up-regulated |
| hsa-miR-19a | Cowden Syndrome | up-regulated |
| hsa-miR-19a | lung cancer | up-regulated |
| hsa-miR-19a | B-cell chronic lymphocytic leukemia | up-regulated |
| hsa-miR-19a | colorectal cancer | up-regulated |
| hsa-miR-19a | head and neck squamous cell carcinoma (HNSCC) | down-regulated |
| hsa-miR-19a | malignant lymphoma | up-regulated |
| hsa-miR-19a | malignant melanoma | up-regulated |
| hsa-miR-19b | malignant lymphoma | up-regulated |
| hsa-miR-19b | prostate cancer | down-regulated |
| hsa-miR-19b-2 | T-cell leukemia | up-regulated |
| hsa-miR-200a | serous ovarian cancer | up-regulated |
| hsa-miR-200a | breast cancer | down-regulated |
| hsa-miR-200a | cancer | down-regulated |
| hsa-miR-200a | epithelial ovarian cancer (EOC) | up-regulated |
| hsa-miR-200a | head and neck squamous cell carcinoma (HNSCC) | up-regulated |
| hsa-miR-200a | hepatocellular carcinoma (HCC) | down-regulated |
| hsa-miR-200a | malignant melanoma | down-regulated |
| hsa-miR-200a | ovarian cancer (OC) | down-regulated |
| hsa-miR-200a | psoriasis | up-regulated |
| hsa-miR-200b | serous ovarian cancer | up-regulated |
| hsa-miR-200b | breast cancer | down-regulated |
| hsa-miR-200b | cholangiocarcinoma | up-regulated |
| hsa-miR-24 | schizophrenia | down-regulated |
| hsa-miR-24-1 | chronic lymphocytic leukemia (CLL) | up-regulated |
| hsa-miR-24-1 | B-cell chronic lymphocytic leukemia | up-regulated |
| hsa-miR-24-1 | colorectal cancer | up-regulated |
| hsa-miR-24-1 | gastric cancer (stomach cancer) | up-regulated |
| hsa-miR-24-1 | glioblastoma | up-regulated |
| hsa-miR-24-1 | pancreatic cancer | up-regulated |
| hsa-miR-24-1 | pancreatic cancer | up-regulated |
| hsa-miR-24-1 | papillary thyroid carcinoma (PTC) | up-regulated |
| hsa-miR-24-1 | pituitary adenoma | down-regulated |
| hsa-miR-24-2 | hepatocellular carcinoma (HCC) | up-regulated |
| hsa-miR-24-2 | colorectal cancer | up-regulated |
| hsa-miR-24-2 | gastric cancer (stomach cancer) | up-regulated |
| hsa-miR-24-2 | glioblastoma | up-regulated |
| hsa-miR-24-2 | lung cancer | up-regulated |
| hsa-miR-24-2 | pancreatic cancer | up-regulated |
| hsa-miR-24-2 | pancreatic cancer | up-regulated |
| hsa-miR-24-2 | papillary thyroid carcinoma (PTC) | up-regulated |
| hsa-miR-25 | colorectal cancer | down-regulated |
| hsa-miR-25 | gastric cancer (stomach cancer) | up-regulated |
| hsa-miR-25 | glioblastoma | up-regulated |
| hsa-miR-25 | pancreatic cancer | up-regulated |
| hsa-miR-25 | prostate cancer | up-regulated |
| hsa-miR-26a | anaplastic thyroid carcinoma (ATC) | down-regulated |
| hsa-miR-26a | acute lymphoblastic leukemia (ALL) | down-regulated |
| hsa-miR-204 | malignant melanoma | down-regulated |
| hsa-miR-206 | breast cancer | down-regulated |
| hsa-let-7a | malignant melanoma | down-regulated |
| hsa-miR-136 | pituitary adenoma | down-regulated |
| hsa-miR-210 | limb-girdle muscular dystrophies types 2A (LGMD2A) | up-regulated |
| hsa-miR-424 | acute lymphoblastic leukemia (ALL) | down-regulated |
| hsa-miR-9 | Alzheimer's disease | down-regulated |
| hsa-miR-122a | hepatocellular carcinoma (HCC) | down-regulated |
| hsa-miR-140 | autism spectrum disorder (ASD) | down-regulated |
| hsa-miR-200b | epithelial ovarian cancer (EOC) | up-regulated |
| hsa-miR-26a | acute myeloid leukemia (AML) | up-regulated |
| hsa-miR-30a-5p | ovarian cancer (OC) | up-regulated |
| hsa-miR-32 | lung cancer | down-regulated |
| hsa-miR-373 | head and neck squamous cell carcinoma (HNSCC) | up-regulated |
| hsa-miR-154 | Duchenne muscular dystrophy (DMD) | up-regulated |
| hsa-miR-125b | malignant melanoma | down-regulated |
| hsa-miR-146b | Becker muscular dystrophy (BMD) | up-regulated |
| hsa-miR-192-2 | hepatocellular carcinoma (HCC) | down-regulated |
| hsa-miR-196b | pancreatic ductal adenocarcinoma (PDAC) | up-regulated |
| hsa-miR-382 | nemaline myopathy (NM) | up-regulated |
| hsa-miR-324-5p | medulloblastoma | down-regulated |
| hsa-miR-23a | lupus nephritis | up-regulated |
| hsa-let-7b | neurodegeneration | up-regulated |
| hsa-miR-30d | chronic lymphocytic leukemia (CLL) | down-regulated |
| hsa-miR-155 | breast cancer | up-regulated |
| hsa-miR-210 | Acute Promyelocytic Leukemia (APL) | down-regulated |
| hsa-miR-200b | hepatocellular carcinoma (HCC) | down-regulated |
| hsa-miR-144 | thalassemia | down-regulated |
| hsa-miR-21 | Hepatocellular carcinoma (HCC) | up-regulated |
| hsa-miR-142-3p | endometriosis | down-regulated |
| hsa-miR-19a | medulloblastoma | up-regulated |
| hsa-miR-223 | chronic lymphocytic leukemia (CLL) | down-regulated |
| hsa-miR-9* | Huntington's disease (HD) | down-regulated |
| hsa-miR-17-5p | diffuse large B-cell lymphoma (DLBCL) | up-regulated |
| hsa-miR-222 | prostate cancer | up-regulated |
| hsa-miR-19a | medulloblastoma | up-regulated |
| hsa-miR-10b | glioma | up-regulated |
| hsa-miR-133a | bladder cancer | down-regulated |
| hsa-miR-152 | hepatocellular carcinoma (HCC) | down-regulated |
| hsa-miR-199a | hepatocellular carcinoma (HCC) | down-regulated |
| hsa-miR-21 | laryngeal carcinoma | up-regulated |
| hsa-miR-21 | renal clear cell carcinoma | up-regulated |
| hsa-miR-21 | non-small cell lung cancer (NSCLC) | up-regulated |
| hsa-miR-21 | bladder cancer | up-regulated |
| hsa-miR-30a-5p | prostate cancer | down-regulated |
| hsa-miR-30a-5p | schizophrenia | down-regulated |
| hsa-miR-30b | cardiac hypertrophy | down-regulated |
| hsa-miR-30b | Duchenne muscular dystrophy (DMD) | down-regulated |
| hsa-miR-30b | head and neck squamous cell carcinoma (HNSCC) | up-regulated |
| hsa-miR-30b | malignant melanoma | down-regulated |
| hsa-miR-30b | nemaline myopathy (NM) | up-regulated |
| hsa-miR-30b | prostate cancer | down-regulated |
| hsa-miR-30b | schizophrenia | down-regulated |
| hsa-miR-30c | cardiac hypertrophy | down-regulated |
| hsa-miR-30c | Cerebellar neurodegeneration | down-regulated |
| hsa-miR-30c | colorectal cancer | up-regulated |
| hsa-miR-30c | colorectal cancer | down-regulated |
| hsa-miR-30c | Duchenne muscular dystrophy (DMD) | down-regulated |
| hsa-miR-30c | miyoshi myopathy (MM) | down-regulated |
| hsa-miR-30c | pancreatic cancer | up-regulated |
| hsa-miR-30c | prostate cancer | up-regulated |
| hsa-miR-30c | prostate cancer | down-regulated |
| hsa-miR-30c | prostate cancer | up-regulated |
| hsa-miR-30c | psoriasis | down-regulated |
| hsa-miR-30c-1 | hepatocellular carcinoma (HCC) | down-regulated |
| hsa-miR-30d | acute myeloid leukemia (AML) | up-regulated |
| hsa-miR-30d | anaplastic thyroid carcinoma (ATC) | down-regulated |
| hsa-miR-30d | cardiac hypertrophy | down-regulated |
| hsa-miR-30d | Duchenne muscular dystrophy (DMD) | down-regulated |
| hsa-miR-101 | glioblastoma multiforme (GBM) | down-regulated |
| hsa-miR-155 | Oral Squamous Cell Carcinoma (OSCC) | down-regulated |
| hsa-miR-155 | pancreatic ductal andenocarcinoma (PDAC) | up-regulated |
| hsa-miR-15a | hepatocellular carcinoma (HCC) | down-regulated |
| hsa-miR-16 | Oral Squamous Cell Carcinoma (OSCC) | down-regulated |
| hsa-miR-16 | hepatocellular carcinoma (HCC) | up-regulated |
| hsa-miR-17-5p | pulmonary hypertension | up-regulated |
| hsa-miR-17-5p | hepatocellular carcinoma (HCC) | up-regulated |
| hsa-miR-18 | hepatocellular carcinoma (HCC) | hepatocellular carcinom |
| hsa-miR-182 | lung cancer | up-regulated |
| hsa-miR-183 | lung cancer | up-regulated |
| hsa-miR-184 | adrenocortical carcinoma | up-regulated |
| hsa-miR-185 | hepatocellular carcinoma (HCC) | down-regulated |
| hsa-miR-18a* | colorectal cancer | down-regulated |
| hsa-miR-18a* | squamous carcinoma | down-regulated |
| hsa-miR-194 | hepatocellular carcinoma (HCC) | down-regulated |
| hsa-miR-195 | hepatocellular carcinoma (HCC) | down-regulated |
| hsa-miR-195 | bladder cancer | down-regulated |
| hsa-miR-195 | hepatocellular carcinoma (HCC) | down-regulated |
| hsa-miR-195* | Malignant mesothelioma (MM) | up-regulated |
| hsa-miR-373 | testicular germ cell tumor | up-regulated |
| hsa-miR-373* | Oral Squamous Cell Carcinoma (OSCC) | down-regulated |
| hsa-miR-373* | ovarian cancer (OC) | up-regulated |
| hsa-miR-373* | prostate cancer | up-regulated |
| hsa-miR-374 | acute myeloid leukemia (AML) | up-regulated |
| hsa-miR-374 | epithelial ovarian cancer (EOC) | down-regulated |
| hsa-miR-374 | head and neck squamous cell carcinoma (HNSCC) | down-regulated |
| hsa-miR-374 | hepatocellular carcinoma (HCC) | up-regulated |
| hsa-miR-374 | malignant melanoma | up-regulated |
| hsa-miR-374 | Oral Squamous Cell Carcinoma (OSCC) | up-regulated |
| hsa-miR-375 | pancreatic cancer | down-regulated |
| hsa-miR-375 | pancreatic ductal adenocarcinoma (PDAC) | down-regulated |
| hsa-miR-376a | epithelial ovarian cancer (EOC) | down-regulated |
| hsa-miR-376a | Duchenne muscular dystrophy (DMD) | up-regulated |
| hsa-miR-376a | limb-girdle muscular dystrophies types 2A (LGMD2A) | up-regulated |
| hsa-miR-376a | miyoshi myopathy (MM) | up-regulated |
| hsa-miR-376a | nemaline myopathy (NM) | up-regulated |
| hsa-miR-376a | pancreatic cancer | up-regulated |
| hsa-miR-376b | epithelial ovarian cancer (EOC) | down-regulated |
| hsa-miR-376b | uterine leiomyoma (ULM) | up-regulated |
| hsa-miR-377 | epithelial ovarian cancer (EOC) | down-regulated |
| hsa-miR-377 | uterine leiomyoma (ULM) | up-regulated |
| hsa-miR-378 | cardiac hypertrophy | down-regulated |
| hsa-miR-154 | facioscapulohumeral muscular dystrophy (FSHD) | up-regulated |
| hsa-miR-154 | limb-girdle muscular dystrophies types 2A (LGMD2A) | up-regulated |
| hsa-miR-154 | malignant melanoma | down-regulated |
| hsa-miR-154 | miyoshi myopathy (MM) | up-regulated |
| hsa-miR-154 | nemaline myopathy (NM) | up-regulated |
| hsa-miR-154 | polymyositis (PM) | up-regulated |
| hsa-miR-154* | glioblastoma multiforme (GBM) | down-regulated |
| hsa-miR-155 | chronic lymphocytic leukemia (CLL) | up-regulated |
| hsa-miR-155 | diffuse large B-cell lymphoma (DLBCL) | up-regulated |
| hsa-miR-155 | lung cancer | up-regulated |
| hsa-miR-155 | acute lymphoblastic leukemia (ALL) | up-regulated |
| hsa-miR-155 | diffuse large B-cell lymphoma (DLBCL) | up-regulated |
| hsa-miR-155 | myeloproliferative disorder | up-regulated |
| hsa-miR-222 | Duchenne muscular dystrophy (DMD) | up-regulated |
| hsa-miR-222 | facioscapulohumeral muscular dystrophy (FSHD) | up-regulated |
| hsa-miR-222 | glioblastoma | up-regulated |
| hsa-miR-222 | hepatocellular carcinoma (HCC) | up-regulated |
| hsa-miR-222 | hepatocellular carcinoma (HCC) | up-regulated |
| hsa-miR-1 | coronary artery disease | up-regulated |
| hsa-miR-1 | hepatocellular carcinoma (HCC) | down-regulated |
| hsa-miR-1 | Cerebellar neurodegeneration | down-regulated |
| hsa-miR-1 | retinitis pigmentosa (RP) | down-regulated |
| hsa-miR-192-2 | pituitary adenoma | down-regulated |
| hsa-miR-192-3 | pituitary adenoma | up-regulated |
| hsa-miR-193a | Oral Squamous Cell Carcinoma (OSCC) | down-regulated |
| hsa-miR-193b | autism spectrum disorder (ASD) | down-regulated |
| hsa-miR-193b | Duchenne muscular dystrophy (DMD) | down-regulated |
| hsa-miR-193b | uveal melanoma | up-regulated |
| hsa-miR-194 | hepatocellular carcinoma (HCC) | down-regulated |
| hsa-miR-194 | cardiac hypertrophy | down-regulated |
| hsa-miR-194 | chronic pancreatitis | up-regulated |
| hsa-miR-195 | chronic lymphocytic leukemia (CLL) | up-regulated |
| hsa-miR-195 | cardiac hypertrophy | up-regulated |
| hsa-miR-195 | acute myeloid leukemia (AML) | up-regulated |
| hsa-miR-195 | cardiac hypertrophy | up-regulated |
| hsa-miR-195 | chronic pancreatitis | up-regulated |
| hsa-miR-195 | head and neck squamous cell carcinoma (HNSCC) | down-regulated |
| hsa-miR-195 | heart failure | up-regulated |
| hsa-miR-195 | hepatocellular carcinoma (HCC) | down-regulated |
| hsa-miR-195 | hepatocellular carcinoma (HCC) | down-regulated |
| hsa-miR-195 | ovarian cancer (OC) | down-regulated |
| hsa-miR-195 | prostate cancer | up-regulated |
| hsa-miR-195 | schizophrenia | down-regulated |
| hsa-miR-196a | breast cancer | up-regulated |
| hsa-miR-196a-2 | B-cell chronic lymphocytic leukemia | up-regulated |
| hsa-miR-20a | pulmonary hypertension | up-regulated |
| hsa-miR-210 | pancreatic ductal adenocarcinoma (PDAC) | up-regulated |
| hsa-miR-218 | prostate cancer | up-regulated |
| hsa-miR-200a | meningioma | down-regulated |
| hsa-miR-200c | kidney cancer | down-regulated |
| hsa-miR-200c | breast cancer | down-regulated |
| hsa-miR-203 | pancreatic cancer | up-regulated |
| hsa-miR-204 | lung cancer | down-regulated |
| hsa-miR-205 | prostate cancer | down-regulated |
| hsa-miR-29c | kidney cancer | up-regulated |
| hsa-miR-29c | chronic lymphocytic leukemia (CLL) | down-regulated |
| hsa-miR-29c | neuroblastoma (NB) | down-regulated |
| hsa-miR-30a-3p | lung cancer | down-regulated |
| hsa-miR-30e-3p | lung cancer | down-regulated |
| hsa-miR-30e-5p | lung cancer | down-regulated |
| hsa-miR-31 | prostate cancer | down-regulated |
| hsa-miR-324-3p | lung cancer | down-regulated |
| hsa-miR-324-5p | lung cancer | up-regulated |
| hsa-miR-330 | prostate cance | down-regulated |
| hsa-miR-335 | lung cancer | down-regulated |
| hsa-miR-338 | lung cancer | down-regulated |
| hsa-miR-339 | lung cancer | up-regulated |
| hsa-miR-340 | kidney cancer | up-regulated |
| hsa-miR-342-3p | kidney cancer | up-regulated |
| hsa-miR-342-3p | prion disease | up-regulated |
| hsa-miR-345 | lung cancer | up-regulated |
| hsa-miR-34a | Alzheimer's disease | down-regulated |
| hsa-miR-221 | coronary artery disease | up-regulated |
| hsa-miR-221 | Oral Squamous Cell Carcinoma (OSCC) | up-regulated |
| hsa-miR-222 | tongue squamous cell carcinoma | down-regulated |
| hsa-miR-222 | coronary artery disease | up-regulated |
| hsa-miR-222 | hepatocellular carcinoma (HCC) | up-regulated |
| hsa-miR-222 | pancreatic ductal adenocarcinoma (PDAC) | up-regulated |
| hsa-miR-224 | hepatocellular carcinoma (HCC) | up-regulated |
| hsa-miR-23b | hepatocellular carcinoma (HCC) | down-regulated |
| hsa-miR-24 | hepatocellular carcinoma (HCC) | up-regulated |
| hsa-miR-25 | hepatocellular carcinoma (HCC) | hepatocellular carcinom |
| hsa-miR-25 | hepatocellular carcinoma (HCC) | up-regulated |
| hsa-miR-26a | hepatocellular carcinoma (HCC) | down-regulated |
| hsa-miR-26a | Oral Squamous Cell Carcinoma (OSCC) | down-regulated |
| hsa-miR-26a | bladder cancer | down-regulated |
| hsa-miR-26a | glioma | up-regulated |
| hsa-miR-27a | hepatocellular carcinoma (HCC) | down-regulated |
| hsa-miR-29a | Oral Squamous Cell Carcinoma (OSCC) | down-regulated |
| hsa-miR-29c | bladder cancer | down-regulated |
| hsa-miR-30-3p | bladder cancer | down-regulated |
| hsa-miR-30b* | Malignant mesothelioma (MM) | up-regulated |
| hsa-miR-30c | bladder cancer | down-regulated |
| hsa-miR-30e-5p | bladder cancer | down-regulated |
| hsa-miR-31 | breast cancer | down-regulated |
| hsa-miR-32* | Malignant mesothelioma (MM) | up-regulated |
| hsa-miR-320 | cardiomyopathy | down-regulated |
| hsa-miR-320 | renal clear cell carcinoma | down-regulated |
| hsa-miR-338 | hepatocellular carcinoma (HCC) | down-regulated |
| hsa-miR-338 | Oral Squamous Cell Carcinoma (OSCC) | up-regulated |
| hsa-miR-340* | Malignant mesothelioma (MM) | down-regulated |
| hsa-miR-345 | Malignant mesothelioma (MM) | up-regulated |
| hsa-miR-34a | retinoblastoma | down-regulated |
| hsa-miR-34a* | Malignant mesothelioma (MM) | down-regulated |
| hsa-miR-34b | retinoblastoma | down-regulated |
| hsa-miR-373 | esophageal cancer | up-regulated |
| hsa-miR-423 | Malignant mesothelioma (MM) | down-regulated |
| hsa-miR-429 | ovarian cancer (OC) | down-regulated |
| hsa-miR-483-3p | Malignant mesothelioma (MM) | up-regulated |
| hsa-miR-485-3p | anxiety disorder | normal |
| hsa-miR-494 | renal clear cell carcinoma | down-regulated |
| hsa-miR-582 | Malignant mesothelioma (MM) | down-regulated |
| hsa-miR-584 | Malignant mesothelioma (MM) | up-regulated |
| hsa-miR-595 | Malignant mesothelioma (MM) | up-regulated |
| hsa-miR-503 | adrenocortical carcinoma | up-regulated |
| hsa-miR-509 | anxiety disorder | normal |
| hsa-miR-511 | adrenocortical carcinoma | down-regulated |
| hsa-miR-512-5p | gastric cancer (stomach cancer) | down-regulated |
| hsa-miR-615-3p | Malignant mesothelioma (MM) | up-regulated |
| hsa-miR-7-1* | Malignant mesothelioma (MM) | down-regulated |
| hsa-miR-765 | anxiety disorder | normal |
| hsa-miR-885-3p | Malignant mesothelioma (MM) | up-regulated |
| hsa-miR-9 | Malignant mesothelioma (MM) | down-regulated |
| hsa-miR-9-1 | colorectal cancer | down-regulated |
| hsa-miR-92a | acute myeloid leukemia (AML) | down-regulated |
| hsa-miR-93 | hepatocellular carcinoma (HCC) | up-regulated |
| hsa-miR-9-3 | breast cancer | down-regulated |
| hsa-miR-143 | lung cancer | down-regulated |
| hsa-miR-143 | colorectal cancer | down-regulated |
| hsa-miR-145 | prostate cancer | down-regulated |
| hsa-miR-149 | prostate cancer | down-regulated |
| hsa-miR-150 | kidney cancer | down-regulated |
| hsa-miR-151-5p | kidney cancer | up-regulated |
| hsa-miR-155 | pancreatic cancer | up-regulated |
| hsa-miR-15a | kidney cancer | up-regulated |
| hsa-miR-153 | glioblastoma | down-regulated |
| hsa-miR-155 | hepatocellular carcinoma (HCC) | up-regulated |
| hsa-miR-15a | non-small cell lung cancer (NSCLC) | down-regulated |
| hsa-miR-15a | schizophrenia | up-regulated |
| hsa-miR-15b | schizophrenia | up-regulated |
| hsa-miR-16 | non-small cell lung cancer (NSCLC) | down-regulated |
| hsa-miR-16 | lung cancer | down-regulated |
| hsa-miR-16 | prostate cancer | down-regulated |
| hsa-miR-17 | kidney cancer | up-regulated |
| hsa-miR-185 | non-small cell lung cancer (NSCLC) | down-regulated |
| hsa-miR-18a | breast cancer | up-regulated |
| hsa-miR-18b | multiple sclerosis | up-regulated |
| hsa-miR-191 | kidney cancer | down-regulated |
| hsa-miR-193b | breast cancer | down-regulated |
| hsa-miR-195 | schizophrenia | up-regulated |
| hsa-miR-195 | breast cancer | up-regulated |
| hsa-miR-195 | lung cancer | down-regulated |
| hsa-miR-196a-2 | breast cancer | up-regulated |
| hsa-miR-197 | lung cancer | up-regulated |
| hsa-miR-199a | kidney cancer | down-regulated |
| hsa-miR-19b | kidney cancer | up-regulated |
| hsa-miR-200b | kidney cancer | down-regulated |
| hsa-miR-9 | ovarian cancer (OC) | down-regulated |
| hsa-miR-9 | hepatocellular carcinoma (HCC) | up-regulated |
| hsa-miR-9 | lung cancer | up-regulated |
| hsa-miR-9* | lung cancer | up-regulated |
| hsa-miR-93 | lung cancer | up-regulated |
| hsa-miR-96 | breast cancer | up-regulated |
| hsa-miR-96 | multiple sclerosis | up-regulated |
| hsa-miR-96 | prostate cancer | up-regulated |
| hsa-miR-98 | lung cancer | up-regulated |
| hsa-miR-182* | lung cancer | up-regulated |
| hsa-let-7c | lung cancer | down-regulated |
| hsa-miR-1 | heart failure | down-regulated |
| hsa-miR-1 | rhabdomyosarcoma | down-regulated |
| hsa-miR-1 | rhabdomyosarcoma | down-regulated |
| hsa-miR-101 | lung cancer | down-regulated |
| hsa-miR-101 | kidney cancer | up-regulated |
| hsa-miR-106a | kidney cancer | up-regulated |
| hsa-miR-106b | kidney cancer | up-regulated |
| hsa-miR-17-5p | lung cancer | up-regulated |
| hsa-miR-181a | chronic lymphocytic leukemia (CLL) | down-regulated |
| hsa-miR-181a | chronic lymphocytic leukemia (CLL) | down-regulated |
| hsa-miR-181b | prostate cancer | down-regulated |
| hsa-miR-182 | breast cancer | up-regulated |
| hsa-miR-182 | kidney cancer | down-regulated |
| hsa-miR-181a-1 | hepatocellular carcinoma (HCC) | up-regulated |
| hsa-miR-181a-2 | hepatocellular carcinoma (HCC) | up-regulated |
| hsa-miR-181b | chronic lymphocytic leukemia (CLL) | down-regulated |
| hsa-miR-181b-1 | hepatocellular carcinoma (HCC) | up-regulated |
| hsa-miR-181b-2 | hepatocellular carcinoma (HCC) | up-regulated |
| hsa-miR-181c | hepatocellular carcinoma (HCC) | up-regulated |
| hsa-miR-27a | breast cancer | up-regulated |
| hsa-miR-27a | hepatocellular carcinoma (HCC) | up-regulated |
| hsa-miR-133a | myeloproliferative disorder | down-regulated |
| hsa-miR-133b | bladder cancer | down-regulated |
| hsa-miR-137 | colorectal cancer | down-regulated |
| hsa-miR-138 | head and neck squamous cell carcinoma (HNSCC) | down-regulated |
| hsa-miR-143 | T-cell leukemia | down-regulated |
| hsa-miR-143 | Oral Squamous Cell Carcinoma (OSCC) | down-regulated |
| hsa-miR-143 | gastric cancer (stomach cancer) | down-regulated |
| hsa-miR-143 | hepatocellular carcinoma (HCC) | down-regulated |
| hsa-miR-143 | hepatocellular carcinoma (HCC) | up-regulated |
| hsa-miR-144* | Malignant mesothelioma (MM) | down-regulated |
| hsa-miR-145 | colorectal cancer | down-regulated |
| hsa-miR-145 | vascular disease | down-regulated |
| hsa-miR-145 | bladder cancer | down-regulated |
| hsa-miR-145 | Oral Squamous Cell Carcinoma (OSCC) | down-regulated |
| hsa-miR-145 | gastric cancer (stomach cancer) | down-regulated |
| hsa-miR-145 | renal clear cell carcinoma | down-regulated |
| hsa-miR-145 | hepatocellular carcinoma (HCC) | down-regulated |
| hsa-miR-145 | bladder cancer | down-regulated |
| hsa-miR-200b | hepatocellular carcinoma (HCC) | down-regulated |
| hsa-miR-200b | malignant melanoma | down-regulated |
| hsa-miR-200b | Oral Squamous Cell Carcinoma (OSCC) | down-regulated |
| hsa-miR-200c | colorectal cancer | up-regulated |
| hsa-miR-200c | breast cancer | down-regulated |
| hsa-miR-200c | cancer | down-regulated |
| hsa-miR-200c | cancer | down-regulated |
| hsa-miR-200c | colorectal cancer | down-regulated |
| hsa-miR-200c | epithelial ovarian cancer (EOC) | up-regulated |
| hsa-miR-200c | malignant melanoma | up-regulated |
| hsa-miR-200c | Oral Squamous Cell Carcinoma (OSCC) | down-regulated |
| hsa-miR-200c | testicular germ cell tumor | up-regulated |
| hsa-miR-202 | breast cancer | up-regulated |
| hsa-miR-202 | prostate cancer | up-regulated |
| hsa-miR-203 | acute lymphoblastic leukemia (ALL) | down-regulated |
| hsa-miR-203 | chronic myeloid leukemia (CML) | down-regulated |
| hsa-miR-203 | psoriasis | up-regulated |
| hsa-miR-203 | skin disease | up-regulated |
| hsa-miR-203 | bladder cancer | up-regulated |
| hsa-miR-203 | breast cancer | up-regulated |
| hsa-miR-203 | colorectal cancer | up-regulated |
| hsa-miR-203 | esophageal cancer | down-regulated |
| hsa-miR-203 | lung cancer | up-regulated |
| hsa-miR-203 | Oral Squamous Cell Carcinoma (OSCC) | down-regulated |
| hsa-miR-26a | acute myeloid leukemia (AML) | down-regulated |
| hsa-miR-26a | cardiac hypertrophy | down-regulated |
| hsa-miR-26a | colorectal cancer | up-regulated |
| hsa-miR-26a | Duchenne muscular dystrophy (DMD) | down-regulated |
| hsa-miR-26a | epithelial ovarian cancer (EOC) | up-regulated |
| hsa-miR-26a | pituitary adenoma | up-regulated |
| hsa-miR-26a | primary biliary cirrhosis (PBC) | down-regulated |
| hsa-miR-26a | prostate cancer | down-regulated |
| hsa-miR-26a | prostate cancer | up-regulated |
| hsa-miR-26a | serous ovarian cancer | down-regulated |
| hsa-miR-26a-1 | hepatocellular carcinoma (HCC) | down-regulated |
| hsa-miR-26a-1 | lung cancer | down-regulated |
| hsa-miR-26a-1 | papillary thyroid carcinoma (PTC) | up-regulated |
| hsa-miR-26a-2 | hepatocellular carcinoma (HCC) | down-regulated |
| hsa-miR-26b | Alzheimer's disease | down-regulated |
| hsa-miR-26b | bladder cancer | up-regulated |
| hsa-miR-26b | cardiac hypertrophy | down-regulated |
| hsa-miR-26b | epithelial ovarian cancer (EOC) | up-regulated |
| hsa-miR-26b | Oral Squamous Cell Carcinoma (OSCC) | down-regulated |
| hsa-miR-26b | pituitary adenoma | up-regulated |
| hsa-miR-26b | prostate cancer | down-regulated |
| hsa-miR-26b | schizophrenia | down-regulated |
| hsa-miR-27a | acute lymphoblastic leukemia (ALL) | down-regulated |
| hsa-miR-27a | acute myeloid leukemia (AML) | up-regulated |
| hsa-miR-32 | pancreatic cancer | up-regulated |
| hsa-miR-32 | prostate cancer | up-regulated |
| hsa-miR-32 | uterine leiomyoma (ULM) | down-regulated |
| hsa-miR-320 | homozygous sickle cell disease (HbSS) | down-regulated |
| hsa-miR-320 | Alzheimer's disease | up-regulated |
| hsa-miR-320 | colorectal cancer | up-regulated |
| hsa-miR-320 | diffuse large B-cell lymphoma (DLBCL) | down-regulated |
| hsa-miR-320 | follicular lymphoma (FL) | down-regulated |
| hsa-miR-320 | prostate cancer | up-regulated |
| hsa-miR-320-2 | hepatocellular carcinoma (HCC) | up-regulated |
| hsa-miR-320a | autism spectrum disorder (ASD) | down-regulated |
| hsa-miR-323 | glioblastoma multiforme (GBM) | down-regulated |
| hsa-miR-323 | malignant melanoma | down-regulated |
| hsa-miR-323 | uterine leiomyoma (ULM) | up-regulated |
| hsa-miR-324-3p | primary biliary cirrhosis (PBC) | down-regulated |
| hsa-miR-324-5p | acute myeloid leukemia (AML) | up-regulated |
| hsa-miR-324-5p | hepatocellular carcinoma (HCC) | up-regulated |
| hsa-miR-324-5p | malignant melanoma | up-regulated |
| hsa-miR-325 | acute myeloid leukemia (AML) | down-regulated |
| hsa-miR-326 | acute myeloid leukemia (AML) | up-regulated |
| hsa-miR-326 | primary biliary cirrhosis (PBC) | down-regulated |
| hsa-miR-326 | psoriasis | down-regulated |
| hsa-miR-325 | malignant melanoma | down-regulated |
| hsa-miR-125b | neuroblastoma (NB) | down-regulated |
| hsa-miR-125b | non-small cell lung cancer (NSCLC) | up-regulated |
| hsa-miR-125b | prostate cancer | down-regulated |
| hsa-miR-125b | prostate cancer | up-regulated |
| hsa-miR-125b | psoriasis | down-regulated |
| hsa-miR-125b | serous ovarian cancer | down-regulated |
| hsa-miR-125b | uterine leiomyoma (ULM) | up-regulated |
| hsa-miR-125b | vascular disease | down-regulated |
| hsa-miR-125b-1 | chronic pancreatitis | up-regulated |
| hsa-miR-125b-1 | epithelial ovarian cancer (EOC) | down-regulated |
| hsa-miR-125b-1 | glioblastoma | down-regulated |
| hsa-miR-125b-1 | hepatocellular carcinoma (HCC) | down-regulated |
| hsa-miR-125b-1 | pancreatic cancer | up-regulated |
| hsa-miR-125b-1 | pancreatic cancer | up-regulated |
| hsa-miR-125b-2 | chronic pancreatitis | up-regulated |
| hsa-miR-125b-2 | Down syndrome (DS) | up-regulated |
| hsa-miR-125b-2 | glioblastoma | down-regulated |
| hsa-miR-125b-2 | hepatocellular carcinoma (HCC) | down-regulated |
| hsa-miR-125b-2 | lung cancer | down-regulated |
| hsa-miR-126 | hepatocellular carcinoma (HCC) | down-regulated |
| hsa-miR-126 | acute promyelocytic leukemia (APL) | down-regulated |
| hsa-miR-126 | breast cancer | down-regulated |
| hsa-miR-126 | non-small cell lung cancer (NSCLC) | down-regulated |
| hsa-miR-126 | chronic pancreatitis | up-regulated |
| hsa-miR-146a | papillary thyroid carcinoma (PTC) | down-regulated |
| hsa-miR-146a | hepatocellular carcinoma (HCC) | down-regulated |
| hsa-miR-146a | pancreatic ductal adenocarcinoma (PDAC) | up-regulated |
| hsa-miR-146a | vascular disease | up-regulated |
| hsa-miR-1228* | Malignant mesothelioma (MM) | up-regulated |
| hsa-miR-122a | hepatocellular carcinoma (HCC) | down-regulated |
| hsa-miR-124a | Oral Squamous Cell Carcinoma (OSCC) | down-regulated |
| hsa-miR-124a | hepatocellular carcinoma (HCC) | down-regulated |
| hsa-miR-124a | acute lymphoblastic leukemia (ALL) | down-regulated |
| hsa-miR-125a | breast cancer | down-regulated |
| hsa-miR-125a | hepatocellular carcinoma (HCC) | down-regulated |
| hsa-miR-125b | bladder cancer | down-regulated |
| hsa-miR-125b | Oral Squamous Cell Carcinoma (OSCC) | down-regulated |
| hsa-miR-125b | Oral Squamous Cell Carcinoma (OSCC) | down-regulated |
| hsa-miR-125b | hepatocellular carcinoma (HCC) | down-regulated |
| hsa-miR-125b | prostate cancer | up-regulated |
| hsa-miR-126 | hepatocellular carcinoma (HCC) | down-regulated |
| hsa-miR-126* | hepatocellular carcinoma (HCC) | down-regulated |
| hsa-miR-128b | hepatocellular carcinoma (HCC) | up-regulated |
| hsa-miR-129 | bladder cancer | down-regulated |
| hsa-miR-129 | hepatocellular carcinoma (HCC) | down-regulated |
| hsa-miR-379 | dermatomyositis (DM) | up-regulated |
| hsa-miR-379 | Duchenne muscular dystrophy (DMD) | up-regulated |
| hsa-miR-379 | facioscapulohumeral muscular dystrophy (FSHD) | up-regulated |
| hsa-miR-379 | limb-girdle muscular dystrophies types 2A (LGMD2A) | up-regulated |
| hsa-miR-379 | miyoshi myopathy (MM) | up-regulated |
| hsa-miR-379 | nemaline myopathy (NM) | up-regulated |
| hsa-miR-379 | polymyositis (PM) | up-regulated |
| hsa-miR-17-3p | malignant melanoma | down-regulated |
| hsa-miR-17-5p | colorectal cancer | down-regulated |
| hsa-miR-17-5p | anaplastic thyroid carcinoma (ATC) | up-regulated |
| hsa-miR-17-5p | breast cancer | down-regulated |
| hsa-miR-17-5p | chronic myeloid leukemia (CML) | down-regulated |
| hsa-miR-17-5p | lung cancer | up-regulated |
| hsa-miR-17-5p | MYC-rearranged lymphoma | up-regulated |
| hsa-miR-17-5p | bladder cancer | up-regulated |
| hsa-miR-17-5p | breast cancer | up-regulated |
| hsa-miR-17-5p | colorectal cancer | up-regulated |
| hsa-miR-17-5p | diffuse large B-cell lymphoma (DLBCL) | up-regulated |
| hsa-miR-17-5p | hepatocellular carcinoma (HCC) | up-regulated |
| hsa-miR-17-5p | lung cancer | up-regulated |
| hsa-miR-17-5p | malignant lymphoma | up-regulated |
| hsa-miR-17-5p | neuroblastoma (NB) | up-regulated |
| hsa-miR-17-5p | pancreatic cancer | up-regulated |
| hsa-miR-17-5p | primary biliary cirrhosis (PBC) | down-regulated |
| hsa-miR-17-5p | prostate cancer | up-regulated |
| hsa-miR-100 | epithelial ovarian cancer (EOC) | down-regulated |
| hsa-miR-100 | epithelial ovarian cancer (EOC) | down-regulated |
| hsa-miR-100 | malignant melanoma | down-regulated |
| hsa-miR-100 | non-small cell lung cancer (NSCLC) | up-regulated |
| hsa-miR-100 | ovarian cancer (OC) | down-regulated |
| hsa-miR-100 | ovarian cancer (OC) | up-regulated |
| hsa-miR-100 | pancreatic cancer | up-regulated |
| hsa-miR-100 | prostate cancer | up-regulated |
| hsa-miR-100 | prostate cancer | down-regulated |
| hsa-miR-100 | prostate cancer | up-regulated |
| hsa-miR-100 | psoriasis | down-regulated |
| hsa-miR-100 | serous ovarian cancer | down-regulated |
| hsa-miR-100-1 | chronic pancreatitis | up-regulated |
| hsa-miR-100-1 | pancreatic cancer | up-regulated |
| hsa-miR-100-1 | pituitary adenoma | down-regulated |
| hsa-miR-100-2 | chronic pancreatitis | up-regulated |
| hsa-miR-100-2 | pancreatic cancer | up-regulated |
| hsa-miR-100-2 | pituitary adenoma | down-regulated |
| hsa-miR-101 | Alzheimer's disease | down-regulated |
| hsa-miR-101 | B-cell chronic lymphocytic leukemia | up-regulated |
| hsa-miR-101 | Duchenne muscular dystrophy (DMD) | down-regulated |
| hsa-miR-101 | epithelial ovarian cancer (EOC) | down-regulated |
| hsa-miR-101 | head and neck squamous cell carcinoma (HNSCC) | down-regulated |
| hsa-miR-101-1 | breast cancer | down-regulated |
| hsa-miR-20a | hepatocellular carcinoma (HCC) | up-regulated |
| hsa-miR-146a | Alzheimer's disease | up-regulated |
| hsa-miR-148b | Oral Squamous Cell Carcinoma (OSCC) | down-regulated |
| hsa-miR-196a-2 | congenital heart disease | up-regulated |
| hsa-miR-199a | Oral Squamous Cell Carcinoma (OSCC) | down-regulated |
| hsa-miR-203 | Oral Squamous Cell Carcinoma (OSCC) | down-regulated |
| hsa-miR-203 | pancreatic ductal adenocarcinoma (PDAC) | up-regulated |
| hsa-miR-207 | hepatocellular carcinoma (HCC) | up-regulated |
| hsa-miR-21 | breast cancer | up-regulated |
| hsa-miR-21 | colorectal cancer | up-regulated |
| hsa-miR-21 | Oral Squamous Cell Carcinoma (OSCC) | up-regulated |
| hsa-miR-21 | pancreatic cancer | up-regulated |
| hsa-miR-21 | lung cancer | up-regulated |
| hsa-miR-21 | tongue squamous cell carcinoma | up-regulated |
| hsa-miR-210 | lung cancer | up-regulated |
| hsa-miR-214 | adrenocortical carcinoma | down-regulated |
| hsa-miR-22 | breast cancer | up-regulated |
| hsa-miR-221 | glioma | up-regulated |
| hsa-miR-221 | hepatocellular carcinoma (HCC) | up-regulated |
| hsa-miR-222 | glioma | up-regulated |
| hsa-miR-186 | hepatocellular carcinoma (HCC) | up-regulated |
| hsa-miR-189 | malignant melanoma | down-regulated |
| hsa-miR-1 | myeloproliferative disorder | down-regulated |
| hsa-miR-107 | pancreatic cancer | up-regulated |
| hsa-miR-107 | hepatocellular carcinoma (HCC) | up-regulated |
| hsa-miR-126* | lung cancer | down-regulated |
| hsa-miR-127 | Burkitt lymphoma | up-regulated |
| hsa-miR-128 | anxiety disorder | normal |
| hsa-miR-129-2 | colorectal cancer | down-regulated |
| hsa-miR-130a | hepatocellular carcinoma (HCC) | up-regulated |
| hsa-miR-199a* | bladder cancer | down-regulated |
| hsa-miR-200a | hepatocellular carcinoma (HCC) | down-regulated |
| hsa-miR-200a | ovarian cancer (OC) | down-regulated |
| hsa-miR-200b | ovarian cancer (OC) | down-regulated |
| hsa-miR-200b | Oral Squamous Cell Carcinoma (OSCC) | up-regulated |
| hsa-miR-200c | breast cancer | up-regulated |
| hsa-miR-200c | endometrial cancer | up-regulated |
| hsa-miR-200c | ovarian cancer (OC) | up-regulated |
| hsa-miR-203 | Malignant mesothelioma (MM) | down-regulated |
| hsa-miR-205 | hepatocellular carcinoma (HCC) | up-regulated |
| hsa-miR-146b | dermatomyositis (DM) | up-regulated |
| hsa-miR-146b | Duchenne muscular dystrophy (DMD) | up-regulated |
| hsa-miR-146b | facioscapulohumeral muscular dystrophy (FSHD) | up-regulated |
| hsa-miR-146b | Inclusion body myositis (IBM) | up-regulated |
| hsa-miR-146b | limb-girdle muscular dystrophies types 2A (LGMD2A) | up-regulated |
| hsa-miR-146b | miyoshi myopathy (MM) | up-regulated |
| hsa-miR-146b | nemaline myopathy (NM) | up-regulated |
| hsa-miR-146b | ovarian cancer (OC) | up-regulated |
| hsa-miR-146b | papillary thyroid carcinoma (PTC) | up-regulated |
| hsa-miR-146b | polymyositis (PM) | up-regulated |
| hsa-miR-146b | psoriasis | up-regulated |
| hsa-miR-147 | acute myeloid leukemia (AML) | down-regulated |
| hsa-miR-147 | epithelial ovarian cancer (EOC) | down-regulated |
| hsa-miR-148a | hepatocellular carcinoma (HCC) | down-regulated |
| hsa-miR-148a | asthma | normal |
| hsa-miR-148a | breast cancer | down-regulated |
| hsa-miR-148a | breast cancer | down-regulated |
| hsa-miR-148a | dermatomyositis (DM) | up-regulated |
| hsa-miR-148a | Duchenne muscular dystrophy (DMD) | up-regulated |
| hsa-miR-148a | head and neck squamous cell carcinoma (HNSCC) | up-regulated |
| hsa-miR-148a | limb-girdle muscular dystrophies types 2A (LGMD2A) | up-regulated |
| hsa-miR-148a | miyoshi myopathy (MM) | up-regulated |
| hsa-miR-148a | nemaline myopathy (NM) | up-regulated |
| hsa-miR-148a | Oral Squamous Cell Carcinoma (OSCC) | down-regulated |
| hsa-miR-148a | pancreatic cancer | down-regulated |
| hsa-miR-1 | cardiomyopathy | down-regulated |
| hsa-miR-192 | nasopharyngeal carcinoma (NPC) | up-regulated |
| hsa-miR-192-1 | hepatocellular carcinoma (HCC) | down-regulated |
| hsa-miR-221 | gastric cancer (stomach cancer) | up-regulated |
| hsa-miR-222 | gastric cancer (stomach cancer) | up-regulated |
| hsa-miR-25 | gastric cancer (stomach cancer) | up-regulated |
| hsa-miR-26a | primary biliary cirrhosis (PBC) | down-regulated |
| hsa-miR-299-5p | primary biliary cirrhosis (PBC) | up-regulated |
| hsa-miR-31 | colorectal cancer | up-regulated |
| hsa-miR-328 | breast cancer | up-regulated |
| hsa-miR-346 | schizophrenia | down-regulated |
| hsa-miR-34a | hepatocellular carcinoma (HCC) | down-regulated |
| hsa-miR-34a | malignant melanoma | down-regulated |
| hsa-miR-34b | acute myeloid leukemia (AML) | down-regulated |
| hsa-miR-375 | head and neck squamous cell carcinoma (HNSCC) | up-regulated |
| hsa-miR-449a | prostate cancer | down-regulated |
| hsa-miR-451 | gastric cancer (stomach cancer) | down-regulated |
| hsa-miR-451 | colorectal cancer | down-regulated |
| hsa-miR-7 | lung cancer | down-regulated |
| hsa-miR-7 | breast cancer | down-regulated |
| hsa-miR-7 | glioblastoma | down-regulated |
| hsa-miR-92 | colorectal cancer | up-regulated |
| hsa-miR-96 | hearing loss | normal |
| hsa-miR-101-1 | lung cancer | down-regulated |
| hsa-miR-101a | Cerebellar neurodegeneration | down-regulated |
| hsa-miR-101b-2 | hepatocellular carcinoma (HCC) | up-regulated |
| hsa-miR-102 | papillary thyroid carcinoma (PTC) | up-regulated |
| hsa-miR-103 | esophageal cancer | up-regulated |
| hsa-miR-103 | acute myeloid leukemia (AML) | down-regulated |
| hsa-miR-103 | cardiac hypertrophy | up-regulated |
| hsa-miR-103 | Cerebellar neurodegeneration | down-regulated |
| hsa-miR-103 | epithelial ovarian cancer (EOC) | up-regulated |
| hsa-miR-103 | pancreatic cancer | up-regulated |
| hsa-miR-103 | pituitary adenoma | up-regulated |
| hsa-miR-103 | prostate cancer | down-regulated |
| hsa-miR-103-1 | bladder cancer | up-regulated |
| hsa-miR-103-2 | hepatocellular carcinoma (HCC) | up-regulated |
| hsa-miR-103-2 | pancreatic cancer | up-regulated |
| hsa-miR-103-2 | pituitary adenoma | up-regulated |
| hsa-miR-104 | malignant melanoma | down-regulated |
| hsa-miR-105 | epithelial ovarian cancer (EOC) | down-regulated |
| hsa-miR-105 | malignant melanoma | down-regulated |
| hsa-miR-106a | colorectal cancer | down-regulated |
| hsa-miR-106a | lung cancer | up-regulated |
| hsa-miR-106a | T-cell leukemia | up-regulated |
| hsa-miR-106a | autism spectrum disorder (ASD) | down-regulated |
| hsa-miR-106a | colorectal cancer | up-regulated |
| hsa-miR-424 | acute myeloid leukemia (AML) | up-regulated |
| hsa-miR-424 | cardiac hypertrophy | up-regulated |
| hsa-miR-424 | head and neck squamous cell carcinoma (HNSCC) | down-regulated |
| hsa-miR-424 | ovarian cancer (OC) | down-regulated |
| hsa-miR-424 | pancreatic cancer | up-regulated |
| hsa-miR-429 | breast cancer | down-regulated |
| hsa-miR-429 | cancer | down-regulated |
| hsa-miR-431 | autism spectrum disorder (ASD) | up-regulated |
| hsa-miR-432 | epithelial ovarian cancer (EOC) | down-regulated |
| hsa-miR-432 | autism spectrum disorder (ASD) | down-regulated |
| hsa-miR-432 | Duchenne muscular dystrophy (DMD) | up-regulated |
| hsa-miR-432 | limb-girdle muscular dystrophies types 2A (LGMD2A) | up-regulated |
| hsa-miR-432 | miyoshi myopathy (MM) | up-regulated |
| hsa-miR-432 | nemaline myopathy (NM) | up-regulated |
| hsa-miR-432* | primary biliary cirrhosis (PBC) | down-regulated |
| hsa-miR-451 | breast cancer | down-regulated |
| hsa-miR-451 | acute lymphoblastic leukemia (ALL) | down-regulated |
| hsa-miR-451 | acute myeloid leukemia (AML) | up-regulated |
| hsa-miR-451 | cardiac hypertrophy | down-regulated |
| hsa-miR-451 | homozygous sickle cell disease (HbSS) | up-regulated |
| hsa-miR-451 | primary biliary cirrhosis (PBC) | up-regulated |
| hsa-miR-451 | uterine leiomyoma (ULM) | down-regulated |
| hsa-miR-452 | dermatomyositis (DM) | up-regulated |
| hsa-miR-122a | malignant melanoma | down-regulated |
| hsa-miR-122a | psoriasis | down-regulated |
| hsa-miR-123 | B-cell chronic lymphocytic leukemia | up-regulated |
| hsa-miR-123 | glioblastoma | up-regulated |
| hsa-miR-123 | hepatocellular carcinoma (HCC) | down-regulated |
| hsa-miR-124a | breast cancer | down-regulated |
| hsa-miR-124a | cancer | down-regulated |
| hsa-miR-124a | chronic lymphocytic leukemia (CLL) | down-regulated |
| hsa-miR-124a | colorectal cancer | down-regulated |
| hsa-miR-124a | glioblastoma multiforme (GBM) | down-regulated |
| hsa-miR-124a | lung cancer | down-regulated |
| hsa-miR-124a | medulloblastoma | down-regulated |
| hsa-miR-126 | hepatocellular carcinoma (HCC) | down-regulated |
| hsa-miR-126 | lung cancer | down-regulated |
| hsa-miR-126 | Oral Squamous Cell Carcinoma (OSCC) | up-regulated |
| hsa-miR-126 | prostate cancer | up-regulated |
| hsa-miR-126* | prostate cancer | down-regulated |
| hsa-miR-126* | hepatocellular carcinoma (HCC) | down-regulated |
| hsa-miR-126* | lung cancer | down-regulated |
| hsa-miR-126-5p | cardiac hypertrophy | down-regulated |
| hsa-miR-127 | cancer | down-regulated |
| hsa-miR-127 | cardiac hypertrophy | up-regulated |
| hsa-miR-127 | Duchenne muscular dystrophy (DMD) | up-regulated |
| hsa-miR-9 | epithelial ovarian cancer (EOC) | down-regulated |
| hsa-miR-9 | follicular lymphoma (FL) | up-regulated |
| hsa-miR-9 | hepatocellular carcinoma (HCC) | up-regulated |
| hsa-miR-9 | lung cancer | down-regulated |
| hsa-miR-9 | malignant melanoma | down-regulated |
| hsa-miR-9 | neuroblastoma (NB) | down-regulated |
| hsa-miR-9 | non-small cell lung cancer (NSCLC) | down-regulated |
| hsa-miR-9* | acute myeloid leukemia (AML) | down-regulated |
| hsa-miR-9* | hepatocellular carcinoma (HCC) | up-regulated |
| hsa-miR-9* | Oral Squamous Cell Carcinoma (OSCC) | up-regulated |
| hsa-miR-9-1 | breast cancer | down-regulated |
| hsa-miR-9-1 | breast cancer | down-regulated |
| hsa-miR-9-1 | Oral Squamous Cell Carcinoma (OSCC) | up-regulated |
| hsa-miR-92 | chronic lymphocytic leukemia (CLL) | down-regulated |
| hsa-miR-92 | Duchenne muscular dystrophy (DMD) | down-regulated |
| hsa-miR-92 | nemaline myopathy (NM) | up-regulated |
| hsa-miR-92 | neuroblastoma (NB) | up-regulated |
| hsa-miR-92 | primary biliary cirrhosis (PBC) | down-regulated |
| hsa-miR-92 | prostate cancer | down-regulated |
| hsa-miR-92 | schizophrenia | down-regulated |
| hsa-miR-9-2 | hepatocellular carcinoma (HCC) | down-regulated |
| hsa-miR-9-2 | glioblastoma | up-regulated |
| hsa-miR-9-2 | Oral Squamous Cell Carcinoma (OSCC) | up-regulated |
| hsa-miR-92-1 | B-cell chronic lymphocytic leukemia | up-regulated |
| hsa-miR-141 | malignant melanoma | down-regulated |
| hsa-miR-141 | pancreatic ductal adenocarcinoma (PDAC) | down-regulated |
| hsa-miR-126 | epithelial ovarian cancer (EOC) | down-regulated |
| hsa-miR-16 | hepatocellular carcinoma (HCC) | up-regulated |
| hsa-miR-17-5p | colorectal cancer | up-regulated |
| hsa-miR-1 | head and neck squamous cell carcinoma (HNSCC) | down-regulated |
| hsa-miR-93 | gastric cancer (stomach cancer) | up-regulated |
| hsa-miR-17 | medulloblastoma | up-regulated |
| hsa-miR-18a | medulloblastoma | up-regulated |
| hsa-miR-19b | medulloblastoma | up-regulated |
| hsa-miR-20a | medulloblastoma | up-regulated |
| hsa-miR-92a | medulloblastoma | up-regulated |
| hsa-let-7a | hepatocellular carcinoma (HCC) | down-regulated |
| hsa-let-7b* | Malignant mesothelioma (MM) | up-regulated |
| hsa-let-7c | prostate cancer | up-regulated |
| hsa-let-7e* | Malignant mesothelioma (MM) | down-regulated |
| hsa-let-7g | renal clear cell carcinoma | up-regulated |
| hsa-miR-100 | Oral Squamous Cell Carcinoma (OSCC) | down-regulated |
| hsa-miR-100 | prostate cancer | up-regulated |
| hsa-miR-106b | hepatocellular carcinoma (HCC) | up-regulated |
| hsa-miR-140 | B-cell chronic lymphocytic leukemia | up-regulated |
| hsa-miR-140 | cardiac hypertrophy | up-regulated |
| hsa-miR-140 | epithelial ovarian cancer (EOC) | down-regulated |
| hsa-miR-140 | epithelial ovarian cancer (EOC) | down-regulated |
| hsa-miR-140 | homozygous sickle cell disease (HbSS) | up-regulated |
| hsa-miR-140 | lung cancer | down-regulated |
| hsa-miR-140 | malignant melanoma | down-regulated |
| hsa-miR-140 | Oral Squamous Cell Carcinoma (OSCC) | up-regulated |
| hsa-miR-140 | testicular germ cell tumor | up-regulated |
| hsa-miR-140* | cardiac hypertrophy | up-regulated |
| hsa-miR-141 | serous ovarian cancer | up-regulated |
| hsa-miR-141 | breast cancer | down-regulated |
| hsa-miR-141 | cancer | down-regulated |
| hsa-miR-141 | cancer | down-regulated |
| hsa-miR-141 | cholangiocarcinoma | up-regulated |
| hsa-miR-141 | B-cell chronic lymphocytic leukemia | up-regulated |
| hsa-miR-141 | epithelial ovarian cancer (EOC) | up-regulated |
| hsa-miR-141 | head and neck squamous cell carcinoma (HNSCC) | down-regulated |
| hsa-miR-141 | hepatocellular carcinoma (HCC) | down-regulated |
| hsa-miR-141 | homozygous sickle cell disease (HbSS) | down-regulated |
| hsa-miR-141 | prostate cancer | down-regulated |
| hsa-miR-141 | psoriasis | up-regulated |
| hsa-miR-142-3p | hepatocellular carcinoma (HCC) | down-regulated |
| hsa-miR-142-3p | ovarian cancer (OC) | up-regulated |
| hsa-miR-124a-3 | lung cancer | down-regulated |
| hsa-miR-223 | endometriosis | up-regulated |
| hsa-miR-23b | neuroblastoma (NB) | up-regulated |
| hsa-miR-27a | gastric cancer (stomach cancer) | up-regulated |
| hsa-miR-298 | Alzheimer's disease | down-regulated |
| hsa-miR-29b | acute myeloid leukemia (AML) | down-regulated |
| hsa-miR-29c | chronic lymphocytic leukemia (CLL) | down-regulated |
| hsa-miR-29c | head and neck squamous cell carcinoma (HNSCC) | up-regulated |
| hsa-miR-29c | endometriosis | up-regulated |
| hsa-miR-31 | bladder cancer | down-regulated |
| hsa-miR-320 | cholangiocarcinoma | down-regulated |
| hsa-miR-320 | acute myeloid leukemia (AML) | down-regulated |
| hsa-miR-328 | Alzheimer's disease | down-regulated |
| hsa-miR-34a | melanoma | down-regulated |
| hsa-miR-34c | endometriosis | down-regulated |
| hsa-miR-363* | Waldenstrom Macroglobulinemia (WM) | up-regulated |
| hsa-miR-365 | endometriosis | up-regulated |
| hsa-miR-377 | diabetic nephropathy | up-regulated |
| hsa-miR-424 | endometriosis | down-regulated |
| hsa-miR-494 | Waldenstrom Macroglobulinemia (WM) | up-regulated |
| hsa-miR-494 | head and neck squamous cell carcinoma (HNSCC) | down-regulated |
| hsa-miR-542-3p | Waldenstrom Macroglobulinemia (WM) | up-regulated |
| hsa-miR-9 | medulloblastoma | down-regulated |
| hsa-miR-145 | lung cancer | down-regulated |
| hsa-miR-196a | colorectal cancer | up-regulated |
| hsa-miR-155 | pancreatic cancer | up-regulated |
| hsa-miR-155 | acute myeloid leukemia (AML) | up-regulated |
| hsa-miR-155 | breast cancer | up-regulated |
| hsa-miR-155 | breast cancer | up-regulated |
| hsa-miR-155 | Burkitt lymphoma | up-regulated |
| hsa-miR-155 | Burkitt lymphoma | down-regulated |
| hsa-miR-155 | Burkitt lymphoma | down-regulated |
| hsa-miR-155 | cardiac hypertrophy | down-regulated |
| hsa-miR-155 | chronic lymphocytic leukemia (CLL) | up-regulated |
| hsa-miR-155 | chronic lymphocytic leukemia (CLL) | up-regulated |
| hsa-miR-155 | colorectal cancer | up-regulated |
| hsa-miR-155 | dermatomyositis (DM) | up-regulated |
| hsa-miR-155 | diffuse large B-cell lymphoma (DLBCL) | up-regulated |
| hsa-miR-155 | diffuse large B-cell lymphoma (DLBCL) | up-regulated |
| hsa-miR-155 | diffuse large B-cell lymphoma (DLBCL) | up-regulated |
| hsa-miR-155 | diffuse large B-cell lymphoma (DLBCL) | up-regulated |
| hsa-miR-155 | Down syndrome (DS) | up-regulated |
| hsa-miR-155 | Duchenne muscular dystrophy (DMD) | up-regulated |
| hsa-miR-155 | epithelial ovarian cancer (EOC) | down-regulated |
| hsa-miR-155 | facioscapulohumeral muscular dystrophy (FSHD) | up-regulated |
| hsa-miR-155 | follicular lymphoma (FL) | up-regulated |
| hsa-miR-155 | hepatocellular carcinoma (HCC) | down-regulated |
| hsa-miR-155 | Hodgkin's lymphoma | up-regulated |
| hsa-miR-155 | glioblastoma multiforme (GBM) | up-regulated |
| hsa-miR-155 | hepatocellular carcinoma (HCC) | up-regulated |
| hsa-miR-27a | acute myeloid leukemia (AML) | up-regulated |
| hsa-miR-328 | acute myeloid leukemia (AML) | up-regulated |
| hsa-miR-17-5p | psoriasis | up-regulated |
| hsa-miR-181a-1 | prostate cancer | up-regulated |
| hsa-miR-92 | medulloblastoma | up-regulated |
| hsa-miR-99a | endometriosis | up-regulated |
| hsa-miR-99b | endometriosis | up-regulated |
| hsa-miR-328 | primary biliary cirrhosis (PBC) | up-regulated |
| hsa-miR-532-5p | malignant melanoma | up-regulated |
| hsa-let-7d | head and neck squamous cell carcinoma (HNSCC) | down-regulated |
| hsa-miR-101 | bladder cancer | down-regulated |
| hsa-miR-101 | prostate cancer | down-regulated |
| hsa-miR-106b | Alzheimer's disease | down-regulated |
| hsa-miR-106b | gastric cancer (stomach cancer) | up-regulated |
| hsa-miR-122a | primary biliary cirrhosis (PBC) | down-regulated |
| hsa-miR-127 | diffuse large B-cell lymphoma (DLBCL) | up-regulated |
| hsa-miR-133a | head and neck squamous cell carcinoma (HNSCC) | down-regulated |
| hsa-miR-141 | gastric cancer (stomach cancer) | down-regulated |
| hsa-miR-143 | colorectal cancer | down-regulated |
| hsa-miR-145 | colorectal cancer | down-regulated |
| hsa-miR-146b | glioma | down-regulated |
| hsa-miR-28 | malignant melanoma | down-regulated |
| hsa-miR-296 | colorectal cancer | up-regulated |
| hsa-miR-221 | melanoma | up-regulated |
| hsa-miR-222 | bladder cancer | up-regulated |
| hsa-miR-452 | bladder cancer | up-regulated |
| hsa-miR-452* | bladder cancer | up-regulated |
| hsa-miR-7 | bladder cancer | up-regulated |
| hsa-miR-9 | Huntington's disease (HD) | down-regulated |
| hsa-miR-9* | Waldenstrom Macroglobulinemia (WM) | down-regulated |
| hsa-miR-183 | colorectal cancer | up-regulated |
| hsa-miR-18a | colorectal cancer | up-regulated |
| hsa-miR-196a | esophageal cancer | up-regulated |
| hsa-miR-199b-5p | medulloblastoma | down-regulated |
| hsa-miR-205 | breast cancer | down-regulated |
| hsa-miR-205 | head and neck squamous cell carcinoma (HNSCC) | down-regulated |
| hsa-miR-20a | colorectal cancer | up-regulated |
| hsa-miR-21 | head and neck squamous cell carcinoma (HNSCC) | up-regulated |
| hsa-miR-21 | prostate cancer | up-regulated |
| hsa-miR-21 | esophageal cancer | up-regulated |
| hsa-miR-221 | prostate cancer | up-regulated |
| hsa-miR-221 | head and neck squamous cell carcinoma (HNSCC) | up-regulated |
| hsa-miR-27a | autism spectrum disorder (ASD) | up-regulated |
| hsa-miR-27a | cardiac hypertrophy | up-regulated |
| hsa-miR-27a | cardiac hypertrophy | up-regulated |
| hsa-miR-27a | colorectal cancer | down-regulated |
| hsa-miR-27a | malignant melanoma | down-regulated |
| hsa-miR-27a | Oral Squamous Cell Carcinoma (OSCC) | down-regulated |
| hsa-miR-27a | prostate cancer | down-regulated |
| hsa-miR-27a | prostate cancer | up-regulated |
| hsa-miR-27a | serous ovarian cancer | up-regulated |
| hsa-miR-27a | uterine leiomyoma (ULM) | up-regulated |
| hsa-miR-27b | acute lymphoblastic leukemia (ALL) | down-regulated |
| hsa-miR-27b | acute myeloid leukemia (AML) | up-regulated |
| hsa-miR-27b | cardiac hypertrophy | up-regulated |
| hsa-miR-27b | cardiac hypertrophy | up-regulated |
| hsa-miR-27b | colorectal cancer | down-regulated |
| hsa-miR-27b | lung cancer | down-regulated |
| hsa-miR-27b | Oral Squamous Cell Carcinoma (OSCC) | down-regulated |
| hsa-miR-27b | prostate cancer | down-regulated |
| hsa-miR-27b | prostate cancer | up-regulated |
| hsa-miR-28 | kidney cancer | up-regulated |
| hsa-miR-296 | Oral Squamous Cell Carcinoma (OSCC) | down-regulated |
| hsa-miR-296 | ovarian cancer (OC) | up-regulated |
| hsa-miR-296 | primary biliary cirrhosis (PBC) | down-regulated |
| hsa-miR-296 | prostate cancer | up-regulated |
| hsa-miR-328 | colorectal cancer | down-regulated |
| hsa-miR-328 | diffuse large B-cell lymphoma (DLBCL) | down-regulated |
| hsa-miR-328 | glioblastoma multiforme (GBM) | down-regulated |
| hsa-miR-328 | primary biliary cirrhosis (PBC) | down-regulated |
| hsa-miR-328-1 | hepatocellular carcinoma (HCC) | up-regulated |
| hsa-miR-33 | B-cell chronic lymphocytic leukemia | up-regulated |
| hsa-miR-33 | lung cancer | down-regulated |
| hsa-miR-330 | follicular lymphoma (FL) | up-regulated |
| hsa-miR-330 | Huntington's disease (HD) | up-regulated |
| hsa-miR-330 | Oral Squamous Cell Carcinoma (OSCC) | down-regulated |
| hsa-miR-331 | acute myeloid leukemia (AML) | up-regulated |
| hsa-miR-331 | Duchenne muscular dystrophy (DMD) | down-regulated |
| hsa-miR-331 | epithelial ovarian cancer (EOC) | down-regulated |
| hsa-miR-331 | head and neck squamous cell carcinoma (HNSCC) | down-regulated |
| hsa-miR-331 | malignant melanoma | up-regulated |
| hsa-miR-331 | Oral Squamous Cell Carcinoma (OSCC) | down-regulated |
| hsa-miR-335 | breast cancer | down-regulated |
| hsa-miR-335 | acute lymphoblastic leukemia (ALL) | down-regulated |
| hsa-miR-335 | acute myeloid leukemia (AML) | up-regulated |
| hsa-miR-335 | dermatomyositis (DM) | up-regulated |
| hsa-miR-335 | Duchenne muscular dystrophy (DMD) | up-regulated |
| hsa-miR-335 | facioscapulohumeral muscular dystrophy (FSHD) | up-regulated |
| hsa-miR-335 | limb-girdle muscular dystrophies types 2A (LGMD2A) | up-regulated |
| hsa-miR-347 | vascular disease | down-regulated |
| hsa-miR-21 | glioblastoma | up-regulated |
| hsa-miR-21 | head and neck squamous cell carcinoma (HNSCC) | up-regulated |
| hsa-miR-1 | endometriosis | up-regulated |
| hsa-miR-1 | cardiac hypertrophy | down-regulated |
| hsa-let-7d | Hepatocellular carcinoma (HCC) | down-regulated |
| hsa-let-7i | ovarian cancer (OC) | down-regulated |
| hsa-miR-1 | Hepatocellular carcinoma (HCC) | down-regulated |
| hsa-miR-100 | endometriosis | up-regulated |
| hsa-miR-101 | Hepatocellular carcinoma (HCC) | down-regulated |
| hsa-miR-101 | Hepatocellular carcinoma (HCC) | down-regulated |
| hsa-miR-101 | prostate cancer | down-regulated |
| hsa-miR-106a | gastric cancer (stomach cancer) | up-regulated |
| hsa-miR-106b | chronic lymphocytic leukemia (CLL) | down-regulated |
| hsa-miR-10a | bladder cancer | up-regulated |
| hsa-miR-10a | chronic myeloid leukemia (CML) | down-regulated |
| hsa-miR-125a | medulloblastoma | down-regulated |
| hsa-miR-125a | endometriosis | up-regulated |
| hsa-miR-125b | endometriosis | up-regulated |
| hsa-miR-126 | breast cancer | down-regulated |
| hsa-miR-126 | endometriosis | up-regulated |
| hsa-miR-141 | kidney cancer | down-regulated |
| hsa-miR-127 | bladder cancer | down-regulated |
| hsa-miR-127 | breast cancer | down-regulated |
| hsa-miR-127 | colorectal cancer | down-regulated |
| hsa-miR-127 | prostate cancer | down-regulated |
| hsa-miR-125b | bladder cancer | up-regulated |
| hsa-miR-133a | Hepatocellular carcinoma (HCC) | down-regulated |
| hsa-miR-141 | endometriosis | down-regulated |
| hsa-miR-142-3p | head and neck squamous cell carcinoma (HNSCC) | up-regulated |
| hsa-miR-143 | bladder cancer | down-regulated |
| hsa-miR-143 | endometriosis | up-regulated |
| hsa-miR-143 | colorectal cancer | down-regulated |
| hsa-miR-145 | endometriosis | up-regulated |
| hsa-miR-146a* | thyroid cancer | normal |
| hsa-miR-146b | head and neck squamous cell carcinoma (HNSCC) | up-regulated |
| hsa-miR-15 | chronic myeloid leukemia (CML) | down-regulated |
| hsa-miR-150 | endometriosis | up-regulated |
| hsa-miR-155 | Waldenstrom Macroglobulinemia (WM) | up-regulated |
| hsa-miR-155 | head and neck squamous cell carcinoma (HNSCC) | up-regulated |
| hsa-miR-15b | glioma | down-regulated |
| hsa-miR-18 | head and neck squamous cell carcinoma (HNSCC) | up-regulated |
| hsa-miR-184 | Waldenstrom Macroglobulinemia (WM) | up-regulated |
| hsa-miR-192 | colorectal cancer | down-regulated |
| hsa-miR-194 | colorectal cancer | down-regulated |
| hsa-miR-17-92 | Burkitt lymphoma | up-regulated |
| hsa-miR-17-92 | colorectal cancer | up-regulated |
| hsa-miR-17-92 | diffuse large B-cell lymphoma (DLBCL) | up-regulated |
| hsa-miR-17-92 | lymphoproliferative disease | up-regulated |
| hsa-miR-18 | hepatocellular carcinoma (HCC) | up-regulated |
| hsa-miR-181a | acute myeloid leukemia (AML) | up-regulated |
| hsa-miR-181a | acute myeloid leukemia (AML) | up-regulated |
| hsa-miR-181a | Cerebellar neurodegeneration | down-regulated |
| hsa-miR-181a | chronic lymphocytic leukemia (CLL) | down-regulated |
| hsa-miR-181a | colorectal cancer | up-regulated |
| hsa-miR-181a | glioblastoma | down-regulated |
| hsa-miR-181a | homozygous sickle cell disease (HbSS) | down-regulated |
| hsa-miR-181a | malignant melanoma | up-regulated |
| hsa-miR-181a | pancreatic cancer | up-regulated |
| hsa-miR-181a | pancreatic cancer | up-regulated |
| hsa-miR-181a | papillary thyroid carcinoma (PTC) | up-regulated |
| hsa-miR-181a* | Oral Squamous Cell Carcinoma (OSCC) | up-regulated |
| hsa-miR-181a-1 | hepatocellular carcinoma (HCC) | down-regulated |
| hsa-miR-148a | prostate cancer | down-regulated |
| hsa-miR-148b | hepatocellular carcinoma (HCC) | down-regulated |
| hsa-miR-188 | B-cell chronic lymphocytic leukemia | up-regulated |
| hsa-miR-22 | prostate cancer | down-regulated |
| hsa-miR-22 | psoriasis | down-regulated |
| hsa-miR-142-3p | psoriasis | up-regulated |
| hsa-miR-142-5p | acute myeloid leukemia (AML) | up-regulated |
| hsa-miR-142-5p | hepatocellular carcinoma (HCC) | down-regulated |
| hsa-miR-142-5p | ovarian cancer (OC) | down-regulated |
| hsa-miR-143 | Burkitt lymphoma | down-regulated |
| hsa-miR-143 | chronic lymphocytic leukemia (CLL) | down-regulated |
| hsa-miR-143 | colorectal cancer | down-regulated |
| hsa-miR-143 | colorectal cancer | down-regulated |
| hsa-miR-143 | diffuse large B-ccll lymphoma (DLBCL) | down-regulated |
| hsa-miR-143 | Obesity | up-regulated |
| hsa-miR-143 | breast cancer | down-regulated |
| hsa-miR-143 | cervical cancer | down-regulated |
| hsa-miR-143 | colorectal cancer | down-regulated |
| hsa-miR-143 | colorectal cancer | down-regulated |
| hsa-miR-143 | epithelial ovarian cancer (EOC) | down-regulated |
| hsa-miR-143 | hepatocellular carcinoma (HCC) | down-regulated |
| hsa-miR-143 | pancreatic cancer | up-regulated |
| hsa-miR-143 | pancreatic ductal adenocarcinoma (PDAC) | up-regulated |
| hsa-miR-143 | prostate cancer | down-regulated |
| hsa-miR-143 | serous ovarian cancer | down-regulated |
| hsa-miR-143 | uveal melanoma | up-regulated |
| hsa-miR-143 | vascular disease | down-regulated |
| hsa-miR-144 | uterine leiomyoma (ULM) | down-regulated |
| hsa-miR-145 | lung cancer | down-regulated |
| hsa-miR-145 | Burkitt lymphoma | down-regulated |
| hsa-miR-213 | malignant melanoma | down-regulated |
| hsa-miR-155 | Inclusion body myositis (IBM) | up-regulated |
| hsa-miR-299-3p | Duchenne muscular dystrophy (DMD) | up-regulated |
| hsa-miR-335 | miyoshi myopathy (MM) | up-regulated |
| hsa-miR-106a | diffuse large B-cell lymphoma (DLBCL) | up-regulated |
| hsa-miR-130 | Spinocerebellar ataxia 1 | down-regulated |
| hsa-miR-19 | Spinocerebellar ataxia 1 | down-regulated |
| hsa-miR-130b | T-cell leukemia | up-regulated |
| hsa-miR-93 | T-cell leukemia | up-regulated |
| hsa-miR-196b | endometriosis | down-regulated |
| hsa-miR-20 | medulloblastoma | up-regulated |
| hsa-miR-200a | endometriosis | down-regulated |
| hsa-miR-200b | endometriosis | down-regulated |
| hsa-miR-200c | kidney cancer | down-regulated |
| hsa-miR-204 | cholangiocarcinoma | down-regulated |
| hsa-miR-205 | Hepatocellular carcinoma (HCC) | down-regulated |
| hsa-miR-205 | squamous carcinoma | up-regulated |
| hsa-miR-205 | head and neck squamous cell carcinoma (HNSCC) | up-regulated |
| hsa-miR-206 | Waldenstrom Macroglobulinemia (WM) | up-regulated |
| hsa-miR-20a | endometriosis | down-regulated |
| hsa-miR-21 | gastric cancer (stomach cancer) | up-regulated |
| hsa-miR-21 | heart failure | up-regulated |
| hsa-miR-215 | colorectal cancer | down-regulated |
| hsa-miR-210 | lung cancer | up-regulated |
| hsa-miR-210 | miyoshi myopathy (MM) | up-regulated |
| hsa-miR-210 | nemaline myopathy (NM) | up-regulated |
| hsa-miR-210 | pancreatic cancer | up-regulated |
| hsa-miR-210 | pancreatic ductal adenocarcinoma (PDAC) | up-regulated |
| hsa-miR-210 | polymyositis (PM) | up-regulated |
| hsa-miR-210 | prostate cancer | up-regulated |
| hsa-miR-211 | epithelial ovarian cancer (EOC) | down-regulated |
| hsa-miR-211 | malignant melanoma | down-regulated |
| hsa-miR-212 | alcoholic liver disease (ALD) | up-regulated |
| hsa-miR-212 | autism spectrum disorder (ASD) | up-regulated |
| hsa-miR-212 | hepatocellular carcinoma (HCC) | up-regulated |
| hsa-miR-212 | lung cancer | up-regulated |
| hsa-miR-212 | nasopharyngeal carcinoma (NPC) | down-regulated |
| hsa-miR-212 | pancreatic cancer | up-regulated |
| hsa-miR-212 | pituitary adenoma | down-regulated |
| hsa-miR-212 | primary biliary cirrhosis (PBC) | down-regulated |
| hsa-miR-212 | schizophrenia | down-regulated |
| hsa-miR-212 | uterine leiomyoma (ULM) | down-regulated |
| hsa-miR-213 | B-cell chronic lymphocytic leukemia | down-regulated |
| hsa-miR-213 | breast cancer | up-regulated |
| hsa-miR-213 | follicular lymphoma (FL) | up-regulated |
| hsa-miR-213 | pancreatic cancer | up-regulated |
| hsa-miR-213 | papillary thyroid carcinoma (PTC) | up-regulated |
| hsa-miR-144 | malignant melanoma | down-regulated |
| hsa-miR-296 | lupus nephritis | down-regulated |
| hsa-miR-30a-5p | lupus nephritis | up-regulated |
| hsa-miR-30d | lupus nephritis | down-regulated |
| hsa-miR-320 | lupus nephritis | up-regulated |
| hsa-miR-324-3p | lupus nephritis | down-regulated |
| hsa-miR-324-5p | lupus nephritis | up-regulated |
| hsa-miR-325 | lupus nephritis | up-regulated |
| hsa-miR-345 | lupus nephritis | down-regulated |
| hsa-miR-346 | lupus nephritis | down-regulated |
| hsa-miR-365 | lupus nephritis | down-regulated |
| hsa-miR-381 | lupus nephritis | down-regulated |
| hsa-miR-423 | lupus nephritis | down-regulated |
| hsa-miR-433 | lupus nephritis | up-regulated |
| hsa-miR-484 | lupus nephritis | down-regulated |
| hsa-miR-486 | lupus nephritis | down-regulated |
| hsa-miR-494 | lupus nephritis | up-regulated |
| hsa-miR-500 | lupus nephritis | down-regulated |
| hsa-miR-513 | lupus nephritis | up-regulated |
| hsa-miR-516-5p | lupus nephritis | up-regulated |
| hsa-miR-518b | lupus nephritis | down-regulated |
| hsa-miR-518c* | lupus nephritis | up-regulated |
| hsa-miR-557 | lupus nephritis | down-regulated |
| hsa-miR-575 | lupus nephritis | up-regulated |
| hsa-miR-155 | limb-girdle muscular dystrophies types 2A (LGMD2A) | up-regulated |
| hsa-miR-155 | lung cancer | up-regulated |
| hsa-miR-155 | miyoshi myopathy (MM) | up-regulated |
| hsa-miR-155 | nemaline myopathy (NM) | up-regulated |
| hsa-miR-155 | Oral Squamous Cell Carcinoma (OSCC) | down-regulated |
| hsa-miR-155 | ovarian cancer (OC) | down-regulated |
| hsa-miR-155 | pancreatic cancer | down-regulated |
| hsa-miR-155 | pancreatic cancer | up-regulated |
| hsa-miR-155 | pancreatic cancer | up-regulated |
| hsa-miR-155 | pancreatic ductal adenocarcinoma (PDAC) | up-regulated |
| hsa-miR-138-2 | papillary thyroid carcinoma (PTC) | up-regulated |
| hsa-miR-138-2 | pituitary adenoma | down-regulated |
| hsa-miR-139 | cardiac hypertrophy | down-regulated |
| hsa-miR-139 | diffuse large B-cell lymphoma (DLBCL) | down-regulated |
| hsa-miR-139 | follicular lymphoma (FL) | down-regulated |
| hsa-miR-139 | hepatocellular carcinoma (HCC) | down-regulated |
| hsa-miR-139 | malignant melanoma | down-regulated |
| hsa-miR-139 | Oral Squamous Cell Carcinoma (OSCC) | down-regulated |
| hsa-miR-145 | prostate cancer | down-regulated |
| hsa-miR-222 | Inclusion body myositis (IBM) | up-regulated |
| hsa-miR-222 | limb-girdle muscular dystrophies types 2A (LGMD2A) | up-regulated |
| hsa-miR-222 | miyoshi myopathy (MM) | up-regulated |
| hsa-miR-222 | nemaline myopathy (NM) | up-regulated |
| hsa-miR-222 | malignant melanoma | up-regulated |
| hsa-miR-128 | neurodegeneration | up-regulated |
| hsa-miR-139-5p | neurodegeneration | up-regulated |
| hsa-miR-146a | neurodegeneration | up-regulated |
| hsa-miR-181a-1* | neurodegeneration | up-regulated |
| hsa-miR-203 | neurodegeneration | up-regulated |
| hsa-miR-320 | neurodegeneration | up-regulated |
| hsa-miR-328 | neurodegeneration | up-regulated |
| hsa-miR-337-3p | neurodegeneration | down-regulated |
| hsa-miR-338-3p | neurodegeneration | down-regulated |
| hsa-miR-339-5p | neurodegeneration | up-regulated |
| hsa-miR-342-3p | neurodegeneration | up-regulated |
| hsa-let-7a | non-small cell lung cancer (NSCLC) | down-regulated |
| hsa-let-7b | non-small cell lung cancer (NSCLC) | down-regulated |
| hsa-let-7d | non-small cell lung cancer (NSCLC) | down-regulated |
| hsa-let-7g | non-small cell lung cancer (NSCLC) | down-regulated |
| hsa-miR-21 | Oral Squamous Cell Carcinoma (OSCC) | up-regulated |
| hsa-miR-15a | Polycystic Kidney Disease | down-regulated |
| hsa-miR-15a | Polycystic liver disease | down-regulated |
| hsa-miR-145 | prostate cancer | down-regulated |
| hsa-miR-15a | prostate cancer | down-regulated |
| hsa-miR-16-1 | prostate cancer | down-regulated |
| hsa-miR-221 | prostate cancer | down-regulated |
| hsa-miR-222 | prostate cancer | down-regulated |
| hsa-miR-302 | teratocarcinoma | up-regulated |
| hsa-miR-302a | malignant melanoma | down-regulated |
| hsa-miR-302b | malignant melanoma | down-regulated |
| hsa-miR-302b* | acute myeloid leukemia (AML) | down-regulated |
| hsa-miR-221 | melanoma | up-regulated |
| hsa-miR-222 | melanoma | up-regulated |
| hsa-let-7f | ulcerative colitis (UC) | up-regulated |
| hsa-miR-126 | ulcerative colitis (UC) | up-regulated |
| hsa-miR-16 | ulcerative colitis (UC) | up-regulated |
| hsa-miR-192 | ulcerative colitis (UC) | down-regulated |
| hsa-miR-195 | ulcerative colitis (UC) | up-regulated |
| hsa-miR-199a* | ulcerative colitis (UC) | up-regulated |
| hsa-miR-203 | ulcerative colitis (UC) | up-regulated |
| hsa-miR-21 | ulcerative colitis (UC) | up-regulated |
| hsa-miR-23a | ulcerative colitis (UC) | up-regulated |
| hsa-miR-23b | ulcerative colitis (UC) | up-regulated |
| hsa-miR-24 | ulcerative colitis (UC) | up-regulated |
| hsa-miR-26a | ulcerative colitis (UC) | up-regulated |
| hsa-miR-29a | ulcerative colitis (UC) | up-regulated |
| hsa-miR-422b | ulcerative colitis (UC) | down-regulated |
| hsa-miR-629 | ulcerative colitis (UC) | down-regulated |
| hsa-miR-299-3p | nemaline myopathy (NM) | up-regulated |
| hsa-miR-299-5p | dermatomyositis (DM) | up-regulated |
| hsa-miR-299-5p | Duchenne muscular dystrophy (DMD) | up-regulated |
| hsa-miR-299-5p | limb-girdle muscular dystrophies types 2A (LGMD2A) | up-regulated |
| hsa-miR-299-5p | miyoshi myopathy (MM) | up-regulated |
| hsa-miR-299-5p | nemaline myopathy (NM) | up-regulated |
| hsa-miR-29a | Alzheimer's disease | down-regulated |
| hsa-miR-29a | lung cancer | down-regulated |
| hsa-miR-29a | type 2 diabetes | up-regulated |
| hsa-miR-29a | cardiac hypertrophy | down-regulated |
| hsa-miR-29a | cardiac hypertrophy | down-regulated |
| hsa-miR-29a | Cerebellar neurodegeneration | down-regulated |
| hsa-miR-29a | Duchenne muscular dystrophy (DMD) | down-regulated |
| hsa-miR-29a | facioscapulohumeral muscular dystrophy (FSHD) | up-regulated |
| hsa-miR-29a | homozygous sickle cell disease (HbSS) | up-regulated |
| hsa-miR-29a | Huntington's disease (HD) | up-regulated |
| hsa-miR-29a | ovarian cancer (OC) | up-regulated |
| hsa-miR-29a | prostate cancer | down-regulated |
| hsa-miR-29a | schizophrenia | down-regulated |
| hsa-miR-29a | serous ovarian cancer | down-regulated |
| hsa-miR-29a-2 | chronic lymphocytic leukemia (CLL) | down-regulated |
| hsa-miR-29a-2 | papillary thyroid carcinoma (PTC) | up-regulated |
| hsa-miR-29b | cholangiocarcinoma | down-regulated |
| hsa-miR-29b | chronic lymphocytic leukemia (CLL) | down-regulated |
| hsa-miR-29b | chronic lymphocytic leukemia (CLL) | down-regulated |
| hsa-miR-452 | Duchenne muscular dystrophy (DMD) | up-regulated |
| hsa-miR-452 | limb-girdle muscular dystrophies types 2A (LGMD2A) | up-regulated |
| hsa-miR-452 | nemaline myopathy (NM) | up-regulated |
| hsa-miR-483 | chronic pancreatitis | up-regulated |
| hsa-miR-483 | primary biliary cirrhosis (PBC) | down-regulated |
| hsa-miR-484 | autism spectrum disorder (ASD) | up-regulated |
| hsa-miR-485-5p | Duchenne muscular dystrophy (DMD) | up-regulated |
| hsa-miR-486 | cardiac hypertrophy | down-regulated |
| hsa-miR-486 | Duchenne muscular dystrophy (DMD) | down-regulated |
| hsa-miR-486 | uterine leiomyoma (ULM) | down-regulated |
| hsa-miR-487b | dermatomyositis (DM) | up-regulated |
| hsa-miR-487b | Duchenne muscular dystrophy (DMD) | up-regulated |
| hsa-miR-487b | limb-girdle muscular dystrophies types 2A (LGMD2A) | up-regulated |
| hsa-miR-487b | miyoshi myopathy (MM) | up-regulated |
| hsa-miR-487b | nemaline myopathy (NM) | up-regulated |
| hsa-miR-487b | ovarian cancer (OC) | up-regulated |
| hsa-miR-491 | prostate cancer | up-regulated |
| hsa-miR-493-3p | Duchenne muscular dystrophy (DMD) | up-regulated |
| hsa-miR-493-3p | limb-girdle muscular dystrophies types 2A (LGMD2A) | up-regulated |
| hsa-miR-493-3p | nemaline myopathy (NM) | up-regulated |
| hsa-miR-493-5p | uterine leiomyoma (ULM) | up-regulated |
| hsa-miR-494 | chronic pancreatitis | up-regulated |
| hsa-miR-494 | ovarian cancer (OC) | up-regulated |
| hsa-miR-494 | primary biliary cirrhosis (PBC) | down-regulated |
| hsa-miR-495 | epithelial ovarian cancer (EOC) | down-regulated |
| hsa-miR-92-1 | malignant lymphoma | up-regulated |
| hsa-miR-92-1 | pancreatic cancer | up-regulated |
| hsa-miR-92-2 | T-cell leukemia | up-regulated |
| hsa-miR-92-2 | gastric cancer (stomach cancer) | up-regulated |
| hsa-miR-92-2 | pancreatic cancer | up-regulated |
| hsa-miR-92-2 | prostate cancer | up-regulated |
| hsa-miR-93 | serous ovarian cancer | up-regulated |
| hsa-miR-93 | gastric cancer (stomach cancer) | up-regulated |
| hsa-miR-93 | vesicular stomatitis | down-regulated |
| hsa-miR-93 | Alzheimer's disease | down-regulated |
| hsa-miR-93 | autism spectrum disorder (ASD) | down-regulated |
| hsa-miR-93 | hepatocellular carcinoma (HCC) | up-regulated |
| hsa-miR-93 | neuroblastoma (NB) | up-regulated |
| hsa-miR-93 | pancreatic ductal adenocarcinoma (PDAC) | up-regulated |
| hsa-miR-9-3 | Oral Squamous Cell Carcinoma (OSCC) | up-regulated |
| hsa-miR-9-3 | papillary thyroid carcinoma (PTC) | up-regulated |
| hsa-miR-9-3 | pituitary adenoma | down-regulated |
| hsa-miR-9-3p | schizophrenia | down-regulated |
| hsa-miR-95 | autism spectrum disorder (ASD) | up-regulated |
| hsa-miR-95 | diffuse large B-cell lymphoma (DLBCL) | down-regulated |
| hsa-miR-95 | Duchenne muscular dystrophy (DMD) | down-regulated |
| hsa-miR-95 | lung cancer | down-regulated |
| hsa-miR-95 | Oral Squamous Cell Carcinoma (OSCC) | down-regulated |
| hsa-miR-96 | chronic pancreatitis | down-regulated |
| hsa-miR-127 | epithelial ovarian cancer (EOC) | down-regulated |
| hsa-miR-127 | malignant melanoma | down-regulated |
| hsa-miR-127 | nemaline myopathy (NM) | up-regulated |
| hsa-miR-127 | polymyositis (PM) | up-regulated |
| hsa-miR-128a | acute lymphoblastic leukemia (ALL) | up-regulated |
| hsa-miR-128a | acute myeloid leukemia (AML) | down-regulated |
| hsa-miR-128a | Alzheimer's disease | up-regulated |
| hsa-miR-128a | autism spectrum disorder (ASD) | up-regulated |
| hsa-miR-128a | glioblastoma | down-regulated |
| hsa-miR-128a | glioblastoma multiforme (GBM) | down-regulated |
| hsa-miR-128a | malignant melanoma | down-regulated |
| hsa-miR-128a | Oral Squamous Cell Carcinoma (OSCC) | down-regulated |
| hsa-miR-128a | pituitary adenoma | down-regulated |
| hsa-miR-128b | lung cancer | down-regulated |
| hsa-miR-128b | acute lymphoblastic leukemia (ALL) | up-regulated |
| hsa-miR-128b | acute myeloid leukemia (AML) | down-regulated |
| hsa-miR-128b | breast cancer | up-regulated |
| hsa-miR-128b | chronic pancreatitis | up-regulated |
| hsa-miR-128b | colorectal cancer | up-regulated |
| hsa-miR-128b | lung cancer | up-regulated |
| hsa-miR-128b | pancreatic cancer | up-regulated |
| hsa-miR-129 | autism spectrum disorder (ASD) | up-regulated |
| hsa-miR-129 | ovarian cancer (OC) | up-regulated |
| hsa-miR-95 | malignant melanoma | down-regulated |
| hsa-miR-220 | B-cell chronic lymphocytic leukemia | down-regulated |
| hsa-miR-326 | medulloblastoma | down-regulated |
| hsa-miR-335 | multiple myeloma (MM) | up-regulated |
| hsa-miR-342 | multiple myeloma (MM) | up-regulated |
| hsa-miR-34a | chronic lymphocytic leukemia (CLL) | down-regulated |
| hsa-miR-34a | Glomerulosclerosis | down-regulated |
| hsa-miR-34a | prostate cancer | down-regulated |
| hsa-miR-34b | Burkitt lymphoma | down-regulated |
| hsa-miR-355 | breast cancer | down-regulated |
| hsa-miR-365 | breast cancer | up-regulated |
| hsa-miR-373* | retinoblastoma | up-regulated |
| hsa-miR-492 | retinoblastoma | up-regulated |
| hsa-miR-494 | retinoblastoma | down-regulated |
| hsa-miR-497 | breast cancer | up-regulated |
| hsa-miR-498 | retinoblastoma | up-regulated |
| hsa-miR-503 | retinoblastoma | up-regulated |
| hsa-miR-513-1 | retinoblastoma | up-regulated |
| hsa-miR-513-2 | retinoblastoma | up-regulated |
| hsa-miR-516-3p | breast cancer | up-regulated |
| hsa-miR-518c* | retinoblastoma | down-regulated |
| hsa-miR-128b | malignant melanoma | down-regulated |
| hsa-miR-129 | colorectal cancer | down-regulated |
| hsa-miR-129 | malignant melanoma | down-regulated |
| hsa-miR-23a | Acute Promyelocytic Leukemia (APL) | up-regulated |
| hsa-miR-17 | B-cell lymphoma | up-regulated |
| hsa-miR-20a | B-cell lymphoma | up-regulated |
| hsa-miR-155 | breast cancer | up-regulated |
| hsa-miR-204 | breast cancer | up-regulated |
| hsa-miR-21 | breast cancer | up-regulated |
| hsa-miR-221 | breast cancer | up-regulated |
| hsa-miR-221 | breast cancer | up-regulated |
| hsa-miR-222 | breast cancer | up-regulated |
| hsa-miR-222 | breast cancer | up-regulated |
| hsa-miR-510 | breast cancer | down-regulated |
| hsa-miR-7 | breast cancer | down-regulated |
| hsa-miR-425 | glioblastoma | down-regulated |
| hsa-miR-451 | glioblastoma | down-regulated |
| hsa-miR-486 | glioblastoma | up-regulated |
| hsa-miR-296 | glioma | down-regulated |
| hsa-miR-30a | glomerular disease | up-regulated |
| hsa-miR-21 | glioblastoma multiforme (GBM) | up-regulated |
| hsa-miR-335 | nemaline myopathy (NM) | down-regulated |
| hsa-miR-335 | ovarian cancer (OC) | up-regulated |
| hsa-miR-335 | uterine leiomyoma (ULM) | down-regulated |
| hsa-miR-337 | epithelial ovarian cancer (EOC) | down-regulated |
| hsa-miR-337 | Oral Squamous Cell Carcinoma (OSCC) | up-regulated |
| hsa-miR-338 | hepatocellular carcinoma (HCC) | up-regulated |
| hsa-miR-338 | follicular lymphoma (FL) | down-regulated |
| hsa-miR-338 | Oral Squamous Cell Carcinoma (OSCC) | up-regulated |
| hsa-miR-339 | chronic pancreatitis | down-regulated |
| hsa-miR-339 | colorectal cancer | up-regulated |
| hsa-miR-340 | acute myeloid leukemia (AML) | up-regulated |
| hsa-miR-340 | Oral Squamous Cell Carcinoma (OSCC) | up-regulated |
| hsa-miR-341 | cardiac hypertrophy | down-regulated |
| hsa-miR-342 | colorectal cancer | up-regulated |
| hsa-miR-342 | acute promyelocytic leukemia (APL) | down-regulated |
| hsa-miR-342 | Oral Squamous Cell Carcinoma (OSCC) | down-regulated |
| hsa-miR-345 | pancreatic cancer | up-regulated |
| hsa-miR-345 | papillary thyroid carcinoma (PTC) | up-regulated |
| hsa-miR-345 | prostate cancer | up-regulated |
| hsa-miR-346 | follicular thyroid carcinoma (FTC) | down-regulated |
| hsa-miR-346 | ovarian cancer (OC) | down-regulated |
| hsa-miR-346 | primary biliary cirrhosis (PBC) | down-regulated |
| hsa-miR-34a | hepatocellular carcinoma (HCC) | up-regulated |
| hsa-miR-220 | glioblastoma | down-regulated |
| hsa-miR-220 | lung cancer | up-regulated |
| hsa-miR-220 | pancreatic cancer | up-regulated |
| hsa-miR-220 | papillary thyroid carcinoma (PTC) | up-regulated |
| hsa-miR-221 | chronic lymphocytic leukemia (CLL) | up-regulated |
| hsa-miR-221 | glioblastoma | up-regulated |
| hsa-miR-221 | hepatocellular carcinoma (HCC) | up-regulated |
| hsa-miR-221 | malignant melanoma | down-regulated |
| hsa-miR-221 | non-small cell lung cancer (NSCLC) | up-regulated |
| hsa-miR-221 | non-small cell lung cancer (NSCLC) | up-regulated |
| hsa-miR-221 | papillary thyroid carcinoma (PTC) | down-regulated |
| hsa-miR-221 | prostate cancer | down-regulated |
| hsa-miR-221 | acute lymphoblastic leukemia (ALL) | up-regulated |
| hsa-miR-221 | acute myeloid leukemia (AML) | up-regulated |
| hsa-miR-221 | acute myeloid leukemia (AML) | up-regulated |
| hsa-miR-221 | Becker muscular dystrophy (BMD) | up-regulated |
| hsa-miR-221 | bladder cancer | up-regulated |
| hsa-miR-221 | cardiac hypertrophy | up-regulated |
| hsa-miR-221 | colorectal cancer | up-regulated |
| hsa-miR-221 | dermatomyositis (DM) | up-regulated |
| hsa-miR-221 | diffuse large B-cell lymphoma (DLBCL) | up-regulated |
| hsa-miR-221 | Duchenne muscular dystrophy (DMD) | up-regulated |
| hsa-miR-221 | facioscapulohumeral muscular dystrophy (FSHD) | up-regulated |
| hsa-miR-221 | gastric cancer (stomach cancer) | down-regulated |
| hsa-miR-222 | prostate cancer | down-regulated |
| hsa-miR-126 | colorectal cancer | down-regulated |
| hsa-miR-127 | breast cancer | down-regulated |
| hsa-miR-127 | hepatocellular carcinoma (HCC) | down-regulated |
| hsa-miR-16a | hepatocellular carcinoma (HCC) | down-regulated |
| hsa-miR-34a | hepatocellular carcinoma (HCC) | down-regulated |
| hsa-miR-183 | lung cancer | up-regulated |
| hsa-let-7a | lupus nephritis | up-regulated |
| hsa-let-7e | lupus nephritis | up-regulated |
| hsa-miR-124a | lupus nephritis | up-regulated |
| hsa-miR-130b | lupus nephritis | down-regulated |
| hsa-miR-133a | lupus nephritis | up-regulated |
| hsa-miR-134 | lupus nephritis | up-regulated |
| hsa-miR-142-5p | lupus nephritis | down-regulated |
| hsa-miR-150 | lupus nephritis | up-regulated |
| hsa-miR-15b | lupus nephritis | up-regulated |
| hsa-miR-184 | lupus nephritis | up-regulated |
| hsa-miR-185 | lupus nephritis | up-regulated |
| hsa-miR-195 | lupus nephritis | up-regulated |
| hsa-miR-197 | lupus nephritis | up-regulated |
| hsa-miR-198 | lupus nephritis | up-regulated |
| hsa-miR-200c | lupus nephritis | up-regulated |
| hsa-miR-628 | head and neck squamous cell carcinoma (HNSCC) | down-regulated |
| hsa-miR-145 | chronic lymphocytic leukemia (CLL) | up-regulated |
| hsa-miR-222 | pancreatic cancer | down-regulated |
| hsa-miR-29b | lung cancer | up-regulated |
| hsa-miR-382 | polymyositis (PM) | up-regulated |
| hsa-miR-383 | chronic pancreatitis | up-regulated |
| hsa-miR-409-3p | chronic pancreatitis | normal |
| hsa-miR-155 | hypertension | down-regulated |
| hsa-let-7a | hepatocellular carcinoma (HCC) | down-regulated |
| hsa-let-7a | malignant melanoma | down-regulated |
| hsa-let-7a | squamous carcinoma | down-regulated |
| hsa-let-7c | Burkitt lymphoma | up-regulated |
| hsa-let-7e | retinoblastoma | up-regulated |
| hsa-let-7f | breast cancer | down-regulated |
| hsa-miR-1 | lung cancer | up-regulated |
| hsa-miR-1 | retinitis pigmentosa (RP) | up-regulated |
| hsa-miR-106b | multiple myeloma (MM) | down-regulated |
| hsa-miR-122 | hepatocellular carcinoma (HCC) | down-regulated |
| hsa-miR-125b | medulloblastoma | up-regulated |
| hsa-miR-126 | acute myeloid leukemia (AML) | up-regulated |
| hsa-miR-126* | acute myeloid leukemia (AML) | up-regulated |
| hsa-let-7a | cholangiocarcinoma | normal |
| hsa-let-7a | adenoma | down-regulated |
| hsa-let-7a | breast cancer | down-regulated |
| hsa-let-7a | Burkitt lymphoma | down-regulated |
| hsa-let-7a | gastric cancer (stomach cancer) | normal |
| hsa-let-7a | hamartoma | up-regulated |
| hsa-let-7a | Hodgkin's lymphoma | normal |
| hsa-let-7a | lipoma | down-regulated |
| hsa-let-7a | lung cancer | down-regulated |
| hsa-let-7a | lung cancer | down-regulated |
| hsa-let-7a | lung cancer | normal |
| hsa-let-7a | myoma | down-regulated |
| hsa-let-7a | non-small cell lung cancer (NSCLC) | down-regulated |
| hsa-let-7a | ovarian cancer (OC) | normal |
| hsa-let-7a | sarcoma | down-regulated |
| hsa-let-7a | acute lymphoblastic leukemia (ALL) | up-regulated |
| hsa-let-7a | acute myeloid leukemia (AML) | down-regulated |
| hsa-let-7a | chronic lymphocytic leukemia (CLL) | down-regulated |
| hsa-let-7a | colorectal cancer | down-regulated |
| hsa-let-7a | epithelial ovarian cancer (EOC) | down-regulated |
| hsa-let-7a | gastric cancer (stomach cancer) | up-regulated |
| hsa-miR-583 | lupus nephritis | down-regulated |
| hsa-miR-23b | prostate cancer | up-regulated |
| hsa-miR-17-5p | neuroblastoma (NB) | up-regulated |
| hsa-miR-20a | colorectal cancer | up-regulated |
| hsa-miR-208 | lupus nephritis | down-regulated |
| hsa-miR-210 | lupus nephritis | down-regulated |
| hsa-miR-223 | lupus nephritis | down-regulated |
| hsa-miR-596 | lupus nephritis | up-regulated |
| hsa-miR-600 | lupus nephritis | up-regulated |
| hsa-miR-601 | lupus nephritis | down-regulated |
| hsa-miR-602 | lupus nephritis | up-regulated |
| hsa-miR-608 | lupus nephritis | down-regulated |
| hsa-miR-611 | lupus nephritis | up-regulated |
| hsa-miR-612 | lupus nephritis | down-regulated |
| hsa-miR-615 | lupus nephritis | up-regulated |
| hsa-miR-622 | lupus nephritis | down-regulated |
| hsa-miR-629 | lupus nephritis | down-regulated |
| hsa-miR-637 | lupus nephritis | up-regulated |
| hsa-miR-638 | lupus nepliritis | down-regulated |
| hsa-miR-642 | lupus nepliritis | down-regulated |
| hsa-miR-654 | lupus nepliritis | up-regulated |
| hsa-miR-657 | lupus nephritis | up-regulated |
| hsa-miR-658 | lupus nephritis | up-regulated |
| hsa-miR-662 | lupus nephritis | down-regulated |
| hsa-miR-663 | lupus nephritis | down-regulated |
| hsa-miR-769-3p | lupus nephritis | down-regulated |
| hsa-miR-92b | lupus nephritis | down-regulated |
| hsa-miR-137 | glioblastoma multiforme (GBM) | up-regulated |
| hsa-miR-137 | non-small cell lung cancer (NSCLC) | down-regulated |
| hsa-miR-137 | Oral Squamous Cell Carcinoma (OSCC) | up-regulated |
| hsa-miR-137 | hepatocellular carcinoma (HCC) | down-regulated |
| hsa-miR-137 | malignant melanoma | down-regulated |
| hsa-miR-138 | anaplastic thyroid carcinoma (ATC) | down-regulated |
| hsa-miR-138 | malignant melanoma | down-regulated |
| hsa-miR-138-1 | Oral Squamous Cell Carcinoma (OSCC) | up-regulated |
| hsa-miR-138-1 | papillary thyroid carcinoma (PTC) | down-regulated |
| hsa-miR-138-2 | Oral Squamous Cell Carcinoma (OSCC) | down-regulated |
| hsa-miR-206 | breast cancer | normal |
| hsa-miR-206 | schizophrenia | up-regulated |
| hsa-miR-206 | breast cancer | down-regulated |
| hsa-miR-206 | ovarian cancer (OC) | up-regulated |
| hsa-miR-207 | hepatocellular carcinoma (HCC) | up-regulated |
| hsa-miR-208 | cardiac hypertrophy | down-regulated |
| hsa-miR-20a | chronic myeloid leukemia (CML) | up-regulated |
| hsa-miR-20a | lung cancer | up-regulated |
| hsa-miR-20a | lung cancer | up-regulated |
| hsa-miR-20a | colorectal cancer | up-regulated |
| hsa-miR-20a | colorectal cancer | up-regulated |
| hsa-miR-20a | hepatocellular carcinoma (HCC) | down-regulated |
| hsa-miR-145 | colorectal cancer | down-regulated |
| hsa-miR-145 | colorectal cancer | down-regulated |
| hsa-miR-145 | colorectal cancer | down-regulated |
| hsa-miR-145 | diffuse large B-cell lymphoma (DLBCL) | down-regulated |
| hsa-miR-148a | pancreatic ductal adenocarcinoma (PDAC) | up-regulated |
| hsa-miR-15b | pancreatic cancer | down-regulated |
| hsa-miR-197 | Inclusion body myositis (IBM) | down-regulated |
| hsa-miR-197 | limb-girdle muscular dystrophies types 2A (LGMD2A) | up-regulated |
| hsa-miR-197 | lung cancer | down-regulated |
| hsa-miR-197 | Oral Squamous Cell Carcinoma (OSCC) | up-regulated |
| hsa-miR-197 | pituitary adenoma | down-regulated |
| hsa-miR-197 | primary biliary cirrhosis (PBC) | down-regulated |
| hsa-miR-197 | psoriasis | down-regulated |
| hsa-miR-197 | uterine leiomyoma (ULM) | normal |
| hsa-miR-198 | schizophrenia | up-regulated |
| hsa-miR-198 | chronic pancreatitis | down-regulated |
| hsa-miR-198 | lung cancer | up-regulated |
| hsa-miR-198 | prostate cancer | down-regulated |
| hsa-miR-199a | cancer | down-regulated |
| hsa-miR-199a | epithelial ovarian cancer (EOC) | down-regulated |
| hsa-miR-199a | acute myeloid leukemia (AML) | up-regulated |
| hsa-miR-199a | cardiac hypertrophy | up-regulated |
| hsa-miR-199a | dermatomyositis (DM) | up-regulated |
| hsa-miR-199a | Duchenne muscular dystrophy (DMD) | down-regulated |
| hsa-miR-199a | epithelial ovarian cancer (EOC) | down-regulated |
| hsa-miR-199b | malignant melanoma | up-regulated |
| hsa-miR-199b | prostate cancer | up-regulated |
| hsa-miR-21 | bladder cancer | up-regulated |
| hsa-miR-21 | breast cancer | up-regulated |
| hsa-miR-21 | glioblastoma | up-regulated |
| hsa-miR-21 | hepatocellular carcinoma (HCC) | up-regulated |
| hsa-miR-21 | non-small cell lung cancer (NSCLC) | up-regulated |
| hsa-miR-210 | breast cancer | up-regulated |
| hsa-miR-217 | pancreatic ductal adenocarcinoma (PDAC) | up-regulated |
| hsa-miR-221 | glioblastoma multiforme (GBM) | up-regulated |
| hsa-miR-25 | multiple myeloma (MM) | down-regulated |
| hsa-miR-26a | Burkitt lymphoma | down-regulated |
| hsa-miR-28 | Glomerulosclerosis | up-regulated |
| hsa-miR-29b | breast cancer | up-regulated |
| hsa-miR-29c | breast cancer | down-regulated |
| hsa-miR-30a-3p | breast cancer | down-regulated |
| hsa-miR-30b | Glomerulosclerosis | down-regulated |
| hsa-miR-30c-1 | Glomerulosclerosis | down-regulated |
| hsa-miR-30c-2 | Glomerulosclerosis | down-regulated |
| hsa-miR-30d | Glomerulosclerosis | up-regulated |
| hsa-miR-32 | multiple myeloma (MM) | down-regulated |
| hsa-miR-23a | colorectal cancer | up-regulated |
| hsa-miR-17-5p | hepatocellular carcinoma (HCC) | down-regulated |
| hsa-miR-17-5p | breast cancer | down-regulated |
| hsa-miR-181a | glioma | up-regulated |
| hsa-miR-181a | multiple myeloma (MM) | up-regulated |
| hsa-miR-181b | breast cancer | down-regulated |
| hsa-miR-181b | glioma | up-regulated |
| hsa-miR-181b | multiple myeloma (MM) | up-regulated |
| hsa-miR-181d | breast cancer | down-regulated |
| hsa-miR-182 | retinitis pigmentosa (RP) | down-regulated |
| hsa-miR-183 | retinitis pigmentosa (RP) | up-regulated |
| hsa-miR-18a | hepatocellular carcinoma (HCC) | up-regulated |
| hsa-miR-196a | esophageal cancer | up-regulated |
| hsa-miR-196a | pancreatic ductal adenocarcinoma (PDAC) | up-regulated |
| hsa-miR-198 | retinoblastoma | up-regulated |
| hsa-miR-19a | hepatocellular carcinoma (HCC) | up-regulated |
| hsa-miR-19a | multiple myeloma (MM) | up-regulated |
| hsa-miR-19b | hepatocellular carcinoma (HCC) | up-regulated |
| hsa-miR-19b | multiple myeloma (MM) | down-regulated |
| hsa-miR-205 | bladder cancer | down-regulated |
| hsa-miR-20a | breast cancer | up-regulated |
| hsa-miR-222 | pancreatic ductal adenocarcinoma (PDAC) | up-regulated |
| hsa-miR-222 | papillary thyroid carcinoma (PTC) | up-regulated |
| hsa-miR-222 | polymyositis (PM) | down-regulated |
| hsa-miR-222 | prostate cancer | down-regulated |
| hsa-miR-223 | chronic lymphocytic leukemia (CLL) | up-regulated |
| hsa-miR-223 | acute promyelocytic leukemia (APL) | up-regulated |
| hsa-miR-223 | acute promyelocytic leukemia (APL) | down-regulated |
| hsa-miR-223 | chronic myeloid leukemia (CML) | down-regulated |
| hsa-miR-223 | hepatocellular carcinoma (HCC) | down-regulated |
| hsa-miR-223 | neutrophilia | up-regulated |
| hsa-miR-223 | recurrent ovarian cancer | down-regulated |
| hsa-miR-223 | acute lymphoblastic leukemia (ALL) | up-regulated |
| hsa-miR-223 | acute myeloid leukemia (AML) | up-regulated |
| hsa-miR-223 | bladder cancer | up-regulated |
| hsa-miR-223 | colorectal cancer | up-regulated |
| hsa-miR-223 | dermatomyositis (DM) | up-regulated |
| hsa-miR-223 | gastric cancer (stomach cancer) | down-regulated |
| hsa-miR-223 | hepatocellular carcinoma (HCC) | up-regulated |
| hsa-miR-223 | Inclusion body myositis (IBM) | up-regulated |
| hsa-miR-223 | limb-girdle muscular dystrophies types 2A (LGMD2A) | up-regulated |
| hsa-miR-223 | miyoshi myopathy (MM) | up-regulated |
| hsa-miR-223 | nemaline myopathy (NM) | down-regulated |
| hsa-miR-223 | Oral Squamous Cell Carcinoma (OSCC) | up-regulated |
| hsa-miR-223 | pancreatic cancer | down-regulated |
| hsa-miR-214 | ovarian cancer (OC) | up-regulated |
| hsa-miR-214 | cardiac hypertrophy | up-regulated |
| hsa-miR-214 | cardiac hypertrophy | up-regulated |
| hsa-miR-214 | dermatomyositis (DM) | up-regulated |
| hsa-miR-214 | Duchenne muscular dystrophy (DMD) | down-regulated |
| hsa-miR-214 | epithelial ovarian cancer (EOC) | up-regulated |
| hsa-miR-214 | facioscapulohumeral muscular dystrophy (FSHD) | up-regulated |
| hsa-miR-214 | gastric cancer (stomach cancer) | up-regulated |
| hsa-miR-214 | head and neck squamous cell carcinoma (HNSCC) | up-regulated |
| hsa-miR-214 | heart failure | down-regulated |
| hsa-miR-214 | hepatocellular carcinoma (HCC) | down-regulated |
| hsa-miR-214 | hepatocellular carcinoma (HCC) | up-regulated |
| hsa-miR-214 | Inclusion body myositis (IBM) | up-regulated |
| hsa-miR-214 | limb-girdle muscular dystrophies types 2A (LGMD2A) | up-regulated |
| hsa-miR-214 | lung cancer | up-regulated |
| hsa-miR-214 | miyoshi myopathy (MM) | up-regulated |
| hsa-miR-214 | nemaline myopathy (NM) | up-regulated |
| hsa-miR-214 | pancreatic cancer | up-regulated |
| hsa-miR-214 | polymyositis (PM) | up-regulated |
| hsa-miR-214 | prostate cancer | down-regulated |
| hsa-miR-214 | serous ovarian cancer | down-regulated |
| hsa-miR-215 | hepatocellular carcinoma (HCC) | down-regulated |
| hsa-miR-215 | Oral Squamous Cell Carcinoma (OSCC) | down-regulated |
| hsa-miR-215 | psoriasis | down-regulated |
| hsa-miR-216 | lung cancer | up-regulated |
| hsa-miR-214 | vascular disease | down-regulated |
| hsa-miR-215 | malignant melanoma | up-regulated |
| hsa-miR-216 | hepatocellular carcinoma (HCC) | up-regulated |
| hsa-miR-495 | dermatomyositis (DM) | up-regulated |
| hsa-miR-96 | colorectal cancer | up-regulated |
| hsa-miR-129 | primary biliary cirrhosis (PBC) | up-regulated |
| hsa-miR-34a | colorectal cancer | up-regulated |
| hsa-miR-221 | glioblastoma | down-regulated |
| hsa-miR-128 | glioma | up-regulated |
| hsa-miR-128a | breast cancer | up-regulated |
| hsa-miR-129-1 | retinoblastoma | up-regulated |
| hsa-miR-129-2 | retinoblastoma | up-regulated |
| hsa-miR-133 | retinitis pigmentosa (RP) | up-regulated |
| hsa-miR-142 | retinitis pigmentosa (RP) | up-regulated |
| hsa-miR-143 | Obesity | down-regulated |
| hsa-miR-145 | colorectal cancer | up-regulated |
| hsa-miR-146a | Alzheimer's disease | up-regulated |
| hsa-miR-146a | breast cancer | up-regulated |
| hsa-miR-146a | hepatocellular carcinoma (HCC) | up-regulated |
| hsa-miR-146a | ovarian cancer (OC) | up-regulated |
| hsa-miR-521 | prostate cancer | up-regulated |
| hsa-miR-194 | endometriosis | up-regulated |
| hsa-miR-495 | Duchenne muscular dystrophy (DMD) | up-regulated |
| hsa-miR-495 | limb-girdle muscular dystrophies types 2A (LGMD2A) | up-regulated |
| hsa-miR-495 | miyoshi myopathy (MM) | up-regulated |
| hsa-miR-495 | nemaline myopathy (NM) | down-regulated |
| hsa-miR-497 | chronic pancreatitis | down-regulated |
| hsa-miR-497 | prostate cancer | up-regulated |
| hsa-miR-498 | head and neck squamous cell carcinoma (HNSCC) | up-regulated |
| hsa-miR-498 | ovarian cancer (OC) | up-regulated |
| hsa-miR-498 | prostate cancer | down-regulated |
| hsa-miR-498 | uterine leiomyoma (ULM) | up-regulated |
| hsa-miR-5 | acute lymphoblastic leukemia (ALL) | down-regulated |
| hsa-miR-5 | acute myeloid leukemia (AML) | up-regulated |
| hsa-miR-501 | dermatomyositis (DM) | up-regulated |
| hsa-miR-501 | limb-girdle muscular dystrophies types 2A (LGMD2A) | up-regulated |
| hsa-miR-501 | nemaline myopathy (NM) | up-regulated |
| hsa-miR-501 | polymyositis (PM) | up-regulated |
| hsa-miR-503 | prostate cancer | up-regulated |
| hsa-miR-508 | ovarian cancer (OC) | normal |
| hsa-miR-510 | diarrhea predominant irritable bowel syndrome (IBS-D) | up-regulated |
| hsa-miR-511 | Alzheimer's disease | up-regulated |
| hsa-miR-513 | prostate cancer | down-regulated |
| hsa-miR-515-3p | ovarian cancer (OC) | down-regulated |
| hsa-miR-516-5p | ovarian cancer (OC) | up-regulated |
| hsa-miR-518a-2* | Duchenne muscular dystrophy (DMD) | up-regulated |
| hsa-miR-518a-2* | limb-girdle muscular dystrophies types 2A (LGMD2A) | down-regulated |
| hsa-miR-10a | colorectal cancer | up-regulated |
| hsa-miR-205 | non-small cell lung cancer (NSCLC) | up-regulated |
| hsa-miR-20a | colorectal cancer | down-regulated |
| hsa-miR-31 | breast cancer | down-regulated |
| hsa-miR-320 | breast cancer | up-regulated |
| hsa-miR-320 | colorectal cancer | up-regulated |
| hsa-miR-320 | retinoblastoma | up-regulated |
| hsa-miR-561 | multiple myeloma (MM) | up-regulated |
| hsa-miR-615-3p | HBV-related cirrhosis | normal |
| hsa-miR-659 | frontotemporal dementia | up-regulated |
| hsa-miR-7 | breast cancer | up-regulated |
| hsa-miR-92 | colorectal cancer | up-regulated |
| hsa-miR-92a | hepatocellular carcinoma (HCC) | up-regulated |
| hsa-miR-93 | multiple myeloma (MM) | down-regulated |
| hsa-miR-96 | retinitis pigmentosa (RP) | up-regulated |
| hsa-miR-98 | breast cancer | up-regulated |
| hsa-miR-196b | acute lymphoblastic leukemia (ALL) | up-regulated |
| hsa-miR-708 | acute lymphoblastic leukemia (ALL) | down-regulated |
| hsa-miR-29b-2 | rhabdomyosarcoma (RMS) | down-regulated |
| hsa-miR-29c | rhabdomyosarcoma (RMS) | down-regulated |
| hsa-miR-101 | Spinocerebellar ataxia 1 | up-regulated |
| hsa-miR-96 | hepatocellular carcinoma (HCC) | up-regulated |
| hsa-miR-96 | hepatocellular carcinoma (HCC) | down-regulated |
| hsa-miR-96 | malignant melanoma | down-regulated |
| hsa-miR-96 | non-small cell lung cancer (NSCLC) | down-regulated |
| hsa-miR-96 | Oral Squamous Cell Carcinoma (OSCC) | down-regulated |
| hsa-miR-96 | pancreatic ductal adenocarcinoma (PDAC) | up-regulated |
| hsa-miR-96 | retinitis pigmentosa (RP) | up-regulated |
| hsa-miR-98 | head and neck squamous cell carcinoma (HNSCC) | up-regulated |
| hsa-miR-99a | chronic pancreatitis | down-regulated |
| hsa-miR-99a | diffuse large B-cell lymphoma (DLBCL) | up-regulated |
| hsa-miR-99a | Down syndrome (DS) | down-regulated |
| hsa-miR-99a | epithelial ovarian cancer (EOC) | down-regulated |
| hsa-miR-99a | epithelial ovarian cancer (EOC) | down-regulated |
| hsa-miR-99a | hepatocellular carcinoma (HCC) | down-regulated |
| hsa-miR-99a | lung cancer | up-regulated |
| hsa-miR-99a | neuroblastoma (NB) | up-regulated |
| hsa-miR-99a | ovarian cancer (OC) | down-regulated |
| hsa-miR-99a | prostate cancer | down-regulated |
| hsa-miR-99a | serous ovarian cancer | up-regulated |
| hsa-miR-99b | dermatomyositis (DM) | up-regulated |
| hsa-miR-99b | facioscapulohumeral muscular dystrophy (FSHD) | up-regulated |
| hsa-miR-99b | limb-girdle muscular dystrophies types 2A (LGMD2A) | up-regulated |
| hsa-miR-99b | miyoshi myopathy (MM) | down-regulated |
| hsa-miR-130a | acute lymphoblastic leukemia (ALL) | up-regulated |
| hsa-miR-130a | acute myeloid leukemia (AML) | up-regulated |
| hsa-miR-130a | dermatomyositis (DM) | up-regulated |
| hsa-miR-130a | Duchenne muscular dystrophy (DMD) | up-regulated |
| hsa-miR-130a | glioblastoma | down-regulated |
| hsa-let-7c | acute lymphoblastic leukemia (ALL) | up-regulated |
| hsa-miR-1 | cardiomyopathy | down-regulated |
| hsa-miR-124a | Cerebellar neurodegeneration | down-regulated |
| hsa-miR-139 | pancreatic cancer | down-regulated |
| hsa-miR-145 | breast cancer | up-regulated |
| hsa-miR-155 | acute myeloid leukemia (AML) | down-regulated |
| hsa-miR-16 | chronic lymphocytic leukemia (CLL) | down-regulated |
| hsa-miR-181a-2 | hepatocellular carcinoma (HCC) | down-regulated |
| hsa-miR-181b | chronic lymphocytic leukemia (CLL) | up-regulated |
| hsa-miR-181b | acute myeloid leukemia (AML) | down-regulated |
| hsa-miR-181b | acute promyelocytic leukemia (APL) | up-regulated |
| hsa-miR-181b | B-cell chronic lymphocytic leukemia | up-regulated |
| hsa-miR-181b | colorectal cancer | up-regulated |
| hsa-miR-181b | colorectal cancer | up-regulated |
| hsa-miR-181b | colorectal cancer | down-regulated |
| hsa-miR-181b | glioblastoma | up-regulated |
| hsa-miR-181b-1 | breast cancer | up-regulated |
| hsa-miR-181b-1 | pancreatic cancer | up-regulated |
| hsa-miR-183 | colorectal cancer | up-regulated |
| hsa-miR-183 | hepatocellular carcinoma (HCC) | down-regulated |
| hsa-miR-183 | malignant melanoma | down-regulated |
| hsa-miR-184 | neuroblastoma (NB) | up-regulated |
| hsa-miR-196a | pancreatic ductal adenocarcinoma (PDAC) | up-regulated |
| hsa-miR-196a-2 | breast cancer | down-regulated |
| hsa-miR-196b | cervical cancer | up-regulated |
| hsa-miR-196b | uterine leiomyoma (ULM) | up-regulated |
| hsa-miR-197 | follicular thyroid carcinoma (FTC) | up-regulated |
| hsa-miR-197 | Alzheimer's disease | up-regulated |
| hsa-miR-197 | chronic pancreatitis | down-regulated |
| hsa-miR-197 | Duchenne muscular dystrophy (DMD) | down-regulated |
| hsa-miR-197 | glioblastoma | down-regulated |
| hsa-miR-197 | head and neck squamous cell carcinoma (HNSCC) | down-regulated |
| hsa-miR-381 | autism spectrum disorder (ASD) | up-regulated |
| hsa-miR-381 | dermatomyositis (DM) | up-regulated |
| hsa-miR-381 | Duchenne muscular dystrophy (DMD) | up-regulated |
| hsa-miR-381 | Inclusion body myositis (IBM) | up-regulated |
| hsa-miR-381 | limb-girdle muscular dystrophies types 2A (LGMD2A) | up-regulated |
| hsa-miR-381 | miyoshi myopathy (MM) | up-regulated |
| hsa-miR-381 | nemaline myopathy (NM) | down-regulated |
| hsa-miR-381 | ovarian cancer (OC) | down-regulated |
| hsa-miR-381 | psoriasis | up-regulated |
| hsa-miR-382 | dermatomyositis (DM) | up-regulated |
| hsa-miR-29b | type 2 diabetes | down-regulated |
| hsa-miR-29b | cardiac hypertrophy | down-regulated |
| hsa-miR-29b | Duchenne muscular dystrophy (DMD) | up-regulated |
| hsa-miR-29b | facioscapulohumeral muscular dystrophy (FSHD) | down-regulated |
| hsa-miR-29b | glioblastoma multiforme (GBM) | down-regulated |
| hsa-miR-29b | head and neck squamous cell carcinoma (HNSCC) | up-regulated |
| hsa-miR-29b | limb-girdle muscular dystrophies types 2A (LGMD2A) | down-regulated |
| hsa-miR-29b | malignant melanoma | down-regulated |
| hsa-miR-29b | nemaline myopathy (NM) | up-regulated |
| hsa-miR-29b | papillary thyroid carcinoma (PTC) | down-regulated |
| hsa-miR-29b | primary biliary cirrhosis (PBC) | down-regulated |
| hsa-miR-29b | prostate cancer | down-regulated |
| hsa-miR-29b | schizophrenia | down-regulated |
| hsa-miR-29b | uterine leiomyoma (ULM) | down-regulated |
| hsa-miR-29b | uterine leiomyoma (ULM) | down-regulated |
| hsa-miR-29b-1 | Alzheimer's disease | down-regulated |
| hsa-miR-29b-2 | chronic lymphocytic leukemia (CLL) | up-regulated |
| hsa-miR-29b-2 | breast cancer | up-regulated |
| hsa-miR-29b-2 | colorectal cancer | down-regulated |
| hsa-miR-29b-2 | lung cancer | up-regulated |
| hsa-miR-29b-2 | pancreatic cancer | up-regulated |
| hsa-miR-29b-2 | prostate cancer | down-regulated |
| hsa-miR-29c | chronic lymphocytic leukemia (CLL) | down-regulated |
| hsa-miR-204 | Oral Squamous Cell Carcinoma (OSCC) | down-regulated |
| hsa-miR-205 | breast cancer | up-regulated |
| hsa-miR-205 | bladder cancer | down-regulated |
| hsa-miR-205 | breast cancer | down-regulated |
| hsa-miR-205 | esophageal cancer | up-regulated |
| hsa-miR-205 | Head and neck cancer | up-regulated |
| hsa-miR-205 | lung cancer | up-regulated |
| hsa-miR-205 | pancreatic cancer | up-regulated |
| hsa-miR-205 | pancreatic ductal adenocarcinoma (PDAC) | up-regulated |
| hsa-miR-205 | prostate cancer | down-regulated |
| hsa-miR-206 | breast cancer | up-regulated |
| hsa-miR-155 | polymyositis (PM) | up-regulated |
| hsa-miR-15a | chronic lymphocytic leukemia (CLL) | up-regulated |
| hsa-miR-15a | hepatocellular carcinoma (HCC) | down-regulated |
| hsa-miR-15a | acute promyelocytic leukemia (APL) | up-regulated |
| hsa-miR-15a | chronic lymphocytic leukemia (CLL) | down-regulated |
| hsa-miR-15a | chronic lymphocytic leukemia (CLL) | down-regulated |
| hsa-miR-15a | chronic lymphocytic leukemia (CLL) | down-regulated |
| hsa-miR-15a | pituitary adenoma | down-regulated |
| hsa-miR-15a | Alzheimer's disease | down-regulated |
| hsa-miR-15a | autism spectrum disorder (ASD) | up-regulated |
| hsa-miR-15a | head and neck squamous cell carcinoma (HNSCC) | up-regulated |
| hsa-miR-99a | lupus nephritis | down-regulated |
| hsa-miR-199a | facioscapulohumeral muscular dystrophy (FSHD) | up-regulated |
| hsa-miR-199a | heart failure | up-regulated |
| hsa-miR-199a | hepatocellular carcinoma (HCC) | down-regulated |
| hsa-miR-199a | limb-girdle muscular dystrophies types 2A (LGMD2A) | up-regulated |
| hsa-miR-199a | malignant melanoma | down-regulated |
| hsa-miR-199a | miyoshi myopathy (MM) | up-regulated |
| hsa-miR-199a | nemaline myopathy (NM) | up-regulated |
| hsa-miR-199a | ovarian cancer (OC) | up-regulated |
| hsa-miR-199a | polymyositis (PM) | up-regulated |
| hsa-miR-199a | prostate cancer | down-regulated |
| hsa-miR-199a | uveal melanoma | up-regulated |
| hsa-miR-199a* | cancer | down-regulated |
| hsa-miR-199a* | cardiac hypertrophy | up-regulated |
| hsa-miR-199a* | dermatomyositis (DM) | up-regulated |
| hsa-miR-199a* | Duchenne muscular dystrophy (DMD) | up-regulated |
| hsa-miR-199a* | facioscapulohumeral muscular dystrophy (FSHD) | up-regulated |
| hsa-miR-199a* | hepatocellular carcinoma (HCC) | down-regulated |
| hsa-miR-199a* | limb-girdle muscular dystrophies types 2A (LGMD2A) | up-regulated |
| hsa-miR-199a* | miyoshi myopathy (MM) | up-regulated |
| hsa-miR-199a* | nemaline myopathy (NM) | up-regulated |
| hsa-miR-199a* | ovarian cancer (OC) | down-regulated |
| hsa-miR-199a* | prostate cancer | down-regulated |
| hsa-miR-199a-1 | chronic pancreatitis | up-regulated |
| hsa-miR-199a-1 | lung cancer | up-regulated |
| hsa-miR-133a | cardiac hypertrophy | down-regulated |
| hsa-miR-223 | pancreatic cancer | up-regulated |
| hsa-miR-189 | tourette's syndrome | normal |
| hsa-miR-18a | anaplastic thyroid carcinoma (ATC) | up-regulated |
| hsa-miR-18a | lung cancer | up-regulated |
| hsa-miR-18a | pancreatic ductal adenocarcinoma (PDAC) | up-regulated |
| hsa-miR-18b | cardiac hypertrophy | up-regulated |
| hsa-miR-190 | B-cell chronic lymphocytic leukemia | up-regulated |
| hsa-miR-190 | malignant melanoma | down-regulated |
| hsa-miR-190 | ovarian cancer (OC) | up-regulated |
| hsa-miR-191 | breast cancer | up-regulated |
| hsa-miR-191 | colorectal cancer | up-regulated |
| hsa-miR-191 | colorectal cancer | down-regulated |
| hsa-miR-191 | colorectal cancer | up-regulated |
| hsa-miR-191 | gastric cancer (stomach cancer) | up-regulated |
| hsa-miR-191 | glioblastoma | up-regulated |
| hsa-miR-191 | head and neck squamous cell carcinoma (HNSCC) | up-regulated |
| hsa-miR-191 | lung cancer | up-regulated |
| hsa-miR-191 | lung cancer | up-regulated |
| hsa-miR-191 | pancreatic cancer | up-regulated |
| hsa-miR-191 | prostate cancer | up-regulated |
| hsa-miR-191 | prostate cancer | up-regulated |
| hsa-miR-192 | B-cell chronic lymphocytic leukemia | down-regulated |
| hsa-miR-192 | lung cancer | up-regulated |
| hsa-let-7a | nemaline myopathy (NM) | up-regulated |
| hsa-let-7a | prostate cancer | down-regulated |
| hsa-let-7a-1 | colorectal cancer | down-regulated |
| hsa-let-7a-1 | hepatocellular carcinoma (HCC) | down-regulated |
| hsa-let-7a-1 | Oral Squamous Cell Carcinoma (OSCC) | down-regulated |
| hsa-let-7a-1 | pituitary adenoma | down-regulated |
| hsa-let-7a-2 | lung cancer | down-regulated |
| hsa-let-7a-2 | hepatocellular carcinoma (HCC) | down-regulated |
| hsa-let-7a-2 | Oral Squamous Cell Carcinoma (OSCC) | down-regulated |
| hsa-let-7a-3 | acute promyelocytic leukemia (APL) | up-regulated |
| hsa-let-7a-3 | epithelial ovarian cancer (EOC) | down-regulated |
| hsa-let-7a-3 | lung cancer | up-regulated |
| hsa-let-7a-3 | breast cancer | down-regulated |
| hsa-let-7a-3 | hepatocellular carcinoma (HCC) | down-regulated |
| hsa-let-7a-3 | Oral Squamous Cell Carcinoma (OSCC) | down-regulated |
| hsa-let-7b | serous ovarian cancer | down-regulated |
| hsa-let-7b | lung cancer | down-regulated |
| hsa-let-7b | lung cancer | down-regulated |
| hsa-let-7b | malignant melanoma | down-regulated |
| hsa-let-7b | acute lymphoblastic leukemia (ALL) | down-regulated |
| hsa-let-7b | acute myeloid leukemia (AML) | up-regulated |
| hsa-let-7b | cardiac hypertrophy | up-regulated |
| hsa-let-7b | cervical cancer | down-regulated |
| hsa-miR-223 | pancreatic ductal adenocarcinoma (PDAC) | up-regulated |
| hsa-miR-223 | pituitary adenoma | down-regulated |
| hsa-miR-223 | primary biliary cirrhosis (PBC) | down-regulated |
| hsa-miR-223 | prostate cancer | up-regulated |
| hsa-miR-224 | hepatocellular carcinoma (HCC) | up-regulated |
| hsa-miR-224 | epithelial ovarian cancer (EOC) | down-regulated |
| hsa-miR-224 | hepatocellular carcinoma (HCC) | up-regulated |
| hsa-miR-224 | hepatocellular carcinoma (HCC) | up-regulated |
| hsa-miR-224 | lung cancer | down-regulated |
| hsa-miR-224 | malignant melanoma | down-regulated |
| hsa-miR-224 | Oral Squamous Cell Carcinoma (OSCC) | up-regulated |
| hsa-miR-224 | pancreatic ductal adenocarcinoma (PDAC) | up-regulated |
| hsa-miR-224 | prostate cancer | up-regulated |
| hsa-miR-23a | hepatocellular carcinoma (HCC) | up-regulated |
| hsa-miR-23a | acute lymphoblastic leukemia (ALL) | down-regulated |
| hsa-miR-23a | acute myeloid leukemia (AML) | up-regulated |
| hsa-miR-23a | autism spectrum disorder (ASD) | up-regulated |
| hsa-miR-23a | bladder cancer | up-regulated |
| hsa-miR-23a | cardiac hypertrophy | up-regulated |
| hsa-miR-23a | glioblastoma | up-regulated |
| hsa-miR-23a | heart failure | up-regulated |
| hsa-miR-23a | hepatocellular carcinoma (HCC) | up-regulated |
| hsa-miR-34a | neuroblastoma (NB) | down-regulated |
| hsa-miR-34a | neuroblastoma (NB) | down-regulated |
| hsa-miR-34a | neuroblastoma (NB) | down-regulated |
| hsa-miR-34a | non-small cell lung cancer (NSCLC) | down-regulated |
| hsa-miR-34a | pancreatic cancer | down-regulated |
| hsa-miR-34a | dermatomyositis (DM) | up-regulated |
| hsa-miR-34a | Duchenne muscular dystrophy (DMD) | up-regulated |
| hsa-miR-34a | facioscapulohumeral muscular dystrophy (FSHD) | up-regulated |
| hsa-miR-34a | Inclusion body myositis (IBM) | up-regulated |
| hsa-miR-34a | limb-girdle muscular dystrophies types 2A (LGMD2A) | up-regulated |
| hsa-miR-34a | miyoshi myopathy (MM) | up-regulated |
| hsa-miR-34a | nemaline myopathy (NM) | up-regulated |
| hsa-miR-34a | Oral Squamous Cell Carcinoma (OSCC) | down-regulated |
| hsa-miR-34a | papillary thyroid carcinoma (PTC) | up-regulated |
| hsa-miR-34a | polymyositis (PM) | up-regulated |
| hsa-miR-34a | uterine leiomyoma (ULM) | up-regulated |
| hsa-miR-34b | colorectal cancer | down-regulated |
| hsa-miR-34b | non-small cell lung cancer (NSCLC) | down-regulated |
| hsa-miR-34b | ovarian cancer (OC) | down-regulated |
| hsa-miR-34b | malignant melanoma | down-regulated |
| hsa-miR-34b | nasopharyngeal carcinoma (NPC) | down-regulated |
| hsa-miR-34b | Oral Squamous Cell Carcinoma (OSCC) | down-regulated |
| hsa-miR-34c | non-small cell lung cancer (NSCLC) | down-regulated |
| hsa-miR-34c | colorectal cancer | down-regulated |
| hsa-miR-145 | breast cancer | down-regulated |
| hsa-miR-145 | colorectal cancer | down-regulated |
| hsa-miR-145 | colorectal cancer | down-regulated |
| hsa-miR-145 | colorectal cancer | down-regulated |
| hsa-miR-145 | diffuse large B-cell lymphoma (DLBCL) | down-regulated |
| hsa-miR-145 | epithelial ovarian cancer (EOC) | down-regulated |
| hsa-miR-145 | epithelial ovarian cancer (EOC) | down-regulated |
| hsa-miR-145 | hepatocellular carcinoma (HCC) | down-regulated |
| hsa-miR-145 | hepatocellular carcinoma (HCC) | down-regulated |
| hsa-miR-145 | pancreatic ductal adenocarcinoma (PDAC) | up-regulated |
| hsa-miR-145 | prostate cancer | down-regulated |
| hsa-miR-145 | prostate cancer | up-regulated |
| hsa-miR-145 | serous ovarian cancer | down-regulated |
| hsa-miR-145 | testicular germ cell tumor | up-regulated |
| hsa-miR-145 | vascular disease | down-regulated |
| hsa-miR-146a | chronic lymphocytic leukemia (CLL) | up-regulated |
| hsa-miR-146a | breast cancer | up-regulated |
| hsa-miR-146a | breast cancer | down-regulated |
| hsa-miR-146a | papillary thyroid carcinoma (PTC) | up-regulated |
| hsa-miR-146a | prostate cancer | down-regulated |
| hsa-miR-146a | breast cancer | up-regulated |
| hsa-miR-146a | cardiac hypertrophy | up-regulated |
| hsa-miR-221 | hepatocellular carcinoma (HCC) | up-regulated |
| hsa-miR-221 | hepatocellular carcinoma (HCC) | up-regulated |
| hsa-miR-221 | hepatocellular carcinoma (HCC) | up-regulated |
| hsa-miR-221 | Inclusion body myositis (IBM) | up-regulated |
| hsa-miR-221 | limb-girdle muscular dystrophies types 2A (LGMD2A) | up-regulated |
| hsa-miR-221 | miyoshi myopathy (MM) | up-regulated |
| hsa-miR-221 | nemaline myopathy (NM) | up-regulated |
| hsa-miR-221 | neuroblastoma (NB) | up-regulated |
| hsa-miR-221 | ovarian cancer (OC) | up-regulated |
| hsa-miR-221 | pancreatic cancer | up-regulated |
| hsa-miR-221 | pancreatic cancer | up-regulated |
| hsa-miR-221 | pancreatic cancer | up-regulated |
| hsa-miR-221 | pancreatic ductal adenocarcinoma (PDAC) | up-regulated |
| hsa-miR-221 | papillary thyroid carcinoma (PTC) | up-regulated |
| hsa-miR-221 | polymyositis (PM) | up-regulated |
| hsa-miR-221 | prostate cancer | down-regulated |
| hsa-miR-222 | glioblastoma | up-regulated |
| hsa-miR-222 | malignant melanoma | up-regulated |
| hsa-miR-222 | non-small cell lung cancer (NSCLC) | up-regulated |
| hsa-miR-222 | papillary thyroid carcinoma (PTC) | up-regulated |
| hsa-miR-222 | acute lymphoblastic leukemia (ALL) | down-regulated |
| hsa-miR-222 | acute myeloid leukemia (AML) | up-regulated |
| hsa-miR-222 | acute myeloid leukemia (AML) | up-regulated |
| hsa-miR-222 | cardiac hypertrophy | up-regulated |
| hsa-miR-132 | polymyositis (PM) | up-regulated |
| hsa-miR-20a | hepatocellular carcinoma (HCC) | up-regulated |
| hsa-miR-216 | malignant melanoma | down-regulated |
| hsa-miR-518b | ovarian cancer (OC) | up-regulated |
| hsa-miR-99b | nemaline myopathy (NM) | up-regulated |
| hsa-miR-181b-1 | pancreatic cancer | up-regulated |
| hsa-miR-199a-l | Oral Squamous Cell Carcinoma (OSCC) | down-regulated |
| hsa-miR-29c | lung cancer | down-regulated |
| hsa-miR-106a | follicular lymphoma (FL) | up-regulated |
| hsa-miR-106a | malignant melanoma | down-regulated |
| hsa-miR-106a | neuroblastoma (NB) | up-regulated |
| hsa-miR-106a | pancreatic cancer | up-regulated |
| hsa-miR-106a | prostate cancer | up-regulated |
| hsa-miR-106a | psoriasis | up-regulated |
| hsa-miR-106a-1 | hepatocellular carcinoma (HCC) | up-regulated |
| hsa-miR-124a | colorectal cancer | down-regulated |
| hsa-miR-124a | epithelial ovarian cancer (EOC) | down-regulated |
| hsa-miR-124a | malignant melanoma | down-regulated |
| hsa-miR-124a | teratocarcinoma | down-regulated |
| hsa-miR-124a-1 | lung cancer | down-regulated |
| hsa-miR-124a-2 | hepatocellular carcinoma (HCC) | down-regulated |
| hsa-miR-124a-2 | hepatocellular carcinoma (HCC) | down-regulated |
| hsa-miR-124a-3 | breast cancer | down-regulated |
| hsa-miR-124a-3 | breast cancer | down-regulated |
| hsa-miR-21 | colorectal cancer | up-regulated |
| hsa-miR-21 | pancreatic cancer | up-regulated |
| hsa-miR-21 | vascular disease | up-regulated |
| hsa-miR-21 | glioma | up-regulated |
| hsa-miR-21 | hepatocellular carcinoma (HCC) | up-regulated |
| hsa-miR-29c | nasopharyngeal carcinoma (NPC) | down-regulated |
| hsa-miR-29c | type 2 diabetes | up-regulated |
| hsa-miR-29c | cardiac hypertrophy | down-regulated |
| hsa-miR-29c | cardiac hypertrophy | down-regulated |
| hsa-miR-29c | Duchenne muscular dystrophy (DMD) | down-regulated |
| hsa-miR-29c | nemaline myopathy (NM) | down-regulated |
| hsa-miR-29c | ovarian cancer (OC) | up-regulated |
| hsa-miR-29c | pancreatic ductal adenocarcinoma (PDAC) | down-regulated |
| hsa-miR-29c | papillary thyroid carcinoma (PTC) | up-regulated |
| hsa-miR-29c | schizophrenia | down-regulated |
| hsa-miR-29c | uterine leiomyoma (ULM) | down-regulated |
| hsa-miR-301 | follicular lymphoma (FL) | up-regulated |
| hsa-miR-301 | hepatocellular carcinoma (HCC) | up-regulated |
| hsa-miR-301 | pancreatic cancer | up-regulated |
| hsa-miR-302a | Oral Squamous Cell Carcinoma (OSCC) | down-regulated |
| hsa-miR-302b | Oral Squamous Cell Carcinoma (OSCC) | down-regulated |
| hsa-miR-20a | malignant lymphoma | up-regulated |
| hsa-miR-20a | pancreatic cancer | up-regulated |
| hsa-miR-20a | primary biliary cirrhosis (PBC) | down-regulated |
| hsa-miR-20a | prostate cancer | up-regulated |
| hsa-miR-20a | psoriasis | up-regulated |
| hsa-miR-20a | serous ovarian cancer | up-regulated |
| hsa-miR-20b | T-cell leukemia | up-regulated |
| hsa-miR-20b | schizophrenia | down-regulated |
| hsa-miR-21 | colorectal cancer | up-regulated |
| hsa-miR-21 | diffuse large B-cell lymphoma (DLBCL) | up-regulated |
| hsa-miR-21 | diffuse large B-cell lymphoma (DLBCL) | up-regulated |
| hsa-miR-21 | lung cancer | up-regulated |
| hsa-miR-21 | pancreatic cancer | up-regulated |
| hsa-miR-21 | breast cancer | up-regulated |
| hsa-miR-21 | breast cancer | up-regulated |
| hsa-miR-21 | breast cancer | up-regulated |
| hsa-miR-21 | breast cancer | up-regulated |
| hsa-miR-21 | breast cancer | up-regulated |
| hsa-miR-21 | cardiac hypertrophy | up-regulated |
| hsa-miR-21 | cholangiocarcinoma | up-regulated |
| hsa-miR-21 | Cowden Syndrome | up-regulated |
| hsa-miR-21 | glioblastoma | up-regulated |
| hsa-miR-21 | hepatocellular carcinoma (HCC) | up-regulated |
| hsa-miR-21 | acute lymphoblastic leukemia (ALL) | down-regulated |
| hsa-miR-216 | nasopharyngeal carcinoma (NPC) | down-regulated |
| hsa-miR-216 | pancreatic ductal adenocarcinoma (PDAC) | down-regulated |
| hsa-miR-217 | B-cell chronic lymphocytic leukemia | up-regulated |
| hsa-miR-217 | nasopharyngeal carcinoma (NPC) | down-regulated |
| hsa-miR-217 | pancreatic ductal adenocarcinoma (PDAC) | down-regulated |
| hsa-miR-218 | cervical cancer | down-regulated |
| hsa-miR-218 | cardiac hypertrophy | down-regulated |
| hsa-miR-218-2 | lung cancer | down-regulated |
| hsa-miR-219 | malignant melanoma | down-regulated |
| hsa-miR-219-1 | hepatocellular carcinoma (HCC) | up-regulated |
| hsa-miR-219-1 | hepatocellular carcinoma (HCC) | up-regulated |
| hsa-miR-219-1 | lung cancer | down-regulated |
| hsa-miR-219-1 | papillary thyroid carcinoma (PTC) | up-regulated |
| hsa-miR-22 | acute lymphoblastic leukemia (ALL) | down-regulated |
| hsa-miR-22 | acute myeloid leukemia (AML) | up-regulated |
| hsa-miR-22 | Alzheimer's disease | down-regulated |
| hsa-miR-22 | Duchenne muscular dystrophy (DMD) | down-regulated |
| hsa-miR-22 | head and neck squamous cell carcinoma (HNSCC) | down-regulated |
| hsa-miR-124a | Alzheimer's disease | down-regulated |
| hsa-miR-16 | chronic lymphocytic leukemia (CLL) | down-regulated |
| hsa-miR-16 | gastric cancer (stomach cancer) | down-regulated |
| hsa-miR-16 | prostate cancer | down-regulated |
| hsa-miR-16 | serous ovarian cancer | up-regulated |
| hsa-miR-16-1 | chronic lymphocytic leukemia (CLL) | up-regulated |
| hsa-miR-199a-1 | pancreatic cancer | up-regulated |
| hsa-miR-199a-1 | pancreatic cancer | up-regulated |
| hsa-miR-199a-1 | prostate cancer | up-regulated |
| hsa-miR-199a-1-5p | hepatocellular carcinoma (HCC) | down-regulated |
| hsa-miR-199a-2 | chronic pancreatitis | up-regulated |
| hsa-miR-199a-2 | Oral Squamous Cell Carcinoma (OSCC) | down-regulated |
| hsa-miR-199a-2 | pancreatic cancer | up-regulated |
| hsa-miR-199a-2-5p | hepatocellular carcinoma (HCC) | down-regulated |
| hsa-miR-199a-3p | serous ovarian cancer | down-regulated |
| hsa-miR-199b | acute lymphoblastic leukemia (ALL) | down-regulated |
| hsa-miR-199b | acute myeloid leukemia (AML) | up-regulated |
| hsa-miR-199b | cardiac hypertrophy | up-regulated |
| hsa-miR-199b | chronic pancreatitis | up-regulated |
| hsa-miR-199b | dermatomyositis (DM) | up-regulated |
| hsa-miR-199b | Duchenne muscular dystrophy (DMD) | up-regulated |
| hsa-miR-199b | epithelial ovarian cancer (EOC) | down-regulated |
| hsa-miR-199b | facioscapulohumeral muscular dystrophy (FSHD) | up-regulated |
| hsa-miR-199b | hepatocellular carcinoma (HCC) | down-regulated |
| hsa-miR-199b | limb-girdle muscular dystrophies types 2A (LGMD2A) | up-regulated |
| hsa-miR-199b | lung cancer | down-regulated |
| hsa-miR-199b | nemaline myopathy (NM) | up-regulated |
| hsa-miR-199b | Oral Squamous Cell Carcinoma (OSCC) | down-regulated |
| hsa-miR-199b | polymyositis (PM) | up-regulated |
| hsa-miR-519a | ovarian cancer (OC) | down-regulated |
| hsa-miR-519d | primary biliary cirrhosis (PBC) | down-regulated |
| hsa-miR-520c | breast cancer | up-regulated |
| hsa-miR-524* | ovarian cancer (OC) | down-regulated |
| hsa-miR-524* | psoriasis | down-regulated |
| hsa-miR-525* | ovarian cancer (OC) | up-regulated |
| hsa-miR-539 | autism spectrum disorder (ASD) | down-regulated |
| hsa-miR-542-3p | ovarian cancer (OC) | down-regulated |
| hsa-miR-542-3p | uterine leiomyoma (ULM) | up-regulated |
| hsa-miR-542-5p | uterine leiomyoma (ULM) | down-regulated |
| hsa-miR-550 | autism spectrum disorder (ASD) | down-regulated |
| hsa-miR-551a | ovarian cancer (OC) | down-regulated |
| hsa-miR-563 | head and neck squamous cell carcinoma (HNSCC) | up-regulated |
| hsa-miR-565 | head and neck squamous cell carcinoma (HNSCC) | down-regulated |
| hsa-miR-572 | head and neck squamous cell carcinoma (HNSCC) | up-regulated |
| hsa-miR-572 | ovarian cancer (OC) | up-regulated |
| hsa-miR-582 | uterine leiomyoma (ULM) | up-regulated |
| hsa-miR-594 | hepatocellular carcinoma (HCC) | down-regulated |
| hsa-miR-594 | ovarian cancer (OC) | up-regulated |
| hsa-miR-598 | autism spectrum disorder (ASD) | up-regulated |
| hsa-miR-605 | ovarian cancer (OC) | up-regulated |
| hsa-miR-608 | ovarian cancer (OC) | down-regulated |
| hsa-miR-611 | ovarian cancer (OC) | up-regulated |
| hsa-miR-622 | ovarian cancer (OC) | down-regulated |
| hsa-miR-627 | ovarian cancer (OC) | down-regulated |
| hsa-miR-99b | polymyositis (PM) | up-regulated |
| hsa-miR-99b | prostate cancer | up-regulated |
| hsa-miR-99b | psoriasis | down-regulated |
| hsa-miR-106b | gastric cancer (stomach cancer) | up-regulated |
| hsa-miR-106b | Alzheimer's disease | down-regulated |
| hsa-miR-106b | autism spectrum disorder (ASD) | down-regulated |
| hsa-miR-106b | ovarian cancer (OC) | down-regulated |
| hsa-miR-106b | schizophrenia | up-regulated |
| hsa-miR-106b-1 | hepatocellular carcinoma (HCC) | up-regulated |
| hsa-miR-107 | esophageal cancer | up-regulated |
| hsa-miR-107 | Alzheimer's disease | down-regulated |
| hsa-miR-107 | cardiac hypertrophy | up-regulated |
| hsa-miR-107 | colorectal cancer | up-regulated |
| hsa-miR-107 | colorectal cancer | down-regulated |
| hsa-miR-107 | gastric cancer (stomach cancer) | up-regulated |
| hsa-miR-107 | malignant melanoma | down-regulated |
| hsa-miR-107 | Oral Squamous Cell Carcinoma (OSCC) | down-regulated |
| hsa-miR-107 | pancreatic cancer | up-regulated |
| hsa-miR-107 | pancreatic cancer | up-regulated |
| hsa-miR-107 | pancreatic cancer | up-regulated |
| hsa-miR-107 | pancreatic cancer | up-regulated |
| hsa-miR-10a | cardiac hypertrophy | down-regulated |
| hsa-miR-10a | colorectal cancer | up-regulated |
| hsa-miR-181b-1 | prostate cancer | up-regulated |
| hsa-miR-181b-2 | pancreatic cancer | up-regulated |
| hsa-miR-181c | acute myeloid leukemia (AML) | up-regulated |
| hsa-miR-181c | Alzheimer's disease | down-regulated |
| hsa-miR-181c | glioblastoma | down-regulated |
| hsa-miR-181c | hepatocellular carcinoma (HCC) | down-regulated |
| hsa-miR-181c | lung cancer | down-regulated |
| hsa-miR-181c | pancreatic cancer | up-regulated |
| hsa-miR-181c | pancreatic cancer | up-regulated |
| hsa-miR-181c | papillary thyroid carcinoma (PTC) | up-regulated |
| hsa-miR-181d | autism spectrum disorder (ASD) | down-regulated |
| hsa-miR-181d | Duchenne muscular dystrophy (DMD) | up-regulated |
| hsa-miR-181d | nemaline myopathy (NM) | up-regulated |
| hsa-miR-181d | pancreatic cancer | up-regulated |
| hsa-miR-182 | acute myeloid leukemia (AML) | down-regulated |
| hsa-miR-182 | epithelial ovarian cancer (EOC) | up-regulated |
| hsa-miR-182 | hepatocellular carcinoma (HCC) | up-regulated |
| hsa-miR-182 | malignant melanoma | down-regulated |
| hsa-miR-182* | non-small cell lung cancer (NSCLC) | up-regulated |
| hsa-miR-182* | hepatocellular carcinoma (HCC) | up-regulated |
| hsa-miR-183 | B-cell chronic lymphocytic leukemia | up-regulated |
| hsa-miR-183 | ovarian cancer (OC) | down-regulated |
| hsa-miR-183 | retinitis pigmentosa (RP) | up-regulated |
| hsa-miR-23a | Oral Squamous Cell Carcinoma (OSCC) | down-regulated |
| hsa-miR-23a | pancreatic cancer | up-regulated |
| hsa-miR-23a | prostate cancer | down-regulated |
| hsa-miR-23a | serous ovarian cancer | up-regulated |
| hsa-miR-23b | chronic lymphocytic leukemia (CLL) | up-regulated |
| hsa-miR-23b | acute lymphoblastic leukemia (ALL) | down-regulated |
| hsa-miR-23b | acute myeloid leukemia (AML) | up-regulated |
| hsa-miR-23b | bladder cancer | up-regulated |
| hsa-miR-23b | cardiac hypertrophy | up-regulated |
| hsa-miR-23b | cervical cancer | down-regulated |
| hsa-miR-23b | glioblastoma | up-regulated |
| hsa-miR-23b | hepatocellular carcinoma (HCC) | up-regulated |
| hsa-miR-23b | malignant melanoma | down-regulated |
| hsa-miR-23b | Oral Squamous Cell Carcinoma (OSCC) | down-regulated |
| hsa-miR-23b | pancreatic cancer | up-regulated |
| hsa-miR-23b | prostate cancer | down-regulated |
| hsa-miR-23b | serous ovarian cancer | up-regulated |
| hsa-miR-23b | uterine leiomyoma (ULM) | up-regulated |
| hsa-miR-24 | vesicular stomatitis | down-regulated |
| hsa-miR-24 | acute lymphoblastic leukemia (ALL) | down-regulated |
| hsa-miR-24 | acute myeloid leukemia (AML) | up-regulated |
| hsa-miR-24 | cardiac hypertrophy | up-regulated |
| hsa-miR-24 | heart failure | up-regulated |
| hsa-miR-24 | hepatocellular carcinoma (HCC) | up-regulated |
| hsa-miR-25 | hepatocellular carcinoma (HCC) | up-regulated |
| hsa-miR-34c | ovarian cancer (OC) | down-regulated |
| hsa-miR-148b | asthma | normal |
| hsa-miR-372 | colorectal cancer | up-regulated |
| hsa-miR-34c | acute myeloid leukemia (AML) | down-regulated |
| hsa-miR-34c | malignant melanoma | down-regulated |
| hsa-miR-34c | nasopharyngeal carcinoma (NPC) | down-regulated |
| hsa-miR-34c | Oral Squamous Cell Carcinoma (OSCC) | down-regulated |
| hsa-miR-34c | ovarian cancer (OC) | down-regulated |
| hsa-miR-351 | cardiac hypertrophy | up-regulated |
| hsa-miR-352 | vascular disease | up-regulated |
| hsa-miR-361 | Duchenne muscular dystrophy (DMD) | down-regulated |
| hsa-miR-361 | nemaline myopathy (NM) | up-regulated |
| hsa-miR-361 | ovarian cancer (OC) | down-regulated |
| hsa-miR-361 | primary biliary cirrhosis (PBC) | down-regulated |
| hsa-miR-362 | dermatomyositis (DM) | up-regulated |
| hsa-miR-362 | limb-girdle muscular dystrophies types 2A (LGMD2A) | up-regulated |
| hsa-miR-362 | miyoshi myopathy (MM) | up-regulated |
| hsa-miR-362 | nemaline myopathy (NM) | up-regulated |
| hsa-miR-362 | polymyositis (PM) | up-regulated |
| hsa-miR-363 | Alzheimer's disease | down-regulated |
| hsa-miR-365 | psoriasis | down-regulated |
| hsa-miR-365 | vascular disease | down-regulated |
| hsa-miR-367 | acute myeloid leukemia (AML) | down-regulated |
| hsa-miR-367 | Oral Squamous Cell Carcinoma (OSCC) | down-regulated |
| hsa-miR-367 | testicular germ cell tumor | up-regulated |
| hsa-miR-368 | epithelial ovarian cancer (EOC) | down-regulated |
| hsa-miR-368 | dermatomyositis (DM) | up-regulated |
| hsa-miR-148b | autism spectrum disorder (ASD) | up-regulated |
| hsa-miR-148b | dermatomyositis (DM) | up-regulated |
| hsa-miR-148b | head and neck squamous cell carcinoma (HNSCC) | down-regulated |
| hsa-miR-148b | limb-girdle muscular dystrophies types 2A (LGMD2A) | up-regulated |
| hsa-miR-148b | nemaline myopathy (NM) | up-regulated |
| hsa-miR-148b | pancreatic cancer | down-regulated |
| hsa-miR-148b | pancreatic ductal adenocarcinoma (PDAC) | down-regulated |
| hsa-miR-149 | breast cancer | up-regulated |
| hsa-miR-149 | cardiac hypertrophy | down-regulated |
| hsa-miR-149 | cardiac hypertrophy | down-regulated |
| hsa-miR-149 | diffuse large B-cell lymphoma (DLBCL) | down-regulated |
| hsa-miR-149 | follicular lymphoma (FL) | down-regulated |
| hsa-miR-149 | glioblastoma multiforme (GBM) | down-regulated |
| hsa-miR-149 | malignant melanoma | down-regulated |
| hsa-miR-149 | pituitary adenoma | up-regulated |
| hsa-miR-15 | chronic lymphocytic leukemia (CLL) | down-regulated |
| hsa-miR-150 | cardiac hypertrophy | down-regulated |
| hsa-miR-150 | cardiac hypertrophy | down-regulated |
| hsa-miR-150 | chronic lymphocytic leukemia (CLL) | down-regulated |
| hsa-miR-150 | diffuse large B-cell lymphoma (DLBCL) | down-regulated |
| hsa-miR-150 | epithelial ovarian cancer (EOC) | down-regulated |
| hsa-miR-150 | hepatocellular carcinoma (HCC) | down-regulated |
| hsa-miR-153 | glioblastoma multiforme (GBM) | down-regulated |
| hsa-miR-15a | malignant melanoma | down-regulated |
| hsa-miR-15a-2 | prostate cancer | up-regulated |
| hsa-miR-15b | gastric cancer (stomach cancer) | down-regulated |
| hsa-miR-15b | acute myeloid leukemia (AML) | down-regulated |
| hsa-miR-15b | acute promyelocytic leukemia (APL) | up-regulated |
| hsa-miR-15b | autism spectrum disorder (ASD) | up-regulated |
| hsa-miR-15b | B-cell chronic lymphocytic leukemia | up-regulated |
| hsa-miR-15b | cardiac hypertrophy | up-regulated |
| hsa-miR-15b | colorectal cancer | down-regulated |
| hsa-miR-15b | colorectal cancer | up-regulated |
| hsa-miR-15b | non-small cell lung cancer (NSCLC) | up-regulated |
| hsa-let-7c | acute myeloid leukemia (AML) | up-regulated |
| hsa-let-7c | cardiac hypertrophy | up-regulated |
| hsa-let-7c | cervical cancer | down-regulated |
| hsa-let-7c | Down syndrome (DS) | up-regulated |
| hsa-let-7c | epithelial ovarian cancer (EOC) | down-regulated |
| hsa-let-7c | hepatocellular carcinoma (HCC) | down-regulated |
| hsa-let-7c | lung cancer | up-regulated |
| hsa-let-7c | lung cancer | down-regulated |
| hsa-let-7c | prostate cancer | up-regulated |
| hsa-let-7c | prostate cancer | down-regulated |
| hsa-let-7c | uterine leiomyoma (ULM) | up-regulated |
| hsa-miR-133a | cardiac hypertrophy | down-regulated |
| hsa-miR-133a | cardiomyopathy | up-regulated |
| hsa-miR-186 | head and neck squamous cell carcinoma (HNSCC) | down-regulated |
| hsa-miR-222 | dermatomyositis (DM) | up-regulated |
| hsa-miR-106a | colorectal cancer | up-regulated |
| hsa-miR-125b-2 | hepatocellular carcinoma (HCC) | down-regulated |
| hsa-miR-147 | acute promyelocytic leukemia (APL) | up-regulated |
| hsa-miR-195 | prostate cancer | down-regulated |
| hsa-miR-433 | Parkinson's disease | normal |
| hsa-miR-518b | psoriasis | down-regulated |
| hsa-miR-9* | follicular lymphoma (FL) | up-regulated |
| hsa-miR-99a | pancreatic cancer | up-regulated |
| hsa-miR-122a | Oral Squamous Cell Carcinoma (OSCC) | down-regulated |
| hsa-miR-130a | hepatocellular carcinoma (HCC) | down-regulated |
| hsa-miR-143 | lung cancer | down-regulated |
| hsa-miR-199a* | uveal melanoma | up-regulated |
| hsa-miR-200c | serous ovarian cancer | up-regulated |
| hsa-miR-155 | papillary thyroid carcinoma (PTC) | up-regulated |
| hsa-let-7f | ovarian cancer (OC) | down-regulated |
| hsa-let-7f | prostate cancer | down-regulated |
| hsa-let-7f-1 | breast cancer | up-regulated |
| hsa-let-7f-1 | colorectal cancer | up-regulated |
| hsa-let-7f-1 | lung cancer | up-regulated |
| hsa-let-7f-1 | pancreatic cancer | up-regulated |
| hsa-let-7f-1 | pituitary adenoma | down-regulated |
| hsa-let-7f-2 | breast cancer | down-regulated |
| hsa-let-7f-2 | hepatocellular carcinoma (HCC) | down-regulated |
| hsa-let-7b | colorectal cancer | up-regulated |
| hsa-let-7b | facioscapulohumeral muscular dystrophy (FSHD) | up-regulated |
| hsa-let-7b | hepatocellular carcinoma (HCC) | down-regulated |
| hsa-let-7b | limb-girdle muscular dystrophies types 2A (LGMD2A) | up-regulated |
| hsa-let-7b | miyoshi myopathy (MM) | up-regulated |
| hsa-let-7b | nemaline myopathy (NM) | up-regulated |
| hsa-let-7b | neuroblastoma (NB) | up-regulated |
| hsa-let-7b | primary biliary cirrhosis (PBC) | down-regulated |
| hsa-let-7b | prostate cancer | down-regulated |
| hsa-let-7b | uveal melanoma | up-regulated |
| hsa-let-7c | acute promyelocytic leukemia (APL) | up-regulated |
| hsa-let-7c | lung cancer | down-regulated |
| hsa-let-7c | ovarian cancer (OC) | down-regulated |
| hsa-let-7c | prostate cancer | down-regulated |
| hsa-let-7c | uterine leiomyoma (ULM) | down-regulated |
| hsa-miR-132 | dermatomyositis (DM) | up-regulated |
| hsa-miR-132 | facioscapulohumeral muscular dystrophy (FSHD) | up-regulated |
| hsa-miR-132 | glioblastoma multiforme (GBM) | down-regulated |
| hsa-miR-132 | hepatocellular carcinoma (HCC) | down-regulated |
| hsa-miR-132 | limb-girdle muscular dystrophies types 2A (LGMD2A) | up-regulated |
| hsa-miR-132 | miyoshi myopathy (MM) | up-regulated |
| hsa-miR-132 | nemaline myopathy (NM) | up-regulated |
| hsa-miR-132 | Oral Squamous Cell Carcinoma (OSCC) | down-regulated |
| hsa-miR-132 | pituitary adenoma | down-regulated |
| hsa-miR-21 | acute myeloid leukemia (AML) | up-regulated |
| hsa-miR-21 | autism spectrum disorder (ASD) | up-regulated |
| hsa-miR-21 | breast cancer | up-regulated |
| hsa-miR-21 | cardiac hypertrophy | up-regulated |
| hsa-miR-21 | cardiac hypertrophy | up-regulated |
| hsa-miR-21 | cervical cancer | up-regulated |
| hsa-miR-21 | chronic lymphocytic leukemia (CLL) | up-regulated |
| hsa-miR-21 | colorectal cancer | up-regulated |
| hsa-miR-21 | colorectal cancer | up-regulated |
| hsa-miR-21 | colorectal cancer | up-regulated |
| hsa-miR-21 | colorectal cancer | up-regulated |
| hsa-miR-21 | dermatomyositis (DM) | up-regulated |
| hsa-miR-21 | Duchenne muscular dystrophy (DMD) | up-regulated |
| hsa-miR-21 | epithelial ovarian cancer (EOC) | down-regulated |
| hsa-miR-21 | esophageal cancer | up-regulated |
| hsa-miR-21 | facioscapulohumeral muscular dystrophy (FSHD) | up-regulated |
| hsa-miR-21 | gastric cancer (stomach cancer) | up-regulated |
| hsa-miR-21 | gastric cancer (stomach cancer) | up-regulated |
| hsa-miR-21 | glioblastoma | up-regulated |
| hsa-miR-21 | glioblastoma multiforme (GBM) | up-regulated |
| hsa-miR-21 | glioma | up-regulated |
| hsa-miR-21 | hepatocellular carcinoma (HCC) | up-regulated |
| hsa-miR-21 | Inclusion body myositis (IBM) | up-regulated |

1. A single-walled carbon nanotube (SWCNT) sensor, comprising:
a SWCNT;
a polymer associated with the SWCNT, wherein the polymer comprises DNA, RNA, a locked nucleic acid (LNA), glycol nucleic acid (GNA), or threose nucleic acid (TNA), and wherein the polymer comprises two or more domains.

2. The sensor of claim 1, wherein the two or more domains comprise:
a first domain comprising a stabilizing domain; and
a second domain comprising a sequence complementary to a target nucleotide sequence.

3. The sensor of claim 2, wherein the two or more domains further comprise:
a third domain that has a sequence complementary to a target sequence (e.g., wherein the first domain and the third domain are positioned on each end of the stability domain).

4. (canceled)

5. The sensor of claim 1, wherein the polymer is single-stranded DNA.

6. The sensor of claim 1, wherein the polymer comprises a single-stranded DNA binding component containing a sequence complementary to a target nucleotide sequence.

7. The sensor of claim 6, wherein the target nucleotide sequence has fewer than 30 nucleotides.

8. The sensor of claim 6, wherein the target nucleotide sequence has 30 or more nucleotides.

9. The sensor of claim 6, wherein the target nucleotide sequence has from about 5 nucleotides to about 30 nucleotides.

10. The sensor of claim 6, wherein the target nucleotide sequence has from about 10 nucleotides to about 25 nucleotides.

11. The sensor of claim 10, wherein a first domain has a sequence complementary to the target nucleotide sequence.

12. The sensor of claim 11, wherein the first domain has a sequence complementary to a target miRNA sequence.

13. (canceled)

14. The sensor of claim 11, wherein the first domain has a sequence complementary to a target DNA sequence.

15. The sensor of claim 11, wherein the second domain is a stabilizing domain.

16. (canceled)

17. The sensor of claim 15, wherein the second domain is an oligonucleotide sequence.

18. The sensor of claim 17, wherein the oligonucleotide sequence comprises a member selected from the group consisting of (GT)6 (SEQ ID NO: 2), (GT)15 (SEQ ID NO: 1), (AT)15 (SEQ ID NO: 3), (TAT)6 (SEQ ID NO: 4), (TCC)10 (SEQ ID NO: 5), (TGA)10 (SEQ ID NO: 6), (CCA)10 (SEQ ID NO: 7), (TTA)4TT (SEQ ID NO: 8), (TTA)3TTGTT (SEQ ID NO: 9), (TTA)5TT (SEQ ID NO: 10), (TAT)4 (SEQ ID NO: 11), (CGT)3C (SEQ ID NO: 12), (ATT)4 (SEQ ID NO: 13), (ATT)4AT (SEQ ID NO: 14), (TATT)2TAT (SEQ ID NO: 15), (ATTT)3 (SEQ ID NO: 16), (GTC)2GT (SEQ ID NO: 17), (CCG)4 (SEQ ID NO: 18), (GTT)3G (SEQ ID NO: 19), (TGT)4T (SEQ ID NO: 20), (TATT)3T (SEQ ID NO: 22), (TCG)10 (SEQ ID NO: 23), (GTC)3 (SEQ ID NO: 24), (TCG)2TC (SEQ ID NO: 25), (TCG)4TC (SEQ ID NO: 26), (GTC)2 (SEQ ID NO: 27), (TGTT)2TGT (SEQ ID NO: 28), (TTTA)3T (SEQ ID NO: 29), (CCG)2CC (SEQ ID NO: 30), (TCG)4TC (SEQ ID NO: 31), T3C6T3 (SEQ ID NO: 32), (GTC)2GT (SEQ ID NO: 33), CTTC2TTC (SEQ ID NO: 34), TTA(TAT)2ATT (SEQ ID NO: 35), TCT(CTC)2TCT (SEQ ID NO: 36), (ATT)4 (SEQ ID NO: 37), GC11 (SEQ ID NO: 38), (TC)3CTCCCT (SEQ ID NO: 39), CTTC3TTC (SEQ ID NO: 40), (GT)20 (SEQ ID NO: 41), CTC3TC (SEQ ID NO: 42), (TCT)2 (SEQ ID NO: 43), C5TC6 (SEQ ID NO: 44), T4C4T4 (SEQ ID NO: 45), and C5TTC5 (SEQ ID NO: 46).

19-22. (canceled)

23. The sensor of claim 1, further comprising a surfactant, wherein the surfactant is selected from a group consisting of SDS, SDBS, SDC, SPAN-80, Brij 52, BSA, Triton X-100, Pluronic, Pyrene-PEG, TPGS, IGEPAL, and Phospholipid-PEG-NH₂.

24. (canceled)

25. A method for detecting a target using a single-walled carbon nanotube (SWCNT) sensor of claim 1, the method comprising:
contacting a sample comprising a species having a target nucleotide sequence with the SWCNT sensor;
exposing the sample to excitation electromagnetic radiation (excitation EMR) to produce an emission of electromagnetic radiation (emission EMR) by the SWCNT sensor;
detecting the emission EMR by the SWCNT sensor.

26-34. (canceled)

35. The method of claim 25, wherein the species having the target nucleotide sequence is microRNA.

36-37. (canceled)

38. The method of claim 25, wherein the sample is a biological sample.

39. The method of claim 25, wherein the sample is a member selected from the group consisting of a cell culture sample, a laboratory sample, a tissue sample, and a bodily fluid sample.

40-48. (canceled)


SEQUENCE LISTING (113 sequences)

SEQ ID NO: 1 - DNA, 30 bp, Artificial Sequence
  source: /note=“Description of Artificial Sequence      Synthetic oligonucleotide”
gtgtgtgtgtgtgtgtgtgtgtgtgtgtgt

SEQ ID NO: 2 - DNA, 12 bp, Artificial Sequence
  source: /note=“Description of Artificial Sequence      Synthetic oligonucleotide”
gtgtgtgtgtgt

SEQ ID NO: 3 - DNA, 30 bp, Artificial Sequence
  source: /note=“Description of Artificial Sequence      Synthetic oligonucleotide”
atatatatatatatatatatatatatatat

SEQ ID NO: 4 - DNA, 18 bp, Artificial Sequence
  source: /note=“Description of Artificial Sequence      Synthetic oligonucleotide”
tattattattattattat

SEQ ID NO: 5 - DNA, 30 bp, Artificial Sequence
  source: /note=“Description of Artificial Sequence      Synthetic oligonucleotide”
tcctcctcctcctcctcctcctcctcctcc

SEQ ID NO: 6 - DNA, 30 bp, Artificial Sequence
  source: /note=“Description of Artificial Sequence      Synthetic oligonucleotide”
tgatgatgatgatgatgatgatgatgatga

SEQ ID NO: 7 - DNA, 30 bp, Artificial Sequence
  source: /note=“Description of Artificial Sequence      Synthetic oligonucleotide”
ccaccaccaccaccaccaccaccaccacca

SEQ ID NO: 8 - DNA, 14 bp, Artificial Sequence
  source: /note=“Description of Artificial Sequence      Synthetic oligonucleotide”
ttattattattatt

SEQ ID NO: 9 - DNA, 14 bp, Artificial Sequence
  source: /note=“Description of Artificial Sequence      Synthetic oligonucleotide”
ttattattattgtt

SEQ ID NO: 10 - DNA, 17 bp, Artificial Sequence
  source: /note=“Description of Artificial Sequence      Synthetic oligonucleotide”
ttattattattattatt

SEQ ID NO: 11 - DNA, 12 bp, Artificial Sequence
  source: /note=“Description of Artificial Sequence      Synthetic oligonucleotide”
tattattattat

SEQ ID NO: 12 - DNA, 10 bp, Artificial Sequence
  source: /note=“Description of Artificial Sequence      Synthetic oligonucleotide”
cgtcgtcgtc

SEQ ID NO: 13 - DNA, 12 bp, Artificial Sequence
  source: /note=“Description of Artificial Sequence      Synthetic oligonucleotide”
attattattatt

SEQ ID NO: 14 - DNA, 14 bp, Artificial Sequence
  source: /note=“Description of Artificial Sequence      Synthetic oligonucleotide”
attattattattat

SEQ ID NO: 15 - DNA, 11 bp, Artificial Sequence
  source: /note=“Description of Artificial Sequence      Synthetic oligonucleotide”
tatttatttat

SEQ ID NO: 16 - DNA, 12 bp, Artificial Sequence
  source: /note=“Description of Artificial Sequence      Synthetic oligonucleotide”
atttatttattt

SEQ ID NO: 17 - DNA, 8 bp, Artificial Sequence
  source: /note=“Description of Artificial Sequence      Synthetic oligonucleotide”
gtcgtcgt

SEQ ID NO: 18 - DNA, 12 bp, Artificial Sequence
  source: /note=“Description of Artificial Sequence      Synthetic oligonucleotide”
ccgccgccgccg

SEQ ID NO: 19 - DNA, 10 bp, Artificial Sequence
  source: /note=“Description of Artificial Sequence      Synthetic oligonucleotide”
gttgttgttg

SEQ ID NO: 20 - DNA, 13 bp, Artificial Sequence
  source: /note=“Description of Artificial Sequence      Synthetic oligonucleotide”
tgttgttgttgtt

SEQ ID NO: 21 - DNA, 79 bp, Artificial Sequence
  source: /note=“Description of Artificial Sequence      Synthetic oligonucleotide”
tcggtcagtgggtcattgctagtttttagtcatcaagcatcaacaattattattattatattgttgatgcttgatgact

SEQ ID NO: 22 - DNA, 13 bp, Artificial Sequence
  source: /note=“Description of Artificial Sequence      Synthetic oligonucleotide”
tatttatttattt

SEQ ID NO: 23 - DNA, 30 bp, Artificial Sequence
  source: /note=“Description of Artificial Sequence      Synthetic oligonucleotide”
tcgtcgtcgtcgtcgtcgtcgtcgtcgtcg

SEQ ID NO: 24 - DNA, 9 bp, Artificial Sequence
  source: /note=“Description of Artificial Sequence      Synthetic oligonucleotide”
gtcgtcgtc

SEQ ID NO: 25 - DNA, 8 bp, Artificial Sequence
  source: /note=“Description of Artificial Sequence      Synthetic oligonucleotide”
tcgtcgtc

SEQ ID NO: 26 - DNA, 14 bp, Artificial Sequence
  source: /note=“Description of Artificial Sequence      Synthetic oligonucleotide”
tcgtcgtcgtcgtc

SEQ ID NO: 27 - DNA, 6 bp, Artificial Sequence
  source: /note=“Description of Artificial Sequence      Synthetic oligonucleotide”
gtcgtc

SEQ ID NO: 28 - DNA, 11 bp, Artificial Sequence
  source: /note=“Description of Artificial Sequence      Synthetic oligonucleotide”
tgtttgtttgt

SEQ ID NO: 29 - DNA, 13 bp, Artificial Sequence
  source: /note=“Description of Artificial Sequence      Synthetic oligonucleotide”
tttatttatttat

SEQ ID NO: 30 - DNA, 8 bp, Artificial Sequence
  source: /note=“Description of Artificial Sequence      Synthetic oligonucleotide”
ccgccgcc

SEQ ID NO: 31 - DNA, 14 bp, Artificial Sequence
  source: /note=“Description of Artificial Sequence      Synthetic oligonucleotide”
tcgtcgtcgtcgtc

SEQ ID NO: 32 - DNA, 12 bp, Artificial Sequence
  source: /note=“Description of Artificial Sequence      Synthetic oligonucleotide”
tttccccccttt

SEQ ID NO: 33 - DNA, 8 bp, Artificial Sequence
  source: /note=“Description of Artificial Sequence      Synthetic oligonucleotide”
gtcgtcgt

SEQ ID NO: 34 - DNA, 8 bp, Artificial Sequence
  source: /note=“Description of Artificial Sequence      Synthetic oligonucleotide”
cttccttc

SEQ ID NO: 35 - DNA, 12 bp, Artificial Sequence
  source: /note=“Description of Artificial Sequence      Synthetic oligonucleotide”
ttatattatatt

SEQ ID NO: 36 - DNA, 12 bp, Artificial Sequence
  source: /note=“Description of Artificial Sequence      Synthetic oligonucleotide”
tctctcctctct

SEQ ID NO: 37 - DNA, 12 bp, Artificial Sequence
  source: /note=“Description of Artificial Sequence      Synthetic oligonucleotide”
attattattatt

SEQ ID NO: 38 - DNA, 12 bp, Artificial Sequence
  source: /note=“Description of Artificial Sequence      Synthetic oligonucleotide”
gccccccccccc

SEQ ID NO: 39 - DNA, 12 bp, Artificial Sequence
  source: /note=“Description of Artificial Sequence      Synthetic oligonucleotide”
tctctcctccct

SEQ ID NO: 40 - DNA, 9 bp, Artificial Sequence
  source: /note=“Description of Artificial Sequence      Synthetic oligonucleotide”
cttcccttc

SEQ ID NO: 41 - DNA, 40 bp, Artificial Sequence
  source: /note=“Description of Artificial Sequence      Synthetic oligonucleotide”
gtgtgtgtgtgtgtgtgtgtgtgtgtgtgtgtgtgtgtgt

SEQ ID NO: 42 - DNA, 7 bp, Artificial Sequence
  source: /note=“Description of Artificial Sequence      Synthetic oligonucleotide”
ctccctc

SEQ ID NO: 43 - DNA, 6 bp, Artificial Sequence
  source: /note=“Description of Artificial Sequence      Synthetic oligonucleotide”
tcttct

SEQ ID NO: 44 - DNA, 12 bp, Artificial Sequence
  source: /note=“Description of Artificial Sequence      Synthetic oligonucleotide”
ccccctcccccc

SEQ ID NO: 45 - DNA, 12 bp, Artificial Sequence
  source: /note=“Description of Artificial Sequence      Synthetic oligonucleotide”
ttttcccctttt

SEQ ID NO: 46 - DNA, 12 bp, Artificial Sequence
  source: /note=“Description of Artificial Sequence      Synthetic oligonucleotide”
cccccttccccc

SEQ ID NO: 47 - DNA, 53 bp, Artificial Sequence
  source: /note=“Description of Artificial Sequence      Synthetic oligonucleotide”
gtgtgtgtgtgtgtgtgtgtgtgtgtgtgttcagttttgcatagatttgcaca

SEQ ID NO: 48 - DNA, 23 bp, Artificial Sequence
  source: /note=“Description of Artificial Sequence      Synthetic oligonucleotide”
tgtgcaaatctatgcaaaactga

SEQ ID NO: 49 - RNA, 23 nt, Artificial Sequence
  source: /note=“Description of Artificial Sequence      Synthetic oligonucleotide”
ugugcaaaucuaugcaaaacuga

SEQ ID NO: 50 - DNA, 23 bp, Artificial Sequence
  source: /note=“Description of Artificial Sequence      Synthetic oligonucleotide”
tgatcgttactgggtgactggct

SEQ ID NO: 51 - RNA, 23 nt, Artificial Sequence
  source: /note=“Description of Artificial Sequence      Synthetic oligonucleotide”
ugaucguuacugggugacuggcu

SEQ ID NO: 52 - DNA, 68 bp, Artificial Sequence
  source: /note=“Description of Artificial Sequence      Synthetic oligonucleotide”
tgatcgttactgggtgactggctagtcaaaacgtatctaaacgtgtgatcgttactgggtgactggct

SEQ ID NO: 53 - DNA, 45 bp, Artificial Sequence
  source: /note=“Description of Artificial Sequence      Synthetic oligonucleotide”
agtcaaaacgtatctaaacgtgtgatcgttactgggtgactggct

SEQ ID NO: 54 - DNA, 46 bp, Artificial Sequence
  source: /note=“Description of Artificial Sequence      Synthetic oligonucleotide”
tgatcgttactgggtgactggctagtcaaaacgtatctaaacgtgt

SEQ ID NO: 55 - DNA, 10 bp, Artificial Sequence
  source: /note=“Description of Artificial Sequence      Synthetic oligonucleotide”
ctaaacgtgt

SEQ ID NO: 56 - DNA, 11 bp, Artificial Sequence
  source: /note=“Description of Artificial Sequence      Synthetic oligonucleotide”
tctaaacgtgt

SEQ ID NO: 57 - DNA, 12 bp, Artificial Sequence
  source: /note=“Description of Artificial Sequence      Synthetic oligonucleotide”
atctaaacgtgt

SEQ ID NO: 58 - DNA, 13 bp, Artificial Sequence
  source: /note=“Description of Artificial Sequence      Synthetic oligonucleotide”
tatctaaacgtgt

SEQ ID NO: 59 - DNA, 14 bp, Artificial Sequence
  source: /note=“Description of Artificial Sequence      Synthetic oligonucleotide”
gtatctaaacgtgt

SEQ ID NO: 60 - DNA, 15 bp, Artificial Sequence
  source: /note=“Description of Artificial Sequence      Synthetic oligonucleotide”
cgtatctaaacgtgt

SEQ ID NO: 61 - DNA, 17 bp, Artificial Sequence
  source: /note=“Description of Artificial Sequence      Synthetic oligonucleotide”
aacgtatctaaacgtgt

SEQ ID NO: 62 - DNA, 23 bp, Artificial Sequence
  source: /note=“Description of Artificial Sequence      Synthetic oligonucleotide”
agtcaaaacgtatctaaacgtgt

SEQ ID NO: 63 - DNA, 51 bp, Artificial Sequence
  source: /note=“Description of Artificial Sequence      Synthetic oligonucleotide”
gtgtgtgtgtgtgtgtgtgtgtgtgtgtgtgcattattactcacggtacga

SEQ ID NO: 64 - DNA, 52 bp, Artificial Sequence
  source: /note=“Description of Artificial Sequence      Synthetic oligonucleotide”
gtgtgtgtgtgtgtgtgtgtgtgtgtgtgttgtgagttctaccattgccaaa

SEQ ID NO: 65 - DNA, 51 bp, Artificial Sequence
  source: /note=“Description of Artificial Sequence      Synthetic oligonucleotide”
gtgtgtgtgtgtgtgtgtgtgtgtgtgtgtccaagttctgtcatgcactga

SEQ ID NO: 66 - DNA, 51 bp, Artificial Sequence
  source: /note=“Description of Artificial Sequence      Synthetic oligonucleotide”
gtgtgtgtgtgtgtgtgtgtgtgtgtgtgttgattgccactgtctgcagta

SEQ ID NO: 67 - DNA, 53 bp, Artificial Sequence
  source: /note=“Description of Artificial Sequence      Synthetic oligonucleotide”
gtgtgtgtgtgtgtgtgtgtgtgtgtgtgtagcaaaaatgtgctagtgccaaa

SEQ ID NO: 68 - DNA, 52 bp, Artificial Sequence
  source: /note=“Description of Artificial Sequence      Synthetic oligonucleotide”
gtgtgtgtgtgtgtgtgtgtgtgtgtgtgtagtgaattctaccagtgccata

SEQ ID NO: 69 - DNA, 52 bp, Artificial Sequence
  source: /note=“Description of Artificial Sequence      Synthetic oligonucleotide”
gtgtgtgtgtgtgtgtgtgtgtgtgtgtgtgaggtttcccgtgtatgtttca

SEQ ID NO: 70 - DNA, 52 bp, Artificial Sequence
  source: /note=“Description of Artificial Sequence      Synthetic oligonucleotide”
gtgtgtgtgtgtgtgtgtgtgtgtgtgtgtcaagctgatttacacccggtga

SEQ ID NO: 71 - DNA, 52 bp, Artificial Sequence
  source: /note=“Description of Artificial Sequence      Synthetic oligonucleotide”
gtgtgtgtgtgtgtgtgtgtgtgtgtgtgttcaacatcagtctgataagcta

SEQ ID NO: 72 - DNA, 52 bp, Artificial Sequence
  source: /note=“Description of Artificial Sequence      Synthetic oligonucleotide”
gtgtgtgtgtgtgtgtgtgtgtgtgtgtgtccatctttaccagacagtgtta

SEQ ID NO: 73 - DNA, 52 bp, Artificial Sequence
  source: /note=“Description of Artificial Sequence      Synthetic oligonucleotide”
gtgtgtgtgtgtgtgtgtgtgtgtgtgtgtacggttttaccagacagtatta

SEQ ID NO: 74 - DNA, 52 bp, Artificial Sequence
  source: /note=“Description of Artificial Sequence      Synthetic oligonucleotide”
gtgtgtgtgtgtgtgtgtgtgtgtgtgtgttcatcattaccaggcagtatta

SEQ ID NO: 75 - DNA, 47 bp, Artificial Sequence
  source: /note=“Description of Artificial Sequence      Synthetic oligonucleotide”
gtgtgtgtgtgtgtgtgtgtgtgtgtgtgttcagttttgcatagatt

SEQ ID NO: 76 - DNA, 35 bp, Artificial Sequence
  source: /note=“Description of Artificial Sequence      Synthetic oligonucleotide”
gtgtgtgtgtgttcagttttgcatagatttgcaca

SEQ ID NO: 77 - DNA, 21 bp, Artificial Sequence
  source: /note=“Description of Artificial Sequence      Synthetic oligonucleotide”
tagcttatcagactgatgttg

SEQ ID NO: 78 - RNA, 21 nt, Artificial Sequence
  source: /note=“Description of Artificial Sequence      Synthetic oligonucleotide”
uagcuuaucagacugauguug

SEQ ID NO: 79 - DNA, 23 bp, Artificial Sequence
  source: /note=“Description of Artificial Sequence      Synthetic oligonucleotide”
tcggtcagtgggtcattgctagt

SEQ ID NO: 80 - RNA, 23 nt, Artificial Sequence
  source: /note=“Description of Artificial Sequence      Synthetic oligonucleotide”
ucggucagugggucauugcuagu

SEQ ID NO: 81 - DNA, 21 bp, Artificial Sequence
  source: /note=“Description of Artificial Sequence      Synthetic oligonucleotide”
tcgtaccgtgagtaataatgc

SEQ ID NO: 82 - DNA, 22 bp, Artificial Sequence
  source: /note=“Description of Artificial Sequence      Synthetic oligonucleotide”
tttggcaatggtagaactcaca

SEQ ID NO: 83 - DNA, 21 bp, Artificial Sequence
  source: /note=“Description of Artificial Sequence      Synthetic oligonucleotide”
tcagtgcatgacagaacttgg

SEQ ID NO: 84 - DNA, 21 bp, Artificial Sequence
  source: /note=“Description of Artificial Sequence      Synthetic oligonucleotide”
tactgcagacagtggcaatca

SEQ ID NO: 85 - DNA, 23 bp, Artificial Sequence
  source: /note=“Description of Artificial Sequence      Synthetic oligonucleotide”
tttggcactagcacatttttgct

SEQ ID NO: 86 - DNA, 22 bp, Artificial Sequence
  source: /note=“Description of Artificial Sequence      Synthetic oligonucleotide”
tatggcactggtagaattcact

SEQ ID NO: 87 - DNA, 22 bp, Artificial Sequence
  source: /note=“Description of Artificial Sequence      Synthetic oligonucleotide”
tgaaacatacacgggaaacctc

SEQ ID NO: 88 - DNA, 22 bp, Artificial Sequence
  source: /note=“Description of Artificial Sequence      Synthetic oligonucleotide”
tcaccgggtgtaaatcagcttg

SEQ ID NO: 89 - DNA, 22 bp, Artificial Sequence
  source: /note=“Description of Artificial Sequence      Synthetic oligonucleotide”
taacactgtctggtaaagatgg

SEQ ID NO: 90 - DNA, 22 bp, Artificial Sequence
  source: /note=“Description of Artificial Sequence      Synthetic oligonucleotide”
taatactgcctggtaatgatga

SEQ ID NO: 91 - DNA, 22 bp, Artificial Sequence
  source: /note=“Description of Artificial Sequence      Synthetic oligonucleotide”
taatactgtctggtaaaaccgt

SEQ ID NO: 92 - DNA, 23 bp, Artificial Sequence
  source: /note=“Description of Artificial Sequence      Synthetic oligonucleotide”
tcagttttgcatagatttgcaca

SEQ ID NO: 93 - DNA, 10 bp, Artificial Sequence
  source: /note=“Description of Artificial Sequence      Synthetic oligonucleotide”
gcaaaactga

SEQ ID NO: 94 - DNA, 11 bp, Artificial Sequence
  source: /note=“Description of Artificial Sequence      Synthetic oligonucleotide”
tgcaaaactga

SEQ ID NO: 95 - DNA, 12 bp, Artificial Sequence
  source: /note=“Description of Artificial Sequence      Synthetic oligonucleotide”
atgcaaaactga

SEQ ID NO: 96 - DNA, 13 bp, Artificial Sequence
  source: /note=“Description of Artificial Sequence      Synthetic oligonucleotide”
tatgcaaaactga

SEQ ID NO: 97 - DNA, 14 bp, Artificial Sequence
  source: /note=“Description of Artificial Sequence      Synthetic oligonucleotide”
ctatgcaaaactga

SEQ ID NO: 98 - DNA, 15 bp, Artificial Sequence
  source: /note=“Description of Artificial Sequence      Synthetic oligonucleotide”
tctatgcaaaactga

SEQ ID NO: 99 - DNA, 10 bp, Artificial Sequence
  source: /note=“Description of Artificial Sequence      Synthetic oligonucleotide”
tgtgcaaatc

SEQ ID NO: 100 - DNA, 11 bp, Artificial Sequence
  source: /note=“Description of Artificial Sequence Synthetic oligonucleotide”
tgtgcaaatct

SEQ ID NO: 101 - DNA, 12 bp, Artificial Sequence
  source: /note=“Description of Artificial Sequence Synthetic oligonucleotide”
tgtgcaaatcta

SEQ ID NO: 102 - DNA, 13 bp, Artificial Sequence
  source: /note=“Description of Artificial Sequence Synthetic oligonucleotide”
tgtgcaaatctat

SEQ ID NO: 103 - DNA, 14 bp, Artificial Sequence
  source: /note=“Description of Artificial Sequence Synthetic oligonucleotide”
tgtgcaaatctatg

SEQ ID NO: 104 - DNA, 15 bp, Artificial Sequence
  source: /note=“Description of Artificial Sequence Synthetic oligonucleotide”
tgtgcaaatctatgc

SEQ ID NO: 105 - DNA, 17 bp, Artificial Sequence
  source: /note=“Description of Artificial Sequence Synthetic oligonucleotide”
tgtgcaaatctatgcaa

SEQ ID NO: 106 - DNA, 68 bp, Artificial Sequence
  source: /note=“Description of Artificial Sequence Synthetic oligonucleotide”
tcggtcagtgggtcattgctagtgtgcaaatctatgcaaaactgatcggtcagtgggtcattgctagt

SEQ ID NO: 107 - DNA, 45 bp, Artificial Sequence
  source: /note=“Description of Artificial Sequence Synthetic oligonucleotide”
tcggtcagtgggtcattgctagtgtgcaaatctatgcaaaactga

SEQ ID NO: 108 - DNA, 46 bp, Artificial Sequence
  source: /note=“Description of Artificial Sequence Synthetic oligonucleotide”
tgtgcaaatctatgcaaaactgatcggtcagtgggtcattgctagt

SEQ ID NO: 109 - DNA, 68 bp, Artificial Sequence
  source: /note=“Description of Artificial Sequence Synthetic oligonucleotide”
tcggtcagtgggtcattgctagtcggtcagtgggtcattgctagttcggtcagtgggtcattgctagt

SEQ ID NO: 110 - DNA, 75 bp, Artificial Sequence
  source: /note=“Description of Artificial Sequence Synthetic oligonucleotide”
tcggtcagtgggtcattgctagtagtcatcaagcatcaacaattattattattatattgttgatgcttgatgact

SEQ ID NO: 111 - DNA, 75 bp, Artificial Sequence
  source: /note=“Description of Artificial Sequence Synthetic oligonucleotide”
tgtgcaaatctatgcaaaactgaagtcatcaagcatcaacaattattattattatattgttgatgcttgatgact

SEQ ID NO: 112 - DNA, 10 bp, Artificial Sequence
  source: /note=“Description of Artificial Sequence Synthetic oligonucleotide”
agtcaaaacg

SEQ ID NO: 113 - DNA, 79 bp, Artificial Sequence
  source: /note=“Description of Artificial Sequence Synthetic oligonucleotide”
agtcaaaacgtatctaaacgtgtttttagtcatcaagcatcaacaattattattattatattgttgatgcttgatgact